(19) United States
(12) Patent Application Publication
(10) Pub. No.: US 2026/0355336 A1
(43) Pub. Date: Nov. 12, 2026

(54) MUSCLE TARGETING COMPLEXES AND USES THEREOF FOR TREATING FACIOSCAPULOHUMERAL MUSCULAR DYSTROPHY

(71) Applicants: Nelson Hsia, WALTHAM, MA (US); TIMOTHY WEEDEN, WALTHAM, MA (US); STEFANO ZANOTTI, WALTHAM, MA (US); OXANA BESKROVNAYA, WALTHAM, MA (US); JOCHEN DECKERT, BAYREUTH (DE); HANS-PETER VOMLOCHER, KULMBACH (DE); ROMESH R SUBRAMANIAN, FRAMINGHAM, MA (US); MOHAMMED T. QATANANI, WALTHAM, MA (US); CODY A. DESJARDINS, WALTHAM, MA (US); BRENDAN QUINN, WALTHAM, MA (US); JOHN NAHIM, WALTHAM, MA (US); MARKUS HOSSBACH, KULMBACH (DE); KATHRIN HULTSCH, KULMBACH (DE); QIFENG QIU, WALTHAM, MA (US); SCOTT HILDERBRAND, WALTHAM, MA (US)

(72) Inventors: Nelson Hsia, WALTHAM, MA (US); TIMOTHY WEEDEN, WALTHAM, MA (US); STEFANO ZANOTTI, WALTHAM, MA (US); OXANA BESKROVNAYA, WALTHAM, MA (US); JOCHEN DECKERT, BAYREUTH (DE); HANS-PETER VOMLOCHER, KULMBACH (DE); ROMESH R SUBRAMANIAN, FRAMINGHAM, MA (US); MOHAMMED T. QATANANI, WALTHAM, MA (US); CODY A. DESJARDINS, WALTHAM, MA (US); BRENDAN QUINN, WALTHAM, MA (US); JOHN NAHIM, WALTHAM, MA (US); MARKUS HOSSBACH, KULMBACH (DE); KATHRIN HULTSCH, KULMBACH (DE); QIFENG QIU, WALTHAM, MA (US); SCOTT HILDERBRAND, WALTHAM, MA (US)

(21) Appl. No.: 18/881,012

(22) PCT Filed: Jul. 5, 2023
(86) PCT No.: PCT/US2023/069652

§ 371 Date: Jan. 3, 2025

(51) Int. Cl. A61K 47/68; C07K 1/107; C07K 16/28; C07K 19/00

(52) U.S. Cl. CPC A61K 47/6807; A61K 47/6849; C07K 1/1077; C07K 16/2881; C07K 19/00

(57) ABSTRACT

Aspects of the disclosure relate to oligonucleotides (e.g., RNAi oligonucleotides such as siRNAs) designed to target DUX4 RNAs and targeting complexes for delivering the oligonucleotides to cells (e.g., muscle cells) and uses thereof, particularly uses relating to treatment of disease (e.g., FSHD).

### RELATED APPLICATIONS

This application claims the benefit under 35 U.S.C. § 119 (e) to U.S. Provisional Application No. 63/367,783, entitled “MUSCLE TARGETING COMPLEXES AND USES THEREOF FOR TREATING FACIOSCAPULOHUMERAL MUSCULAR DYSTROPHY”, filed on Jul. 6, 2022 and to U.S. Provisional Application No. 63/477,160, entitled “MUSCLE TARGETING COMPLEXES AND USES THEREOF FOR TREATING FACIOSCAPULOHUMERAL MUSCULAR DYSTROPHY”, filed on Dec. 23, 2022; the contents of each of which are incorporated herein by reference in their entirety.

### FIELD OF THE INVENTION

The present application relates to oligonucleotides designed to target DUX4 RNAs and targeting complexes for delivering molecular payloads (e.g., oligonucleotides) to cells and uses thereof, particularly uses relating to treatment of disease.

### REFERENCE TO AN ELECTRONIC SEQUENCE LISTING

The contents of the electronic sequence listing (D082470078WO00-SEQ-CBD.xml; Size: 339,190 bytes; and Date of Creation: Jun. 30, 2023) is herein incorporated by reference in its entirety.

### BACKGROUND

Muscular dystrophies (MDs) are a group of diseases characterized by the progressive weakness and loss of muscle mass. These diseases are caused by mutations in genes which encode proteins needed for healthy muscle tissue. Facioscapulohumeral muscular dystrophy (FSHD) is a dominantly inherited type of MD which primarily affects muscles of the face, shoulder blades, and upper arms. Other symptoms of FSHD include abdominal muscle weakness, retinal abnormalities, hearing loss, and joint pain and inflammation. FSHD is the most prevalent of the nine types of MD affecting both adults and children, with a worldwide incidence of about 1 in 8,300 people. FSHD is caused by aberrant production of double homeobox 4 (DUX4), a protein that regulates gene expression. The DUX4 gene, which encodes the DUX4 protein, is located in the D4Z4 repeat region on chromosome 4 and is typically expressed in adult testes and thymus, and in 2-cell stage embryos, after which it is repressed by hypermethylation of the D4Z4 repeats which surround and compact the DUX4 gene. Two types of FSHD, Type 1 and Type 2 have been described. Type 1, which accounts for about 95% of cases, is associated with deletions of D4Z4 repeats in the subtelomeric region of chromosome 4. Unaffected individuals generally have more than 10 repeats arrayed in the subtelomeric region of chromosome 4, whereas the most common form of FSHD (FSHD1) is caused by a contraction of the array to fewer than 10 repeats, associated with decreased epigenetic repression and variegated expression of DUX4 in skeletal muscle. Two allelic variants of chromosome 4q (4qA and 4qB) exist in the most distal unit of the D4Z4 repeats. 4qA is in cis with a functional polyadenylation consensus site. Contractions on 4qA alleles are pathogenic because the DUX4 transcript is polyadenylated and stable. Type 2 FSHD, which accounts for about 5% of cases, is associated with mutations of the SMCHD1 gene on chromosome 18. Type 2 FSHD may also be associated with the DNMT3B gene or LRIF1 gene. Besides supportive care and treatments to address the symptoms of the disease, there are no effective therapies for FSHD.

### SUMMARY

Some aspects of the present disclosure provide oligonucleotides designed to target DUX4 RNAs. In some embodiments, the disclosure provides oligonucleotides complementary with DUX4 RNA that are useful for reducing levels of DUX4 RNA and/or protein. In some embodiments, the disclosure provides oligonucleotides that are complementary with exon 3 of DUX4 RNA that are useful for reducing levels of DUX4 RNA. In some embodiments, the oligonucleotides provided herein are designed to direct RNAi mediated degradation of DUX4 RNA. In some embodiments, the oligonucleotides are designed to efficiently engage the RNA-induced silencing complex (RISC) for degradation of the DUX4 RNA but also have reduced off-target effect. In some embodiments, the oligonucleotides are designed to have desirable bioavailability and/or serum-stability properties. In some embodiments, the oligonucleotides are designed to have desirable binding affinity properties. In some embodiments, the oligonucleotides are designed to have desirable toxicity and/or immunogenicity profiles. Aberrant (e.g., increased) expression of DUX4 RNA and/or protein in muscle is associated with features of facioscapulohumeral muscular dystrophy (FSHD) pathology, including muscle atrophy, in inflammation, and decreased differentiation potential and oxidative stress. In some embodiments, the oligonucleotides described herein that reduce DUX4 RNA and/or protein levels are effective in treating FSHD.

According to some aspects, the disclosure provides complexes that target muscle cells (e.g., primary myoblasts) for purposes of delivering molecular payloads (e.g., the oligonucleotides described herein) to those cells. In some embodiments, complexes provided herein are particularly useful for delivering molecular payloads that inhibit the expression or activity of DUX4, e.g., in a subject having or suspected of having FSHD. Accordingly, in some embodiments, complexes provided herein comprise muscle-targeting agents (e.g., muscle targeting antibodies) that specifically bind to receptors on the surface of muscle cells for purposes of delivering molecular payloads to the muscle cells. In some embodiments, the complexes are taken up into the cells via a receptor mediated internalization, following which the molecular payload may be released to perform a function inside the cells. For example, complexes engineered to deliver oligonucleotides may release the oligonucleotides such that the oligonucleotides can inhibit DUX4 gene expression in the muscle cells. In some embodiments, the oligonucleotides are released by endosomal cleavage of covalent linkers connecting oligonucleotides and muscle-targeting agents of the complexes.

Some aspects of the present disclosure provide complexes comprising a muscle-targeting agent covalently linked to an oligonucleotide targeting a double homeobox 4 (DUX4) RNA (e.g., mRNA), wherein the oligonucleotide comprises an antisense strand of 18-25 nucleotides in length and comprises a region of complementarity to a target sequence as set forth in SEQ ID NOs: 174-235, and wherein the region of complementarity is at least 16 consecutive nucleosides in length.

Some aspects of the present disclosure provide complexes comprising a muscle-targeting agent covalently linked to an oligonucleotide targeting a double homeobox 4 (DUX4) RNA (e.g., mRNA), wherein the oligonucleotide comprises an antisense strand of 18-25 nucleotides in length and comprises a region of complementarity to a target sequence as set forth in SEQ ID NOs: 200, 191, 189, 186, 190, 174-185, 187, 188, 192-199, and 201-235, and wherein the region of complementarity is at least 16 consecutive nucleosides in length.

In some embodiments, the muscle-targeting agent is an anti-transferrin receptor 1 (TfR1) antibody.

In some embodiments, the oligonucleotide is an RNAi oligonucleotide.

In some embodiments, the antisense strand comprises the nucleotide sequence of any one of SEQ ID NOs: 236-266.

In some embodiments, the antisense strand comprises the nucleotide sequence of any one of SEQ ID NOs: 262, 253, 251, 248, 252, 236-247, 249, 250, 254-261, 263-266.

In some embodiments, the oligonucleotide further comprises a sense strand which comprises at least 18 consecutive nucleosides complementary to the antisense strand.

In some embodiments, the sense strand comprises 21 consecutive nucleosides complementary to the antisense strand.

In some embodiments, the sense strand comprises the nucleotide sequence of any one of SEQ ID NOs: 205-235.

In some embodiments, the sense strand comprises the nucleotide sequence of any one of SEQ ID NOs: 231, 222, 220, 217, 221, 205-216, 218, 219, 223, 230, 232-235.

In some embodiments, the muscle-targeting agent is covalently linked to the 5′ end or the 3′ end of the sense strand.

In some embodiments, the antisense strand of the RNAi oligonucleotide further comprises a 5′-(E)-vinylphosphonate.

In some embodiments, the oligonucleotide comprises one or more modified nucleosides.

In some embodiments, the one or more modified nucleosides are 2′ modified nucleotides, optionally wherein the one or more 2′ modified nucleosides are selected from: 2′-fluoro (2′-F), 2′-O-methyl(2′—O-Me), 2′-O-methoxyethyl(2′-MOE), 2′-O-aminopropyl(2′—O-AP), 2′-O-dimethylaminoethyl(2′—O-DMAOE), 2′-O-dimethylaminopropyl(2′—O-DMAP), 2′-O-dimethylaminoethyloxyethyl(2′—O-DMAEOE), 2′—O—N-methylacetamido (2′-O-NMA)).

In some embodiments, each 2′ modified nucleotide is 2′-O-methyl(2′—O-Me) or 2′-fluoro (2′-F). In some embodiments, the 2′ modified nucleotide is 2′-O-methyl(2′—O-Me). In some embodiments, the 2′ modified nucleotide is 2′-fluoro (2′-F).

In some embodiments, the oligonucleotide comprises one or more phosphorothioate internucleoside linkages.

In some embodiments, the one or more phosphorothioate internucleoside linkage are present on the antisense strand of the oligonucleotide.

In some embodiments, the two internucleoside linkages at the 3′ end of the antisense strands are phosphorothioate internucleoside linkages. In some embodiments, the two internucleoside linkages at the 5′ end of the antisense strands are phosphorothioate internucleoside linkages. In some embodiments, the two internucleoside linkages at the 3′ end of the antisense strands and the two internucleoside linkages at the 5′ end of the antisense strands are phosphorothioate internucleoside linkages.

In some embodiments, the one or more phosphorothioate internucleoside linkage are present on the sense strand of the oligonucleotide.

In some embodiments, the two internucleoside linkages at the 3′ end of the sense strands are phosphorothioate internucleoside linkages. In some embodiments, the two internucleoside linkages at the 5′ end of the sense strands are phosphorothioate internucleoside linkages. In some embodiments, the two internucleoside linkages at the 3′ end of the sense strands and the two internucleoside linkages at the 5′ end of the sense strands are phosphorothioate internucleoside linkages.

In some embodiments, one or more cytidines of the oligonucleotide is a 2′-modified 5-methyl-cytidine, optionally wherein the 2′-modified 5-methyl-cytidine is a 2′-O-Me modified 5-methyl-cytidine or a 2′-F modified 5-methyl-cytidine.

In some embodiments, the antisense strand of an oligonucleotide described herein is selected from the modified versions (e.g., MAS1-MAS4) of SEQ ID NOs: 236-266 listed in Table 8. For example, in some embodiments, the antisense strand is selected from MAS1-236, MAS1-237, MAS1-238, MAS2-236, MAS2-237, MAS2-238, MAS2-239, MAS2-240, MAS2-241, MAS2-242, MAS2-243, MAS2-244, MAS2-245, MAS2-246, MAS2-247, MAS2-248, MAS2-249, MAS2-250, MAS2-251, MAS2-252, MAS2-253, MAS2-254, MAS2-255, MAS2-256, MAS2-257, MAS3-236, MAS3-237, MAS3-238, MAS3-239, MAS3-240, MAS3-241, MAS3-242, MAS3-243, MAS3-244, MAS3-245, MAS3-246, MAS3-247, MAS3-248, MAS3-249, MAS3-250, MAS3-251, MAS3-252, MAS3-253, MAS3-254, MAS3-255, MAS3-256, MAS3-257, MAS3-258, MAS3-259, MAS3-260, MAS3-261, MAS3-262, MAS3-263, MAS3-264, MAS3-265, MAS3-266, MAS4-236, MAS4-237, MAS4-238, MAS4-239, MAS4-240, MAS4-241, MAS4-242, MAS4-243, MAS4-244, MAS4-245, MAS4-246, MAS4-247, MAS4-248, MAS4-249, MAS4-250, MAS4-251, MAS4-252, MAS4-253, MAS4-254, MAS4-255, MAS4-256, and MAS4-257.

In some embodiments, the sense strand of an oligonucleotide described herein is selected from the modified versions (e.g., MS1-MS6) of SEQ ID NOs: 205-235 listed in Table 8. For example, in some embodiments, the sense strand is selected from MS1-205, MS1-206, MS1-207, MS2-208, MS2-209, MS2-210, MS2-211, MS2-212, MS2-213, MS2-214, MS2-215, MS2-216, MS2-217, MS2-218, MS2-219, MS2-220, MS2-221, MS2-222, MS2-223, MS2-224, MS2-225, MS2-226, MS3-205, MS3-206, MS3-207, MS3-208, MS3-209, MS3-210, MS3-211, MS3-212, MS3-213, MS3-214, MS3-215, MS3-216, MS3-217, MS3-218, MS3-219, MS3-220, MS3-221, MS3-222, MS3-223, MS3-224, MS3-225, MS3-226, MS3-227, MS3-228, MS3-229, MS3-230, MS3-231, MS3-232, MS3-233, MS3-234, MS3-235, MS4-205, MS4-206, MS4-207, MS4-208, MS4-209, MS4-210, MS4-211, MS4-212, MS4-213, MS4-214, MS4-215, MS4-216, MS4-217, MS4-218, MS4-219, MS4-220, MS4-221, MS4-222, MS4-223, MS4-224, MS4-225, MS4-226, MS5-205, MS5-206, MS5-207, MS5-208, MS5-209, MS5-210, MS5-211, MS5-212, MS5-213, MS5-214, MS5-215, MS5-216, MS5-217, MS5-218, MS5-219, MS5-220, MS5-221, MS5-222, MS5-223, MS5-224, MS5-225, MS5-226, MS6-205, MS6-206, MS6-207, MS6-208, MS6-209, MS6-210, MS6-211, MS6-212, MS6-213, MS6-214, MS6-215, MS6-216, MS6-217, MS6-218, MS6-219, MS6-220, MS6-221, MS6-222, MS6-223, MS6-224, MS6-225, and MS6-226.

In some embodiments, the antisense strand is selected from the modified versions of SEQ ID NOs: 262, 253, 251, 248, and 252 (e.g., VP-MAS5, VP-MAS6, and VP-MAS7) listed in Table 9 and/or wherein the sense strand is selected from the modified versions of SEQ ID NOs: 231, 222, 220, 217, and 221 (e.g., MS7-MS9) listed in Table 9.

In some embodiments, the oligonucleotide is an siRNA molecule selected from the siRNAs listed in Table 8.

In some embodiments, the oligonucleotide is an siRNA molecule selected from the siRNAs listed in Table 9.

In some embodiments, the anti-TfR1 antibody comprises a heavy chain complementarity determining region 1 (CDR-H1), a heavy chain complementarity determining region 2 (CDR-H2), a heavy chain complementarity determining region 3 (CDR-H3), a light chain complementarity determining region 1 (CDR-L1), a light chain complementarity determining region 2 (CDR-L2), a light chain complementarity determining region 3 (CDR-L3) of any of the anti-TfR1 antibodies listed in Table 2.

In some embodiments, the anti-TfR1 antibody comprises a heavy chain variable region (VH) and a light chain variable region (VL) of any of the anti-TfR1 antibodies listed in Table 3.

In some embodiments, the anti-TfR1 antibody is a Fab, optionally wherein the Fab comprises a heavy chain and a light chain of any of the anti-TfR1 Fabs listed in Table 5.

In some embodiments, the anti-TfR1 antibody comprises:
(i) a CDR-H1 comprising the amino acid sequence of SEQ ID NO: 27, a CDR-H2 comprising the amino acid sequence of SEQ ID NO: 28, a CDR-H3 comprising the amino acid sequence of SEQ ID NO: 29, a CDR-L1 comprising the amino acid sequence of SEQ ID NO: 30, a CDR-L2 comprising the amino acid sequence of SEQ ID NO: 31, and a CDR-L3 comprising the amino acid sequence of SEQ ID NO: 32;
(ii) a CDR-H1 comprising the amino acid sequence of SEQ ID NO: 33, a CDR-H2 comprising the amino acid sequence of SEQ ID NO: 34, a CDR-H3 comprising the amino acid sequence of SEQ ID NO: 35, a CDR-L1 comprising the amino acid sequence of SEQ ID NO: 36, a CDR-L2 comprising the amino acid sequence of SEQ ID NO: 37, and a CDR-L3 comprising the amino acid sequence of SEQ ID NO: 32; or
(ii) a CDR-H1 comprising the amino acid sequence of SEQ ID NO: 38, a CDR-H2 comprising the amino acid sequence of SEQ ID NO: 39, a CDR-H3 comprising the amino acid sequence of SEQ ID NO: 40, a CDR-L1 comprising the amino acid sequence of SEQ ID NO: 41, a CDR-L2 comprising the amino acid sequence of SEQ ID NO: 31, and a CDR-L3 comprising the amino acid sequence of SEQ ID NO: 42.

In some embodiments, the anti-TfR1 antibody comprises a VH comprising the amino acid sequence of SEQ ID NO: 76, and a VL comprising the amino acid sequence of SEQ ID NO: 75.

In some embodiments, the anti-TfR1 antibody is a Fab and comprises a heavy chain comprising the amino acid sequence of SEQ ID NO: 101 and a light chain comprising the amino acid sequence of SEQ ID NO: 90.

In some embodiments, the muscle targeting agent and the antisense oligonucleotide are covalently linked via a linker, optionally wherein the linker comprises a valine-citrulline sequence.

Further provided herein are methods of reducing DUX4 expression in a muscle cell, the method comprising contacting the muscle cell with an effective amount of the complex described herein for promoting internalization of the oligonucleotide to the muscle cell. In some embodiments, reducing DUX4 expression comprises reducing DUX4 protein and/or mRNA levels.

Further provided herein are methods of treating facioscapulohumeral muscular dystrophy (FSHD), the method comprising administering to a subject in need thereof an effective amount of the complex provided herein. In some embodiments, the subject has aberrant production of DUX4 protein.

Other aspects of the present disclosure provide oligonucleotides comprising an siRNA oligonucleotide selected from the siRNA oligonucleotides listed in Table 8.

Other aspects of the present disclosure provide oligonucleotides comprising an siRNA oligonucleotide selected from the siRNA oligonucleotides listed in Table 9.

Further provided herein are methods of producing a complex comprising an anti-transferrin receptor 1 (TfR1) antibody covalently linked to an oligonucleotide, the method comprising: (i) obtaining a compound comprising a structure of formula (B), wherein the oligonucleotide comprises a sense strand of a siRNA oligonucleotide, optionally wherein the siRNA oligonucleotide targets a DUX4 RNA; (ii) annealing an antisense strand of the siRNA oligonucleotide to the sense strand; (iii) reacting the compound comprising a structure of formula (B) with a compound comprising a structure of formula (C) to obtain a compound comprising a structure of formula (D); and (iv) covalently linking an anti-TfR1 antibody to the compound comprising the structure of formula (D) with a muscle target agent to obtain a compound comprising a structure of formula (E), optionally wherein the annealing of step (ii) is performed at 30 °C, further optionally wherein the method further comprises isolating the compound comprising a structure of formula (D) after step (iii) and before step (iv); further optionally wherein the anti-TfR1 antibody is covalently linked to the 5′ end of the sense strand of the siRNA oligonucleotide targeting a DUX4 RNA.

Further provided herein are methods of producing a complex comprising an anti-transferrin receptor 1 (TfR1) antibody covalently linked to an oligonucleotide, the method comprising: (i) obtaining a compound comprising a structure of formula (B), wherein the oligonucleotide comprises a sense strand of a siRNA oligonucleotide, optionally wherein the siRNA oligonucleotide targets a DUX4 RNA; (ii) annealing an antisense strand of the siRNA oligonucleotide to the sense strand; (iii) covalently linking an anti-TfR1 antibody to a compound comprising a structure of formula (C) to obtain a compound comprising a structure of formula (F); and (iv) reacting the compound comprising the structure of formula (F) with the compound comprising a structure of formula (B) to obtain a compound comprising a structure of formula (E), optionally wherein the annealing of step (ii) is performed at 30 °C, further optionally wherein the anti-TfR1 antibody is covalently linked to the 5′ end of the sense strand of the siRNA oligonucleotide targeting a DUX4 RNA.

### BRIEF DESCRIPTION OF THE DRAWINGS

FIG. 1 depicts a non-limiting schematic showing the effect of transfecting cells with an siRNA. The graphs show the effect of siRNAs on HPRT mRNA levels.

FIG. 2 depicts a non-limiting schematic showing the activity of a muscle targeting complex comprising an siRNA. In vitro activity of the complexes was measured on HPRT mRNA levels in cultured muscle cells. The complexes comprise an siRNA linked to a muscle targeting antibody.

FIGS. 3A-3B depict non-limiting schematics showing the activity of in mouse muscle tissues (gastrocnemius and heart) in vivo, relative to vehicle-treated controls. (N=4 C57BL/6 WT mice) In vivo activity of the complexes was measured. The complexes comprise an siRNA linked to a muscle targeting antibody.

FIGS. 4A-4E depict non-limiting schematics showing the tissue selectivity of a muscle targeting complex comprising an siRNA. The complexes comprise an siRNA linked to a muscle targeting antibody.

FIG. 5 shows a composite score of the mRNA levels of three DUX4 transcriptome markers (MBD3L2, TRIM43, and ZSCAN4) in AB1080 immortalized FSHD patient-derived myotubes, following incubation with siRNA conjugates containing an anti-TfR Fab 3M12 VH4/VK3 covalently linked to siRNA-A, siRNA-B, or siRNA-C. The anti-TfR Fab was covalently linked to the 3′ end of the sense strand of each siRNA via a linker, and the corresponding antisense strand was annealed to the sense strand.

FIG. 6 shows a composite score of the mRNA levels of DUX4 transcriptome markers MBD3L2, TRIM43, and ZSCAN4 in FSHD patient-derived C6 myotubes, following transfection with an siRNA selected from siRNA 7, 49, 50, 53, 54, 136, 19, 114, 137, 138, 32, 35, 33, 36, 73, 74, 77, 76, 75, 78, 83, 68, 103, 104, 107, 106, 105, 108, 85, 86, 89, 88, 87, 90, 133, 135, 140, 97, 128, 132, 134, 47, 91, 92, 95, 94, 93, and 96 at a concentration of 0.2 nM or 20 nM. The siRNA numbers correspond to the siRNA numbers in Table 8.

FIGS. 7A-7C show dose response curves generated for siRNAs 7, 136, 137, 74, 77, 78, 104, 107, 106, 86, 89, 133, 97, 128, 92, 95, and 94. The siRNA numbers correspond to the siRNA numbers in Table 8. The dose response curves were generated using the composite scores of DUX4 transcriptome markers MBD3L2, TRIM43, and ZSCAN4 in cells treated with the indicated siRNA with a range of concentrations (2 μM to 40 nM).

FIG. 8 shows a composite score of the mRNA levels of three DUX4 transcriptome markers (MBD3L2, TRIM43, and ZSCAN4) in FSHD patient-derived C6 myotubes, following treatment with siRNA-antibody complexes at a concentration of 10 nM, 100 nM, or 1000 nM. The siRNA-antibody complexes contained an anti-TfR1 Fab 3M12 VH4/VK3 covalently linked to a DUX4-targeting siRNA. siRNAs tested were siRNAs 142-148. The siRNA numbers correspond to the siRNA numbers in Table 9. An exon 1 targeting siRNA was used as a positive control.

FIGS. 9A-9B show a composite score of the mRNA levels of three DUX4 mouse transcriptome markers (Wfdc3, Sord, Serpinb6c) in mouse muscle quadriceps (FIG. 9A) and gastrocnemius (FIG. 9B) muscles relative to vehicle-treated controls. Tissues were collected 4 weeks after a single intravenous injection of DUX4-targeting siRNA complexes covalently linked to anti-TfR1 Fab 3M12 VH4/VK3 at a dose of 10 mg/kg of siRNA. siRNAs tested were siRNA145, siRNA148, siRNA144, siRNA143, and siRNA142. The siRNA numbers correspond to the siRNA numbers in Table 9.

FIG. 10 shows an example of a schematic illustrating how an anti-TfR1-siRNA complex described herein is made.

### DETAILED DESCRIPTION

Some aspects of the present disclosure provide oligonucleotides designed to target DUX4 RNAs. In some embodiments, the disclosure provides oligonucleotides complementary with DUX4 RNA that are useful for reducing levels of DUX4 RNA and/or protein. In some embodiments, the oligonucleotides provided herein are designed to direct RNAi mediated degradation of DUX4 RNA. In some embodiments, the oligonucleotides are designed to efficiently engage the RNA-induced silencing complex (RISC) for degradation of the DUX4 RNA but also have reduced off-target effect. In some embodiments, the oligonucleotides are designed to have desirable bioavailability and/or serum-stability properties. In some embodiments, the oligonucleotides are designed to have desirable binding affinity properties. In some embodiments, the oligonucleotides are designed to have desirable toxicity and/or immunogenicity profiles. Aberrant (e.g., increased) expression of DUX4 RNA and/or protein is associated with features of facioscapulohumeral muscular dystrophy (FSHD) pathology, including muscle atrophy, inflammation, and decreased differentiation potential and oxidative stress. In some embodiments, the oligonucleotides described herein that reduce DUX4 RNA and/or protein levels are effective in treating FSHD.

In some aspects, the present disclosure provides complexes comprising muscle-targeting agents covalently linked to oligonucleotides for effective delivery of the oligonucleotides to muscle cells. In some embodiments, the complexes are particularly useful for delivering molecular payloads that inhibit the expression or activity of target genes in muscle cells, e.g., in a subject having or suspected of having a rare muscle disease. For example, in some embodiments, complexes are provided for treating subjects having FSHD. In some embodiments, complexes are provided for treating subjects having FSHD1. In some embodiments, complexes are provided for treating subjects having FSHD2. In some embodiments, complexes are provided for targeting DUX4 to treat subjects having FSHD. In some embodiments, complexes provided herein comprise oligonucleotides that inhibit expression of DUX4 in a subject that has one or more D4Z4 repeat deletions on chromosome 4. In some embodiments, complexes provided herein comprises oligonucleotides inhibit expression of DUX4 in a subject that has a mutation in SMCHD1 or another DUX4 regulatory gene.

Further aspects of the disclosure, including a description of defined terms, are provided below.

### I. Definitions

Administering: As used herein, the terms “administering” or “administration” means to provide a complex to a subject in a manner that is physiologically and/or (e.g., and) pharmacologically useful (e.g., to treat a condition in the subject).

Approximately: As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Antibody: As used herein, the term “antibody” refers to a polypeptide that includes at least one immunoglobulin variable domain or at least one antigenic determinant, e.g., paratope that specifically binds to an antigen. In some embodiments, an antibody is a full-length antibody. In some embodiments, an antibody is a chimeric antibody. In some embodiments, an antibody is a humanized antibody. However, in some embodiments, an antibody is a Fab fragment, a Fab′ fragment, a F(ab′) 2 fragment, a Fv fragment or a scFv fragment. In some embodiments, an antibody is a nanobody derived from a camelid antibody or a nanobody derived from shark antibody. In some embodiments, an antibody is a diabody. In some embodiments, an antibody comprises a framework having a human germline sequence. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. In some embodiments, an antibody comprises a heavy (H) chain variable region (abbreviated herein as VH), and/or (e.g., and) a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (α), delta (Δ), epsilon (ε), gamma (γ) or mu (μ) heavy chain. In some embodiments, the heavy chain of an antibody described herein can comprise a human alpha (α), delta (Δ), epsilon (ε), gamma (γ) or mu (μ) heavy chain. In a particular embodiment, an antibody described herein comprises a human gamma 1 CH1, CH2, and/or (e.g., and) CH3 domain. In some embodiments, the amino acid sequence of the VH domain comprises the amino acid sequence of a human gamma (γ) heavy chain constant region, such as any known in the art. Non-limiting examples of human constant region sequences have been described in the art, e.g., see U.S. Pat. No. 5,693,780 and Kabat E A et al., (1991) supra. In some embodiments, the VH domain comprises an amino acid sequence that is at least 70%, 75%, 80%, 85%, 90%, 95%, 98%, or at least 99% identical to any of the variable chain constant regions provided herein. In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecule(s). In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Examples of linker polypeptides have been reported (see e.g., Holliger, P., et al. (1993) Proc. Natl. Acad. Sci. USA 90:6444-6448; Poljak, R. J., et al. (1994) Structure 2:1121-1123). Still further, an antibody may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

CDR: As used herein, the term “CDR” refers to the complementarity determining region within antibody variable sequences. A typical antibody molecule comprises a heavy chain variable region (VH) and a light chain variable region (VL), which are usually involved in antigen binding. The VH and VL regions can be further subdivided into regions of hypervariability, also known as “complementarity determining regions” (“CDR”), interspersed with regions that are more conserved, which are known as “framework regions” (“FR”). Each VH and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the IMGT definition, the Chothia definition, the AbM definition, and/or (e.g., and) the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242; IMGT®, the international ImMunoGeneTics information system® www.imgt.org, Lefranc, M.-P. et al., Nucleic Acids Res., 27:209-212 (1999); Ruiz, M. et al., Nucleic Acids Res., 28:219-221 (2000); Lefranc, M.-P., Nucleic Acids Res., 29:207-209 (2001); Lefranc, M.-P., Nucleic Acids Res., 31:307-310 (2003); Lefranc, M.-P. et al., In Silico Biol., 5, 0006 (2004) [Epub], 5:45-60 (2005); Lefranc, M.-P. et al., Nucleic Acids Res., 33: D593-597 (2005); Lefranc, M.-P. et al., Nucleic Acids Res., 37: D1006-1012 (2009); Lefranc, M.-P. et al., Nucleic Acids Res., 43: D413-422 (2015); Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs. As used herein, a CDR may refer to the CDR defined by any method known in the art. Two antibodies having the same CDR means that the two antibodies have the same amino acid sequence of that CDR as determined by the same method, for example, the IMGT definition.

There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Sub-portions of CDRs may be designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262 (5): 732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems. Examples of CDR definition systems are provided in Table 1.

TABLE 1
CDR Definitions

| | IMGT¹ | Kabat² | Chothia³ |
|---|---|---|---|
| CDR-H1 | 27-38 | 31-35 | 26-32 |
| CDR-H2 | 56-65 | 50-65 | 53-55 |
| CDR-H3 | 105-116/117 | 95-102 | 96-101 |
| CDR-L1 | 27-38 | 24-34 | 26-32 |
| CDR-L2 | 56-65 | 50-56 | 50-52 |
| CDR-L3 | 105-116/117 | 89-97 | 91-96 |

¹IMGT ®, the international ImMunoGeneTics information system ®, imgt.org, Lefranc, M. -P. et al., Nucleic Acids Res., 27: 209-212 (1999)
²Kabat et al. (1991) Sequences of Proteins of Immunological Interest, Fifth Edition, U.S. Department of Health and Human Services, NIH Publication No. 91-3242
³Chothia et al., J. Mol. Biol. 196: 901-917 (1987))

CDR-grafted antibody: The term “CDR-grafted antibody” refers to antibodies which comprise heavy and light chain variable region sequences from one species but in which the sequences of one or more of the CDR regions of VH and/or (e.g., and) VL are replaced with CDR sequences of another species, such as antibodies having murine heavy and light chain variable regions in which one or more of the murine CDRs (e.g., CDR3) has been replaced with human CDR sequences.

Chimeric antibody: The term “chimeric antibody” refers to antibodies which comprise heavy and light chain variable region sequences from one species and constant region sequences from another species, such as antibodies having murine heavy and light chain variable regions linked to human constant regions.

Complementary: As used herein, the term “complementary” refers to the capacity for precise pairing between two nucleosides or two sets of nucleosides. In particular, complementary is a term that characterizes an extent of hydrogen bond pairing that brings about binding between two nucleosides or two sets of nucleosides. For example, if a base at one position of an oligonucleotide is capable of hydrogen bonding with a base at the corresponding position of a target nucleic acid (e.g., an mRNA), then the bases are considered to be complementary to each other at that position. Base pairings may include both canonical Watson-Crick base pairing and non-Watson-Crick base pairing (e.g., Wobble base pairing and Hoogsteen base pairing). For example, in some embodiments, for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

Conservative amino acid substitution: As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Covalently linked: As used herein, the term “covalently linked” refers to a characteristic of two or more molecules being linked together via at least one covalent bond. In some embodiments, two molecules can be covalently linked together by a single bond, e.g., a disulfide bond or disulfide bridge, that serves as a linker between the molecules. However, in some embodiments, two or more molecules can be covalently linked together via a molecule that serves as a linker that joins the two or more molecules together through multiple covalent bonds. In some embodiments, a linker may be a cleavable linker. However, in some embodiments, a linker may be a non-cleavable linker.

Cross-reactive: As used herein and in the context of a targeting agent (e.g., antibody), the term “cross-reactive,” refers to a property of the agent being capable of specifically binding to more than one antigen of a similar type or class (e.g., antigens of multiple homologs, paralogs, or orthologs) with similar affinity or avidity. For example, in some embodiments, an antibody that is cross-reactive against human and non-human primate antigens of a similar type or class (e.g., a human transferrin receptor and non-human primate transferrin receptor) is capable of binding to the human antigen and non-human primate antigens with a similar affinity or avidity. In some embodiments, an antibody is cross-reactive against a human antigen and a rodent antigen of a similar type or class. In some embodiments, an antibody is cross-reactive against a rodent antigen and a non-human primate antigen of a similar type or class. In some embodiments, an antibody is cross-reactive against a human antigen, a non-human primate antigen, and a rodent antigen of a similar type or class.

DUX4: As used herein, the term “DUX4” refers to a gene that encodes double homeobox 4, a protein which is generally expressed during fetal development and in the testes of adult males. In some embodiments, DUX4 may be a human (Gene ID: 100288687), non-human primate (e.g., Gene ID: 750891, Gene ID: 100405864), or rodent gene (e.g., Gene ID: 306226). In humans, expression of the DUX4 gene outside of fetal development and the testes is associated with facioscapulohumeral muscular dystrophy. In addition, multiple human transcript variants (e.g., as annotated under GenBank RefSeq Accession Numbers: NM_001293798.2, NM_001306068.3, NM_001363820.1) have been characterized that encode different protein isoforms.

Facioscapulohumeral muscular dystrophy (FSHD): As used herein, the term “facioscapulohumeral muscular dystrophy (FSHD)” refers to a genetic disease caused by mutations in the DUX4 gene, SMCHD1 gene, DNMT3B gene, or LRIF1 gene that is characterized by muscle mass loss and muscle atrophy, primarily in the muscles of the face, shoulder blades, and upper arms. Two types of the disease, Type 1 and Type 2, have been described. Type 1 is associated with deletions in D4Z4 repeat regions on chromosome 4 which contains the DUX4 gene. In some embodiments, Type 1 is associated with deletions in D4Z4 repeat regions on chromosome 4 allelic variant 4qA which contains the DUX4 gene. Type 2 is associated with mutations in the SMCHD1 gene, DNMT3B gene, or LRIF1 gene (see, e.g. Jia et al., “Facioscapulohumeral muscular dystrophy type 2: an update on the clinical, genetic, and molecular findings” Neuromuscul Disord. (2021), 31 (11): 1101-1112. Both Type 1 and Type 2 FSHD are characterized by aberrant production of the DUX4 protein after fetal development outside of the testes. Facioscapulohumeral dystrophy, the genetic basis for the disease, and related symptoms are described in the art (see, e.g. Campbell, A. E., et al.,

“Facioscapulohumeral dystrophy: Activating an early embryonic transcriptional program in human skeletal muscle” Human Mol Genet. (2018); and Tawil, R. “Facioscapulohumeral muscular dystrophy” Handbook Clin. Neurol. (2018), 148:541-548.) FSHD Type 1 is associated with Online Mendelian Inheritance in Man (OMIM) Entry #158900. FSHD Type 2 is associated with OMIM Entry #158901.

Framework: As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region. Human heavy chain and light chain acceptor sequences are known in the art. In one embodiment, the acceptor sequences known in the art may be used in the antibodies disclosed herein.

Human antibody: The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

Humanized antibody: The term “humanized antibody” refers to antibodies which comprise heavy and light chain variable region sequences from a non-human species (e.g., a mouse) but in which at least a portion of the VH and/or (e.g., and) VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding non-human CDR sequences. In one embodiment, humanized anti-TfR1 antibodies and antigen binding portions are provided. Such antibodies may be generated by obtaining murine anti-TfR1 monoclonal antibodies using traditional hybridoma technology followed by humanization using in vitro genetic engineering, such as those disclosed in Kasaian et al PCT publication No. WO 2005/123126 A2.

Internalizing cell surface receptor: As used herein, the term, “internalizing cell surface receptor” refers to a cell surface receptor that is internalized by cells, e.g., upon external stimulation, e.g., ligand binding to the receptor. In some embodiments, an internalizing cell surface receptor is internalized by endocytosis. In some embodiments, an internalizing cell surface receptor is internalized by clathrin-mediated endocytosis. However, in some embodiments, an internalizing cell surface receptor is internalized by a clathrin-independent pathway, such as, for example, phagocytosis, macropinocytosis, caveolae- and raft-mediated uptake or constitutive clathrin-independent endocytosis. In some embodiments, the internalizing cell surface receptor comprises an intracellular domain, a transmembrane domain, and/or (e.g., and) an extracellular domain, which may optionally further comprise a ligand-binding domain. In some embodiments, a cell surface receptor becomes internalized by a cell after ligand binding. In some embodiments, a ligand may be a muscle-targeting agent or a muscle-targeting antibody. In some embodiments, an internalizing cell surface receptor is a transferrin receptor.

Isolated antibody: An “isolated antibody”, as used herein, is intended to refer to an antibody that is substantially free of other antibodies having different antigenic specificities (e.g., an isolated antibody that specifically binds transferrin receptor is substantially free of antibodies that specifically bind antigens other than transferrin receptor). An isolated antibody that specifically binds transferrin receptor complex may, however, have cross-reactivity to other antigens, such as transferrin receptor molecules from other species. Moreover, an isolated antibody may be substantially free of other cellular material and/or (e.g., and) chemicals.

Kabat numbering: The terms “Kabat numbering”, “Kabat definitions and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

Molecular payload: As used herein, the term “molecular payload” refers to a molecule or species that functions to modulate a biological outcome. In some embodiments, a molecular payload is linked to, or otherwise associated with a muscle-targeting agent. In some embodiments, the molecular payload is a small molecule, a protein, a peptide, a nucleic acid, or an oligonucleotide. In some embodiments, the molecular payload functions to modulate the transcription of a DNA sequence, to modulate the expression of a protein, or to modulate the activity of a protein. In some embodiments, the molecular payload is an oligonucleotide that comprises a strand having a region of complementarity to a target gene.

Muscle-targeting agent: As used herein, the term, “muscle-targeting agent,” refers to a molecule that specifically binds to an antigen expressed on muscle cells. The antigen in or on muscle cells may be a membrane protein, for example an integral membrane protein or a peripheral membrane protein. Typically, a muscle-targeting agent specifically binds to an antigen on muscle cells that facilitates internalization of the muscle-targeting agent (and any associated molecular payload) into the muscle cells. In some embodiments, a muscle-targeting agent specifically binds to an internalizing, cell surface receptor on muscles and is capable of being internalized into muscle cells through receptor mediated internalization. In some embodiments, the muscle-targeting agent is a small molecule, a protein, a peptide, a nucleic acid (e.g., an aptamer), or an antibody. In some embodiments, the muscle-targeting agent is linked to a molecular payload.

Muscle-targeting antibody: As used herein, the term, “muscle-targeting antibody,” refers to a muscle-targeting agent that is an antibody that specifically binds to an antigen found in or on muscle cells. In some embodiments, a muscle-targeting antibody specifically binds to an antigen on muscle cells that facilitates internalization of the muscle-targeting antibody (and any associated molecular payload) into the muscle cells. In some embodiments, the muscle-targeting antibody specifically binds to an internalizing, cell surface receptor present on muscle cells. In some embodiments, the muscle-targeting antibody is an antibody that specifically binds to a transferrin receptor.

Oligonucleotide: As used herein, the term “oligonucleotide” refers to an oligomeric nucleic acid compound of up to 200 nucleotides in length. Examples of oligonucleotides include, but are not limited to, RNAi oligonucleotides (e.g., siRNAs, shRNAs), microRNAs, gapmers, mixmers, phosphorodiamidate morpholinos, peptide nucleic acids, aptamers, guide nucleic acids (e.g., Cas9 guide RNAs), etc. Oligonucleotides may be single-stranded or double-stranded. In some embodiments, an oligonucleotide may comprise one or more modified nucleosides (e.g., 2′-O-methyl sugar modifications, purine or pyrimidine modifications). In some embodiments, an oligonucleotide may comprise one or more modified internucleoside linkages. In some embodiments, an oligonucleotide may comprise one or more phosphorothioate linkages, which may be in the Rp or Sp stereochemical conformation.

Recombinant antibody: The term “recombinant human antibody”, as used herein, is intended to include all human antibodies that are prepared, expressed, created or isolated by recombinant means, such as antibodies expressed using a recombinant expression vector transfected into a host cell (described in more details in this disclosure), antibodies isolated from a recombinant, combinatorial human antibody library (Hoogenboom H. R., (1997) TIB Tech. 15:62-70; Azzazy H., and Highsmith W. E., (2002) Clin. Biochem. 35:425-445; Gavilondo J. V., and Larrick J. W. (2002) BioTechniques 29:128-145; Hoogenboom H., and Chames P. (2000) Immunology Today 21:371-378), antibodies isolated from an animal (e.g., a mouse) that is transgenic for human immunoglobulin genes (see e.g., Taylor, L. D., et al. (1992) Nucl. Acids Res. 20:6287-6295; Kellermann S-A., and Green L. L. (2002) Current Opinion in Biotechnology 13:593-597; Little M. et al (2000) Immunology Today 21:364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. One embodiment of the disclosure provides fully human antibodies capable of binding human transferrin receptor which can be generated using techniques well known in the art, such as, but not limited to, using human Ig phage libraries such as those disclosed in Jermutus et al., PCT publication No. WO 2005/007699 A2.

Region of complementarity: As used herein, the term “region of complementarity” refers to a nucleotide sequence, e.g., of an oligonucleotide, that is sufficiently complementary to a cognate nucleotide sequence, e.g., of a target nucleic acid, such that the two nucleotide sequences are capable of annealing to one another under physiological conditions (e.g., in a cell). In some embodiments, a region of complementarity is fully complementary to a cognate nucleotide sequence of target nucleic acid. However, in some embodiments, a region of complementarity is partially complementary to a cognate nucleotide sequence of target nucleic acid (e.g., at least 80%, 90%, 95% or 99% complementarity). In some embodiments, a region of complementarity contains 1, 2, 3, or 4 mismatches compared with a cognate nucleotide sequence of a target nucleic acid.

Specifically binds: As used herein, the term “specifically binds” refers to the ability of a molecule to bind to a binding partner with a degree of affinity or avidity that enables the molecule to be used to distinguish the binding partner from an appropriate control in a binding assay or other binding context. With respect to an antibody, the term, “specifically binds”, refers to the ability of the antibody to bind to a specific antigen with a degree of affinity or avidity, compared with an appropriate reference antigen or antigens, that enables the antibody to be used to distinguish the specific antigen from others, e.g., to an extent that permits preferential targeting to certain cells, e.g., muscle cells, through binding to the antigen, as described herein. In some embodiments, an antibody specifically binds to a target if the antibody has a K_{D} for binding the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, an antibody specifically binds to the transferrin receptor, e.g., an epitope of the apical domain of transferrin receptor.

Subject: As used herein, the term “subject” refers to a mammal. In some embodiments, a subject is non-human primate, or rodent. In some embodiments, a subject is a human. In some embodiments, a subject is a patient, e.g., a human patient that has or is suspected of having a disease. In some embodiments, the subject is a human patient who has or is suspected of having FSHD.

Transferrin receptor: As used herein, the term, “transferrin receptor” (also known as TFRC, CD71, p90, TFR1) refers to an internalizing cell surface receptor that binds transferrin to facilitate iron uptake by endocytosis. In some embodiments, a transferrin receptor may be of human (NCBI Gene ID 7037), non-human primate (e.g., NCBI Gene ID 711568 or NCBI Gene ID 102136007), or rodent (e.g., NCBI Gene ID 22042) origin. In addition, multiple human transcript variants have been characterized that encoded different isoforms of the receptor (e.g., as annotated under GenBank RefSeq Accession Numbers: NP_001121620.1, NP_003225.2, NP_001300894.1, and NP_001300895.1).

2′-modified nucleoside: As used herein, the terms “2′-modified nucleoside” and “2′-modified ribonucleoside” are used interchangeably and refer to a nucleoside having a sugar moiety modified at the 2′ position. In some embodiments, the 2′-modified nucleoside is a 2′-4′ bicyclic nucleoside, where the 2′ and 4′ positions of the sugar are bridged (e.g., via a methylene, an ethylene, or a (S)-constrained ethyl bridge). In some embodiments, the 2′-modified nucleoside is a non-bicyclic 2′-modified nucleoside, e.g., where the 2′ position of the sugar moiety is substituted. Non-limiting examples of 2′-modified nucleosides include: 2′-deoxy, 2′-fluoro (2′-F), 2′-O-methyl(2′—O-Me), 2′-O-methoxyethyl(2′-MOE), 2′-O-aminopropyl(2′—O-AP), 2′-O-dimethylaminoethyl(2′—O-DMAOE), 2′-O-dimethylaminopropyl(2′—O-DMAP), 2′-O-dimethylaminoethyloxyethyl(2′—O-DMAEOE), 2′-O—N-methylacetamido (2′-O-NMA), locked nucleic acid (LNA, methylene-bridged nucleic acid), ethylene-bridged nucleic acid (ENA), and (S)-constrained ethyl-bridged nucleic acid (cEt). In some embodiments, the 2′-modified nucleosides described herein are high-affinity modified nucleosides and oligonucleotides comprising the 2′-modified nucleosides have increased affinity to target sequences, relative to an unmodified oligonucleotide. Examples of structures of 2′-modified nucleosides are provided below:

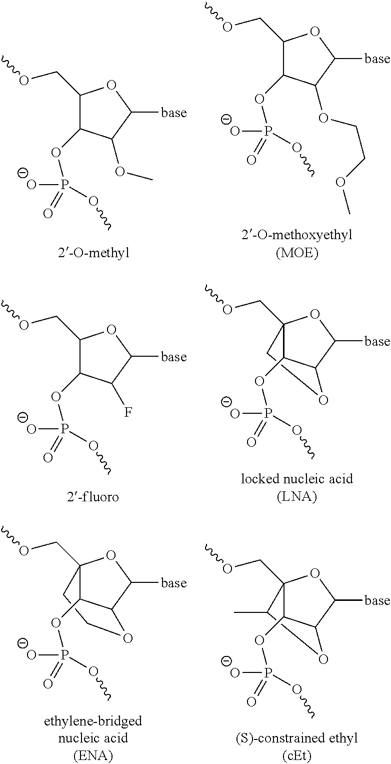

These examples are shown with phosphate groups, but any internucleoside linkages are contemplated between 2′-modified nucleosides.

### II. Complexes

Provided herein are complexes that comprise a targeting agent, e.g., an antibody, covalently linked to a molecular payload. In some embodiments, a complex comprises a muscle-targeting antibody covalently linked to an oligonucleotide. A complex may comprise an antibody that specifically binds a single antigenic site or that binds to at least two antigenic sites that may exist on the same or different antigens.

A complex may be used to modulate the activity or function of at least one gene, protein, and/or (e.g., and) nucleic acid. In some embodiments, the molecular payload present with a complex is responsible for the modulation of a gene, protein, and/or (e.g., and) nucleic acids. A molecular payload may be a small molecule, protein, nucleic acid, oligonucleotide, or any molecular entity capable of modulating the activity or function of a gene, protein, and/or (e.g., and) nucleic acid in a cell. In some embodiments, a molecular payload is an oligonucleotide that targets a DUX4 in muscle cells.

In some embodiments, a complex comprises a muscle-targeting agent, e.g. an anti-transferrin receptor antibody, covalently linked to a molecular payload, e.g. an antisense oligonucleotide that targets a DUX4. In some embodiments, a complex comprises a muscle-targeting agent, e.g. an anti-transferrin receptor antibody, covalently linked to a molecular payload, e.g. an siRNA that targets a DUX4.

### A. Muscle-Targeting Agents

Some aspects of the disclosure provide muscle-targeting agents, e.g., for delivering a molecular payload to a muscle cell. In some embodiments, such muscle-targeting agents are capable of binding to a muscle cell, e.g., via specifically binding to an antigen on the muscle cell, and delivering an associated molecular payload to the muscle cell. In some embodiments, the molecular payload is bound (e.g., covalently bound) to the muscle targeting agent and is internalized into the muscle cell upon binding of the muscle targeting agent to an antigen on the muscle cell, e.g., via endocytosis. It should be appreciated that various types of muscle-targeting agents may be used in accordance with the disclosure. For example, the muscle-targeting agent may comprise, or consist of, a nucleic acid (e.g., DNA or RNA), a peptide (e.g., an antibody), a lipid (e.g., a microvesicle), or a sugar moiety (e.g., a polysaccharide). Exemplary muscle-targeting agents are described in further detail herein, however, it should be appreciated that the exemplary muscle-targeting agents provided herein are not meant to be limiting.

Some aspects of the disclosure provide muscle-targeting agents that specifically bind to an antigen on muscle, such as skeletal muscle, smooth muscle, or cardiac muscle. In some embodiments, any of the muscle-targeting agents provided herein bind to (e.g., specifically bind to) an antigen on a skeletal muscle cell, a smooth muscle cell, and/or (e.g., and) a cardiac muscle cell.

By interacting with muscle-specific cell surface recognition elements (e.g., cell membrane proteins), both tissue localization and selective uptake into muscle cells can be achieved. In some embodiments, molecules that are substrates for muscle uptake transporters are useful for delivering a molecular payload into muscle tissue. Binding to muscle surface recognition elements followed by endocytosis can allow even large molecules such as antibodies to enter muscle cells. As another example molecular payloads conjugated to transferrin or anti-TfR1 antibodies can be taken up by muscle cells via binding to transferrin receptor, which may then be endocytosed, e.g., via clathrin-mediated endocytosis.

The use of muscle-targeting agents may be useful for concentrating a molecular payload (e.g., oligonucleotide) in muscle while reducing toxicity associated with effects in other tissues. In some embodiments, the muscle-targeting agent concentrates a bound molecular payload in muscle cells as compared to another cell type within a subject. In some embodiments, the muscle-targeting agent concentrates a bound molecular payload in muscle cells (e.g., skeletal, smooth, or cardiac muscle cells) in an amount that is at least 1, 2, 3, 4, 5, 6, 7, 8, 9, 10, 15, 20, 30, 40, 50, 60, 70, 80, 90, or 100 times greater than an amount in non-muscle cells (e.g., liver, neuronal, blood, or fat cells). In some embodiments, a toxicity of the molecular payload in a subject is reduced by at least 1%, 2%, 3%, 4%, 5%, 10%, 15%, 20%, 25%, 30%, 35%, 40%, 45%, 50%, 55%, 60%, 65%, 70%, 75%, 80%, 90%, or 95% when it is delivered to the subject when bound to the muscle-targeting agent.

In some embodiments, to achieve muscle selectivity, a muscle recognition element (e.g., a muscle cell antigen) may be required. As one example, a muscle-targeting agent may be a small molecule that is a substrate for a muscle-specific uptake transporter. As another example, a muscle-targeting agent may be an antibody that enters a muscle cell via transporter-mediated endocytosis. As another example, a muscle targeting agent may be a ligand that binds to cell surface receptor on a muscle cell. It should be appreciated that while transporter-based approaches provide a direct path for cellular entry, receptor-based targeting may involve stimulated endocytosis to reach the desired site of action.

i. Muscle-Targeting Antibodies

In some embodiments, the muscle-targeting agent is an antibody. Generally, the high specificity of antibodies for their target antigen provides the potential for selectively targeting muscle cells (e.g., skeletal, smooth, and/or (e.g., and) cardiac muscle cells). This specificity may also limit off-target toxicity. Examples of antibodies that are capable of targeting a surface antigen of muscle cells have been reported and are within the scope of the disclosure. For example, antibodies that target the surface of muscle cells are described in Arahata K., et al. “Immunostaining of skeletal and cardiac muscle surface membrane with antibody against Duchenne muscular dystrophy peptide” Nature 1988; 333:861-3; Song K. S., et al. “Expression of caveolin-3 in skeletal, cardiac, and smooth muscle cells. Caveolin-3 is a component of the sarcolemma and co-fractionates with dystrophin and dystrophin-associated glycoproteins” J Biol Chem 1996; 271:15160-5; and Weisbart R. H. et al., “Cell type specific targeted intracellular delivery into muscle of a monoclonal antibody that binds myosin IIb” Mol Immunol. 2003 Mar, 39 (13): 78309; the entire contents of each of which are incorporated herein by reference.

### a. Anti-Transferrin Receptor (TfR) Antibodies

Some aspects of the disclosure are based on the recognition that agents binding to transferrin receptor, e.g., anti-transferrin-receptor antibodies, are capable of targeting muscle cell. Transferrin receptors are internalizing cell surface receptors that transport transferrin across the cellular membrane and participate in the regulation and homeostasis of intracellular iron levels. Some aspects of the disclosure provide transferrin receptor binding proteins, which are capable of binding to transferrin receptor. Accordingly, aspects of the disclosure provide binding proteins (e.g., antibodies) that bind to transferrin receptor. In some embodiments, binding proteins that bind to transferrin receptor are internalized, along with any bound molecular payload, into a muscle cell. As used herein, an antibody that binds to a transferrin receptor may be referred to interchangeably as a transferrin receptor antibody, an anti-transferrin receptor antibody, or an anti-TfR1 antibody. Antibodies that bind, e.g. specifically bind, to a transferrin receptor may be internalized into the cell, e.g. through receptor-mediated endocytosis, upon binding to a transferrin receptor.

It should be appreciated that anti-TfR1 antibodies may be produced, synthesized, and/or (e.g., and) derivatized using several known methodologies, e.g. library design using phage display. Exemplary methodologies have been characterized in the art and are incorporated by reference (Díez, P. et al. “High-throughput phage-display screening in array format”, Enzyme and microbial technology, 2015, 79, 34-41.; Christoph M. H. and Stanley, J. R. “Antibody Phage Display: Technique and Applications” J Invest Dermatol. 2014, 134:2.; Engleman, Edgar (Ed.) “Human Hybridomas and Monoclonal Antibodies.” 1985, Springer.). In other embodiments, an anti-TfR1 antibody has been previously characterized or disclosed. Antibodies that specifically bind to transferrin receptor are known in the art (see, e.g. U.S. Pat. No. 4,364,934, filed Dec. 4, 1979, “Monoclonal antibody to a human early thymocyte antigen and methods for preparing same”; U.S. Pat. No. 8,409,573, filed Jun. 14, 2006, “Anti-CD71 monoclonal antibodies and uses thereof for treating malignant tumor cells”; U.S. Pat. No. 9,708,406, filed May 20, 2014, “Anti-Transferrin receptor antibodies and methods of use”; U.S. Pat. No. 9,611,323, filed Dec. 19, 2014, “Low affinity blood brain barrier receptor antibodies and uses therefor”; WO 2015/098989, filed Dec. 24, 2014, “Novel anti-Transferrin receptor antibody that passes through blood-brain barrier”; Schneider C. et al. “Structural features of the cell surface receptor for transferrin that is recognized by the monoclonal antibody OKT9.” J Biol Chem. 1982, 257:14, 8516-8522.; Lee et al. “Targeting Rat Anti-Mouse Transferrin Receptor Monoclonal Antibodies through Blood-Brain Barrier in Mouse” 2000, J Pharmacol. Exp. Ther., 292:1048-1052).

In some embodiments, the anti-TfR1 antibody described herein binds to transferrin receptor with high specificity and affinity. In some embodiments, the anti-TfR1 antibody described herein specifically binds to any extracellular epitope of a transferrin receptor or an epitope that becomes exposed to an antibody. In some embodiments, anti-TfR1 antibodies provided herein bind specifically to transferrin receptor from human, non-human primates, mouse, rat, etc. In some embodiments, anti-TfR1 antibodies provided herein bind to human transferrin receptor. In some embodiments, the anti-TfR1 antibody described herein binds to an amino acid segment of a human or non-human primate transferrin receptor, as provided in SEQ ID NOs: 105-108. In some embodiments, the anti-TfR1 antibody described herein binds to an amino acid segment corresponding to amino acids 90-96 of a human transferrin receptor as set forth in SEQ ID NO: 105, which is not in the apical domain of the transferrin receptor. In some embodiments, the humanized anti-TfR1 antibodies described herein binds to TfR1 but does not bind to TfR2.

In some embodiments, the anti-TfR1 antibodies described herein (e.g., Anti-TfR clone 8 in Table 2 below) bind an epitope in TfR1, wherein the epitope comprises residues in amino acids 214-241 and/or amino acids 354-381 of SEQ ID NO: 105. In some embodiments, the anti-TfR1 antibodies described herein bind an epitope comprising residues in amino acids 214-241 and amino acids 354-381 of SEQ ID NO: 105. In some embodiments, the anti-TfR1 antibodies described herein bind an epitope comprising one or more of residues Y222, T227, K231, H234, T367, S368, S370, T376, and S378 of human TfR1 as set forth in SEQ ID NO: 105. In some embodiments, the anti-TfR1 antibodies described herein bind an epitope comprising residues Y222, T227, K231, H234, T367, S368, S370, T376, and S378 of human TfR1 as set forth in SEQ ID NO: 105.

In some embodiments, the anti-TfR1 antibody described herein (e.g., 3M12 in Table 2 below and its variants) bind an epitope in TfR1, wherein the epitope comprises residues in amino acids 258-291 and/or amino acids 358-381 of SEQ ID NO: 105. In some embodiments, the anti-TfR1 antibodies (e.g., 3M12 in Table 2 below and its variants) described herein bind an epitope comprising residues in amino acids amino acids 258-291 and amino acids 358-381 of SEQ ID NO: 105. In some embodiments, the anti-TfR1 antibodies described herein (e.g., 3M12 in Table 2 below and its variants) bind an epitope comprising one or more of residues K261, S273, Y282, T362, S368, S370, and K371 of human TfR1 as set forth in SEQ ID NO: 105. In some embodiments, the anti-TfR1 antibodies described herein (e.g., 3M12 in Table 2 below and its variants) bind an epitope comprising residues K261, S273, Y282, T362, S368, S370, and K371 of human TfR1 as set forth in SEQ ID NO: 105.

An example human transferrin receptor amino acid sequence, corresponding to NCBI sequence NP_003225.2 (transferrin receptor protein 1 isoform 1, Homo sapiens) is as follows:

(SEQ ID NO: 105)
MMDQARSAFSNLFGGEPLSYTRFSLARQVDGDNSHVEMKLAVDEEENADN
NTKANVTKPKRCSGSICYGTIAVIVFFLIGFMIGYLGYCKGVEPKTECER
LAGTESPVREEPGEDFPAARRLYWDDLKRKLSEKLDSTDFTGTIKLLNEN
SYVPREAGSQKDENLALYVENQFREFKLSKVWRDQHFVKIQVKDSAQNSV
IIVDKNGRLVYLVENPGGYVAYSKAATVTGKLVHANFGTKKDFEDLYTPV
NGSIVIVRAGKITFAEKVANAESLNAIGVLIYMDQTKFPIVNAELSFFGH
AHLGTGDPYTPGFPSFNHTQFPPSRSSGLPNIPVQTISRAAAEKLFGNME
GDCPSDWKTDSTCRMVTSESKNVKLTVSNVLKEIKILNIFGVIKGFVEPD
HYVVVGAQRDAWGPGAAKSGVGTALLLKLAQMFSDMVLKDGFQPSRSIIF
ASWSAGDFGSVGATEWLEGYLSSLHLKAFTYINLDKAVLGTSNFKVSASP
LLYTLIEKTMQNVKHPVTGQFLYQDSNWASKVEKLTLDNAAFPFLAYSGI
PAVSFCFCEDTDYPYLGTTMDTYKELIERIPELNKVARAAAEVAGQFVIK
LTHDVELNLDYERYNSQLLSFVRDLNQYRADIKEMGLSLQWLYSARGDFF
RATSRLTTDFGNAEKTDRFVMKKLNDRVMRVEYHFLSPYVSPKESPFRHV
FWGSGSHTLPALLENLKLRKQNNGAFNETLFRNQLALATWTIQGAANALS
GDVWDIDNEF.

An example non-human primate transferrin receptor amino acid sequence, corresponding to NCBI sequence NP_001244232.1 (transferrin receptor protein 1, Macaca mulatta) is as follows:

(SEQ ID NO: 106)
MMDQARSAFSNLFGGEPLSYTRFSLARQVDGDNSHVEMKLGVDEEENTDN
NTKPNGTKPKRCGGNICYGTIAVIIFFLIGFMIGYLGYCKGVEPKTECER
LAGTESPAREEPEEDFPAAPRLYWDDLKRKLSEKLDTTDFTSTIKLLNEN
LYVPREAGSQKDENLALYIENQFREFKLSKVWRDQHFVKIQVKDSAQNSV
IIVDKNGGLVYLVENPGGYVAYSKAATVTGKLVHANFGTKKDFEDLDSPV
NGSIVIVRAGKITFAEKVANAESLNAIGVLIYMDQTKFPIVKADLSFFGH
AHLGTGDPYTPGFPSFNHTQFPPSQSSGLPNIPVQTISRAAAEKLFGNME
GDCPSDWKTDSTCKMVTSENKSVKLTVSNVLKETKILNIFGVIKGFVEPD
HYVVVGAQRDAWGPGAAKSSVGTALLLKLAQMFSDMVLKDGFQPSRSIIF
ASWSAGDFGSVGATEWLEGYLSSLHLKAFTYINLDKAVLGTSNFKVSASP
LLYTLIEKTMQDVKHPVTGRSLYQDSNWASKVEKLTLDNAAFPFLAYSGI
PAVSFCFCEDTDYPYLGTTMDTYKELVERIPELNKVARAAAEVAGQFVIK
LTHDTELNLDYERYNSQLLLFLRDLNQYRADVKEMGLSLQWLYSARGDFF
RATSRLTTDFRNAEKRDKFVMKKLNDRVMRVEYYFLSPYVSPKESPFRHV
FWGSGSHTLSALLESLKLRRQNNSAFNETLFRNQLALATWTIQGAANALS
GDVWDIDNEF.

An example non-human primate transferrin receptor amino acid sequence, corresponding to NCBI sequence XP_005545315.1 (transferrin receptor protein 1, Macaca fascicularis) is as follows:

(SEQ ID NO: 107)
MMDQARSAFSNLFGGEPLSYTRFSLARQVDGDNSHVEMKLGVDEEENTDN
NTKANGTKPKRCGGNICYGTIAVIIFFLIGFMIGYLGYCKGVEPKTECER
LAGTESPAREEPEEDFPAAPRLYWDDLKRKLSEKLDTTDFTSTIKLLNEN
LYVPREAGSQKDENLALYIENQFREFKLSKVWRDQHFVKIQVKDSAQNSV
IIVDKNGGLVYLVENPGGYVAYSKAATVTGKLVHANFGTKKDFEDLDSPV
NGSIVIVRAGKITFAEKVANAESLNAIGVLIYMDQTKFPIVKADLSFFGH
AHLGTGDPYTPGFPSFNHTQFPPSQSSGLPNIPVQTISRAAAEKLFGNME
GDCPSDWKTDSTCKMVTSENKSVKLTVSNVLKETKILNIFGVIKGFVEPD
HYVVVGAQRDAWGPGAAKSSVGTALLLKLAQMFSDMVLKDGFQPSRSIIF
ASWSAGDFGSVGATEWLEGYLSSLHLKAFTYINLDKAVLGTSNFKVSASP
LLYTLIEKTMQDVKHPVTGRSLYQDSNWASKVEKLTLDNAAFPFLAYSGI
PAVSFCFCEDTDYPYLGTTMDTYKELVERIPELNKVARAAAEVAGQFVIK
LTHDTELNLDYERYNSQLLLFLRDLNQYRADVKEMGLSLQWLYSARGDFF
RATSRLTTDFRNAEKRDKFVMKKLNDRVMRVEYYFLSPYVSPKESPFRHV
FWGSGSHTLSALLESLKLRRQNNSAFNETLFRNQLALATWTIQGAANALS
GDVWDIDNEF.

An example mouse transferrin receptor amino acid sequence, corresponding to NCBI sequence NP_001344227.1 (transferrin receptor protein 1, Mus musculus) is as follows:

(SEQ ID NO: 108)
MMDQARSAFSNLFGGEPLSYTRFSLARQVDGDNSHVEMKLAADEEENADN
NMKASVRKPKRFNGRLCFAAIALVIFFLIGFMSGYLGYCKRVEQKEECVK
LAETEETDKSETMETEDVPTSSRLYWADLKTLLSEKLNSIEFADTIKQLS
QNTYTPREAGSQKDESLAYYIENQFHEFKFSKVWRDEHYVKIQVKSSIGQ
NMVTIVQSNGNLDPVESPEGYVAFSKPTEVSGKLVHANFGTKKDFEELSY
SVNGSLVIVRAGEITFAEKVANAQSFNAIGVLIYMDKNKFPVVEADLALF
GHAHLGTGDPYTPGFPSFNHTQFPPSQSSGLPNIPVQTISRAAAEKLFGK
MEGSCPARWNIDSSCKLELSQNQNVKLIVKNVLKERRILNIFGVIKGYEE
PDRYVVVGAQRDALGAGVAAKSSVGTGLLLKLAQVFSDMISKDGFRPSRS
IIFASWTAGDFGAVGATEWLEGYLSSLHLKAFTYINLDKVVLGTSNFKVS
ASPLLYTLMGKIMQDVKHPVDGKSLYRDSNWISKVEKLSFDNAAYPFLAY
SGIPAVSFCFCEDADYPYLGTRLDTYEALTQKVPQLNQMVRTAAEVAGQL
IIKLTHDVELNLDYEMYNSKLLSFMKDLNQFKTDIRDMGLSLQWLYSARG
DYFRATSRLTTDFHNAEKTNRFVMREINDRIMKVEYHFLSPYVSPRESPF
RHIFWGSGSHTLSALVENLKLRQKNITAFNETLFRNQLALATWTIQGVAN
ALSGDIWNIDNEF.

In some embodiments, an anti-TfR1 antibody binds to an amino acid segment of the receptor as follows:
FVKIQVKDSAQNSVIIVDKNGRLVYLVENPGGYVAYSKAATVTGKLVHANFGTKKDF EDLYTPVNGSIVIVRAGKITFAEKVANAESLNAIGVLIYMDQTKFPIVNAELSFFGHAH LGTGDPYTPGFPSFNHTQFPPSRSSGLPNIPVQTISRAAAEKLFGNMEGDCPSDWKTDS TCRMVTSESKNVKLTVSNVLKE (SEQ ID NO: 109) and does not inhibit the binding interactions between transferrin receptors and transferrin and/or (e.g., and) human hemochromatosis protein (also known as HFE). In some embodiments, the anti-TfR1 antibody described herein does not bind an epitope in SEQ ID NO: 109.

Appropriate methodologies may be used to obtain and/or (e.g., and) produce antibodies, antibody fragments, or antigen-binding agents, e.g., through the use of recombinant DNA protocols. In some embodiments, an antibody may also be produced through the generation of hybridomas (see, e.g., Kohler, G and Milstein, C. “Continuous cultures of fused cells secreting antibody of predefined specificity” Nature, 1975, 256:495-497). The antigen-of-interest may be used as the immunogen in any form or entity, e.g., recombinant or a naturally occurring form or entity. Hybridomas are screened using standard methods, e.g., ELISA screening, to find at least one hybridoma that produces an antibody that targets a particular antigen. Antibodies may also be produced through screening of protein expression libraries that express antibodies, e.g., phage display libraries. Phage display library design may also be used, in some embodiments, (see, e.g. U.S. Pat. No. 5,223,409, filed Mar. 1, 1991, “Directed evolution of novel binding proteins”; WO 1992/18619, filed Apr. 10, 1992, “Heterodimeric receptor libraries using phagemids”; WO 1991/17271, filed May 1, 1991, “Recombinant library screening methods”; WO 1992/20791, filed May 15, 1992, “Methods for producing members of specific binding pairs”; WO 1992/15679, filed Feb. 28, 1992, and “Improved epitope displaying phage”). In some embodiments, an antigen-of-interest may be used to immunize a non-human animal, e.g., a rodent or a goat. In some embodiments, an antibody is then obtained from the non-human animal, and may be optionally modified using a number of methodologies, e.g., using recombinant DNA techniques. Additional examples of antibody production and methodologies are known in the art (see, e.g. Harlow et al. “Antibodies: A Laboratory Manual”, Cold Spring Harbor Laboratory, 1988.).

In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecules are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, there are about 1-10, about 1-5, about 5-10, about 1-4, about 1-3, or about 2 sugar molecules. In some embodiments, a glycosylated antibody is fully or partially glycosylated. In some embodiments, an antibody is glycosylated by chemical reactions or by enzymatic means. In some embodiments, an antibody is glycosylated in vitro or inside a cell, which may optionally be deficient in an enzyme in the N- or O-glycosylation pathway, e.g. a glycosyltransferase. In some embodiments, an antibody is functionalized with sugar or carbohydrate molecules as described in International Patent Application Publication WO2014065661, published on May 1, 2014, entitled, “Modified antibody, antibody-conjugate and process for the preparation thereof”.

In some embodiments, the anti-TfR1 antibody of the present disclosure comprises a VL domain and/or (e.g., and) a VH domain of any one of the anti-TfR1 antibodies selected from any one of Tables 2-7, and comprises a constant region comprising the amino acid sequences of the constant regions of an IgG, IgE, IgM, IgD, IgA or IgY immunoglobulin molecule, any class (e.g., IgG1, IgG2, IgG3, IgG4, IgAQ1 and IgA2), or any subclass (e.g., IgG2a and IgG2b) of immunoglobulin molecule. Non-limiting examples of human constant regions are described in the art, e.g., see Kabat E A et al., (1991) supra.

In some embodiments, agents binding to transferrin receptor, e.g., anti-TfR1 antibodies, are capable of targeting muscle cell and/or (e.g., and) mediate the transportation of an agent across the blood brain barrier. Transferrin receptors are internalizing cell surface receptors that transport transferrin across the cellular membrane and participate in the regulation and homeostasis of intracellular iron levels. Some aspects of the disclosure provide transferrin receptor binding proteins, which are capable of binding to transferrin receptor. Antibodies that bind, e.g. specifically bind, to a transferrin receptor may be internalized into the cell, e.g. through receptor-mediated endocytosis, upon binding to a transferrin receptor.

In some embodiments, an anti-TFR1 antibody specifically binds a TfR1 (e.g., a human or non-human primate TfR1) with binding affinity (e.g., as indicated by Kd) of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, the anti-TfR1 antibodies described herein bind to TfR1 with a KD of sub-nanomolar range. In some embodiments, the anti-TfR1 antibodies described herein selectively bind to transferrin receptor 1 (TfR1) but do not bind to transferrin receptor 2 (TfR2). In some embodiments, the anti-TfR1 antibodies described herein bind to human TfR1 and cyno TfR1 (e.g., with a Kd of 10⁻⁷ M, 10⁻⁸ M, 10⁻⁹ M, 10⁻¹⁰ M, 10⁻¹¹ M, 10⁻¹² M, 10⁻¹³ M, or less), but do not bind to a mouse TfR1. The affinity and binding kinetics of the anti-TfR1 antibody can be tested using any suitable method including but not limited to biosensor technology (e.g., OCTET or BIACORE). In some embodiments, binding of any one of the anti-TfR1 antibody described herein does not complete with or inhibit transferrin binding to the TfR1. In some embodiments, binding of any one of the anti-TfR1 antibodies described herein does not complete with or inhibit HFE-beta-2-microglobulin binding to the TfR1. Non-limiting examples of anti-TfR1 antibodies are provided in Table 2.

TABLE 2
Examples of Anti-TfR1 Antibodies

| | No. | | | |
|---|---|---|---|---|
| Ab | system | IMGT | Kabat | Chothia |
| 3-A4 | CDR- | GFNIKDDY | DDYMY | GFNIKDD |
| | H1 | (SEQ ID NO: 1) | (SEQ ID NO: 7) | (SEQ ID NO: 12) |
| | CDR- | IDPENGDT (SEQ ID NO: | WIDPENGDTEYASKFQD | ENG |
| | H2 | 2) | (SEQ ID NO: 8) | (SEQ ID NO: 13) |
| | CDR- | TLWLRRGLDY (SEQ ID | WLRRGLDY (SEQ ID NO: | LRRGLD |
| | H3 | NO: 3) | 9) | (SEQ ID NO: 14) |
| | CDR- | KSLLHSNGYTY (SEQ ID | RSSKSLLHSNGYTYLF | SKSLLHSNGYTY (SEQ ID |
| | L1 | NO: 4) | (SEQ ID NO: 10) | NO: 15) |
| | CDR- | RMS | RMSNLAS (SEQ ID NO: | RMS |
| | L2 | (SEQ ID NO: 5) | 11) | (SEQ ID NO: 5) |
| | CDR- | MQHLEYPFT (SEQ ID | MQHLEYPFT (SEQ ID | HLEYPF |
| | L3 | NO: 6) | NO: 6) | (SEQ ID NO: 16) |
| | VH | EVQLQQSGAELVRPGASVKLSCTASGFNIKDDYMYWVKQRPEQGLEWIGWIDPEN | | |
| | | GDTEYASKFQDKATVTADTSSNTAYLQLSSLTSEDTAVYYCTLWLRRGLDYWGQG | | |
| | | TSVTVSS (SEQ ID NO: 17) | | |
| | VL | DIVMTQAAPSVPVTPGESVSISCRSSKSLLHSNGYTYLFWFLQRPGQSPQLLIYRMSN | | |
| | | LASGVPDRFSGSGSGTAFTLRISRVEAEDVGVYYCMQHLEYPFTFGGGTKLEIK | | |
| | | (SEQ ID NO: 18) | | |
| 3-A4 | CDR- | GFNIKDDY (SEQ ID NO: | DDYMY (SEQ ID NO: 7) | GFNIKDD (SEQ ID NO: |
| N54T* | H1 | 1) | | 12) |
| | CDR- | IDPETGDT (SEQ ID NO: | WIDPETGDTEYASKFQD | ETG (SEQ ID NO: 21) |
| | H2 | 19) | (SEQ ID NO: 20) | |
| | CDR- | TLWLRRGLDY (SEQ ID | WLRRGLDY (SEQ ID NO: | LRRGLD (SEQ ID NO: 14) |
| | H3 | NO: 3) | 9) | |
| | CDR- | KSLLHSNGYTY (SEQ ID | RSSKSLLHSNGYTYLF | SKSLLHSNGYTY (SEQ ID |
| | L1 | NO: 4) | (SEQ ID NO: 10) | NO: 15) |
| | CDR- | RMS (SEQ ID NO: 5) | RMSNLAS (SEQ ID NO: | RMS (SEQ ID NO: 5) |
| | L2 | | 11) | |
| | CDR- | MQHLEYPFT (SEQ ID | MQHLEYPFT (SEQ ID | HLEYPF (SEQ ID NO: 16) |
| | L3 | NO: 6) | NO: 6) | |
| | VH | EVQLQQSGAELVRPGASVKLSCTASGFNIKDDYMYWVKQRPEQGLEWIGWIDPET | | |
| | | GDTEYASKFQDKATVTADTSSNTAYLQLSSLTSEDTAVYYCTLWLRRGLDYWGQG | | |
| | | TSVTVSS (SEQ ID NO: 22) | | |
| | VL | DIVMTQAAPSVPVTPGESVSISCRSSKSLLHSNGYTYLFWFLQRPGQSPQLLIYRMSN | | |
| | | LASGVPDRFSGSGSGTAFTLRISRVEAEDVGVYYCMQHLEYPFTFGGGTKLEIK | | |
| | | (SEQ ID NO: 18) | | |
| 3-A4 | CDR- | GFNIKDDY (SEQ ID NO: | DDYMY (SEQ ID NO: 7) | GFNIKDD (SEQ ID NO: |
| N54S* | H1 | 1) | | 12) |
| | CDR- | IDPESGDT (SEQ ID NO: | WIDPESGDTEYASKFQD | ESG (SEQ ID NO: 25) |
| | H2 | 23) | (SEQ ID NO: 24) | |
| | CDR- | TLWLRRGLDY (SEQ ID | WLRRGLDY (SEQ ID NO: | LRRGLD (SEQ ID NO: 14) |
| | H3 | NO: 3) | 9) | |
| | CDR- | KSLLHSNGYTY (SEQ ID | RSSKSLLHSNGYTYLF | SKSLLHSNGYTY (SEQ ID |
| | L1 | NO: 4) | (SEQ ID NO: 10) | NO: 15) |
| | CDR- | RMS (SEQ ID NO: 5) | RMSNLAS (SEQ ID NO: | RMS (SEQ ID NO: 5) |
| | L2 | | 11) | |
| | CDR- | MQHLEYPFT (SEQ ID | MQHLEYPFT (SEQ ID | HLEYPF (SEQ ID NO: 16) |
| | L3 | NO: 6) | NO: 6) | |
| | VH | EVQLQQSGAELVRPGASVKLSCTASGFNIKDDYMYWVKQRPEQGLEWIGWIDPES | | |
| | | GDTEYASKFQDKATVTADTSSNTAYLQLSSLTSEDTAVYYCTLWLRRGLDYWGQG | | |
| | | TSVTVSS (SEQ ID NO: 26) | | |
| | VL | DIVMTQAAPSVPVTPGESVSISCRSSKSLLHSNGYTYLFWFLQRPGQSPQLLIYRMSN | | |
| | | LASGVPDRFSGSGSGTAFTLRISRVEAEDVGVYYCMQHLEYPFTFGGGTKLEIK | | |
| | | (SEQ ID NO: 18) | | |
| 3-M12 | CDR- | GYSITSGYY (SEQ ID NO: | SGYYWN (SEQ ID NO: | GYSITSGY (SEQ ID NO: |
| | H1 | 27) | 33) | 38) |
| | CDR- | ITFDGAN (SEQ ID NO: | YITFDGANNYNPSLKN | FDG (SEQ ID NO: 39) |
| | H2 | 28) | (SEQ ID NO: 34) | |
| | CDR- | TRSSYDYDVLDY (SEQ | SSYDYDVLDY (SEQ ID | SYDYDVLD (SEQ ID NO: |
| | H3 | ID NO: 29) | NO: 35) | 40) |
| | CDR- | QDISNF (SEQ ID NO: 30) | RASQDISNFLN (SEQ ID | SQDISNF (SEQ ID NO: 41) |
| | L1 | | NO: 36) | |
| | CDR- | YTS (SEQ ID NO: 31) | YTSRLHS (SEQ ID NO: | YTS (SEQ ID NO: 31) |
| | L2 | | 37) | |
| | CDR- | QQGHTLPYT (SEQ ID | QQGHTLPYT (SEQ ID | GHTLPY (SEQ ID NO: 42) |
| | L3 | NO: 32) | NO: 32) | |
| | VH | DVQLQESGPGLVKPSQSLSLTCSVTGYSITSGYYWNWIRQFPGNKLEWMGYITFDG | | |
| | | ANNYNPSLKNRISITRDTSKNQFFLKLTSVTTEDTATYYCTRSSYDYDVLDYWGQG | | |
| | | TTLTVSS (SEQ ID NO: 43) | | |
| | VL | DIQMTQTTSSLSASLGDRVTISCRASQDISNFLNWYQQRPDGTVKLLIYYTSRLHSG | | |
| | | VPSRFSGSGSGTDFSLTVSNLEQEDIATYFCQQGHTLPYTFGGGTKLEIK (SEQ ID | | |
| | | NO: 44) | | |
| 5-H12 | CDR- | GYSFTDYC (SEQ ID NO: | DYCIN (SEQ ID NO: 51) | GYSFTDY (SEQ ID NO: |
| | H1 | 45) | | 56) |
| | CDR- | IYPGSGNT (SEQ ID NO: | WIYPGSGNTRYSERFKG | GSG (SEQ ID NO: 57) |
| | H2 | 46) | (SEQ ID NO: 52) | |
| | CDR- | AREDYYPYHGMDY | EDYYPYHGMDY (SEQ ID | DYYPYHGMD (SEQ ID |
| | H3 | (SEQ ID NO: 47) | NO: 53) | NO: 58) |
| | CDR- | ESVDGYDNSF (SEQ ID | RASESVDGYDNSFMH | SESVDGYDNSF (SEQ ID |
| | L1 | NO: 48) | (SEQ ID NO: 54) | NO: 59) |
| | CDR- | RAS (SEQ ID NO: 49) | RASNLES (SEQ ID NO: | RAS (SEQ ID NO: 49) |
| | L2 | | 55) | |
| | CDR- | QQSSEDPWT (SEQ ID | QQSSEDPWT (SEQ ID | SSEDPW (SEQ ID NO: 60) |
| | L3 | NO: 50) | NO: 50) | |
| | VH | QIQLQQSGPELVRPGASVKISCKASGYSFTDYCINWVNQRPGQGLEWIGWIYPGSGN | | |
| | | TRYSERFKGKATLTVDTSSNTAYMQLSSLTSEDSAVYFCAREDYYPYHGMDYWGQ | | |
| | | GTSVTVSS (SEQ ID NO: 61) | | |
| | VL | DIVLTQSPTSLAVSLGQRATISCRASESVDGYDNSFMHWYQQKPGQPPKLLIFRASN | | |
| | | LESGIPARFSGSGSRTDFTLTINPVEAADVATYYCQQSSEDPWTFGGGTKLEIK (SEQ | | |
| | | ID NO: 62) | | |
| 5-H12 | CDR- | GYSFTDYY (SEQ ID NO: | DYYIN (SEQ ID NO: 64) | GYSFTDY (SEQ ID NO: |
| C33Y* | H1 | 63) | | 56) |
| | CDR- | IYPGSGNT (SEQ ID NO: | WIYPGSGNTRYSERFKG | GSG (SEQ ID NO: 57) |
| | H2 | 46) | (SEQ ID NO: 52) | |
| | CDR- | AREDYYPYHGMDY | EDYYPYHGMDY (SEQ ID | DYYPYHGMD (SEQ ID |
| | H3 | (SEQ ID NO: 47) | NO: 53) | NO: 58) |
| | CDR- | ESVDGYDNSF (SEQ ID | RASESVDGYDNSFMH | SESVDGYDNSF (SEQ ID |
| | L1 | NO: 48) | (SEQ ID NO: 54) | NO: 59) |
| | CDR- | RAS (SEQ ID NO: 49) | RASNLES (SEQ ID NO: | RAS (SEQ ID NO: 49) |
| | L2 | | 55) | |
| | CDR- | QQSSEDPWT (SEQ ID | QQSSEDPWT (SEQ ID | SSEDPW (SEQ ID NO: 60) |
| | L3 | NO: 50) | NO: 50) | |
| | VH | QIQLQQSGPELVRPGASVKISCKASGYSFTDYYINWVNQRPGQGLEWIGWIYPGSG | | |
| | | NTRYSERFKGKATLTVDTSSNTAYMQLSSLTSEDSAVYFCAREDYYPYHGMDYWG | | |
| | | QGTSVTVSS (SEQ ID NO: 65) | | |
| | VL | DIVLTQSPTSLAVSLGQRATISCRASESVDGYDNSFMHWYQQKPGQPPKLLIFRASN | | |
| | | LESGIPARFSGSGSRTDFTLTINPVEAADVATYYCQQSSEDPWTFGGGTKLEIK (SEQ | | |
| | | ID NO: 62) | | |
| 5-H12 | CDR- | GYSFTDYD (SEQ ID NO: | DYDIN (SEQ ID NO: 67) | GYSFTDY (SEQ ID NO: |
| C33D* | H1 | 66) | | 56) |
| | CDR- | IYPGSGNT (SEQ ID NO: | WIYPGSGNTRYSERFKG | GSG (SEQ ID NO: 57) |
| | H2 | 46) | (SEQ ID NO: 52) | |
| | CDR- | AREDYYPYHGMDY | EDYYPYHGMDY (SEQ ID | DYYPYHGMD (SEQ ID |
| | H3 | (SEQ ID NO: 47) | NO: 53) | NO: 58) |
| | CDR- | ESVDGYDNSF (SEQ ID | RASESVDGYDNSFMH | SESVDGYDNSF (SEQ ID |
| | L1 | NO: 48) | (SEQ ID NO: 54) | NO: 59) |
| | CDR- | RAS (SEQ ID NO: 49) | RASNLES (SEQ ID NO: | RAS (SEQ ID NO: 49) |
| | L2 | | 55) | |
| | CDR- | QQSSEDPWT (SEQ ID | QQSSEDPWT (SEQ ID | SSEDPW (SEQ ID NO: 60) |
| | L3 | NO: 50) | NO: 50) | |
| | VH | QIQLQQSGPELVRPGASVKISCKASGYSFTDYDINWVNQRPGQGLEWIGWIYPGSGNT | | |
| | | RYSERFKGKATLTVDTSSNTAYMQLSSLTSEDSAVYFCAREDYYPYHGMDYWGQGT | | |
| | | SVTVSS (SEQ ID NO: 68) | | |
| | VL | DIVLTQSPTSLAVSLGQRATISCRASESVDGYDNSFMHWYQQKPGQPPKLLIFRASN | | |
| | | LESGIPARFSGSGSRTDFTLTINPVEAADVATYYCQQSSEDPWTFGGGTKLEIK (SEQ | | |
| | | ID NO: 62) | | |
| Anti- | CDR- | GYSFTSYW (SEQ ID NO: | SYWIG (SEQ ID NO: 144) | GYSFTSY (SEQ ID NO: |
| TfR | H1 | 138) | | 149) |
| clone 8 | CDR- | IYPGDSDT (SEQ ID NO: | IIYPGDSDTRYSPSFQGQ | GDS (SEQ ID NO: 150) |
| | H2 | 139) | (SEQ ID NO: 145) | |
| | CDR- | ARFPYDSSGYYSFDY | FPYDSSGYYSFDY (SEQ | PYDSSGYYSFD (SEQ ID |
| | H3 | (SEQ ID NO: 140) | ID NO: 146) | NO: 151) |
| | CDR- | QSISSY | RASQSISSYLN (SEQ ID | SQSISSY (SEQ ID NO: |
| | L1 | (SEQ ID NO: 141) | NO: 147) | 152) |
| | CDR- | AAS (SEQ ID NO: 142) | AASSLQS (SEQ ID NO: | AAS (SEQ ID NO: 142) |
| | L2 | | 148) | |
| | CDR- | QQSYSTPLT (SEQ ID NO: | QQSYSTPLT (SEQ ID NO: | SYSTPL (SEQ ID NO: 153) |
| | L3 | 143) | 143) | |

*mutation positions are according to Kabat numbering of the respective VH sequences containing the mutations

In some embodiments, the anti-TfR1 antibody of the present disclosure is a variant of any one of the anti-TfR1 antibodies provided in Table 2. In some embodiments, the anti-TfR1 antibody of the present disclosure comprises a CDR-H1, a CDR-H2, a CDR-H3, a CDR-L1, a CDR-L2, and a CDR-L3 that are the same as the CDR-H1, CDR-H2, and CDR-H3 in any one of the anti-TfR1 antibodies provided in Table 2, and comprises a humanized heavy chain variable region and/or (e.g., and) a humanized light chain variable region.

Examples of amino acid sequences of the anti-TfR1 antibodies described herein are provided in Table 3.

TABLE 3
Variable Regions of Anti-TfR1 Antibodies

| Antibody | Variable Region Amino Acid Sequence |
|---|---|
| 3A4 | VH: |
| VH3 (N54T*)/Vκ4 | EVQLVQSGSELKKPGASVKVSCTASGFNIKDDYMYWVRQPPGKGLEWIGWIDPE |
| | TGDTEYASKFQDRVTVTADTSTNTAYMELSSLRSEDTAVYYCTLWLRRGLDY |
| | WGQGTLVTVSS (SEQ ID NO: 69) |
| | VL: |
| | DIVMTQSPLSLPVTPGEPASISCRSSKSLLHSNGYTYLFWFQQRPGQSPRLLIYRM |
| | SNLASGVPDRFSGSGSGTDFTLKISRVEAEDVGVYYCMQHLEYPFTFGGGTKVEI |
| | K (SEQ ID NO: 70) |
| 3A4 | VH: |
| VH3 (N54S*)/Vκ4 | EVQLVQSGSELKKPGASVKVSCTASGFNIKDDYMYWVRQPPGKGLEWIGWIDPE |
| | SGDTEYASKFQDRVTVTADTSTNTAYMELSSLRSEDTAVYYCTLWLRRGLDY |
| | WGQGTLVTVSS (SEQ ID NO: 71) |
| | VL: |
| | DIVMTQSPLSLPVTPGEPASISCRSSKSLLHSNGYTYLFWFQQRPGQSPRLLIYRM |
| | SNLASGVPDRFSGSGSGTDFTLKISRVEAEDVGVYYCMQHLEYPFTFGGGTKVEI |
| | K (SEQ ID NO: 70) |
| 3A4 | VH: |
| VH3/Vκ4 | EVQLVQSGSELKKPGASVKVSCTASGFNIKDDYMYWVRQPPGKGLEWIGWIDPE |
| | NGDTEYASKFQDRVTVTADTSTNTAYMELSSLRSEDTAVYYCTLWLRRGLDY |
| | WGQGTLVTVSS (SEQ ID NO: 72) |
| | VL: |
| | DIVMTQSPLSLPVTPGEPASISCRSSKSLLHSNGYTYLFWFQQRPGQSPRLLIYRM |
| | SNLASGVPDRFSGSGSGTDFTLKISRVEAEDVGVYYCMQHLEYPFTFGGGTKVEI |
| | K (SEQ ID NO: 70) |
| 3M12 | VH: |
| VH3/Vκ2 | QVQLQESGPGLVKPSQTLSLTCSVTGYSITSGYYWNWIRQPPGKGLEWMGYITF |
| | DGANNYNPSLKNRVSISRDTSKNQFSLKLSSVTAEDTATYYCTRSSYDYDVLDY |
| | WGQGTTVTVSS (SEQ ID NO: 73) |
| | VL: |
| | DIQMTQSPSSLSASVGDRVTITCRASQDISNFLNWYQQKPGQPVKLLIYYTSRLH |
| | SGVPSRFSGSGSGTDFTLTISSLQPEDFATYFCQQGHTLPYTFGQGTKLEIK (SEQ |
| | ID NO: 74) |
| 3M12 | VH: |
| VH3/Vκ3 | QVQLQESGPGLVKPSQTLSLTCSVTGYSITSGYYWNWIRQPPGKGLEWMGYITF |
| | DGANNYNPSLKNRVSISRDTSKNQFSLKLSSVTAEDTATYYCTRSSYDYDVLDY |
| | WGQGTTVTVSS (SEQ ID NO: 73) |
| | VL: |
| | DIQMTQSPSSLSASVGDRVTITCRASQDISNFLNWYQQKPGQPVKLLIYYTSRLH |
| | SGVPSRFSGSGSGTDFTLTISSLQPEDFATYYCQQGHTLPYTFGQGTKLEIK (SEQ |
| | ID NO: 75) |
| 3M12 | VH: |
| VH4/Vκ2 | QVQLQESGPGLVKPSQTLSLTCTVTGYSITSGYYWNWIRQPPGKGLEWIGYITFD |
| | GANNYNPSLKNRVSISRDTSKNQFSLKLSSVTAEDTATYYCTRSSYDYDVLDYW |
| | GQGTTVTVSS (SEQ ID NO: 76) |
| | VL: |
| | DIQMTQSPSSLSASVGDRVTITCRASQDISNFLNWYQQKPGQPVKLLIYYTSRLH |
| | SGVPSRFSGSGSGTDFTLTISSLQPEDFATYFCQQGHTLPYTFGQGTKLEIK (SEQ |
| | ID NO: 74) |
| 3M12 | VH: |
| VH4/Vκ3 | QVQLQESGPGLVKPSQTLSLTCTVTGYSITSGYYWNWIRQPPGKGLEWIGYITFD |
| | GANNYNPSLKNRVSISRDTSKNQFSLKLSSVTAEDTATYYCTRSSYDYDVLDYW |
| | GQGTTVTVSS (SEQ ID NO: 76) |
| | VL: |
| | DIQMTQSPSSLSASVGDRVTITCRASQDISNFLNWYQQKPGQPVKLLIYYTSRLH |
| | SGVPSRFSGSGSGTDFTLTISSLQPEDFATYYCQQGHTLPYTFGQGTKLEIK (SEQ |
| | ID NO: 75) |
| 5H12 | VH: |
| VH5 (C33Y*)/Vκ3 | QVQLVQSGAEVKKPGASVKVSCKASGYSFTDYYINWVRQAPGQGLEWMGWIY |
| | PGSGNTRYSERFKGRVTITRDTSASTAYMELSSLRSEDTAVYYCAREDYYPYHG |
| | MDYWGQGTLVTVSS (SEQ ID NO: 77) |
| | VL: |
| | DIVLTQSPDSLAVSLGERATINCRASESVDGYDNSFMHWYQQKPGQPPKLLIFRA |
| | SNLESGVPDRFSGSGSRTDFTLTISSLQAEDVAVYYCQQSSEDPWTFGQGTKLEI |
| | K (SEQ ID NO: 78) |
| 5H12 | VH: |
| VH5 (C33D*)/Vκ4 | QVQLVQSGAEVKKPGASVKVSCKASGYSFTDYDINWVRQAPGQGLEWMGWIY |
| | PGSGNTRYSERFKGRVTITRDTSASTAYMELSSLRSEDTAVYYCAREDYYPYHG |
| | MDYWGQGTLVTVSS (SEQ ID NO: 79) |
| | VL: |
| | DIVMTQSPDSLAVSLGERATINCRASESVDGYDNSFMHWYQQKPGQPPKLLIFR |
| | ASNLESGVPDRFSGSGSGTDFTLTISSLQAEDVAVYYCQQSSEDPWTFGQGTKLE |
| | IK (SEQ ID NO: 80) |
| 5H12 | VH: |
| VH5 (C33Y*)/Vκ4 | QVQLVQSGAEVKKPGASVKVSCKASGYSFTDYYINWVRQAPGQGLEWMGWIY |
| | PGSGNTRYSERFKGRVTITRDTSASTAYMELSSLRSEDTAVYYCAREDYYPYHG |
| | MDYWGQGTLVTVSS (SEQ ID NO: 77) |
| | VL: |
| | DIVMTQSPDSLAVSLGERATINCRASESVDGYDNSFMHWYQQKPGQPPKLLIFR |
| | ASNLESGVPDRFSGSGSGTDFTLTISSLQAEDVAVYYCQQSSEDPWTFGQGTKLE |
| | IK (SEQ ID NO: 80) |
| Anti-TfR clone 8 | VH: |
| | QVQLVQSGAEVKKPGESLKISCKGSGYSFTSYWIGWVRQMPGKGLEWMGIIYPG |
| | DSDTRYSPSFQGQVTISADKSISTAYLQWSSLKASDTAMYYCARFPYDSSGYYSF |
| | DYWGQGTLVTVSS (SEQ ID NO: 154) |
| | VL: |
| | DIQMTQSPSSLSASVGDRVTITCRASQSISSYLNWYQQKPGKAPKLLIYAASSLQS |
| | GVPSRFSGSGSGTDFTLTISSLQPEDFATYYCQQSYSTPLTFGGGTKVEIK (SEQ ID |
| | NO: 155) |

*mutation positions are according to Kabat numbering of the respective VH sequences containing the mutations
**CDRs according to the Kabat numbering system are bolded

In some embodiments, the anti-TfR1 antibody of the present disclosure comprises a VH comprising the CDR-H1, CDR-H2, and CDR-H3 of any one of the anti-TfR1 antibodies provided in Table 3 and comprises one or more (e.g., 1, 2, 3, 4, 5, 6, 7, 8, 9, 10 or more) amino acid variations in the framework regions as compared with the respective VH provided in Table 3. Alternatively or in addition (e.g., in addition), the anti-TfR1 antibody of the present disclosure comprises a VL comprising the CDR-L1, CDR-L2, and CDR-L3 of any one of the anti-TfR1 antibodies provided in Table 3 and comprises one or more (e.g., 1, 2, 3, 4, 5, 6, 7, 8, 9, 10 or more) amino acid variations in the framework regions as compared with the respective VL provided in Table 3.

In some embodiments, the anti-TfR1 antibody of the present disclosure comprises a VH comprising the CDR-H1, CDR-H2, and CDR-H3 of any one of the anti-TfR1 antibodies provided in Table 3 and comprising an amino acid sequence that is at least 70% (e.g., at least 70%, at least 75%, at least 80%, at least 85%, at least 90%, at least 95%, at least 99%) identical in the framework regions as compared with the respective VH provided in Table 3. Alternatively or in addition (e.g., in addition), the anti-TfR1 antibody of the present disclosure comprises a VL comprising the CDR-L1, CDR-L2, and CDR-L3 of any one of the anti-TfR1 antibodies provided in Table 3 and comprising an amino acid sequence that is at least 70% (e.g., at least 70%, at least 75%, at least 80%, at least 85%, at least 90%, at least 95%, at least 99%) identical in the framework regions as compared with the respective VL provided in Table 3.

In some embodiments, the anti-TfR1 antibody of the present disclosure comprises a VH comprising the amino acid sequence of SEQ ID NO: 69 and a VL comprising the amino acid sequence of SEQ ID NO: 70.

In some embodiments, the anti-TfR1 antibody of the present disclosure comprises a VH comprising the amino acid sequence of SEQ ID NO: 71 and a VL comprising the amino acid sequence of SEQ ID NO: 70.

In some embodiments, the anti-TfR1 antibody of the present disclosure comprises a VH comprising the amino acid sequence of SEQ ID NO: 72 and a VL comprising the amino acid sequence of SEQ ID NO: 70.

In some embodiments, the anti-TfR1 antibody of the present disclosure comprises a VH comprising the amino acid sequence of SEQ ID NO: 73 and a VL comprising the amino acid sequence of SEQ ID NO: 74.

In some embodiments, the anti-TfR1 antibody of the present disclosure comprises a VH comprising the amino acid sequence of SEQ ID NO: 73 and a VL comprising the amino acid sequence of SEQ ID NO: 75.

In some embodiments, the anti-TfR1 antibody of the present disclosure comprises a VH comprising the amino acid sequence of SEQ ID NO: 76 and a VL comprising the amino acid sequence of SEQ ID NO: 74.

In some embodiments, the anti-TfR1 antibody of the present disclosure comprises a VH comprising the amino acid sequence of SEQ ID NO: 76 and a VL comprising the amino acid sequence of SEQ ID NO: 75.

In some embodiments, the anti-TfR1 antibody of the present disclosure comprises a VH comprising the amino acid sequence of SEQ ID NO: 77 and a VL comprising the amino acid sequence of SEQ ID NO: 78.

In some embodiments, the anti-TfR1 antibody of the present disclosure comprises a VH comprising the amino acid sequence of SEQ ID NO: 79 and a VL comprising the amino acid sequence of SEQ ID NO: 80.

In some embodiments, the anti-TfR1 antibody of the present disclosure comprises a VH comprising the amino acid sequence of SEQ ID NO: 77 and a VL comprising the amino acid sequence of SEQ ID NO: 80.

In some embodiments, the anti-TfR1 antibody of the present disclosure comprises a VH comprising the amino acid sequence of SEQ ID NO: 154 and a VL comprising the amino acid sequence of SEQ ID NO: 155.

In some embodiments, the anti-TfR1 antibody described herein is a full-length IgG, which can include a heavy constant region and a light constant region from a human antibody. In some embodiments, the heavy chain of any of the anti-TfR1 antibodies as described herein may comprise a heavy chain constant region (CH) or a portion thereof (e.g., CH1, CH2, CH3, or a combination thereof). The heavy chain constant region can be of any suitable origin, e.g., human, mouse, rat, or rabbit. In one specific example, the heavy chain constant region is from a human IgG (a gamma heavy chain), e.g., IgG1, IgG2, or IgG4. An example of a human IgG1 constant region is given below:

(SEQ ID NO: 81)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK.

In some embodiments, the heavy chain of any of the anti-TfR1 antibodies described herein comprises a mutant human IgG1 constant region. For example, the introduction of LALA mutations (a mutant derived from mAb b12 that has been mutated to replace the lower hinge residues Leu234 Leu235 with Ala234 and Ala235) in the CH2 domain of human IgG1 is known to reduce Fcγ receptor binding (Bruhns, P., et al. (2009) and Xu, D. et al. (2000)). The mutant human IgG1 constant region is provided below (mutations bonded and underlined):

(SEQ ID NO: 82)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK.

In some embodiments, the light chain of any of the anti-TfR1 antibodies described herein may further comprise a light chain constant region (CL), which can be any CL known in the art. In some examples, the CL is a kappa light chain. In other examples, the CL is a lambda light chain. In some embodiments, the CL is a kappa light chain, the sequence of which is provided below:

(SEQ ID NO: 83)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.

Other antibody heavy and light chain constant regions are well known in the art, e.g., those provided in the IMGT database (www.imgt.org) or at www.vbase2.org/vbstat.php., both of which are incorporated by reference herein.

In some embodiments, the anti-TfR1 antibody described herein comprises a heavy chain comprising any one of the VH as listed in Table 3 or any variants thereof and a heavy chain constant region that is at least 80%, at least 85%, at least 90%, at least 95%, or at least 99% identical to SEQ ID NO: 81 or SEQ ID NO: 82. In some embodiments, the anti-TfR1 antibody described herein comprises a heavy chain comprising any one of the VH as listed in Table 3 or any variants thereof and a heavy chain constant region that contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 81 or SEQ ID NO: 82. In some embodiments, the anti-TfR1 antibody described herein comprises a heavy chain comprising any one of the VH as listed in Table 3 or any variants thereof and a heavy chain constant region as set forth in SEQ ID NO: 81. In some embodiments, the anti-TfR1 antibody described herein comprises heavy chain comprising any one of the VH as listed in Table 3 or any variants thereof and a heavy chain constant region as set forth in SEQ ID NO: 82.

In some embodiments, the anti-TfR1 antibody described herein comprises a light chain comprising any one of the VL as listed in Table 3 or any variants thereof and a light chain constant region that is at least 80%, at least 85%, at least 90%, at least 95%, or at least 99% identical to SEQ ID NO: 83. In some embodiments, the anti-TfR1 antibody described herein comprises a light chain comprising any one of the VL as listed in Table 3 or any variants thereof and a light chain constant region contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 83. In some embodiments, the anti-TfR1 antibody described herein comprises a light chain comprising any one of the VL as listed in Table 3 or any variants thereof and a light chain constant region set forth in SEQ ID NO: 83.

Examples of IgG heavy chain and light chain amino acid sequences of the anti-TfR1 antibodies described are provided in Table 4 below.

TABLE 4
Heavy chain and light chain sequences of examples of anti-TfR1 IgGs

| Antibody | IgG Heavy Chain/Light Chain Sequences** |
|---|---|
| 3A4 | Heavy Chain (with wild type human IgG1 constant region): |
| VH3 (N54T*)/Vκ4 | EVQLVQSGSELKKPGASVKVSCTASGFNIKDDYMYWVRQPPGKGLEWIGWIDPET |
| | GDTEYASKFQDRVTVTADTSTNTAYMELSSLRSEDTAVYYCTLWLRRGLDYWG |
| | QGTLVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDK |
| | THTCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEK |
| | TISKAKGQPREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQKSLSLSPGK |
| | (SEQ ID NO: 84) |
| | Light Chain (with kappa light chain constant region): |
| | DIVMTQSPLSLPVTPGEPASISCRSSKSLLHSNGYTYLFWFQQRPGQSPRLLIYRMS |
| | NLASGVPDRFSGSGSGTDFTLKISRVEAEDVGVYYCMQHLEYPFTFGGGTKVEIKR |
| | TVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC (SEQ ID |
| | NO: 85) |
| 3A4 | Heavy Chain (with wild type human IgG1 constant region): |
| VH3 (N54S*)/Vκ4 | EVQLVQSGSELKKPGASVKVSCTASGFNIKDDYMYWVRQPPGKGLEWIGWIDPES |
| | GDTEYASKFQDRVTVTADTSTNTAYMELSSLRSEDTAVYYCTLWLRRGLDYWG |
| | QGTLVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDK |
| | THTCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEK |
| | TISKAKGQPREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQKSLSLSPGK |
| | (SEQ ID NO: 86) |
| | Light Chain (with kappa light chain constant region): |
| | DIVMTQSPLSLPVTPGEPASISCRSSKSLLHSNGYTYLFWFQQRPGQSPRLLIYRMS |
| | NLASGVPDRFSGSGSGTDFTLKISRVEAEDVGVYYCMQHLEYPFTFGGGTKVEIKR |
| | TVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC (SEQ ID |
| | NO: 85) |
| 3A4 | Heavy Chain (with wild type human IgG1 constant region): |
| VH3/Vκ4 | EVQLVQSGSELKKPGASVKVSCTASGFNIKDDYMYWVRQPPGKGLEWIGWIDPEN |
| | GDTEYASKFQDRVTVTADTSTNTAYMELSSLRSEDTAVYYCTLWLRRGLDYWG |
| | QGTLVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDK |
| | THTCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEK |
| | TISKAKGQPREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQKSLSLSPGK |
| | (SEQ ID NO: 87) |
| | Light Chain (with kappa light chain constant region): |
| | DIVMTQSPLSLPVTPGEPASISCRSSKSLLHSNGYTYLFWFQQRPGQSPRLLIYRMS |
| | NLASGVPDRFSGSGSGTDFTLKISRVEAEDVGVYYCMQHLEYPFTFGGGTKVEIKR |
| | TVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC (SEQ ID |
| | NO: 85) |
| 3M12 | Heavy Chain (with wild type human IgG1 constant region): |
| VH3/Vκ2 | QVQLQESGPGLVKPSQTLSLTCSVTGYSITSGYYWNWIRQPPGKGLEWMGYITFD |
| | GANNYNPSLKNRVSISRDTSKNQFSLKLSSVTAEDTATYYCTRSSYDYDVLDYWG |
| | QGTTVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDK |
| | THTCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEK |
| | TISKAKGQPREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQKSLSLSPGK |
| | (SEQ ID NO: 88) |
| | Light Chain (with kappa light chain constant region): |
| | DIQMTQSPSSLSASVGDRVTITCRASQDISNFLNWYQQKPGQPVKLLIYYTSRLHS |
| | GVPSRFSGSGSGTDFTLTISSLQPEDFATYFCQQGHTLPYTFGQGTKLEIKRTVAAP |
| | SVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSFNRGEC (SEQ ID NO: 89) |
| 3M12 | Heavy Chain (with wild type human IgG1 constant region): |
| VH3/Vκ3 | QVQLQESGPGLVKPSQTLSLTCSVTGYSITSGYYWNWIRQPPGKGLEWMGYITfD |
| | GANNYNPSLKNRVSISRDTSKNQFSLKLSSVTAEDTATYYCTRSSYDYDVLDYWG |
| | QGTTVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDK |
| | THTCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEK |
| | TISKAKGQPREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQKSLSLSPGK |
| | (SEQ ID NO: 88) |
| | Light Chain (with kappa light chain constant region): |
| | DIQMTQSPSSLSASVGDRVTITCRASQDISNFLNWYQQKPGQPVKLLIYYTSRLHS |
| | GVPSRFSGSGSGTDFTLTISSLQPEDFATYYCQQGHTLPYTFGQGTKLEIKRTVAAP |
| | SVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSFNRGEC (SEQ ID NO: 90) |
| 3M12 | Heavy Chain (with wild type human IgG1 constant region): |
| VH4/Vκ2 | QVQLQESGPGLVKPSQTLSLTCTVTGYSITSGYYWNWIRQPPGKGLEWIGYITFDG |
| | ANNYNPSLKNRVSISRDTSKNQFSLKLSSVTAEDTATYYCTRSSYDYDVLDYWGQ |
| | GTTVTVSSASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKKVEPKSCDKT |
| | HTCPPCPAPELLGGPSVFLFPPKPKDTLMISRTPEVTCVVVDVSHEDPEVKFNWYVD |
| | GVEVHNAKTKPREEQYNSTYRVVSVLTVLHQDWLNGKEYKCKVSNKALPAPIEKT |
| | ISKAKGQPREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQKSLSLSPGK |
| | (SEQ ID NO: 91) |
| | Light Chain (with kappa light chain constant region): |
| | DIQMTQSPSSLSASVGDRVTITCRASQDISNFLNWYQQKPGQPVKLLIYYTSRLHS |
| | GVPSRFSGSGSGTDFTLTISSLQPEDFATYFCQQGHTLPYTFGQGTKLEIKRTVAAP |
| | SVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSFNRGEC (SEQ ID NO: 89) |
| 3M12 | Heavy Chain (with wild type human IgG1 constant region): |
| VH4/Vκ3 | QVQLQESGPGLVKPSQTLSLTCTVTGYSITSGYYWNWIRQPPGKGLEWIGYITFDG |
| | ANNYNPSLKNRVSISRDTSKNQFSLKLSSVTAEDTATYYCTRSSYDYDVLDYWGQ |
| | GTTVTVSSASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKKVEPKSCDKT |
| | HTCPPCPAPELLGGPSVFLFPPKPKDTLMISRTPEVTCVVVDVSHEDPEVKFNWYVD |
| | GVEVHNAKTKPREEQYNSTYRVVSVLTVLHQDWLNGKEYKCKVSNKALPAPIEKT |
| | ISKAKGQPREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQKSLSLSPGK |
| | (SEQ ID NO: 91) |
| | Light Chain (with kappa light chain constant region): |
| | DIQMTQSPSSLSASVGDRVTITCRASQDISNFLNWYQQKPGQPVKLLIYYTSRLHS |
| | GVPSRFSGSGSGTDFTLTISSLQPEDFATYYCQQGHTLPYTFGQGTKLEIKRTVAAP |
| | SVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSFNRGEC (SEQ ID NO: 90) |
| 5H12 | Heavy Chain (with wild type human IgG1 constant region): |
| VH5 (C33Y*)/Vκ3 | QVQLVQSGAEVKKPGASVKVSCKASGYSFTDYYINWVRQAPGQGLEWMGWIYP |
| | GSGNTRYSERFKGRVTITRDTSASTAYMELSSLRSEDTAVYYCAREDYYPYHGM |
| | DYWGQGTLVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKVEP |
| | KSCDKTHTCPPCPAPELLGGPSVFLFPPKPKDTLMISRTPEVTCVVVDVSHEDPEVK |
| | FNWYVDGVEVHNAKTKPREEQYNSTYRVVSVLTVLHQDWLNGKEYKCKVSNKA |
| | LPAPIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGK (SEQ ID NO: 92) |
| | Light Chain (with kappa light chain constant region): |
| | DIVLTQSPDSLAVSLGERATINCRASESVDGYDNSFMHWYQQKPGQPPKLLIFRAS |
| | NLESGVPDRFSGSGSRTDFTLTISSLQAEDVAVYYCQQSSEDPWTFGQGTKLEIKR |
| | TVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC (SEQ ID |
| | NO: 93) |
| 5H12 | Heavy Chain (with wild type human IgG1 constant region): |
| VH5 (C33D*)/Vκ4 | QVQLVQSGAEVKKPGASVKVSCKASGYSFTDYDINWVRQAPGQGLEWMGWIYP |
| | GSGNTRYSERFKGRVTITRDTSASTAYMELSSLRSEDTAVYYCAREDYYPYHGM |
| | DYWGQGTLVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKVEP |
| | KSCDKTHTCPPCPAPELLGGPSVFLFPPKPKDTLMISRTPEVTCVVVDVSHEDPEVK |
| | FNWYVDGVEVHNAKTKPREEQYNSTYRVVSVLTVLHQDWLNGKEYKCKVSNKA |
| | LPAPIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGK (SEQ ID NO: 94) |
| | Light Chain (with kappa light chain constant region): |
| | DIVMTQSPDSLAVSLGERATINCRASESVDGYDNSFMHWYQQKPGQPPKLLIFRAS |
| | NLESGVPDRFSGSGSGTDFTLTISSLQAEDVAVYYCQQSSEDPWTFGQGTKLEIKR |
| | TVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC (SEQ ID |
| | NO: 95) |
| 5H12 | Heavy Chain (with wild type human IgG1 constant region): |
| VH5 (C33Y*)/Vκ4 | QVQLVQSGAEVKKPGASVKVSCKASGYSFTDYYINWVRQAPGQGLEWMGWIYP |
| | GSGNTRYSERFKGRVTITRDTSASTAYMELSSLRSEDTAVYYCAREDYYPYHGM |
| | DYWGQGTLVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKVEP |
| | KSCDKTHTCPPCPAPELLGGPSVFLFPPKPKDTLMISRTPEVTCVVVDVSHEDPEVK |
| | FNWYVDGVEVHNAKTKPREEQYNSTYRVVSVLTVLHQDWLNGKEYKCKVSNKA |
| | LPAPIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGK (SEQ ID NO: 92) |
| | Light Chain (with kappa light chain constant region): |
| | DIVMTQSPDSLAVSLGERATINCRASESVDGYDNSFMHWYQQKPGQPPKLLIFRAS |
| | NLESGVPDRFSGSGSGTDFTLTISSLQAEDVAVYYCQQSSEDPWTFGQGTKLEIKR |
| | TVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC (SEQ ID |
| | NO: 95) |
| Anti-TfR clone 8 | Heavy chain (with wild type human IgG1 constant region): |
| | QVQLVQSGAEVKKPGESLKISCKGSGYSFTSYWIGWVRQMPGKGLEWMGIIYPGD |
| | SDTRYSPSFQGQVTISADKSISTAYLQWSSLKASDTAMYYCARFPYDSSGYYSFDY |
| | WGQGTLVTVSSASTKGPSVFPLAPSSKSTSGGTAALGCLVKDYFPEPVTVSWNSGA |
| | LTSGVHTFPAVLQSSGLYSLSSVVTVPSSSLGTQTYICNVNHKPSNTKVDKKVEPKS |
| | CDKTHTCPPCPAPELLGGPSVFLFPPKPKDTLMISRTPEVTCVVVDVSHEDPEVKEN |
| | WYVDGVEVHNAKTKPREEQYNSTYRVVSVLTVLHQDWLNGKEYKCKVSNKALP |
| | APIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGK (SEQ ID NO: 156) |
| | Light chain (with kappa light chain constant region): |
| | DIQMTQSPSSLSASVGDRVTITCRASQSISSYLNWYQQKPGKAPKLLIYAASSLQSG |
| | VPSRFSGSGSGTDFTLTISSLQPEDFATYYCQQSYSTPLTFGGGTKVEIKRTVAAPSV |
| | FIFPPSDEQLKSGTASVVCLLNNFYPREAKVQWKVDNALQSGNSQESVTEQDSKDS |
| | TYSLSSTLTLSKADYEKHKVYACEVTHQGLSSPVTKSFNRGEC (SEQ ID NO: 157) |

*mutation positions are according to Kabat numbering of the respective VH sequences containing the mutations
**CDRs according to the Kabat numbering system are bolded; VH/VL sequences underlined

In some embodiments, the anti-TfR1 antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 84, 86, 87, 88, 91, 92, 94, and 156. Alternatively or in addition (e.g., in addition), the anti-TfR1 antibody of the present disclosure comprises a light chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the light chain as set forth in any one of SEQ ID NOs: 85, 89, 90, 93, 95, and 157.

In some embodiments, the anti-TfR1 antibody described herein comprises a heavy chain comprising an amino acid sequence that is at least 75% (e.g., 75%, 80%, 85%, 90%, 95%, 98%, or 99%) identical to any one of SEQ ID NOs: 84, 86, 87, 88, 91, 92, 94, and 156. Alternatively or in addition (e.g., in addition), the anti-TfR1 antibody described herein comprises a light chain comprising an amino acid sequence that is at least 75% (e.g., 75%, 80%, 85%, 90%, 95%, 98%, or 99%) identical to any one of SEQ ID NOs: 85, 89, 90, 93, 95, and 157. In some embodiments, the anti-TfR1 antibody described herein comprises a heavy chain comprising the amino acid sequence of any one of SEQ ID NOs: 84, 86, 87, 88, 91, 92, 94, and 156. Alternatively or in addition (e.g., in addition), the anti-TfR1 antibody described herein comprises a light chain comprising the amino acid sequence of any one of SEQ ID NOs: 85, 89, 90, 93, 95 and 157.

In some embodiments, the anti-TfR1 antibody of the present disclosure comprises a heavy chain comprising the amino acid sequence of SEQ ID NO: 84 and a light chain comprising the amino acid sequence of SEQ ID NO: 85.

In some embodiments, the anti-TfR1 antibody of the present disclosure comprises a heavy chain comprising the amino acid sequence of SEQ ID NO: 86 and a light chain comprising the amino acid sequence of SEQ ID NO: 85.

In some embodiments, the anti-TfR1 antibody of the present disclosure comprises a heavy chain comprising the amino acid sequence of SEQ ID NO: 87 and a light chain comprising the amino acid sequence of SEQ ID NO: 85.

In some embodiments, the anti-TfR1 antibody of the present disclosure comprises a heavy chain comprising the amino acid sequence of SEQ ID NO: 88 and a light chain comprising the amino acid sequence of SEQ ID NO: 89.

In some embodiments, the anti-TfR1 antibody of the present disclosure comprises a heavy chain comprising the amino acid sequence of SEQ ID NO: 88 and a light chain comprising the amino acid sequence of SEQ ID NO: 90.

In some embodiments, the anti-TfR1 antibody of the present disclosure comprises a heavy chain comprising the amino acid sequence of SEQ ID NO: 91 and a light chain comprising the amino acid sequence of SEQ ID NO: 89.

In some embodiments, the anti-TfR1 antibody of the present disclosure comprises a heavy chain comprising the amino acid sequence of SEQ ID NO: 91 and a light chain comprising the amino acid sequence of SEQ ID NO: 90.

In some embodiments, the anti-TfR1 antibody of the present disclosure comprises a heavy chain comprising the amino acid sequence of SEQ ID NO: 92 and a light chain comprising the amino acid sequence of SEQ ID NO: 93.

In some embodiments, the anti-TfR1 antibody of the present disclosure comprises a heavy chain comprising the amino acid sequence of SEQ ID NO: 94 and a light chain comprising the amino acid sequence of SEQ ID NO: 95.

In some embodiments, the anti-TfR1 antibody of the present disclosure comprises a heavy chain comprising the amino acid sequence of SEQ ID NO: 92 and a light chain comprising the amino acid sequence of SEQ ID NO: 95.

In some embodiments, the anti-TfR1 antibody of the present disclosure comprises a heavy chain comprising the amino acid sequence of SEQ ID NO: 156 and a light chain comprising the amino acid sequence of SEQ ID NO: 157.

In some embodiments, the anti-TfR1 antibody is a Fab fragment, Fab′ fragment, or F(ab′) 2 fragment of an intact antibody (full-length antibody). Antigen binding fragment of an intact antibody (full-length antibody) can be prepared via routine methods (e.g., recombinantly or by digesting the heavy chain constant region of a full length IgG using an enzyme such as papain). For example, F(ab′) 2 fragments can be produced by pepsin or papain digestion of an antibody molecule, and Fab fragments that can be generated by reducing the disulfide bridges of F(ab′) 2 fragments. In some embodiments, a heavy chain constant region in a Fab′ fragment of the anti-TfR1 antibody described herein comprises the amino acid sequence of:
ASTKGPSVFPLAPSSKSTSGGTAALGCLVKDYFPEPVTVSWNSGALTSGVHTFPAVLQSSGLYSLSSVVT VPSSSLGTQTYICNVNHKPSNTKVDKKVEPKSCDKTHT (SEQ ID NO: 96).

In some embodiments, the anti-TfR1 antibody described herein comprises a heavy chain comprising any one of the VH as listed in Table 3 or any variants thereof and a heavy chain constant region that is at least 80%, at least 85%, at least 90%, at least 95%, or at least 99% identical to SEQ ID NO: 96. In some embodiments, the anti-TfR1 antibody described herein comprises a heavy chain comprising any one of the VH as listed in Table 3 or any variants thereof and a heavy chain constant region that contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 96. In some embodiments, the anti-TfR1 antibody described herein comprises a heavy chain comprising any one of the VH as listed in Table 3 or any variants thereof and a heavy chain constant region as set forth in SEQ ID NO: 96.

In some embodiments, the anti-TfR1 antibody described herein comprises a light chain comprising any one of the VL as listed in Table 3 or any variants thereof and a light chain constant region that is at least 80%, at least 85%, at least 90%, at least 95%, or at least 99% identical to SEQ ID NO: 83. In some embodiments, the anti-TfR1 antibody described herein comprises a light chain comprising any one of the VL as listed in Table 3 or any variants thereof and a light chain constant region contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 83. In some embodiments, the anti-TfR1 antibody described herein comprises a light chain comprising any one of the VL as listed in Table 3 or any variants thereof and a light chain constant region set forth in SEQ ID NO: 83.

Examples of Fab heavy chain and light chain amino acid sequences of the anti-TfR1 antibodies described are provided in Table 5 below.

TABLE 5
Heavy chain and light chain sequences of examples of anti-TfR1 Fabs

| Antibody | Fab Heavy Chain/Light Chain Sequences** |
|---|---|
| 3A4 | Heavy Chain (with partial human IgG1 constant region): |
| VH3 (N54T*)/Vκ4 | EVQLVQSGSELKKPGASVKVSCTASGFNIKDDYMYWVRQPPGKGLEWIGWIDPET |
| | GDTEYASKFQDRVTVTADTSTNTAYMELSSLRSEDTAVYYCTLWLRRGLDYWG |
| | QGTLVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDK |
| | THT (SEQ ID NO: 97) |
| | Light Chain (with kappa light chain constant region): |
| | DIVMTQSPLSLPVTPGEPASISCRSSKSLLHSNGYTYLFWFQQRPGQSPRLLIYRMS |
| | NLASGVPDRFSGSGSGTDFTLKISRVEAEDVGVYYCMQHLEYPFTFGGGTKVEIKR |
| | TVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC (SEQ ID |
| | NO: 85) |
| 3A4 | Heavy Chain (with partial human IgG1 constant region): |
| VH3 (N54S*)/Vκ4 | EVQLVQSGSELKKPGASVKVSCTASGFNIKDDYMYWVRQPPGKGLEWIGWIDPES |
| | GDTEYASKFQDRVTVTADTSTNTAYMELSSLRSEDTAVYYCTLWLRRGLDYWG |
| | QGTLVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDK |
| | THT (SEQ ID NO: 98) |
| | Light Chain (with kappa light chain constant region): |
| | DIVMTQSPLSLPVTPGEPASISCRSSKSLLHSNGYTYLFWFQQRPGQSPRLLIYRMS |
| | NLASGVPDRFSGSGSGTDFTLKISRVEAEDVGVYYCMQHLEYPFTFGGGTKVEIKR |
| | TVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC (SEQ ID |
| | NO: 85) |
| 3A4 | Heavy Chain (with partial human IgG1 constant region): |
| VH3/Vκ4 | EVQLVQSGSELKKPGASVKVSCTASGFNIKDDYMYWVRQPPGKGLEWIGWIDPEN |
| | GDTEYASKFQDRVTVTADTSTNTAYMELSSLRSEDTAVYYCTLWLRRGLDYWG |
| | QGTLVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDK |
| | THT (SEQ ID NO: 99) |
| | Light Chain (with kappa light chain constant region): |
| | DIVMTQSPLSLPVTPGEPASISCRSSKSLLHSNGYTYLFWFQQRPGQSPRLLIYRMS |
| | NLASGVPDRFSGSGSGTDFTLKISRVEAEDVGVYYCMQHLEYPFTFGGGTKVEIKR |
| | TVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC (SEQ ID |
| | NO: 85) |
| 3M12 | Heavy Chain (with partial human IgG1 constant region): |
| VH3/Vκ2 | QVQLQESGPGLVKPSQTLSLTCSVTGYSITSGYYWNWIRQPPGKGLEWMGYITFD |
| | GANNYNPSLKNRVSISRDTSKNQFSLKLSSVTAEDTATYYCTRSSYDYDVLDYWG |
| | QGTTVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDK |
| | THT (SEQ ID NO: 100) |
| | Light Chain (with kappa light chain constant region): |
| | DIQMTQSPSSLSASVGDRVTITCRASQDISNFLNWYQQKPGQPVKLLIYYTSRLHS |
| | GVPSRFSGSGSGTDFTLTISSLQPEDFATYFCQQGHTLPYTFGQGTKLEIKRTVAAP |
| | SVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSFNRGEC (SEQ ID NO: 89) |
| 3M12 | Heavy Chain (with partial human IgG1 constant region): |
| VH3/Vκ3 | QVQLQESGPGLVKPSQTLSLTCSVTGYSITSGYYWNWIRQPPGKGLEWMGYITFD |
| | GANNYNPSLKNRVSISRDTSKNQFSLKLSSVTAEDTATYYCTRSSYDYDVLDYWG |
| | QGTTVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDK |
| | THT (SEQ ID NO: 100) |
| | Light Chain (with kappa light chain constant region): |
| | DIQMTQSPSSLSASVGDRVTITCRASQDISNFLNWYQQKPGQPVKLLIYYTSRLHS |
| | GVPSRFSGSGSGTDFTLTISSLQPEDFATYYCQQGHTLPYTFGQGTKLEIKRTVAAP |
| | SVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSFNRGEC (SEQ ID NO: 90) |
| 3M12 | Heavy Chain (with partial human IgG1 constant region): |
| VH4/Vκ2 | QVQLQESGPGLVKPSQTLSLTCTVTGYSITSGYYWNWIRQPPGKGLEWIGYITFDG |
| | ANNYNPSLKNRVSISRDTSKNQFSLKLSSVTAEDTATYYCTRSSYDYDVLDYWGQ |
| | GTTVTVSSASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKKVEPKSCDKT |
| | HT (SEQ ID NO: 101) |
| | Light Chain (with kappa light chain constant region): |
| | DIQMTQSPSSLSASVGDRVTITCRASQDISNFLNWYQQKPGQPVKLLIYYTSRLHS |
| | GVPSRFSGSGSGTDFTLTISSLQPEDFATYFCQQGHTLPYTFGQGTKLEIKRTVAAP |
| | SVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSFNRGEC (SEQ ID NO: 89) |
| 3M12 | Heavy Chain (with partial human IgG1 constant region): |
| VH4/Vκ3 | QVQLQESGPGLVKPSQTLSLTCTVTGYSITSGYYWNWIRQPPGKGLEWIGYITFDG |
| | ANNYNPSLKNRVSISRDTSKNQFSLKLSSVTAEDTATYYCTRSSYDYDVLDYWGQ |
| | GTTVTVSSASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKKVEPKSCDKT |
| | HT (SEQ ID NO: 101) |
| | Light Chain (with kappa light chain constant region): |
| | DIQMTQSPSSLSASVGDRVTITCRASQDISNFLNWYQQKPGQPVKLLIYYTSRLHS |
| | GVPSRFSGSGSGTDFTLTISSLQPEDFATYYCQQGHTLPYTFGQGTKLEIKRTVAAP |
| | SVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSFNRGEC (SEQ ID NO: 90) |
| 5H12 | Heavy Chain (with partial human IgG1 constant region): |
| VH5 (C33Y*)/Vκ3 | QVQLVQSGAEVKKPGASVKVSCKASGYSFTDYYINWVRQAPGQGLEWMGWIYP |
| | GSGNTRYSERFKGRVTITRDTSASTAYMELSSLRSEDTAVYYCAREDYYPYHGM |
| | DYWGQGTLVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKVEP |
| | KSCDKTHT (SEQ ID NO: 102) |
| | Light Chain (with kappa light chain constant region): |
| | DIVLTQSPDSLAVSLGERATINCRASESVDGYDNSFMHWYQQKPGQPPKLLIFRAS |
| | NLESGVPDRFSGSGSRTDFTLTISSLQAEDVAVYYCQQSSEDPWTFGQGTKLEIKR |
| | TVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC (SEQ ID |
| | NO: 93) |
| 5H12 | Heavy Chain (with partial human IgG1 constant region): |
| VH5 (C33D*)/Vκ4 | QVQLVQSGAEVKKPGASVKVSCKASGYSFTDYDINWVRQAPGQGLEWMGWIYP |
| | GSGNTRYSERFKGRVTITRDTSASTAYMELSSLRSEDTAVYYCAREDYYPYHGM |
| | DYWGQGTLVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKVEP |
| | KSCDKTHT (SEQ ID NO: 103) |
| | Light Chain (with kappa light chain constant region): |
| | DIVMTQSPDSLAVSLGERATINCRASESVDGYDNSFMHWYQQKPGQPPKLLIFRAS |
| | NLESGVPDRFSGSGSGTDFTLTISSLQAEDVAVYYCQQSSEDPWTFGQGTKLEIKR |
| | TVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC (SEQ ID |
| | NO: 95) |
| 5H12 | Heavy Chain (with partial human IgG1 constant region): |
| VH5 (C33Y*)/Vκ4 | QVQLVQSGAEVKKPGASVKVSCKASGYSFTDYYINWVRQAPGQGLEWMGWIYP |
| | GSGNTRYSERFKGRVTITRDTSASTAYMELSSLRSEDTAVYYCAREDYYPYHGM |
| | DYWGQGTLVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKVEP |
| | KSCDKTHT (SEQ ID NO: 102) |
| | Light Chain (with kappa light chain constant region): |
| | DIVMTQSPDSLAVSLGERATINCRASESVDGYDNSFMHWYQQKPGQPPKLLIFRAS |
| | NLESGVPDRFSGSGSGTDFTLTISSLQAEDVAVYYCQQSSEDPWTFGQGTKLEIKR |
| | TVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC (SEQ ID |
| | NO: 95) |
| Anti-TfR clone 8 | Heavy chain (with partial human IgG1 constant region): |
| Version 1 | QVQLVQSGAEVKKPGESLKISCKGSGYSFTSYWIGWVRQMPGKGLEWMGIIYPGD |
| | SDTRYSPSFQGQVTISADKSISTAYLQWSSLKASDTAMYYCARFPYDSSGYYSFDY |
| | WGQGTLVTVSSASTKGPSVFPLAPSSKSTSGGTAALGCLVKDYFPEPVTVSWNSGA |
| | LTSGVHTFPAVLQSSGLYSLSSVVTVPSSSLGTQTYICNVNHKPSNTKVDKKVEPKS |
| | CDKTHTCP (SEQ ID NO: 158) |
| | Light chain (with kappa light chain constant region): |
| | DIQMTQSPSSLSASVGDRVTITCRASQSISSYLNWYQQKPGKAPKLLIYAASSLQSG |
| | VPSRFSGSGSGTDFTLTISSLQPEDFATYYCQQSYSTPLTFGGGTKVEIKRTVAAPSV |
| | FIFPPSDEQLKSGTASVVCLLNNFYPREAKVQWKVDNALQSGNSQESVTEQDSKDS |
| | TYSLSSTLTLSKADYEKHKVYACEVTHQGLSSPVTKSFNRGEC (SEQ ID NO: 157) |
| Anti-TfR clone 8 | Heavy chain (with partial human IgG1 constant region): |
| Version 2 | QVQLVQSGAEVKKPGESLKISCKGSGYSFTSYWIGWVRQMPGKGLEWMGIIYPGD |
| | SDTRYSPSFQGQVTISADKSISTAYLQWSSLKASDTAMYYCARFPYDSSGYYSFDY |
| | WGQGTLVTVSSASTKGPSVFPLAPSSKSTSGGTAALGCLVKDYFPEPVTVSWNSGA |
| | LTSGVHTFPAVLQSSGLYSLSSVVTVPSSSLGTQTYICNVNHKPSNTKVDKKVEPKS |
| | CDKTHT (SEQ ID NO: 159) |
| | Light chain (with kappa light chain constant region): |
| | DIQMTQSPSSLSASVGDRVTITCRASQSISSYLNWYQQKPGKAPKLLIYAASSLQSG |
| | VPSRFSGSGSGTDFTLTISSLQPEDFATYYCQQSYSTPLTFGGGTKVEIKRTVAAPSV |
| | FIFPPSDEQLKSGTASVVCLLNNFYPREAKVQWKVDNALQSGNSQESVTEQDSKDS |
| | TYSLSSTLTLSKADYEKHKVYACEVTHQGLSSPVTKSFNRGEC (SEQ ID NO: 157) |

*mutation positions are according to Kabat numbering of the respective VH sequences containing the mutations
**CDRs according to the Kabat numbering system are bolded; VH/VL sequences underlined

In some embodiments, the anti-TfR1 antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 97-103, 158 and 159. Alternatively or in addition (e.g., in addition), the anti-TfR1 antibody of the present disclosure comprises a light chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the light chain as set forth in any one of SEQ ID NOs: 85, 89, 90, 93, 95, and 157.

In some embodiments, the anti-TfR1 antibody described herein comprises a heavy chain comprising an amino acid sequence that is at least 75% (e.g., 75%, 80%, 85%, 90%, 95%, 98%, or 99%) identical to any one of SEQ ID NOs: 97-103, 158 and 159. Alternatively or in addition (e.g., in addition), the anti-TfR1 antibody described herein comprises a light chain comprising an amino acid sequence that is at least 75% (e.g., 75%, 80%, 85%, 90%, 95%, 98%, or 99%) identical to any one of SEQ ID NOs: 85, 89, 90, 93, 95, and 157. In some embodiments, the anti-TfR1 antibody described herein comprises a heavy chain comprising the amino acid sequence of any one of SEQ ID NOs: 97-103, 158 and 159. Alternatively or in addition (e.g., in addition), the anti-TfR1 antibody described herein comprises a light chain comprising the amino acid sequence of any one of SEQ ID NOs: 85, 89, 90, 93, 95, and 157.

In some embodiments, the anti-TfR1 antibody of the present disclosure comprises a heavy chain comprising the amino acid sequence of SEQ ID NO: 97 and a light chain comprising the amino acid sequence of SEQ ID NO: 85.

In some embodiments, the anti-TfR1 antibody of the present disclosure comprises a heavy chain comprising the amino acid sequence of SEQ ID NO: 98 and a light chain comprising the amino acid sequence of SEQ ID NO: 85.

In some embodiments, the anti-TfR1 antibody of the present disclosure comprises a heavy chain comprising the amino acid sequence of SEQ ID NO: 99 and a light chain comprising the amino acid sequence of SEQ ID NO: 85.

In some embodiments, the anti-TfR1 antibody of the present disclosure comprises a heavy chain comprising the amino acid sequence of SEQ ID NO: 100 and a light chain comprising the amino acid sequence of SEQ ID NO: 89.

In some embodiments, the anti-TfR1 antibody of the present disclosure comprises a heavy chain comprising the amino acid sequence of SEQ ID NO: 100 and a light chain comprising the amino acid sequence of SEQ ID NO: 90.

In some embodiments, the anti-TfR1 antibody of the present disclosure comprises a heavy chain comprising the amino acid sequence of SEQ ID NO: 101 and a light chain comprising the amino acid sequence of SEQ ID NO: 89.

In some embodiments, the anti-TfR1 antibody of the present disclosure comprises a heavy chain comprising the amino acid sequence of SEQ ID NO: 101 and a light chain comprising the amino acid sequence of SEQ ID NO: 90.

In some embodiments, the anti-TfR1 antibody of the present disclosure comprises a heavy chain comprising the amino acid sequence of SEQ ID NO: 102 and a light chain comprising the amino acid sequence of SEQ ID NO: 93.

In some embodiments, the anti-TfR1 antibody of the present disclosure comprises a heavy chain comprising the amino acid sequence of SEQ ID NO: 103 and a light chain comprising the amino acid sequence of SEQ ID NO: 95.

In some embodiments, the anti-TfR1 antibody of the present disclosure comprises a heavy chain comprising the amino acid sequence of SEQ ID NO: 102 and a light chain comprising the amino acid sequence of SEQ ID NO: 95.

In some embodiments, the anti-TfR1 antibody of the present disclosure comprises a heavy chain comprising the amino acid sequence of SEQ ID NO: 158 and a light chain comprising the amino acid sequence of SEQ ID NO: 157.

In some embodiments, the anti-TfR1 antibody of the present disclosure comprises a heavy chain comprising the amino acid sequence of SEQ ID NO: 159 and a light chain comprising the amino acid sequence of SEQ ID NO: 157.

### Other Known Anti-TfR1 Antibodies

Any other appropriate anti-TfR1 antibodies known in the art may be used as the muscle-targeting agent in the complexes disclosed herein. Examples of known anti-TfR1 antibodies, including associated references and binding epitopes, are listed in Table 6. In some embodiments, the anti-TfR1 antibody comprises the complementarity determining regions (CDR-H1, CDR-H2, CDR-H3, CDR-L1, CDR-L2, and CDR-L3) of any of the anti-TfR1 antibodies provided herein, e.g., anti-TfR1 antibodies listed in Table 6.

TABLE 6
List of anti-TfR1 antibody clones, including associated references and
binding epitope information.

| Antibody | | |
|---|---|---|
| Clone Name | Reference(s) | Epitope/Notes |
| OKT9 | U.S. Pat. No. 4,364,934, filed Dec. 04, 1979, | Apical domain of |
| | entitled “MONOCLONAL ANTIBODY | TfR1 (residues 305- |
| | TO A HUMAN EARLY THYMOCYTE | 366 of human TfR1 |
| | ANTIGEN AND METHODS FOR | sequence |
| | PREPARING SAME” | XM_052730.3, |
| | Schneider C. et al. “Structural features of | available in GenBank) |
| | the cell surface receptor for transferrin that | |
| | is recognized by the monoclonal antibody | |
| | OKT9.” J Biol Chem. 1982, 257: 14, 8516- | |
| | 8522. | |
| (From JCR) | WO 2015/098989, filed | Apical domain |
| Clone M11 | Dec. 24, 2014, “Novel anti-Transferrin | (residues 230-244 and |
| Clone M23 | receptor antibody that passes through | 326-347 of TfR1) and |
| Clone M27 | blood-brain barrier” | protease-like domain |
| Clone B84 | U.S. Pat. No. 9,994,641, filed | (residues 461-473) |
| | Dec. 24, 2014, “Novel anti-Transferrin | |
| | receptor antibody that passes through | |
| | blood-brain barrier” | |
| (From | WO 2016/081643, filed May 26, 2016, | Apical domain and |
| Genentech) | entitled “ANTI-TRANSFERRIN | non-apical regions |
| 7A4, 8A2, | RECEPTOR ANTIBODIES AND | |
| 15D2, 10D11, | METHODS OF USE” | |
| 7B10, 15G11, | U.S. Pat. No. 9,708,406, filed | |
| 16G5, 13C3, | May 20, 2014, “Anti-transferrin receptor | |
| 16G4, 16F6, | antibodies and methods of use” | |
| 7G7, 4C2, | | |
| 1B12, and | | |
| 13D4 | | |
| (From | Lee et al. “Targeting Rat Anti- | |
| Armagen) | Mouse Transferrin Receptor Monoclonal | |
| 8D3 | Antibodies through Blood-Brain Barrier in | |
| | Mouse” 2000, J Pharmacol. Exp. Ther., | |
| | 292: 1048-1052. | |
| | U.S. patent application 2010/077498, filed | |
| | Sep. 11, 2008, entitled “COMPOSITIONS | |
| | AND METHODS FOR BLOOD-BRAIN | |
| | BARRIER DELIVERY IN THE MOUSE” | |
| OX26 | Haobam, B. et al. 2014. Rab17- | |
| | mediated recycling endosomes contribute | |
| | to autophagosome formation in response to | |
| | Group A Streptococcus invasion. Cellular | |
| | microbiology. 16: 1806-21. | |
| DF1513 | Ortiz-Zapater E et al. Trafficking | |
| | of the human transferrin receptor in plant | |
| | cells: effects of tyrphostin A23 and | |
| | brefeldin A. Plant J 48: 757-70 (2006). | |
| 1A1B2, | Commercially available anti- | Novus Biologicals |
| 66IG10, | transferrin receptor antibodies. | 8100 Southpark Way, |
| MEM-189, | | A-8 Littleton CO |
| JF0956, 29806, | | 80120 |
| 1A1B2, | | |
| TFRC/1818, | | |
| 1E6, 66Ig10, | | |
| TFRC/1059, | | |
| Q1/71, 23D10, | | |
| 13E4, | | |
| TFRC/1149, | | |
| ER-MP21, | | |
| YTA74.4, | | |
| BU54, 2B6, | | |
| RI7 217 | | |
| (From | U.S. patent application 2011/0311544A1, | Does not compete |
| INSERM) | filed Jun. 15, 2005, entitled “ANTI-CD71 | with OKT9 |
| BA120g | MONOCLONAL ANTIBODIES AND | |
| | USES THEREOF FOR TREATING | |
| | MALIGNANT TUMOR CELLS” | |
| LUCA31 | U.S. Pat. No. 7,572,895, filed | “LUCA31 epitope” |
| | Jun. 07, 2004, entitled “TRANSFERRIN | |
| | RECEPTOR ANTIBODIES” | |
| (Salk Institute) | Trowbridge, I. S. et al. “Anti-transferrin | |
| B3/25 | receptor monoclonal antibody and | |
| T58/30 | toxin-antibody conjugates affect | |
| | growth of human tumour cells.” | |
| | Nature, 1981, volume 294, pages 171- | |
| | 173 | |
| R17 217.1.3, | Commercially available anti- | BioXcell |
| 5E9C11, | transferrin receptor antibodies. | 10 Technology Dr., |
| OKT9 | | Suite 2B |
| (BE0023 | | West Lebanon, NH |
| clone) | | 03784-1671 USA |
| BK19.9, | Gatter, K. C. et al. “Transferrin | |
| B3/25, T56/14 | receptors in human tissues: their | |
| and T58/1 | distribution and possible clinical | |
| | relevance.” J Clin Pathol. 1983 | |
| | May; 36(5): 539-45. | |

In some embodiments, anti-TfR1 antibodies of the present disclosure include one or more of the CDR-H (e.g., CDR-H1, CDR-H2, and CDR-H3) amino acid sequences from any one of the anti-TfR1 antibodies selected from Table 6. In some embodiments, anti-TfR1 antibodies include the CDR-L1, CDR-L2, and CDR-L3 as provided for any one of the anti-TfR1 antibodies selected from Table 6. In some embodiments, anti-TfR1 antibodies include the CDR-H1, CDR-H2, CDR-H3, CDR-L1, CDR-L2, and CDR-L3 as provided for any one of the anti-TfR1 antibodies selected from Table 6.

In some embodiments, anti-TfR1 antibodies of the disclosure include any antibody that includes a heavy chain variable domain and/or (e.g., and) a light chain variable domain of any anti-TfR1 antibody, such as any one of the anti-TfR1 antibodies selected from Table 6. In some embodiments, anti-TfR1 antibodies of the disclosure include any antibody that includes the heavy chain variable and light chain variable pairs of any anti-TfR1 antibody, such as any one of the anti-TfR1 antibodies selected from Table 6.

Aspects of the disclosure provide anti-TfR1 antibodies having a heavy chain variable (VH) and/or (e.g., and) a light chain variable (VL) domain amino acid sequence homologous to any of those described herein. In some embodiments, the anti-TfR1 antibody comprises a heavy chain variable sequence or a light chain variable sequence that is at least 75% (e.g., 80%, 85%, 90%, 95%, 98%, or 99%) identical to the heavy chain variable sequence and/or any light chain variable sequence of any anti-TfR1 antibody, such as any one of the anti-TfR1 antibodies selected from Table 6. In some embodiments, the homologous heavy chain variable and/or (e.g., and) a light chain variable amino acid sequences do not vary within any of the CDR sequences provided herein. For example, in some embodiments, the degree of sequence variation (e.g., 75%, 80%, 85%, 90%, 95%, 98%, or 99%) may occur within a heavy chain variable and/or (e.g., and) a light chain variable sequence excluding any of the CDR sequences provided herein. In some embodiments, any of the anti-TfR1 antibodies provided herein comprise a heavy chain variable sequence and a light chain variable sequence that comprises a framework sequence that is at least 75%, 80%, 85%, 90%, 95%, 98%, or 99% identical to the framework sequence of any anti-TfR1 antibody, such as any one of the anti-TfR1 antibodies selected from Table 6.

An example of a transferrin receptor antibody that may be used in accordance with the present disclosure is described in International Application Publication WO 2016/081643, incorporated herein by reference. The amino acid sequences of this antibody are provided in Table 7.

TABLE 7
Heavy chain and light chain CDRs of an example of a known anti-TfR1 antibody

| Sequence Type | Kabat | Chothia | Contact |
|---|---|---|---|
| CDR-H1 | SYWMH (SEQ ID | GYTFTSY (SEQ ID | TSYWMH (SEQ ID NO: 118) |
| | NO: 110) | NO: 116) | |
| CDR-H2 | EINPTNGRTNYIE | NPTNGR (SEQ ID | WIGEINPTNGRTN (SEQ ID |
| | KFKS (SEQ ID | NO: 117) | NO: 119) |
| | NO: 111) | | |
| CDR-H3 | GTRAYHY (SEQ | GTRAYHY (SEQ ID NO: | ARGTRA |
| | ID NO: 112) | 112) | (SEQ ID NO: 120) |
| CDR-L1 | RASDNLYSNLA | RASDNLYSNLA (SEQ ID | YSNLAWY (SEQ ID NO: 121) |
| | (SEQ ID NO: 113) | NO: 113) | |
| CDR-L2 | DATNLAD (SEQ | DATNLAD (SEQ ID NO: | LLVYDATNLA (SEQ ID NO: |
| | ID NO: 114) | 114) | 122) |
| CDR-L3 | QHFWGTPLT | QHFWGTPLT (SEQ ID | QHFWGTPL (SEQ ID NO: |
| | (SEQ ID NO: 115) | NO: 115) | 123) |

| | |
|---|---|
| Murine VH | QVQLQQPGAELVKPGASVKLSCKASGYTFTSYWMHWVKQRPGQGLEWIGEINP |
| | TNGRTNYIEKFKSKATLTVDKSSSTAYMQLSSLTSEDSAVYYCARGTRAYHYW |
| | GQGTSVTVSS (SEQ ID NO: 124) |
| Murine VL | DIQMTQSPASLSVSVGETVTITCRASDNLYSNLAWYQQKQGKSPQLLVYDATNL |
| | ADGVPSRFSGSGSGTQYSLKINSLQSEDFGTYYCQHFWGTPLTFGAGTKLELK |
| | (SEQ ID NO: 125) |
| Humanized VH | EVQLVQSGAEVKKPGASVKVSCKASGYTFTSYWMHWVRQAPGQRLEWIGEIN |
| | PTNGRTNYIEKFKSRATLTVDKSASTAYMELSSLRSEDTAVYYCARGTRAYHY |
| | WGQGTMVTVSS (SEQ ID NO: 128) |
| Humanized VL | DIQMTQSPSSLSASVGDRVTITCRASDNLYSNLAWYQQKPGKSPKLLVYDATNL |
| | ADGVPSRFSGSGSGTDYTLTISSLQPEDFATYYCQHFWGTPLTFGQGTKVEIK |
| | (SEQ ID NO: 129) |
| HC of chimeric | QVQLQQPGAELVKPGASVKLSCKASGYTFTSYWMHWVKQRPGQGLEWIGEINP |
| full-length IgG1 | TNGRTNYIEKFKSKATLTVDKSSSTAYMQLSSLTSEDSAVYYCARGTRAYHYW |
| | GQGTSVTVSSASTKGPSVFPLAPSSKSTSGGTAALGCLVKDYFPEPVTVSWNSG |
| | ALTSGVHTFPAVLQSSGLYSLSSVVTVPSSSLGTQTYICNVNHKPSNTKVDKKVE |
| | PKSCDKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGFYPSDIAV |
| | EWESNGQPENNYKTTPPVLDSDGSFFLYSKLTVDKSRWQQGNVFSCSVMHEAL |
| | HNHYTQKSLSLSPGK (SEQ ID NO: 132) |
| LC of chimeric | DIQMTQSPASLSVSVGETVTITCRASDNLYSNLAWYQQKQGKSPQLLVYDATNL |
| full-length IgG1 | ADGVPSRFSGSGSGTQYSLKINSLQSEDFGTYYCQHFWGTPLTFGAGTKLELKR |
| | TVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| | (SEQ ID NO: 133) |
| HC of fully human | EVQLVQSGAEVKKPGASVKVSCKASGYTFTSYWMHWVRQAPGQRLEWIGEIN |
| full-length IgG1 | PTNGRTNYIEKFKSRATLTVDKSASTAYMELSSLRSEDTAVYYCARGTRAYHY |
| | WGQGTMVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKV |
| | EPKSCDKTHTCPPCPAPELLGGPSVFLFPPKPKDTLMISRTPEVTCVVVDVSHEDP |
| | EVKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGFYPSDIA |
| | VEWESNGQPENNYKTTPPVLDSDGSFFLYSKLTVDKSRWQQGNVFSCSVMHEA |
| | LHNHYTQKSLSLSPGK (SEQ ID NO: 134) |
| LC of fully human | DIQMTQSPSSLSASVGDRVTITCRASDNLYSNLAWYQQKPGKSPKLLVYDATNL |
| full-length IgG1 | ADGVPSRFSGSGSGTDYTLTISSLQPEDFATYYCQHFWGTPLTFGQGTKVEIKRT |
| | VAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| | (SEQ ID NO: 135) |
| HC of chimeric | QVQLQQPGAELVKPGASVKLSCKASGYTFTSYWMHWVKQRPGQGLEWIGEINP |
| Fab | TNGRTNYIEKFKSKATLTVDKSSSTAYMQLSSLTSEDSAVYYCARGTRAYHYW |
| | GQGTSVTVSSASTKGPSVFPLAPSSKSTSGGTAALGCLVKDYFPEPVTVSWNSG |
| | ALTSGVHTFPAVLQSSGLYSLSSVVTVPSSSLGTQTYICNVNHKPSNTKVDKKVE |
| | PKSCDKTHTCP (SEQ ID NO: 136) |
| HC of fully human | EVQLVQSGAEVKKPGASVKVSCKASGYTFTSYWMHWVRQAPGQRLEWIGEIN |
| Fab | PTNGRTNYIEKFKSRATLTVDKSASTAYMELSSLRSEDTAVYYCARGTRAYHY |
| | WGQGTMVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKV |
| | EPKSCDKTHTCP (SEQ ID NO: 137) |

In some embodiments, the anti-TfR1 antibody of the present disclosure comprises a CDR-H1, a CDR-H2, and a CDR-H3 that are the same as the CDR-H1, CDR-H2, and CDR-H3 shown in Table 7. Alternatively or in addition (e.g., in addition), the anti-TfR1 antibody of the present disclosure comprises a CDR-L1, a CDR-L2, and a CDR-L3 that are the same as the CDR-L1, CDR-L2, and CDR-L3 shown in Table 7.

In some embodiments, the anti-TfR1 antibody of the present disclosure comprises a CDR-L3, which contains no more than 3 amino acid variations (e.g., no more than 3, 2, or 1 amino acid variation) as compared with the CDR-L3 as shown in Table 7. In some embodiments, the anti-TfR1 antibody of the present disclosure comprises a CDR-L3 containing one amino acid variation as compared with the CDR-L3 as shown in Table 7. In some embodiments, the anti-TfR1 antibody of the present disclosure comprises a CDR-L3 of QHFAGTPLT (SEQ ID NO: 126) according to the Kabat and Chothia definition system) or QHFAGTPL (SEQ ID NO: 127) according to the Contact definition system). In some embodiments, the anti-TfR1 antibody of the present disclosure comprises a CDR-H1, a CDR-H2, a CDR-H3, a CDR-L1 and a CDR-L2 that are the same as the CDR-H1, CDR-H2, and CDR-H3 shown in Table 7, and comprises a CDR-L3 of QHFAGTPLT (SEQ ID NO: 126) according to the Kabat and Chothia definition system) or QHFAGTPL (SEQ ID NO: 127) according to the Contact definition system).

In some embodiments, the anti-TfR1 antibody of the present disclosure comprises heavy chain CDRs that collectively are at least 80% (e.g., 80%, 85%, 90%, 95%, or 98%) identical to the heavy chain CDRs as shown in Table 7. Alternatively or in addition (e.g., in addition), the anti-TfR1 antibody of the present disclosure comprises light chain CDRs that collectively are at least 80% (e.g., 80%, 85%, 90%, 95%, or 98%) identical to the light chain CDRs as shown in Table 7.

In some embodiments, the anti-TfR1 antibody of the present disclosure comprises a VH comprising the amino acid sequence of SEQ ID NO: 124. Alternatively or in addition (e.g., in addition), the anti-TfR1 antibody of the present disclosure comprises a VL comprising the amino acid sequence of SEQ ID NO: 125.

In some embodiments, the anti-TfR1 antibody of the present disclosure comprises a VH comprising the amino acid sequence of SEQ ID NO: 128. Alternatively or in addition (e.g., in addition), the anti-TfR1 antibody of the present disclosure comprises a VL comprising the amino acid sequence of SEQ ID NO: 129.

In some embodiments, the anti-TfR1 antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 128. Alternatively or in addition (e.g., in addition), the anti-TfR1 antibody of the present disclosure comprises a VL containing no more than 15 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 129.

In some embodiments, the anti-TfR1 antibody of the present disclosure is a full-length IgG1 antibody, which can include a heavy constant region and a light constant region from a human antibody. In some embodiments, the heavy chain of any of the anti-TfR1 antibodies as described herein may comprises a heavy chain constant region (CH) or a portion thereof (e.g., CH1, CH2, CH3, or a combination thereof). The heavy chain constant region can of any suitable origin, e.g., human, mouse, rat, or rabbit. In one specific example, the heavy chain constant region is from a human IgG (a gamma heavy chain), e.g., IgG1, IgG2, or IgG4. An example of human IgG1 constant region is given below:

(SEQ ID NO: 81)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

In some embodiments, the light chain of any of the anti-TfR1 antibodies described herein may further comprise a light chain constant region (CL), which can be any CL known in the art. In some examples, the CL is a kappa light chain. In other examples, the CL is a lambda light chain. In some embodiments, the CL is a kappa light chain, the sequence of which is provided below:

(SEQ ID NO: 83)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

In some embodiments, the anti-TfR1 antibody described herein is a chimeric antibody that comprises a heavy chain comprising the amino acid sequence of SEQ ID NO: 132. Alternatively or in addition (e.g., in addition), the anti-TfR1 antibody described herein comprises a light chain comprising the amino acid sequence of SEQ ID NO: 133.

In some embodiments, the anti-TfR1 antibody described herein is a fully human antibody that comprises a heavy chain comprising the amino acid sequence of SEQ ID NO: 134. Alternatively or in addition (e.g., in addition), the anti-TfR1 antibody described herein comprises a light chain comprising the amino acid sequence of SEQ ID NO: 135.

In some embodiments, the anti-TfR1 antibody is an antigen binding fragment (Fab) of an intact antibody (full-length antibody). In some embodiments, the anti-TfR1 Fab described herein comprises a heavy chain comprising the amino acid sequence of SEQ ID NO: 136. Alternatively or in addition (e.g., in addition), the anti-TfR1 Fab described herein comprises a light chain comprising the amino acid sequence of SEQ ID NO: 133. In some embodiments, the anti-TfR1 Fab described herein comprises a heavy chain comprising the amino acid sequence of SEQ ID NO: 137. Alternatively or in addition (e.g., in addition), the anti-TfR1 Fab described herein comprises a light chain comprising the amino acid sequence of SEQ ID NO: 135.

The anti-TfR1 antibodies described herein can be in any antibody form, including, but not limited to, intact (i.e., full-length) antibodies, antigen-binding fragments thereof (such as Fab, Fab′, F(ab′) 2, Fv), single chain antibodies, bi-specific antibodies, or nanobodies. In some embodiments, the anti-TfR1 antibody described herein is a scFv. In some embodiments, the anti-TfR1 antibody described herein is a scFv-Fab (e.g., scFv fused to a portion of a constant region). In some embodiments, the anti-TfR1 antibody described herein is an scFv fused to a constant region (e.g., human IgG1 constant region as set forth in SEQ ID NO: 81).

In some embodiments, conservative mutations can be introduced into antibody sequences (e.g., CDRs or framework sequences) at positions where the residues are not likely to be involved in interacting with a target antigen (e.g., transferrin receptor), for example, as determined based on a crystal structure. In some embodiments, one, two or more mutations (e.g., amino acid substitutions) are introduced into the Fc region of an anti-TfR1 antibody described herein (e.g., in a CH2 domain (residues 231-340 of human IgG1) and/or (e.g., and) CH3 domain (residues 341-447 of human IgG1) and/or (e.g., and) the hinge region, with numbering according to the Kabat numbering system (e.g., the EU index in Kabat)) to alter one or more functional properties of the antibody, such as serum half-life, complement fixation, Fc receptor binding and/or (e.g., and) antigen-dependent cellular cytotoxicity.

In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more mutations (e.g., amino acid substitutions) are introduced into the Fc region of a muscle-targeting antibody described herein (e.g., in a CH2 domain (residues 231-340 of human IgG1) and/or (e.g., and) CH3 domain (residues 341-447 of human IgG1) and/or (e.g., and) the hinge region, with numbering according to the Kabat numbering system (e.g., the EU index in Kabat)) to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109:6181-6186, U.S. Pat. No. 6,737,056, and International Publication Nos. WO 02/060919; WO 98/23289; and WO 97/34631, which are incorporated herein by reference.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the anti-TfR1 antibody in vivo. See, e.g., International Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to decrease the half-life of the anti-TfR1 antibody in vivo. In some embodiments, one, two or more amino acid mutations (i.e., substitutions, insertions or deletions) are introduced into an IgG constant domain, or FcRn-binding fragment thereof (preferably an Fc or hinge-Fc domain fragment) to increase the half-life of the antibody in vivo. In some embodiments, the antibodies can have one or more amino acid mutations (e.g., substitutions) in the second constant (CH2) domain (residues 231-340 of human IgG1) and/or (e.g., and) the third constant (CH3) domain (residues 341-447 of human IgG1), with numbering according to the EU index in Kabat (Kabat E A et al., (1991) supra). In some embodiments, the constant region of the IgG1 of an antibody described herein comprises a methionine (M) to tyrosine (Y) substitution in position 252, a serine (S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see Dall'Acqua W F et al., (2006) J Biol Chem 281:23514-24). In some embodiments, an antibody comprises an IgG constant domain comprising one, two, three or more amino acid substitutions of amino acid residues at positions 251-257, 285-290, 308-314, 385-389, and 428-436, numbered according to the EU index as in Kabat.

In some embodiments, one, two or more amino acid substitutions are introduced into an IgG constant domain Fc region to alter the effector function(s) of the anti-TfR1 antibody. The effector ligand to which affinity is altered can be, for example, an Fc receptor or the C1 component of complement. This approach is described in further detail in U.S. Pat. Nos. 5,624,821 and 5,648,260. In some embodiments, the deletion or inactivation (through point mutations or other means) of a constant region domain can reduce Fc receptor binding of the circulating antibody thereby increasing tumor localization. See, e.g., U.S. Pat. Nos. 5,585,097 and 8,591,886 for a description of mutations that delete or inactivate the constant domain and thereby increase tumor localization. In some embodiments, one or more amino acid substitutions may be introduced into the Fc region of an antibody described herein to remove potential glycosylation sites on Fc region, which may reduce Fc receptor binding (see, e.g., Shields R L et al., (2001) J Biol Chem 276:6591-604).

In some embodiments, one or more amino in the constant region of an anti-TfR1 antibody described herein can be replaced with a different amino acid residue such that the antibody has altered C1q binding and/or (e.g., and) reduced or abolished complement dependent cytotoxicity (CDC). This approach is described in further detail in U.S. Pat. No. 6,194,551 (Idusogie et al). In some embodiments, one or more amino acid residues in the N-terminal region of the CH2 domain of an antibody described herein are altered to thereby alter the ability of the antibody to fix complement. This approach is described further in International Publication No. WO 94/29351. In some embodiments, the Fc region of an antibody described herein is modified to increase the ability of the antibody to mediate antibody dependent cellular cytotoxicity (ADCC) and/or (e.g., and) to increase the affinity of the antibody for an Fcγ receptor. This approach is described further in International Publication No. WO 00/42072.

In some embodiments, the heavy and/or (e.g., and) light chain variable domain(s) sequence(s) of the antibodies provided herein can be used to generate, for example, CDR-grafted, chimeric, humanized, or composite human antibodies or antigen-binding fragments, as described elsewhere herein. As understood by one of ordinary skill in the art, any variant, CDR-grafted, chimeric, humanized, or composite antibodies derived from any of the antibodies provided herein may be useful in the compositions and methods described herein and will maintain the ability to specifically bind transferrin receptor, such that the variant, CDR-grafted, chimeric, humanized, or composite antibody has at least 50%, at least 60%, at least 70%, at least 80%, at least 90%, at least 95% or more binding to transferrin receptor relative to the original antibody from which it is derived.

In some embodiments, the antibodies provided herein comprise mutations that confer desirable properties to the antibodies. For example, to avoid potential complications due to Fab-arm exchange, which is known to occur with native IgG4 mAbs, the antibodies provided herein may comprise a stabilizing ‘Adair’ mutation (Angal S., et al., “A single amino acid substitution abolishes the heterogeneity of chimeric mouse/human (IgG4) antibody,” Mol Immunol 30, 105-108; 1993), where serine 228 (EU numbering; residue 241 Kabat numbering) is converted to proline resulting in an IgG1-like hinge sequence. Accordingly, any of the antibodies may include a stabilizing ‘Adair’ mutation.

In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecules are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, there are about 1-10, about 1-5, about 5-10, about 1-4, about 1-3, or about 2 sugar molecules. In some embodiments, a glycosylated antibody is fully or partially glycosylated. In some embodiments, an antibody is glycosylated by chemical reactions or by enzymatic means. In some embodiments, an antibody is glycosylated in vitro or inside a cell, which may optionally be deficient in an enzyme in the N- or O-glycosylation pathway, e.g. a glycosyltransferase. In some embodiments, an antibody is functionalized with sugar or carbohydrate molecules as described in International Patent Application Publication WO2014065661, published on May 1, 2014, entitled, “Modified antibody, antibody-conjugate and process for the preparation thereof”.

In some embodiments, any one of the anti-TfR1 antibodies described herein may comprise a signal peptide in the heavy and/or (e.g., and) light chain sequence (e.g., a N-terminal signal peptide). In some embodiments, the anti-TfR1 antibody described herein comprises any one of the VH and VL sequences, any one of the IgG heavy chain and light chain sequences, or any one of the F(ab′) heavy chain and light chain sequences described herein, and further comprises a signal peptide (e.g., a N-terminal signal peptide). In some embodiments, the signal peptide comprises the amino acid sequence of MGWSCIILFLVATATGVHS (SEQ ID NO: 104).

In some embodiments, an antibody provided herein may have one or more post-translational modifications. In some embodiments, N-terminal cyclization, also called pyroglutamate formation (pyro-Glu), may occur in the antibody at N-terminal Glutamate (Glu) and/or Glutamine (Gln) residues during production. As such, it should be appreciated that an antibody specified as having a sequence comprising an N-terminal glutamate or glutamine residue encompasses antibodies that have undergone pyroglutamate formation resulting from a post-translational modification. In some embodiments, pyroglutamate formation occurs in a heavy chain sequence. In some embodiments, pyroglutamate formation occurs in a light chain sequence.

### b. Other Muscle-Targeting Antibodies

In some embodiments, the muscle-targeting antibody is an antibody that specifically binds hemojuvelin, caveolin-3, Duchenne muscular dystrophy peptide, myosin IIb or CD63. In some embodiments, the muscle-targeting antibody is an antibody that specifically binds a myogenic precursor protein. Exemplary myogenic precursor proteins include, without limitation, ABCG2, M-Cadherin/Cadherin-15, Caveolin-1, CD34, FoxK1, Integrin alpha 7, Integrin alpha 7 beta 1, MYF-5, MyoD, Myogenin, NCAM-1/CD56, Pax3, Pax7, and Pax9. In some embodiments, the muscle-targeting antibody is an antibody that specifically binds a skeletal muscle protein. Exemplary skeletal muscle proteins include, without limitation, alpha-Sarcoglycan, beta-Sarcoglycan, Calpain Inhibitors, Creatine Kinase MM/CKMM, eIF5A, Enolase 2/Neuron-specific Enolase, epsilon-Sarcoglycan, FABP3/H-FABP, GDF-8/Myostatin, GDF-11/GDF-8, Integrin alpha 7, Integrin alpha 7 beta 1, Integrin beta 1/CD29, MCAM/CD146, MyoD, Myogenin, Myosin Light Chain Kinase Inhibitors, NCAM-1/CD56, and Troponin I. In some embodiments, the muscle-targeting antibody is an antibody that specifically binds a smooth muscle protein. Exemplary smooth muscle proteins include, without limitation, alpha-Smooth Muscle Actin, VE-Cadherin, Caldesmon/CALD1, Calponin 1, Desmin, Histamine H2 R, Motilin R/GPR38, Transgelin/TAGLN, and Vimentin. However, it should be appreciated that antibodies to additional targets are within the scope of this disclosure and the exemplary lists of targets provided herein are not meant to be limiting.

### c. Antibody Features/Alterations

In some embodiments, conservative mutations can be introduced into antibody sequences (e.g., CDRs or framework sequences) at positions where the residues are not likely to be involved in interacting with a target antigen (e.g., transferrin receptor), for example, as determined based on a crystal structure. In some embodiments, one, two or more mutations (e.g., amino acid substitutions) are introduced into the Fc region of a muscle-targeting antibody described herein (e.g., in a CH2 domain (residues 231-340 of human IgG1) and/or (e.g., and) CH3 domain (residues 341-447 of human IgG1) and/or (e.g., and) the hinge region, with numbering according to the Kabat numbering system (e.g., the EU index in Kabat)) to alter one or more functional properties of the antibody, such as serum half-life, complement fixation, Fc receptor binding and/or (e.g., and) antigen-dependent cellular cytotoxicity.

In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more mutations (e.g., amino acid substitutions) are introduced into the Fc region of a muscle-targeting antibody described herein (e.g., in a CH2 domain (residues 231-340 of human IgG1) and/or (e.g., and) CH3 domain (residues 341-447 of human IgG1) and/or (e.g., and) the hinge region, with numbering according to the Kabat numbering system (e.g., the EU index in Kabat)) to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109:6181-6186, U.S. Pat. No. 6,737,056, and International Publication Nos. WO 02/060919; WO 98/23289; and WO 97/34631, which are incorporated herein by reference.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody in vivo. See, e.g., International Publication Nos. WO 02/060919; WO 98/23289; and WO 97/34631; and U.S. Pat. Nos. 5,869,046, 6,121,022, 6,277,375, and 6,165,745 for examples of mutations that will alter (e.g., decrease or increase) the half-life of an antibody in vivo.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to decrease the half-life of the anti-transferrin receptor antibody in vivo. In some embodiments, one, two or more amino acid mutations (i.e., substitutions, insertions or deletions) are introduced into an IgG constant domain, or FcRn-binding fragment thereof (preferably an Fc or hinge-Fc domain fragment) to increase the half-life of the antibody in vivo. In some embodiments, the antibodies can have one or more amino acid mutations (e.g., substitutions) in the second constant (CH2) domain (residues 231-340 of human IgG1) and/or (e.g., and) the third constant (CH3) domain (residues 341-447 of human IgG1), with numbering according to the EU index in Kabat (Kabat E A et al., (1991) supra). In some embodiments, the constant region of the IgG1 of an antibody described herein comprises a methionine (M) to tyrosine (Y) substitution in position 252, a serine (S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see Dall'Acqua W F et al., (2006) J Biol Chem 281:23514-24). In some embodiments, an antibody comprises an IgG constant domain comprising one, two, three or more amino acid substitutions of amino acid residues at positions 251-257, 285-290, 308-314, 385-389, and 428-436, numbered according to the EU index as in Kabat.

In some embodiments, one, two or more amino acid substitutions are introduced into an IgG constant domain Fc region to alter the effector function(s) of the anti-transferrin receptor antibody. The effector ligand to which affinity is altered can be, for example, an Fc receptor or the C1 component of complement. This approach is described in further detail in U.S. Pat. Nos. 5,624,821 and 5,648,260. In some embodiments, the deletion or inactivation (through point mutations or other means) of a constant region domain can reduce Fc receptor binding of the circulating antibody thereby increasing tumor localization. See, e.g., U.S. Pat. Nos. 5,585,097 and 8,591,886 for a description of mutations that delete or inactivate the constant domain and thereby increase tumor localization. In some embodiments, one or more amino acid substitutions may be introduced into the Fc region of an antibody described herein to remove potential glycosylation sites on Fc region, which may reduce Fc receptor binding (see, e.g., Shields R L et al., (2001) J Biol Chem 276:6591-604).

In some embodiments, one or more amino in the constant region of a muscle-targeting antibody described herein can be replaced with a different amino acid residue such that the antibody has altered C1q binding and/or (e.g., and) reduced or abolished complement dependent cytotoxicity (CDC). This approach is described in further detail in U.S. Pat. No. 6,194,551 (Idusogie et al). In some embodiments, one or more amino acid residues in the N-terminal region of the CH2 domain of an antibody described herein are altered to thereby alter the ability of the antibody to fix complement. This approach is described further in International Publication No. WO 94/29351. In some embodiments, the Fc region of an antibody described herein is modified to increase the ability of the antibody to mediate antibody dependent cellular cytotoxicity (ADCC) and/or (e.g., and) to increase the affinity of the antibody for an Fcγ receptor. This approach is described further in International Publication No. WO 00/42072.

In some embodiments, the heavy and/or (e.g., and) light chain variable domain(s) sequence(s) of the antibodies provided herein can be used to generate, for example, CDR-grafted, chimeric, humanized, or composite human antibodies or antigen-binding fragments, as described elsewhere herein. As understood by one of ordinary skill in the art, any variant, CDR-grafted, chimeric, humanized, or composite antibodies derived from any of the antibodies provided herein may be useful in the compositions and methods described herein and will maintain the ability to specifically bind transferrin receptor, such that the variant, CDR-grafted, chimeric, humanized, or composite antibody has at least 50%, at least 60%, at least 70%, at least 80%, at least 90%, at least 95% or more binding to transferrin receptor relative to the original antibody from which it is derived.

In some embodiments, the antibodies provided herein comprise mutations that confer desirable properties to the antibodies. For example, to avoid potential complications due to Fab-arm exchange, which is known to occur with native IgG4 mAbs, the antibodies provided herein may comprise a stabilizing ‘Adair’ mutation (Angal S., et al., “A single amino acid substitution abolishes the heterogeneity of chimeric mouse/human (IgG4) antibody,” Mol Immunol 30, 105-108; 1993), where serine 228 (EU numbering; residue 241 Kabat numbering) is converted to proline resulting in an IgG1-like hinge sequence. Accordingly, any of the antibodies may include a stabilizing ‘Adair’ mutation.

As provided herein, antibodies of this disclosure may optionally comprise constant regions or parts thereof. For example, a VL domain may be attached at its C-terminal end to a light chain constant domain like Ck or CA. Similarly, a VH domain or portion thereof may be attached to all or part of a heavy chain like IgA, IgD, IgE, IgG, and IgM, and any isotype subclass. Antibodies may include suitable constant regions (see, for example, Kabat et al., Sequences of Proteins of Immunological Interest, No. 91-3242, National Institutes of Health Publications, Bethesda, Md. (1991)). Therefore, antibodies within the scope of this may disclosure include VH and VL domains, or an antigen binding portion thereof, combined with any suitable constant regions.

### ii. Muscle-Targeting Peptides

Some aspects of the disclosure provide muscle-targeting peptides as muscle-targeting agents. Short peptide sequences (e.g., peptide sequences of 5-20 amino acids in length) that bind to specific cell types have been described. For example, cell-targeting peptides have been described in Vines e., et al., A. “Cell-penetrating and cell-targeting peptides in drug delivery” Biochim Biophys Acta 2008, 1786:126-38; Jarver P., et al., “In vivo biodistribution and efficacy of peptide mediated delivery” Trends Pharmacol Sci 2010; 31:528-35; Samoylova T. I., et al., “Elucidation of muscle-binding peptides by phage display screening” Muscle Nerve 1999; 22:460-6; U.S. Pat. No. 6,329,501, issued on Dec. 11, 2001, entitled “METHODS AND COMPOSITIONS FOR TARGETING COMPOUNDS TO MUSCLE”; and Samoylov A. M., et al., “Recognition of cell-specific binding of phage display derived peptides using an acoustic wave sensor.” Biomol Eng 2002; 18:269-72; the entire contents of each of which are incorporated herein by reference. By designing peptides to interact with specific cell surface antigens (e.g., receptors), selectivity for a desired tissue, e.g., muscle, can be achieved. Skeletal muscle-targeting has been investigated and a range of molecular payloads are able to be delivered. These approaches may have high selectivity for muscle tissue without many of the practical disadvantages of a large antibody or viral particle. Accordingly, in some embodiments, the muscle-targeting agent is a muscle-targeting peptide that is from 4 to 50 amino acids in length. In some embodiments, the muscle-targeting peptide is 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. Muscle-targeting peptides can be generated using any of several methods, such as phage display.

In some embodiments, a muscle-targeting peptide may bind to an internalizing cell surface receptor that is overexpressed or relatively highly expressed in muscle cells, e.g. a transferrin receptor, compared with certain other cells. In some embodiments, a muscle-targeting peptide may target, e.g., bind to, a transferrin receptor. In some embodiments, a peptide that targets a transferrin receptor may comprise a segment of a naturally occurring ligand, e.g., transferrin. In some embodiments, a peptide that targets a transferrin receptor is as described in U.S. Pat. No. 6,743,893, filed Nov. 30, 2000, “RECEPTOR-MEDIATED UPTAKE OF PEPTIDES THAT BIND THE HUMAN TRANSFERRIN RECEPTOR”. In some embodiments, a peptide that targets a transferrin receptor is as described in Kawamoto, M. et al, “A novel transferrin receptor-targeted hybrid peptide disintegrates cancer cell membrane to induce rapid killing of cancer cells.” BMC Cancer. 2011 Aug. 18; 11:359. In some embodiments, a peptide that targets a transferrin receptor is as described in U.S. Pat. No. 8,399,653, filed May 20, 2011, “TRANSFERRIN/TRANSFERRIN RECEPTOR-MEDIATED SIRNA DELIVERY”.

As discussed above, examples of muscle targeting peptides have been reported. For example, muscle-specific peptides were identified using phage display library presenting surface heptapeptides. As one example a peptide having the amino acid sequence ASSLNIA (SEQ ID NO: 167) bound to C2C12 murine myotubes in vitro, and bound to mouse muscle tissue in vivo. Accordingly, in some embodiments, the muscle-targeting agent comprises the amino acid sequence ASSLNIA (SEQ ID NO: 167). This peptide displayed improved specificity for binding to heart and skeletal muscle tissue after intravenous injection in mice with reduced binding to liver, kidney, and brain. Additional muscle-specific peptides have been identified using phage display. For example, a 12 amino acid peptide was identified by phage display library for muscle targeting in the context of treatment for DMD. See, Yoshida D., et al., “Targeting of salicylate to skin and muscle following topical injections in rats.” Int J Pharm 2002; 231:177-84; the entire contents of which are hereby incorporated by reference. Here, a 12 amino acid peptide having the sequence SKTFNTHPQSTP (SEQ ID NO: 168) was identified and this muscle-targeting peptide showed improved binding to C2C12 cells relative to the ASSLNIA (SEQ ID NO: 167) peptide.

An additional method for identifying peptides selective for muscle (e.g., skeletal muscle) over other cell types includes in vitro selection, which has been described in Ghosh D., et al., “Selection of muscle-binding peptides from context-specific peptide-presenting phage libraries for adenoviral vector targeting” J Virol 2005; 79:13667-72; the entire contents of which are incorporated herein by reference. By pre-incubating a random 12-mer peptide phage display library with a mixture of non-muscle cell types, non-specific cell binders were selected out. Following rounds of selection the 12 amino acid peptide TARGEHKEEELI (SEQ ID NO: 169) appeared most frequently. Accordingly, in some embodiments, the muscle-targeting agent comprises the amino acid sequence TARGEHKEEELI (SEQ ID NO: 169).

A muscle-targeting agent may an amino acid-containing molecule or peptide. A muscle-targeting peptide may correspond to a sequence of a protein that preferentially binds to a protein receptor found in muscle cells. In some embodiments, a muscle-targeting peptide contains a high propensity of hydrophobic amino acids, e.g. valine, such that the peptide preferentially targets muscle cells. In some embodiments, a muscle-targeting peptide has not been previously characterized or disclosed. These peptides may be conceived of, produced, synthesized, and/or (e.g., and) derivatized using any of several methodologies, e.g. phage displayed peptide libraries, one-bead one-compound peptide libraries, or positional scanning synthetic peptide combinatorial libraries. Exemplary methodologies have been characterized in the art and are incorporated by reference (Gray, B. P. and Brown, K. C. “Combinatorial Peptide Libraries: Mining for Cell-Binding Peptides” Chem Rev. 2014, 114:2, 1020-1081.; Samoylova, T. I. and Smith, B. F. “Elucidation of muscle-binding peptides by phage display screening.” Muscle Nerve, 1999, 22:4. 460-6.). In some embodiments, a muscle-targeting peptide has been previously disclosed (see, e.g. Writer M. J. et al. “Targeted gene delivery to human airway epithelial cells with synthetic vectors incorporating novel targeting peptides selected by phage display.” J. Drug Targeting. 2004; 12:185; Cai, D. “BDNF-mediated enhancement of inflammation and injury in the aging heart.” Physiol Genomics. 2006, 24:3, 191-7.; Zhang, L. “Molecular profiling of heart endothelial cells.” Circulation, 2005, 112:11, 1601-11.; McGuire, M. J. et al. “In vitro selection of a peptide with high selectivity for cardiomyocytes in vivo.” J Mol Biol. 2004, 342:1, 171-82.). Exemplary muscle-targeting peptides comprise an amino acid sequence of the following group: CQAQGQLVC (SEQ ID NO: 170), CSERSMNFC (SEQ ID NO: 171), CPKTRRVPC (SEQ ID NO: 130), WLSEAGPVVTVRALRGTGSW (SEQ ID NO: 172), ASSLNIA (SEQ ID NO: 167), CMQHSMRVC (SEQ ID NO: 173), and DDTRHWG (SEQ ID NO: 131). In some embodiments, a muscle-targeting peptide may comprise about 2-25 amino acids, about 2-20 amino acids, about 2-15 amino acids, about 2-10 amino acids, or about 2-5 amino acids. Muscle-targeting peptides may comprise naturally-occurring amino acids, e.g. cysteine, alanine, or non-naturally-occurring or modified amino acids. Non-naturally occurring amino acids include β-amino acids, homo-amino acids, proline derivatives, 3-substituted alanine derivatives, linear core amino acids, N-methyl amino acids, and others known in the art. In some embodiments, a muscle-targeting peptide may be linear; in other embodiments, a muscle-targeting peptide may be cyclic, e.g. bicyclic (see, e.g. Silvana, M. G. et al. Mol. Therapy, 2018, 26:1, 132-147.).

### iii. Muscle-Targeting Receptor Ligands

A muscle-targeting agent may be a ligand, e.g. a ligand that binds to a receptor protein. A muscle-targeting ligand may be a protein, e.g. transferrin, which binds to an internalizing cell surface receptor expressed by a muscle cell. Accordingly, in some embodiments, the muscle-targeting agent is transferrin, or a derivative thereof that binds to a transferrin receptor. A muscle-targeting ligand may alternatively be a small molecule, e.g. a lipophilic small molecule that preferentially targets muscle cells relative to other cell types. Exemplary lipophilic small molecules that may target muscle cells include compounds comprising cholesterol, cholesteryl, stearic acid, palmitic acid, oleic acid, oleyl, linolene, linoleic acid, myristic acid, sterols, dihydrotestosterone, testosterone derivatives, glycerine, alkyl chains, trityl groups, and alkoxy acids.

### iv. Muscle-Targeting Aptamers

A muscle-targeting agent may be an aptamer, e.g. an RNA aptamer, which preferentially targets muscle cells relative to other cell types. In some embodiments, a muscle-targeting aptamer has not been previously characterized or disclosed. These aptamers may be conceived of, produced, synthesized, and/or (e.g., and) derivatized using any of several methodologies, e.g. Systematic Evolution of Ligands by Exponential Enrichment. Exemplary methodologies have been characterized in the art and are incorporated by reference (Yan, A. C. and Levy, M. “Aptamers and aptamer targeted delivery” RNA biology, 2009, 6:3, 316-20.; Germer, K. et al. “RNA aptamers and their therapeutic and diagnostic applications.” Int. J. Biochem. Mol. Biol. 2013; 4:27-40.). In some embodiments, a muscle-targeting aptamer has been previously disclosed (see, e.g. Phillippou, S. et al. “Selection and Identification of Skeletal-Muscle-Targeted RNA Aptamers.” Mol Ther Nucleic Acids. 2018, 10:199-214.; Thiel, W. H. et al. “Smooth Muscle Cell-targeted RNA Aptamer Inhibits Neointimal Formation.” Mol Ther. 2016, 24:4, 779-87.). Exemplary muscle-targeting aptamers include the A01B RNA aptamer and RNA Apt 14. In some embodiments, an aptamer is a nucleic acid-based aptamer, an oligonucleotide aptamer or a peptide aptamer. In some embodiments, an aptamer may be about 5-15 kDa, about 5-10 kDa, about 10⁻¹⁵ kDa, about 1-5 Da, about 1-3 kDa, or smaller.

### v. Other Muscle-Targeting Agents

One strategy for targeting a muscle cell (e.g., a skeletal muscle cell) is to use a substrate of a muscle transporter protein, such as a transporter protein expressed on the sarcolemma. In some embodiments, the muscle-targeting agent is a substrate of an influx transporter that is specific to muscle tissue. In some embodiments, the influx transporter is specific to skeletal muscle tissue. Two main classes of transporters are expressed on the skeletal muscle sarcolemma, (1) the adenosine triphosphate (ATP) binding cassette (ABC) superfamily, which facilitate efflux from skeletal muscle tissue and (2) the solute carrier (SLC) superfamily, which can facilitate the influx of substrates into skeletal muscle. In some embodiments, the muscle-targeting agent is a substrate that binds to an ABC superfamily or an SLC superfamily of transporters. In some embodiments, the substrate that binds to the ABC or SLC superfamily of transporters is a naturally-occurring substrate. In some embodiments, the substrate that binds to the ABC or SLC superfamily of transporters is a non-naturally occurring substrate, for example, a synthetic derivative thereof that binds to the ABC or SLC superfamily of transporters.

In some embodiments, the muscle-targeting agent is any muscle targeting agent described herein (e.g., antibodies, nucleic acids, small molecules, peptides, aptamers, lipids, sugar moieties) that target SLC superfamily of transporters. In some embodiments, the muscle-targeting agent is a substrate of an SLC superfamily of transporters. SLC transporters are either equilibrative or use proton or sodium ion gradients created across the membrane to drive transport of substrates. Exemplary SLC transporters that have high skeletal muscle expression include, without limitation, the SATT transporter (ASCT1; SLC1A4), GLUT4 transporter (SLC2A4), GLUT7 transporter (GLUT7; SLC2A7), ATRC2 transporter (CAT-2; SLC7A2), LAT3 transporter (KIAA0245; SLC7A6), PHT1 transporter (PTR4; SLC15A4), OATP-J transporter (OATP5A1; SLC21A15), OCT3 transporter (EMT; SLC22A3), OCTN2 transporter (FLJ46769; SLC22A5), ENT transporters (ENT1; SLC29A1 and ENT2; SLC29A2), PAT2 transporter (SLC36A2), and SAT2 transporter (KIAA1382; SLC38A2). These transporters can facilitate the influx of substrates into skeletal muscle, providing opportunities for muscle targeting.

In some embodiments, the muscle-targeting agent is a substrate of an equilibrative nucleoside transporter 2 (ENT2) transporter. Relative to other transporters, ENT2 has one of the highest mRNA expressions in skeletal muscle. While human ENT2 (hENT2) is expressed in most body organs such as brain, heart, placenta, thymus, pancreas, prostate, and kidney, it is especially abundant in skeletal muscle. Human ENT2 facilitates the uptake of its substrates depending on their concentration gradient. ENT2 plays a role in maintaining nucleoside homeostasis by transporting a wide range of purine and pyrimidine nucleobases. The hENT2 transporter has a low affinity for all nucleosides (adenosine, guanosine, uridine, thymidine, and cytidine) except for inosine. Accordingly, in some embodiments, the muscle-targeting agent is an ENT2 substrate. Exemplary ENT2 substrates include, without limitation, inosine, 2′,3′-dideoxyinosine, and calofarabine. In some embodiments, any of the muscle-targeting agents provided herein are associated with a molecular payload (e.g., oligonucleotide payload). In some embodiments, the muscle-targeting agent is covalently linked to the molecular payload. In some embodiments, the muscle-targeting agent is non-covalently linked to the molecular payload.

In some embodiments, the muscle-targeting agent is a substrate of an organic cation/carnitine transporter (OCTN2), which is a sodium ion-dependent, high affinity carnitine transporter. In some embodiments, the muscle-targeting agent is carnitine, mildronate, acetylcarnitine, or any derivative thereof that binds to OCTN2. In some embodiments, the carnitine, mildronate, acetylcarnitine, or derivative thereof is covalently linked to the molecular payload (e.g., oligonucleotide payload).

A muscle-targeting agent may be a protein that is protein that exists in at least one soluble form that targets muscle cells. In some embodiments, a muscle-targeting protein may be hemojuvelin (also known as repulsive guidance molecule C or hemochromatosis type 2 protein), a protein involved in iron overload and homeostasis. In some embodiments, hemojuvelin may be full length or a fragment, or a mutant with at least 75%, at least 80%, at least 85%, at least 90%, at least 95%, at least 98% or at least 99% sequence identity to a functional hemojuvelin protein. In some embodiments, a hemojuvelin mutant may be a soluble fragment, may lack a N-terminal signaling, and/or (e.g., and) lack a C-terminal anchoring domain. In some embodiments, hemojuvelin may be annotated under GenBank RefSeq Accession Numbers NM_001316767.1, NM_145277.4, NM_202004.3, NM_213652.3, or NM_213653.3. It should be appreciated that a hemojuvelin may be of human, non-human primate, or rodent origin.

### B. Molecular Payloads

Some aspects of the disclosure provide molecular payloads, e.g., oligonucleotides designed to target DUX4 RNAs to modulate the expression or the activity of DUX4. In some embodiments, the disclosure provides oligonucleotides complementary with DUX4 RNA that are useful for reducing levels of DUX4 mRNA and/or protein associated with features of facioscapulohumeral muscular dystrophy (FSHD) pathology, including muscle atrophy, inflammation, and decreased differentiation potential and oxidative stress. In some embodiments, the oligonucleotides provided herein are designed to direct RNAi mediated degradation of DUX4 RNA. In some embodiments, the oligonucleotides are designed to efficiently engage the RNA-induced silencing complex (RISC) for degradation of the DUX4 RNA but also have reduced off-target effect. In some embodiments, the oligonucleotides are designed to have desirable bioavailability and/or serum-stability properties. In some embodiments, the oligonucleotides are designed to have desirable binding affinity properties. In some embodiments, the oligonucleotides are designed to have desirable toxicity and/or immunogenicity profiles.

In some embodiments, the oligonucleotide comprises a strand having a region of complementarity to a DUX4 RNA. Exemplary oligonucleotides are described in further detail herein, however, it should be appreciated that the exemplary oligonucleotides provided herein are not meant to be limiting.

### i. Oligonucleotides

In some embodiments, the oligonucleotides provided herein are designed to cause RNAi mediated degradation of DUX4 mRNA. In some embodiments, the oligonucleotide provided herein comprises an antisense strand that is complementary to a DUX4 mRNA. In some embodiments, the oligonucleotide provided herein further comprises a sense strand that forms a double-stranded oligonucleotide (e.g., siRNA). It should be appreciated that, in some embodiments, oligonucleotides in one format (e.g., antisense oligonucleotides) may be suitably adapted to another format (e.g., siRNA oligonucleotides) by incorporating functional sequences (e.g., antisense strand sequences) from one format to the other format.

Any suitable oligonucleotide may be used as a molecular payload, as described herein. Examples of oligonucleotides useful for targeting DUX4 are provided in U.S. Pat. No. 9,988,628, published on Feb. 2, 2017, entitled “AGENTS USEFUL IN TREATING FACIOSCAPULOHUMERAL MUSCULAR DYSTROPHY”; U.S. Pat. No. 9,469,851, published Oct. 30, 2014, entitled “RECOMBINANT VIRUS PRODUCTS AND METHODS FOR INHIBITING EXPRESSION OF DUX4”; US Patent Application Publication 20120225034, published on Sep. 6, 2012, entitled “AGENTS USEFUL IN TREATING FACIOSCAPULOHUMERAL MUSCULAR DYSTROPHY”; PCT Patent Application Publication Number WO 2013/120038, published on Aug. 15, 2013, entitled “MORPHOLINO TARGETING DUX4 FOR TREATING FSHD”; Chen et al., “Morpholino-mediated Knockdown of DUX4 Toward Facioscapulohumeral Muscular Dystrophy Therapeutics,” Molecular Therapy, 2016, 24:8, 1405-1411.; and Ansseau et al., “Antisense Oligonucleotides Used to Target the DUX4 mRNA as Therapeutic Approaches in Facioscapulohumeral Muscular Dystrophy (FSHD),” Genes, 2017, 8, 93; the contents of each of which are incorporated herein in their entireties. In some embodiments, the oligonucleotide is an antisense oligonucleotide, a morpholino, an siRNA, a shRNA, or another oligonucleotide which hybridizes with the target DUX4 gene or mRNA. In some embodiments, the oligonucleotide is an siRNA oligonucleotide.

In some embodiments, the oligonucleotides described herein have a region of complementarity to a sequence as set forth as: Human DUX4, corresponding to NCBI sequence NM_001293798.2 (SEQ ID NO: 160) or NCBI Sequence: NM_001306068.3 (SEQ ID NO: 161) as below and/or (e.g., and) Mouse DUX4, corresponding to NCBI sequence NM_001081954.1 (SEQ ID NO: 162), as below. Other non-limiting exemplary human DUX4 mRNA include NCBI Sequence: NM_033178, GenBank accession numbers FJ439133, AF117653, HM101229, HM101230, HM101232, HM101233, HM101234, HM101235, HM101240, HM101241, HM101242, HM101243, HM101244, HM101245, HM101246, HM101247, HM101248, HM101249, HM101250, HM101251 and HM190160, HM190161, HM190162, HM190163, HM190164, HM190165, HM190166, HM190167, HM190168, HM190169, HM190170, HM190171, HM190172, HM190173, HM190174, HM190175, HM190176, HM190177, HM190178, HM190179, HM190180, HM190181, HM190182, HM190183, HM190184, HM190185, HM190186, HM190187, HM190188, HM190189, HM190190, HM190191, HM190192, HM190193, HM190194, HM190195, HM190196, each of which is incorporated herein by reference. In some embodiments, the oligonucleotide may have a region of complementarity to a hypomethylated, contracted D4Z4 repeat, as in Daxinger, et al., “Genetic and Epigenetic Contributors to FSHD,” published in Curr Opin Genet Dev in 2015, Lim J-W, et al., DICER/AGO-dependent epigenetic silencing of D4Z4 repeats enhanced by exogenous siRNA suggests mechanisms and therapies for FSHD Hum Mol Genet. 2015 Sep. 1; 24 (17): 4817-4828, the contents of each of which are incorporated in their entireties.

In some embodiments, oligonucleotides may have a region of complementarity to a sequence set forth as follows, which is an example human DUX4 gene sequence (NM_001293798.2) (SEQ ID NO: 160):

ATGGCCCTCCCGACACCCTCGGACAGCACCCTCCCCGCGGAAGCCCGGGG
ACGAGGACGGCGACGGAGACTCGTTTGGACCCCGAGCCAAAGCGAGGCCC
TGCGAGCCTGCTTTGAGCGGAACCCGTACCCGGGCATCGCCACCAGAGAA
CGGCTGGCCCAGGCCATCGGCATTCCGGAGCCCAGGGTCCAGATTTGGTT
TCAGAATGAGAGGTCACGCCAGCTGAGGCAGCACCGGCGGGAATCTCGGC
CCTGGCCCGGGAGACGCGGCCCGCCAGAAGGCCGGCGAAAGCGGACCGCC
GTCACCGGATCCCAGACCGCCCTGCTCCTCCGAGCCTTTGAGAAGGATCG
CTTTCCAGGCATCGCCGCCCGGGAGGAGCTGGCCAGAGAGACGGGCCTCC
CGGAGTCCAGGATTCAGATCTGGTTTCAGAATCGAAGGGCCAGGCACCCG
GGACAGGGTGGCAGGGCGCCCGCGCAGGCAGGCGGCCTGTGCAGCGCGGC
CCCCGGCGGGGGTCACCCTGCTCCCTCGTGGGTCGCCTTCGCCCACACCG
GCGCGTGGGGAACGGGGCTTCCCGCACCCCACGTGCCCTGCGCGCCTGGG
GCTCTCCCACAGGGGGCTTTCGTGAGCCAGGCAGCGAGGGCCGCCCCCGC
GCTGCAGCCCAGCCAGGCCGCGCCGGCAGAGGGGATCTCCCAACCTGCCC
CGGCGCGCGGGGATTTCGCCTACGCCGCCCCGGCTCCTCCGGACGGGGCG
CTCTCCCACCCTCAGGCTCCTCGCTGGCCTCCGCACCCGGGCAAAAGCCG
GGAGGACCGGGACCCGCAGCGCGACGGCCTGCCGGGCCCCTGCGCGGTGG
CACAGCCTGGGCCCGCTCAAGCGGGGCCGCAGGGCCAAGGGGTGCTTGCG
CCACCCACGTCCCAGGGGAGTCCGTGGTGGGGCTGGGGCCGGGGTCCCCA
GGTCGCCGGGGCGGCGTGGGAACCCCAAGCCGGGGCAGCTCCACCTCCCC
AGCCCGCGCCCCCGGACGCCTCCGCCTCCGCGCGGCAGGGGCAGATGCAA
GGCATCCCGGCGCCCTCCCAGGCGCTCCAGGAGCCGGCGCCCTGGTCTGC
ACTCCCCTGCGGCCTGCTGCTGGATGAGCTCCTGGCGAGCCCGGAGTTTC
TGCAGCAGGCGCAACCTCTCCTAGAAACGGAGGCCCCGGGGGAGCTGGAG
GCCTCGGAAGAGGCCGCCTCGCTGGAAGCACCCCTCAGCGAGGAAGAATA
CCGGGCTCTGCTGGAGGAGCTTTAGGACGCGGGGTCTAGGCCCGGTGAGA
GACTCCACACCGCGGAGAACTGCCATTCTTTCCTGGGCATCCCGGGGATC
CCAGAGCCGGCCCAGGTACCAGCAGACCTGCGCGCAGTGCGCACCCCGGC
TGACGTGCAAGGGAGCTCGCTGGCCTCTCTGTGCCCTTGTTCTTCCGTGA
AATTCTGGCTGAATGTCTCCCCCCACCTTCCGACGCTGTCTAGGCAAACC
TGGATTAGAGTTACATCTCCTGGATGATTAGTTCAGAGATATATTAAAAT
GCCCCCTCCCTGTGGATCCTATAG

In some embodiments, oligonucleotides may have a region of complementarity to a sequence set forth as follows, which is an example human DUX4 gene sequence (NM_001306068.3) (SEQ ID NO: 161):

ATGGCCCTCCCGACACCCTCGGACAGCACCCTCCCCGCGGAAGCCCGGGG
ACGAGGACGGCGACGGAGACTCGTTTGGACCCCGAGCCAAAGCGAGGCCC
TGCGAGCCTGCTTTGAGCGGAACCCGTACCCGGGCATCGCCACCAGAGAA
CGGCTGGCCCAGGCCATCGGCATTCCGGAGCCCAGGGTCCAGATTTGGTT
TCAGAATGAGAGGTCACGCCAGCTGAGGCAGCACCGGCGGGAATCTCGGC
CCTGGCCCGGGAGACGCGGCCCGCCAGAAGGCCGGCGAAAGCGGACCGCC
GTCACCGGATCCCAGACCGCCCTGCTCCTCCGAGCCTTTGAGAAGGATCG
CTTTCCAGGCATCGCCGCCCGGGAGGAGCTGGCCAGAGAGACGGGCCTCC
CGGAGTCCAGGATTCAGATCTGGTTTCAGAATCGAAGGGCCAGGCACCCG
GGACAGGGTGGCAGGGCGCCCGCGCAGGCAGGCGGCCTGTGCAGCGCGGC
CCCCGGCGGGGGTCACCCTGCTCCCTCGTGGGTCGCCTTCGCCCACACCG
GCGCGTGGGGAACGGGGCTTCCCGCACCCCACGTGCCCTGCGCGCCTGGG
GCTCTCCCACAGGGGGCTTTCGTGAGCCAGGCAGCGAGGGCCGCCCCCGC
GCTGCAGCCCAGCCAGGCCGCGCCGGCAGAGGGGATCTCCCAACCTGCCC
CGGCGCGCGGGGATTTCGCCTACGCCGCCCCGGCTCCTCCGGACGGGGCG
CTCTCCCACCCTCAGGCTCCTCGGTGGCCTCCGCACCCGGGCAAAAGCCG
GGAGGACCGGGACCCGCAGCGCGACGGCCTGCCGGGCCCCTGCGCGGTGG
CACAGCCTGGGCCCGCTCAAGCGGGGCCGCAGGGCCAAGGGGTGCTTGCG
CCACCCACGTCCCAGGGGAGTCCGTGGTGGGGCTGGGGCCGGGGTCCCCA
GGTCGCCGGGGCGGCGTGGGAACCCCAAGCCGGGGCAGCTCCACCTCCCC
AGCCCGCGCCCCCGGACGCCTCCGCCTCCGCGCGGCAGGGGCAGATGCAA
GGCATCCCGGCGCCCTCCCAGGCGCTCCAGGAGCCGGCGCCCTGGTCTGC
ACTCCCCTGCGGCCTGCTGCTGGATGAGCTCCTGGCGAGCCCGGAGTTTC
TGCAGCAGGCGCAACCTCTCCTAGAAACGGAGGCCCCGGGGGAGCTGGAG
GCCTCGGAAGAGGCCGCCTCGCTGGAAGCACCCCTCAGCGAGGAAGAATA
CCGGGCTCTGCTGGAGGAGCTTTAGGACGCGGGGTTGGGACGGGGTCGGG
TGGTTCGGGGCAGGGCGGTGGCCTCTCTTTCGCGGGGAACACCTGGCTGG
CTACGGAGGGGCGTGTCTCCGCCCCGCCCCCTCCACCGGGCTGACCGGCC
TGGGATTCCTGCCTTCTAGGTCTAGGCCCGGTGAGAGACTCCACTCCGCG
GAGAACTGCCTTTCTTTCCTGGGCATCCCGGGGATCCCAGAGCCGGCCCA
GGTACCAGCAGACCTGCGCGCAGTGCGCACCCCGGCTGACGTGCAAGGGA
GCTCGCTGGCCTCTCTGTGCCCTTGTTCTTCCGTGAAATTCTGGCTGAAT
GTCTCCCCCCACCTTCCGACGCTGTCTAGGCAAACCTGGATTAGAGTTAC
ATCTCCTGGATGATTAGTTCAGAGATATATTAAAATGCCCCCTCCCTGTG
GATCCTATAG.

In some embodiments, oligonucleotides may have a region of complementarity to a sequence set forth as follows, which is an example mouse DUX4 gene sequence (SEQ ID NO: 162) (NM 001081954.1):

ATGGCAGAAGCTGGCAGCCCTGTTGGTGGCAGTGGTGTGGCACGGGAATC
CCGGCGGCGCAGGAAGACGGTTTGGCAGGCCTGGCAAGAGCAGGCCCTGC
TATCAACTTTCAAGAAGAAGAGATACCTGAGCTTCAAGGAGAGGAAGGAG
CTGGCCAAGCGAATGGGGGTCTCAGATTGCCGCATCCGCGTGTGGTTTCA
GAACCGCAGGAATCGCAGTGGAGAGGAGGGGCATGCCTCAAAGAGGTCCA
TCAGAGGCTCCAGGCGGCTAGCCTCGCCACAGCTCCAGGAAGAGCTTGGA
TCCAGGCCACAGGGTAGAGGCATGCGCTCATCTGGCAGAAGGCCTCGCAC
TCGACTCACCTCGCTACAGCTCAGGATCCTAGGGCAAGCCTTTGAGAGGA
ACCCACGACCAGGCTTTGCTACCAGGGAGGAGCTGGCGCGTGACACAGGG
TTGCCCGAGGACACGATCCACATATGGTTTCAAAACCGAAGAGCTCGGCG
GCGCCACAGGAGGGGCAGGCCCACAGCTCAAGATCAAGACTTGCTGGCGT
CACAAGGGTCGGATGGGGCCCCTGCAGGTCCGGAAGGCAGAGAGCGTGAA
GGTGCCCAGGAGAACTTGTTGCCACAGGAAGAAGCAGGAAGTACGGGCAT
GGATACCTCGAGCCCTAGCGACTTGCCCTCCTTCTGCGGAGAGTCCCAGC
CTTTCCAAGTGGCACAGCCCCGTGGAGCAGGCCAACAAGAGGCCCCCACT
CGAGCAGGCAACGCAGGCTCTCTGGAACCCCTCCTTGATCAGCTGCTGGA
TGAAGTCCAAGTAGAAGAGCCTGCTCCAGCCCCTCTGAATTTGGATGGAG
ACCCTGGTGGCAGGGTGCATGAAGGTTCCCAGGAGAGCTTTTGGCCACAG
GAAGAAGCAGGAAGTACAGGCATGGATACTTCTAGCCCCAGCGACTCAAA
CTCCTTCTGCAGAGAGTCCCAGCCTTCCCAAGTGGCACAGCCCTGTGGAG
CGGGCCAAGAAGATGCCCGCACTCAAGCAGACAGCACAGGCCCTCTGGAA
CTCCTCCTCCTTGATCAACTGCTGGACGAAGTCCAAAAGGAAGAGCATGT
GCCAGTCCCACTGGATTGGGGTAGAAATCCTGGCAGCAGGGAGCATGAAG
GTTCCCAGGACAGCTTACTGCCCCTGGAGGAAGCAGTAAATTCGGGCATG
GATACCTCGATCCCTAGCATCTGGCCAACCTTCTGCAGAGAATCCCAGCC
TCCCCAAGTGGCACAGCCCTCTGGACCAGGCCAAGCACAGGCCCCCACTC
AAGGTGGGAACACGGACCCCCTGGAGCTCTTCCTCTATCAACTGTTGGAT
GAAGTCCAAGTAGAAGAGCATGCTCCAGCCCCTCTGAATTGGGATGTAGA
TCCTGGTGGCAGGGTGCATGAAGGTTCGTGGGAGAGCTTTTGGCCACAGG
AAGAAGCAGGAAGTACAGGCCTGGATACTTCAAGCCCCAGCGACTCAAAC
TCCTTCTTCAGAGAGTCCAAGCCTTCCCAAGTGGCACAGCGCCGTGGAGC
GGGCCAAGAAGATGCCCGCACTCAAGCAGACAGCACAGGCCCTCTGGAAC
TCCTCCTCTTTGATCAACTGCTGGACGAAGTCCAAAAGGAAGAGCATGTG
CCAGCCCCACTGGATTGGGGTAGAAATCCTGGCAGCATGGAGCATGAAGG
TTCCCAGGACAGCTTACTGCCCCTGGAGGAAGCAGCAAATTCGGGCAGGG
ATACCTCGATCCCTAGCATCTGGCCAGCCTTCTGCAGAAAATCCCAGCCT
CCCCAAGTGGCACAGCCCTCTGGACCAGGCCAAGCACAGGCCCCCATTCA
AGGTGGGAACACGGACCCCCTGGAGCTCTTCCTTGATCAACTGCTGACCG
AAGTCCAACTTGAGGAGCAGGGGCCTGCCCCTGTGAATGTGGAGGAAACA
TGGGAGCAAATGGACACAACACCTATCTGCCTCTCACTTCAGAAGAATAT
CAGACTCTTCTAGATATGCTCTGA.

In some embodiments, an oligonucleotide may have a region of complementarity to DUX4 gene sequences of multiple species, e.g., selected from human, mouse and non-human species. In some embodiments, the non-human species is a cynomolgus monkey.

### ii. Oligonucleotide Size/Sequence

Oligonucleotides may be of a variety of different lengths, e.g., depending on the format. In some embodiments, an oligonucleotide is 7, 8, 9, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, the oligonucleotide is 8 to 50 nucleotides in length, 8 to 40 nucleotides in length, 8 to 32 nucleotides in length, 10 to 15 nucleotides in length, 10 to 20 nucleotides in length, 15 to 25 nucleotides in length, 21 to 23 nucleotides in lengths, etc. In some embodiments, the oligonucleotide is 8 to 32 nucleotides, 15 to 29 nucleotides, 15 to 27 nucleotides, 15 to 20 nucleotides, 20 to 25 nucleotides, 21 to 27 nucleotides, 23 to 27 nucleotides, 25 to 30 nucleotides, or 25-32 nucleotides in length.

In some embodiments, a complementary nucleic acid sequence of an oligonucleotide for purposes of the present disclosure is specifically hybridizable or specific for the target nucleic acid when binding of the sequence to the target molecule (e.g., mRNA) interferes with the normal function of the target (e.g., mRNA) to cause a loss of activity (e.g., inhibiting translation) or expression (e.g., degrading a target mRNA) and there is a sufficient degree of complementarity to avoid non-specific binding of the sequence to non-target sequences under conditions in which avoidance of non-specific binding is desired, e.g., under physiological conditions in the case of in vivo assays or therapeutic treatment, and in the case of in vitro assays, under conditions in which the assays are performed under suitable conditions of stringency. Thus, in some embodiments, an oligonucleotide may be at least 80%, at least 85%, at least 90%, at least 91%, at least 92%, at least 93%, at least 94%, at least 95%, at least 96%, at least 97%, at least 98%, at least 99% or 100% complementary to the consecutive nucleotides of a target nucleic acid. In some embodiments a complementary nucleotide sequence need not be 100% complementary to that of its target to be specifically hybridizable or specific for a target nucleic acid. In some embodiments, oligonucleotides comprise one or more mismatched nucleobases relative to the target nucleic acid. In some embodiments, activity relating to the target is reduced by such mismatch, but activity relating to a non-target is reduced by a greater amount (i.e., selectivity for the target nucleic acid is increased and off-target effects are decreased). In some embodiments, the target nucleic acid is a pre-mRNA molecule or an mRNA molecule.

In some embodiments, an oligonucleotide comprises region of complementarity to a target nucleic acid that is in the range of 8 to 15, 8 to 30, 8 to 40, or 10 to 50, or 5 to 50, or 5 to 40 nucleotides in length. In some embodiments, an oligonucleotide comprises region of complementarity to a target nucleic acid that is in the range of 8-32, 15-29, 15-27, 21-27, 23-27 nucleotides in length. In some embodiments, an oligonucleotide comprises a region of complementarity to a target nucleic acid that is in the range of 15-29, 15-27, 15 to 20, 20 to 25, 21-27, 23-27, 25-27, or 25-32 nucleotides in length. In some embodiments, a region of complementarity of an oligonucleotide to a target nucleic acid is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity is complementary with at least 8 consecutive nucleotides of a target nucleic acid. In some embodiments, the region of complementarity is complementary with at least 12 consecutive nucleotides of a target nucleic acid. In some embodiments, the region of complementarity is complementary with at least 16 consecutive nucleotides of a target nucleic acid. In some embodiments, an oligonucleotide may contain 1, 2 or 3 base mismatches compared to the portion of the consecutive nucleotides of target nucleic acid. In some embodiments the oligonucleotide may have up to 3 mismatches over 15 bases, or up to 2 mismatches over 10 bases.

In some embodiments, an oligonucleotide comprises at least 10, 11, 12, 13, 14, 15, 16, 17, 18, 19, or 20 consecutive nucleotides of a sequence comprising any one of SEQ ID NOs: 236-266. In some embodiments, an oligonucleotide comprises a sequence comprising any one of SEQ ID NOs: 236-266. In some embodiments, an oligonucleotide comprises a sequence that shares at least 70%, 75%, 80%, 85%, 90%, 95%, or 97% sequence identity with at least 12 or at least 15 consecutive nucleotides of any one of SEQ ID NOs: 236-266.

In some embodiments, an oligonucleotide comprises a region of complementarity to a target sequence as set forth in any one of SEQ ID NO: 174-235. In some embodiments, an oligonucleotide comprises region of complementarity that is at least 70%, 75%, 80%, 85%, 90%, 95%, 97%; 99%, or 100% complementary with at least 12 or at least 15 consecutive nucleotides of a target sequence as set forth of any one of SEQ ID NO: 174-235. In some embodiments, the region of complementarity is at least 8, at least 9, at least 10, at least 11, at least 12, at least 13, at least 14, at least 15, at least 16, at least 17, at least 19 or at least 20 nucleotides in length. In some embodiments, the region of complementarity is 8, 9, 10, 11, 12, 13, 14, 15, 16, 17, 18, 19, or 20 nucleotides in length. In some embodiments, the region of complementarity is in the range of 8 to 20, 10 to 20 or 15 to 20 nucleotides in length. In some embodiments, the region of complementarity is fully complementary with all or a portion of its target sequence. In some embodiments, the region of complementarity includes 1, 2, 3 or more mismatches.

In some embodiments, the oligonucleotide is complementary (e.g., at least 85% at least 90%, at least 95%, or 100%) to a target sequence of any one of the oligonucleotides provided herein (e.g., the oligonucleotides listed in Table 8). In some embodiments, the oligonucleotide is complementary (e.g., at least 85% at least 90%, at least 95%, or 100%) to a target sequence of any one of the oligonucleotides provided herein (e.g., the oligonucleotides listed in Table 9). In some embodiments, such target sequence is 100% complementary to the oligonucleotide listed in Table 8. In some embodiments, such target sequence is 100% complementary to the oligonucleotide listed in Table 9. In some embodiments, the oligonucleotide is complementary (e.g., at least 85% at least 90%, at least 95%, or 100%) to a target sequence of any one of the oligonucleotides provided herein (e.g., the oligonucleotides comprising any one of SEQ ID NOs: 236-266). In some embodiments, the oligonucleotide is complementary (e.g., at least 85% at least 90%, at least 95%, or 100%) to a target sequence of any one of the oligonucleotides provided herein (e.g., the oligonucleotides comprising any one of SEQ ID NOs: 248, 251-253 and 262). In some embodiments, such target sequence is 100% complementary to the oligonucleotide described herein (e.g., the oligonucleotides comprising any one of SEQ ID NOs: 236-266). In some embodiments, such target sequence is 100% complementary to the oligonucleotide described herein (e.g., the oligonucleotides comprising any one of SEQ ID NOs: 248, 251-253 and 262).

In some embodiments, it should be appreciated that methylation of the nucleobase uracil at the C5 position forms thymine. Thus, in some embodiments, a nucleotide or nucleoside having a C5 methylated uracil (or 5-methyl-uracil) may be equivalently identified as a thymine nucleotide or nucleoside.

In some embodiments, one or more of the thymine bases (T's) in any one of the oligonucleotides provided herein may independently and optionally be uracil bases (U's), and/or any one or more of the U's may independently and optionally be T's. In some embodiments, one or more of the thymine bases (T's) in any one of the oligonucleotides listed in Table 8 may independently and optionally be uracil bases (U's), and/or any one or more of the U's may independently and optionally be T's. In some embodiments, one or more of the thymine bases (T's) in any one of the oligonucleotides listed in Table 9 may independently and optionally be uracil bases (U's), and/or any one or more of the U's may independently and optionally be T's.

### b. Oligonucleotide Modifications

The oligonucleotides described herein may be modified, e.g., comprise a modified sugar moiety, a modified internucleoside linkage, a modified nucleotide or nucleoside and/or (e.g., and) combinations thereof. In addition, in some embodiments, oligonucleotides may exhibit one or more of the following properties: do not mediate alternative splicing; are not immune stimulatory; are nuclease resistant; have improved cell uptake compared to unmodified oligonucleotides; are not toxic to cells or mammals; have improved endosomal exit internally in a cell; minimizes TLR stimulation; or avoid pattern recognition receptors. Any of the modified chemistries or formats of oligonucleotides described herein can be combined with each other. For example, one, two, three, four, five, or more different types of modifications can be included within the same oligonucleotide.

In some embodiments, certain nucleotide or nucleoside modifications may be used that make an oligonucleotide into which they are incorporated more resistant to nuclease digestion than the native oligodeoxynucleotide or oligoribonucleotide molecules; these modified oligonucleotides survive intact for a longer time than unmodified oligonucleotides. Specific examples of modified oligonucleotides include those comprising modified backbones, for example, modified internucleoside linkages such as phosphorothioates, phosphotriesters, methyl phosphonates, short chain alkyl or cycloalkyl intersugar linkages or short chain heteroatomic or heterocyclic intersugar linkages. Accordingly, oligonucleotides of the disclosure can be stabilized against nucleolytic degradation such as by the incorporation of a modification, e.g., a nucleotide or nucleoside modification.

In some embodiments, an oligonucleotide may be of up to 50 or up to 100 nucleotides in length in which 2 to 10, 2 to 15, 2 to 16, 2 to 17, 2 to 18, 2 to 19, 2 to 20, 2 to 25, 2 to 30, 2 to 40, 2 to 45, or more nucleotides or nucleosides of the oligonucleotide are modified nucleotides/nucleosides. The oligonucleotide may be of 8 to 30 nucleotides in length in which 2 to 10, 2 to 15, 2 to 16, 2 to 17, 2 to 18, 2 to 19, 2 to 20, 2 to 25, 2 to 30 nucleotides or nucleosides of the oligonucleotide are modified nucleotides/nucleosides. The oligonucleotide may be of 8 to 15 nucleotides in length in which 2 to 4, 2 to 5, 2 to 6, 2 to 7, 2 to 8, 2 to 9, 2 to 10, 2 to 11, 2 to 12, 2 to 13, 2 to 14 nucleotides or nucleosides of the oligonucleotide are modified nucleotides/nucleosides. Optionally, the oligonucleotides may have every nucleotide or nucleoside except 1, 2, 3, 4, 5, 6, 7, 8, 9, or 10 nucleotides/nucleosides modified. Oligonucleotide modifications are described further herein.

### c. Modified Nucleosides

In some embodiments, the oligonucleotide described herein comprises at least one nucleoside modified at the 2′ position of the sugar. In some embodiments, an oligonucleotide comprises at least one 2′-modified nucleoside. In some embodiments, all of the nucleosides in the oligonucleotide are 2′-modified nucleosides.

In some embodiments, the oligonucleotide described herein comprises one or more non-bicyclic 2′-modified nucleosides, e.g., 2′-deoxy, 2′-fluoro (2′-F), 2′-O-methyl(2′—O-Me), 2′-O-methoxyethyl(2′-MOE), 2′-O-aminopropyl(2′—O-AP), 2′-O-dimethylaminoethyl(2′—O-DMAOE), 2′-O-dimethylaminopropyl(2′—O-DMAP), 2′-O-dimethylaminoethyloxyethyl(2′—O-DMAEOE), or 2′—O—N-methylacetamido (2′-O-NMA) modified nucleoside.

In some embodiments, the oligonucleotide described herein comprises one or more 2′-fluoro (2′-F) modified nucleoside. In some embodiments, the oligonucleotide described herein comprises at least two 2′-fluoro (2′-F) modified nucleosides. In some embodiments, the oligonucleotide described herein comprises at least four 2′-fluoro (2′-F) modified nucleosides. In some embodiments, the oligonucleotide described herein comprises at least six 2′-fluoro (2′-F) modified nucleosides. In some embodiments, the oligonucleotide described herein comprises one or more 2′-O-methyl(2′—O-Me) modified nucleoside.

In some embodiments, the oligonucleotide described herein comprises one or more 2′-4′ bicyclic nucleosides in which the ribose ring comprises a bridge moiety connecting two atoms in the ring, e.g., connecting the 2′-O atom to the 4′-C atom via a methylene (LNA) bridge, an ethylene (ENA) bridge, or a (S)-constrained ethyl (cEt) bridge. Examples of LNAs are described in International Patent Application Publication WO/2008/043753, published on Apr. 17, 2008, and entitled “RNA Antagonist Compounds For The Modulation Of PCSK9”, the contents of which are incorporated herein by reference in its entirety. Examples of ENAs are provided in International Patent Publication No. WO 2005/042777, published on May 12, 2005, and entitled “APP/ENA Antisense”; Morita et al., Nucleic Acid Res., Suppl 1:241-242, 2001; Surono et al., Hum. Gene Ther., 15:749-757, 2004; Koizumi, Curr. Opin. Mol. Ther., 8:144-149, 2006 and Horie et al., Nucleic Acids Symp. Ser (Oxf), 49:171-172, 2005; the disclosures of which are incorporated herein by reference in their entireties. Examples of cEt are provided in U.S. Pat. Nos. 7,101,993; 7,399,845 and 7,569,686, each of which is herein incorporated by reference in its entirety.

In some embodiments, the oligonucleotide comprises a modified nucleoside disclosed in one of the following United States Patent or Patent Application Publications: U.S. Pat. No. 7,399,845, issued on Jul. 15, 2008, and entitled “6-Modified Bicyclic Nucleic Acid Analogs”; U.S. Pat. No. 7,741,457, issued on Jun. 22, 2010, and entitled “6-Modified Bicyclic Nucleic Acid Analogs”; U.S. Pat. No. 8,022,193, issued on Sep. 20, 2011, and entitled “6-Modified Bicyclic Nucleic Acid Analogs”; U.S. Pat. No. 7,569,686, issued on Aug. 4, 2009, and entitled “Compounds And Methods For Synthesis Of Bicyclic Nucleic Acid Analogs”; U.S. Pat. No. 7,335,765, issued on Feb. 26, 2008, and entitled “Novel Nucleoside And Oligonucleotide Analogues”; U.S. Pat. No. 7,314,923, issued on Jan. 1, 2008, and entitled “Novel Nucleoside And Oligonucleotide Analogues”; U.S. Pat. No. 7,816,333, issued on Oct. 19, 2010, and entitled “Oligonucleotide Analogues And Methods Utilizing The Same” and US Publication Number 2011/0009471 now U.S. Pat. No. 8,957,201, issued on Feb. 17, 2015, and entitled “Oligonucleotide Analogues And Methods Utilizing The Same”, the entire contents of each of which are incorporated herein by reference for all purposes.

In some embodiments, the oligonucleotide comprises at least one modified nucleoside that results in an increase in Tm of the oligonucleotide in a range of 1 °C, 2 °C, 3 °C, 4 °C, or 5 °C compared with an oligonucleotide that does not have the at least one modified nucleoside. The oligonucleotide may have a plurality of modified nucleosides that result in a total increase in Tm of the oligonucleotide in a range of 2 °C, 3 °C, 4 °C, 5 °C, 6 °C, 7 °C, 8 °C, 9 °C, 10 °C, 15 °C, 20 °C, 25 °C, 30 °C, 35 °C, 40 °C, 45 °C or more compared with an oligonucleotide that does not have the modified nucleoside.

The oligonucleotide may comprise a mix of nucleosides of different kinds. For example, an oligonucleotide may comprise a mix of 2′-deoxyribonucleosides or ribonucleosides and 2′-fluoro modified nucleosides. An oligonucleotide may comprise a mix of deoxyribonucleosides or ribonucleosides and 2′-O-Me modified nucleosides. An oligonucleotide may comprise a mix of 2′-fluoro modified nucleosides and 2′-O-methyl modified nucleosides. An oligonucleotide may comprise a mix of bridged nucleosides and 2′-fluoro or 2′-O-methyl modified nucleosides. An oligonucleotide may comprise a mix of non-bicyclic 2′-modified nucleosides (e.g., 2′-O-MOE) and 2′-4′ bicyclic nucleosides (e.g., LNA, ENA, cEt). An oligonucleotide may comprise a mix of 2′-fluoro modified nucleosides and 2′-O-Me modified nucleosides. An oligonucleotide may comprise a mix of 2′-4′ bicyclic nucleosides and 2′-MOE, 2′-fluoro, or 2′-O-Me modified nucleosides. An oligonucleotide may comprise a mix of non-bicyclic 2′-modified nucleosides (e.g., 2′-MOE, 2′-fluoro, or 2′-O-Me) and 2′-4′ bicyclic nucleosides (e.g., LNA, ENA, cEt).

The oligonucleotide may comprise alternating nucleosides of different kinds. For example, an oligonucleotide may comprise alternating 2′-deoxyribonucleosides or ribonucleosides and 2′-fluoro modified nucleosides. An oligonucleotide may comprise alternating deoxyribonucleosides or ribonucleosides and 2′-O-Me modified nucleosides. An oligonucleotide may comprise alternating 2′-fluoro modified nucleosides and 2′-O-Me modified nucleosides. An oligonucleotide may comprise alternating bridged nucleosides and 2′-fluoro or 2′-O-methyl modified nucleosides. An oligonucleotide may comprise alternating non-bicyclic 2′-modified nucleosides (e.g., 2′-O-MOE) and 2′-4′ bicyclic nucleosides (e.g., LNA, ENA, cEt). An oligonucleotide may comprise alternating 2′-4′ bicyclic nucleosides and 2′-MOE, 2′-fluoro, or 2′-O-Me modified nucleosides. An oligonucleotide may comprise alternating non-bicyclic 2′-modified nucleosides (e.g., 2′-MOE, 2′-fluoro, or 2′-O-Me) and 2′-4′ bicyclic nucleosides (e.g., LNA, ENA, cEt).

In some embodiments, an oligonucleotide described herein comprises a 5′-vinylphosphonate modification, one or more abasic residues, and/or one or more inverted abasic residues. In some embodiments, an oligonucleotide described herein is an siRNA oligonucleotide comprising a sense strand and an antisense strand, wherein the antisense strand or the sense strand comprises a 5′-vinylphosphonate (e.g., 5′-(E)-vinylphosphonate modification). In some embodiments, an oligonucleotide described herein is an siRNA oligonucleotide comprising a sense strand and an antisense strand, wherein the antisense strand comprises a 5′-vinylphosphonate (e.g., 5′-(E)-vinylphosphonate) modification).

### d. Internucleoside Linkages/Backbones

In some embodiments, oligonucleotide may contain a phosphorothioate or other modified internucleoside linkage. In some embodiments, the oligonucleotide comprises phosphorothioate internucleoside linkages. In some embodiments, the oligonucleotide comprises phosphorothioate internucleoside linkages between at least two nucleosides. In some embodiments, the oligonucleotide comprises phosphorothioate internucleoside linkages between all nucleosides. For example, in some embodiments, oligonucleotides comprise modified internucleoside linkages at the first, second, and/or (e.g., and) third internucleoside linkage at the 5′ or 3′ end of the nucleotide sequence.

Phosphorus-containing linkages that may be used include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

In some embodiments, oligonucleotides may have heteroatom backbones, such as methylene(methylimino) or MMI backbones; amide backbones (see De Mesmaeker et al. Ace. Chem. Res. 1995, 28:366-374); morpholino backbones (see Summerton and Weller, U.S. Pat. No. 5,034,506); or peptide nucleic acid (PNA) backbones (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497).

### e. Stereospecific Oligonucleotides

In some embodiments, internucleotidic phosphorus atoms of oligonucleotides are chiral, and the properties of the oligonucleotides are adjusted based on the configuration of the chiral phosphorus atoms. In some embodiments, appropriate methods may be used to synthesize P-chiral oligonucleotide analogs in a stereocontrolled manner (e.g., as described in Oka N, Wada T, Stereocontrolled synthesis of oligonucleotide analogs containing chiral internucleotidic phosphorus atoms. Chem Soc Rev. 2011 December; 40 (12): 5829-43.) In some embodiments, phosphorothioate containing oligonucleotides are provided that comprise nucleoside units that are joined together by either substantially all Sp or substantially all Rp phosphorothioate intersugar linkages. In some embodiments, such phosphorothioate oligonucleotides having substantially chirally pure intersugar linkages are prepared by enzymatic or chemical synthesis, as described, for example, in U.S. Pat. No. 5,587,261, issued on Dec. 12, 1996, the contents of which are incorporated herein by reference in their entirety. In some embodiments, chirally controlled oligonucleotides provide selective cleavage patterns of a target nucleic acid. For example, in some embodiments, a chirally controlled oligonucleotide provides single site cleavage within a complementary sequence of a nucleic acid, as described, for example, in US Patent Application Publication 20170037399 A1, published on Feb. 2, 2017, entitled “CHIRAL DESIGN”, the contents of which are incorporated herein by reference in their entirety.

### f. Morpholinos

In some embodiments, the oligonucleotide may be a morpholino-based compounds. Morpholino-based oligomeric compounds are described in Dwaine A. Braasch and David R. Corey, Biochemistry, 2002, 41 (14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties).

### h. Gapmers

In some embodiments, an oligonucleotide described herein is a gapmer. A gapmer oligonucleotide generally has the formula 5′-X—Y-Z-3′, with X and Z as flanking regions around a gap region Y. In some embodiments, flanking region X of formula 5′-X—Y-Z-3′ is also referred to as X region, flanking sequence X, 5′ wing region X, or 5′ wing segment. In some embodiments, flanking region Z of formula 5′-X—Y-Z-3′ is also referred to as Z region, flanking sequence Z, 3′ wing region Z, or 3′ wing segment. In some embodiments, gap region Y of formula 5′-X—Y-Z-3′ is also referred to as Y region, Y segment, or gap-segment Y. In some embodiments, each nucleoside in the gap region Y is a 2′-deoxyribonucleoside, and neither the 5′ wing region X or the 3′ wing region Z contains any 2′-deoxyribonucleosides.

In some embodiments, the Y region is a contiguous stretch of nucleotides, e.g., a region of 6 or more DNA nucleotides, which are capable of recruiting an RNAse, such as RNAse H. In some embodiments, the gapmer binds to the target nucleic acid, at which point an RNAse is recruited and can then cleave the target nucleic acid. In some embodiments, the Y region is flanked both 5′ and 3′ by regions X and Z comprising high-affinity modified nucleosides, e.g., one to six high-affinity modified nucleosides. Examples of high affinity modified nucleosides include, but are not limited to, 2′-modified nucleosides (e.g., 2′-MOE, 2′O-Me, 2′-F) or 2′-4′ bicyclic nucleosides (e.g., LNA, cEt, ENA). In some embodiments, the flanking sequences X and Z may be of 1-20 nucleotides, 1-8 nucleotides, or 1-5 nucleotides in length. The flanking sequences X and Z may be of similar length or of dissimilar lengths. In some embodiments, the gap-segment Y may be a nucleotide sequence of 5-20 nucleotides, 5-15 twelve nucleotides, or 6-10 nucleotides in length.

In some embodiments, the gap region of the gapmer oligonucleotides may contain modified nucleotides known to be acceptable for efficient Rnase H action in addition to DNA nucleotides, such as C4′-substituted nucleotides, acyclic nucleotides, and arabino-configured nucleotides. In some embodiments, the gap region comprises one or more unmodified internucleoside linkages. In some embodiments, one or both flanking regions each independently comprise one or more phosphorothioate internucleoside linkages (e.g., phosphorothioate internucleoside linkages or other linkages) between at least two, at least three, at least four, at least five or more nucleotides. In some embodiments, the gap region and two flanking regions each independently comprise modified internucleoside linkages (e.g., phosphorothioate internucleoside linkages or other linkages) between at least two, at least three, at least four, at least five or more nucleotides.

A gapmer may be produced using appropriate methods. Representative U.S. patents, U.S. patent publications, and PCT publications that teach the preparation of gapmers include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; 5,700,922; 5,898,031; 7,015,315; 7,101,993; 7,399,845; 7,432,250; 7,569,686; 7,683,036; 7,750,131; 8,580,756; 9,045,754; 9,428,534; 9,695,418; 10,017,764; 10,260,069; 9,428,534; 8,580,756; U.S. patent publication Nos. US20050074801, US20090221685; US20090286969, US20100197762, and US20110112170; PCT publication Nos. WO2004069991; WO2005023825; WO2008049085 and WO2009090182; and EP Patent No. EP2,149,605, each of which is herein incorporated by reference in its entirety.

In some embodiments, a gapmer is 10⁻⁴⁰ nucleosides in length. For example, the gapmer may be 10^{-40, 10}-35, 10-30, 10-25, 10-20, 10-15, 15-40, 15-35, 15-30, 15-25, 15-20, 20-40, 20-35, 20-30, 20-25, 25-40, 25-35, 25-30, 30-40, 30-35, or 35-40 nucleosides in length. In some embodiments, a gapmer is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleosides in length.

In some embodiments, the gap region Y in a gapmer is 5-20 nucleosides in length. For example, the gap region Y may be 5-20, 5-15, 5-10, 10-20, 10-15, or 15-20 nucleosides in length. In some embodiments, the gap region Y is 5, 6, 7, 8, 9, 10, 11, 12, 13, 14, 15, 16, 17, 18, 19, or 20 nucleosides in length. In some embodiments, each nucleoside in the gap region Y is a 2′-deoxyribonucleoside. In some embodiments, all nucleosides in the gap region Y are 2′-deoxyribonucleosides. In some embodiments, one or more of the nucleosides in the gap region Y is a modified nucleoside (e.g., a 2′ modified nucleoside such as those described herein). In some embodiments, one or more cytosines in the gap region Y are optionally 5-methyl-cytosines. In some embodiments, each cytosine in the gap region Y is a 5-methyl-cytosines.

In some embodiments, the 5′wing region of a gapmer (X in the 5′-X—Y-Z-3′ formula) and the 3′wing region of a gapmer (Z in the 5′-X—Y-Z-3′ formula) are independently 1-20 nucleosides long. For example, the 5′wing region of a gapmer (X in the 5′-X—Y-Z-3′ formula) and the 3′wing region of the gapmer (Z in the 5′-X—Y-Z-3′ formula) may be independently 1-20, 1-15, 1-10, 1-7, 1-5, 1-3, 1-2, 2-5, 2-7, 3-5, 3-7, 5-20, 5-15, 5-10, 10-20, 10-15, or 15-20 nucleosides long. In some embodiments, the 5′wing region of the gapmer (X in the 5′-X—Y-Z-3′ formula) and the 3′wing region of the gapmer (Z in the 5′-X—Y-Z-3′ formula) are independently 1, 2, 3, 4, 5, 6, 7, 8, 9, 10, 11, 12, 13, 14, 15, 16, 17, 18, 19, or 20 nucleosides long. In some embodiments, the 5′wing region of the gapmer (X in the 5′-X—Y-Z-3′ formula) and the 3′wing region of the gapmer (Z in the 5′-X—Y-Z-3′ formula) are of the same length. In some embodiments, the 5′wing region of the gapmer (X in the 5′-X—Y-Z-3′ formula) and the 3′wing region of the gapmer (Z in the 5′-X—Y-Z-3′ formula) are of different lengths. In some embodiments, the 5′wing region of the gapmer (X in the 5′-X—Y-Z-3′ formula) is longer than the 3′wing region of the gapmer (Z in the 5′-X—Y-Z-3′ formula). In some embodiments, the 5′wing region of the gapmer (X in the 5′-X—Y-Z-3′ formula) is shorter than the 3′wing region of the gapmer (Z in the 5′-X—Y-Z-3′ formula).

In some embodiments, a gapmer comprises a 5′-X—Y-Z-3′ of 5-10-5, 4-12-4, 3-14-3, 2-16-2, 1-18-1, 3-10-3, 2-10-2, 1-10-1, 2-8-2, 4-6-4, 3-6-3, 2-6-2, 4-7-4, 3-7-3, 2-7-2, 4-8-4, 3-8-3, 2-8-2, 1-8-1, 2-9-2, 1-9-1, 2-10-2, 1-10-1, 1-12-1, 1-16-1, 2-15-1, 1-15-2, 1-14-3, 3-14-1, 2-14-2, 1-13-4, 4-13-1, 2-13-3, 3-13-2, 1-12-5, 5-12-1, 2-12-4, 4-12-2, 3-12-3, 1-11-6, 6-11-1, 2-11-5, 5-11-2, 3-11-4, 4-11-3, 1-17-1, 2-16-1, 1-16-2, 1-15-3, 3-15-1, 2-15-2, 1-14-4, 4-14-1, 2-14-3, 3-14-2, 1-13-5, 5-13-1, 2-13-4, 4-13-2, 3-13-3, 1-12-6, 6-12-1, 2-12-5, 5-12-2, 3-12-4, 4-12-3, 1-11-7, 7-11-1, 2-11-6, 6-11-2, 3-11-5, 5-11-3, 4-11-4, 1-18-1, 1-17-2, 2-17-1, 1-16-3, 1-16-3, 2-16-2, 1-15-4, 4-15-1, 2-15-3, 3-15-2, 1-14-5, 5-14-1, 2-14-4, 4-14-2, 3-14-3, 1-13-6, 6-13-1, 2-13-5, 5-13-2, 3-13-4, 4-13-3, 1-12-7, 7-12-1, 2-12-6, 6-12-2, 3-12-5, 5-12-3, 1-11-8, 8-11-1, 2-11-7, 7-11-2, 3-11-6, 6-11-3, 4-11-5, 5-11-4, 1-18-1, 1-17-2, 2-17-1, 1-16-3, 3-16-1, 2-16-2, 1-15-4, 4-15-1, 2-15-3, 3-15-2, 1-14-5, 2-14-4, 4-14-2, 3-14-3, 1-13-6, 6-13-1, 2-13-5, 5-13-2, 3-13-4, 4-13-3, 1-12-7, 7-12-1, 2-12-6, 6-12-2, 3-12-5, 5-12-3, 1-11-8, 8-11-1, 2-11-7, 7-11-2, 3-11-6, 6-11-3, 4-11-5, 5-11-4, 1-19-1, 1-18-2, 2-18-1, 1-17-3, 3-17-1, 2-17-2, 1-16-4, 4-16-1, 2-16-3, 3-16-2, 1-15-5, 2-15-4, 4-15-2, 3-15-3, 1-14-6, 6-14-1, 2-14-5, 5-14-2, 3-14-4, 4-14-3, 1-13-7, 7-13-1, 2-13-6, 6-13-2, 3-13-5, 5-13-3, 4-13-4, 1-12-8, 8-12-1, 2-12-7, 7-12-2, 3-12-6, 6-12-3, 4-12-5, 5-12-4, 2-11-8, 8-11-2, 3-11-7, 7-11-3, 4-11-6, 6-11-4, 5-11-5, 1-20-1, 1-19-2, 2-19-1, 1-18-3, 3-18-1, 2-18-2, 1-17-4, 4-17-1, 2-17-3, 3-17-2, 1-16-5, 2-16-4, 4-16-2, 3-16-3, 1-15-6, 6-15-1, 2-15-5, 5-15-2, 3-15-4, 4-15-3, 1-14-7, 7-14-1, 2-14-6, 6-14-2, 3-14-5, 5-14-3, 4-14-4, 1-13-8, 8-13-1, 2-13-7, 7-13-2, 3-13-6, 6-13-3, 4-13-5, 5-13-4, 2-12-8, 8-12-2, 3-12-7, 7-12-3, 4-12-6, 6-12-4, 5-12-5, 3-11-8, 8-11-3, 4-11-7, 7-11-4, 5-11-6, 6-11-5, 1-21-1, 1-20-2, 2-20-1, 1-20-3, 3-19-1, 2-19-2, 1-18-4, 4-18-1, 2-18-3, 3-18-2, 1-17-5, 2-17-4, 4-17-2, 3-17-3, 1-16-6, 6-16-1, 2-16-5, 5-16-2, 3-16-4, 4-16-3, 1-15-7, 7-15-1, 2-15-6, 6-15-2, 3-15-5, 5-15-3, 4-15-4, 1-14-8, 8-14-1, 2-14-7, 7-14-2, 3-14-6, 6-14-3, 4-14-5, 5-14-4, 2-13-8, 8-13-2, 3-13-7, 7-13-3, 4-13-6, 6-13-4, 5-13-5, Jan. 12, 2010, 10-12-1, 2-12-9, 9-12-2, 3-12-8, 8-12-3, 4-12-7, 7-12-4, 5-12-6, 6-12-5, 4-11-8, 8-11-4, 5-11-7, 7-11-5, 6-11-6, 1-22-1, 1-21-2, 2-21-1, 1-21-3, 3-20-1, 2-20-2, 1-19-4, 4-19-1, 2-19-3, 3-19-2, 1-18-5, 2-18-4, 4-18-2, 3-18-3, 1-17-6, 6-17-1, 2-17-5, 5-17-2, 3-17-4, 4-17-3, 1-16-7, 7-16-1, 2-16-6, 6-16-2, 3-16-5, 5-16-3, 4-16-4, 1-15-8, 8-15-1, 2-15-7, 7-15-2, 3-15-6, 6-15-3, 4-15-5, 5-15-4, 2-14-8, 8-14-2, 3-14-7, 7-14-3, 4-14-6, 6-14-4, 5-14-5, 3-13-8, 8-13-3, 4-13-7, 7-13-4, 5-13-6, 6-13-5, 4-12-8, 8-12-4, 5-12-7, 7-12-5, 6-12-6, 5-11-8, 8-11-5, 6-11-7, or 7-11-6. The numbers indicate the number of nucleosides in X, Y, and Z regions in the 5′-X—Y-Z-3′ gapmer.

In some embodiments, one or more nucleosides in the 5′wing region of the gapmer (X in the 5′-X—Y-Z-3′ formula) or the 3′wing region of a gapmer (Z in the 5′-X—Y-Z-3′ formula) are modified nucleosides (e.g., high-affinity modified nucleosides). In some embodiments, the modified nucleoside (e.g., high-affinity modified nucleosides) is a 2′-modified nucleoside. In some embodiments, the 2′-modified nucleoside is a 2′-4′ bicyclic nucleoside or a non-bicyclic 2′-modified nucleoside. In some embodiments, the high-affinity modified nucleoside is a 2′-4′ bicyclic nucleoside (e.g., LNA, cEt, or ENA) or a non-bicyclic 2′-modified nucleoside (e.g., 2′-fluoro (2′-F), 2′-O-methyl(2′—O-Me), 2′-O-methoxyethyl(2′-MOE), 2′-O-aminopropyl(2′—O-AP), 2′-O-dimethylaminoethyl(2′—O-DMAOE), 2′-O-dimethylaminopropyl(2′—O-DMAP), 2′-O-dimethylaminoethyloxyethyl(2′—O-DMAEOE), or 2′—O—N-methylacetamido (2′-O-NMA)).

In some embodiments, one or more nucleosides in the 5′wing region of a gapmer (X in the 5′-X—Y-Z-3′ formula) are high-affinity modified nucleosides. In some embodiments, each nucleoside in the 5′wing region of the gapmer (X in the 5′-X—Y-Z-3′ formula) is a high-affinity modified nucleoside. In some embodiments, one or more nucleosides in the 3′wing region of a gapmer (Z in the 5′-X—Y-Z-3′ formula) are high-affinity modified nucleosides. In some embodiments, each nucleoside in the 3′wing region of the gapmer (Z in the 5′-X—Y-Z-3′ formula) is a high-affinity modified nucleoside. In some embodiments, one or more nucleosides in the 5′wing region of the gapmer (X in the 5′-X—Y-Z-3′ formula) are high-affinity modified nucleosides and one or more nucleosides in the 3′wing region of the gapmer (Z in the 5′-X—Y-Z-3′ formula) are high-affinity modified nucleosides. In some embodiments, each nucleoside in the 5′wing region of the gapmer (X in the 5′-X—Y-Z-3′ formula) is a high-affinity modified nucleoside and each nucleoside in the 3′wing region of the gapmer (Z in the 5′-X—Y-Z-3′ formula) is high-affinity modified nucleoside.

In some embodiments, the 5′wing region of a gapmer (X in the 5′-X—Y-Z-3′ formula) comprises the same high affinity nucleosides as the 3′wing region of the gapmer (Z in the 5′-X—Y-Z-3′ formula). For example, the 5′wing region of the gapmer (X in the 5′-X—Y-Z-3′ formula) and the 3′wing region of the gapmer (Z in the 5′-X—Y-Z-3′ formula) may comprise one or more non-bicyclic 2′-modified nucleosides (e.g., 2′-MOE or 2′-O-Me). In another example, the 5′wing region of the gapmer (X in the 5′-X—Y-Z-3′ formula) and the 3′wing region of the gapmer (Z in the 5′-X—Y-Z-3′ formula) may comprise one or more 2′-4′ bicyclic nucleosides (e.g., LNA or cEt). In some embodiments, each nucleoside in the 5′wing region of the gapmer (X in the 5′-X—Y-Z-3′ formula) and the 3′wing region of the gapmer (Z in the 5′-X—Y-Z-3′ formula) is a non-bicyclic 2′-modified nucleosides (e.g., 2′-MOE or 2′-O-Me). In some embodiments, each nucleoside in the 5′wing region of the gapmer (X in the 5′-X—Y-Z-3′ formula) and the 3′wing region of the gapmer (Z in the 5′-X—Y-Z-3′ formula) is a 2′-4′ bicyclic nucleosides (e.g., LNA or cEt).

In some embodiments, a gapmer comprises a 5′-X—Y-Z-3′ configuration, wherein X and Z is independently 1-7 (e.g., 1, 2, 3, 4, 5, 6, or 7) nucleosides in length and Y is 6-10 (e.g., 6, 7, 8, 9, or 10) nucleosides in length, wherein each nucleoside in X and Z is a non-bicyclic 2′-modified nucleosides (e.g., 2′-MOE or 2′-O-Me) and each nucleoside in Y is a 2′-deoxyribonucleoside. In some embodiments, the gapmer comprises a 5′-X—Y-Z-3′ configuration, wherein X and Z is independently 1-7 (e.g., 1, 2, 3, 4, 5, 6, or 7) nucleosides in length and Y is 6-10 (e.g., 6, 7, 8, 9, or 10) nucleosides in length, wherein each nucleoside in X and Z is a 2′-4′ bicyclic nucleosides (e.g., LNA or cEt) and each nucleoside in Y is a 2′-deoxyribonucleoside. In some embodiments, the 5′wing region of the gapmer (X in the 5′-X—Y-Z-3′ formula) comprises different high affinity nucleosides as the 3′wing region of the gapmer (Z in the 5′-X—Y-Z-3′ formula). For example, the 5′wing region of the gapmer (X in the 5′-X—Y-Z-3′ formula) may comprise one or more non-bicyclic 2′-modified nucleosides (e.g., 2′-MOE or 2′-O-Me) and the 3′wing region of the gapmer (Z in the 5′-X—Y-Z-3′ formula) may comprise one or more 2′-4′ bicyclic nucleosides (e.g., LNA or cEt). In another example, the 3′wing region of the gapmer (Z in the 5′-X—Y-Z-3′ formula) may comprise one or more non-bicyclic 2′-modified nucleosides (e.g., 2′-MOE or 2′-O-Me) and the 5′wing region of the gapmer (X in the 5′-X—Y-Z-3′ formula) may comprise one or more 2′-4′ bicyclic nucleosides (e.g., LNA or cEt).

In some embodiments, a gapmer comprises a 5′-X—Y-Z-3′ configuration, wherein X and Z is independently 1-7 (e.g., 1, 2, 3, 4, 5, 6, or 7) nucleosides in length and Y is 6-10 (e.g., 6, 7, 8, 9, or 10) nucleosides in length, wherein each nucleoside in X is a non-bicyclic 2′-modified nucleoside (e.g., 2′-MOE or 2′-O-Me), each nucleoside in Z is a 2′-4′ bicyclic nucleoside (e.g., LNA or cEt), and each nucleoside in Y is a 2′-deoxyribonucleoside. In some embodiments, the gapmer comprises a 5′-X—Y-Z-3′ configuration, wherein X and Z is independently 1-7 (e.g., 1, 2, 3, 4, 5, 6, or 7) nucleosides in length and Y is 6-10 (e.g., 6, 7, 8, 9, or 10) nucleosides in length, wherein each nucleoside in X is a 2′-4′ bicyclic nucleoside (e.g., LNA or cEt), each nucleoside in Z is a non-bicyclic 2′-modified nucleoside (e.g., 2′-MOE or 2′-O-Me) and each nucleoside in Y is a 2′-deoxyribonucleoside.

In some embodiments, the 5′wing region of a gapmer (X in the 5′-X—Y-Z-3′ formula) comprises one or more non-bicyclic 2′-modified nucleosides (e.g., 2′-MOE or 2′-O-Me) and one or more 2′-4′ bicyclic nucleosides (e.g., LNA or cEt). In some embodiments, the 3′wing region of the gapmer (Z in the 5′-X—Y-Z-3′ formula) comprises one or more non-bicyclic 2′-modified nucleosides (e.g., 2′-MOE or 2′-O-Me) and one or more 2′-4′ bicyclic nucleosides (e.g., LNA or cEt). In some embodiments, both the 5′wing region of the gapmer (X in the 5′-X—Y-Z-3′ formula) and the 3′wing region of the gapmer (Z in the 5′-X—Y-Z-3′ formula) comprise one or more non-bicyclic 2′-modified nucleosides (e.g., 2′-MOE or 2′-O-Me) and one or more 2′-4′ bicyclic nucleosides (e.g., LNA or cEt).

In some embodiments, a gapmer comprises a 5′-X—Y-Z-3′ configuration, wherein X and Z are independently 2-7 (e.g., 2, 3, 4, 5, 6, or 7) nucleosides in length and Y is 6-10 (e.g., 6, 7, 8, 9, or 10) nucleosides in length, wherein at least one but not all (e.g., 1, 2, 3, 4, 5, or 6) of positions 1, 2, 3, 4, 5, 6, or 7 in X (the 5′ most position is position 1) is a non-bicyclic 2′-modified nucleoside (e.g., 2′-MOE or 2′-O-Me), wherein the rest of the nucleosides in both X and Z are 2′-4′ bicyclic nucleosides (e.g., LNA or cEt), and wherein each nucleoside in Y is a 2′deoxyribonucleoside. In some embodiments, the gapmer comprises a 5′-X—Y-Z-3′ configuration, wherein X and Z are independently 2-7 (e.g., 2, 3, 4, 5, 6, or 7) nucleosides in length and Y is 6-10 (e.g., 6, 7, 8, 9, or 10) nucleosides in length, wherein at least one but not all (e.g., 1, 2, 3, 4, 5, or 6) of positions 1, 2, 3, 4, 5, 6, or 7 in Z (the 5′ most position is position 1) is a non-bicyclic 2′-modified nucleoside (e.g., 2′-MOE or 2′-O-Me), wherein the rest of the nucleosides in both X and Z are 2′-4′ bicyclic nucleosides (e.g., LNA or cEt), and wherein each nucleoside in Y is a 2′deoxyribonucleoside. In some embodiments, the gapmer comprises a 5′-X—Y-Z-3′ configuration, wherein X and Z are independently 2-7 (e.g., 2, 3, 4, 5, 6, or 7) nucleosides in length and Y is 6-10 (e.g., 6, 7, 8, 9, or 10) nucleosides in length, wherein at least one but not all (e.g., 1, 2, 3, 4, 5, or 6) of positions 1, 2, 3, 4, 5, 6, or 7 in X and at least one of positions but not all (e.g., 1, 2, 3, 4, 5, or 6) of positions 1, 2, 3, 4, 5, 6, or 7 in Z (the 5′ most position is position 1) is a non-bicyclic 2′-modified nucleoside (e.g., 2′-MOE or 2′-O-Me), wherein the rest of the nucleosides in both X and Z are 2′-4′ bicyclic nucleosides (e.g., LNA or cEt), and wherein each nucleoside in Y is a 2′deoxyribonucleoside.

Non-limiting examples of gapmers configurations with a mix of non-bicyclic 2′-modified nucleoside (e.g., 2′-MOE or 2′-O-Me) and 2′-4′ bicyclic nucleosides (e.g., LNA or cEt) in the 5′wing region of the gapmer (X in the 5′-X—Y-Z-3′ formula) and/or the 3′wing region of the gapmer (Z in the 5′-X—Y-Z-3′ formula) include: BBB-(D)n-BBBAA; KKK-(D)n-KKKAA; LLL-(D)n-LLLAA; BBB-(D)n-BBBEE; KKK-(D)n-KKKEE; LLL-(D)n-LLLEE; BBB-(D)n-BBBAA; KKK-(D)n-KKKAA; LLL-(D)n-LLLAA; BBB-(D)n-BBBEE; KKK-(D)n-KKKEE; LLL-(D)n-LLLEE; BBB-(D)n-BBBAAA; KKK-(D)n-KKKAAA; LLL-(D)n-LLLAAA; BBB-(D)n-BBBEEE; KKK-(D)n-KKKEEE; LLL-(D)n-LLLEEE; BBB-(D)n-BBBAAA; KKK-(D)n-KKKAAA; LLL-(D)n-LLLAAA; BBB-(D)n-BBBEEE; KKK-(D)n-KKKEEE; LLL-(D)n-LLLEEE; BABA-(D)n-ABAB; KAKA-(D)n-AKAK; LALA-(D)n-ALAL; BEBE-(D)n-EBEB; KEKE-(D)n-EKEK; LELE-(D)n-ELEL; BABA-(D)n-ABAB; KAKA-(D)n-AKAK; LALA-(D)n-ALAL; BEBE-(D)n-EBEB; KEKE-(D)n-EKEK; LELE-(D)n-ELEL; ABAB-(D)n-ABAB; AKAK-(D)n-AKAK; ALAL-(D)n-ALAL; EBEB-(D)n-EBEB; EKEK-(D)n-EKEK; ELEL-(D)n-ELEL; ABAB-(D)n-ABAB; AKAK-(D)n-AKAK; ALAL-(D)n-ALAL; EBEB-(D)n-EBEB; EKEK-(D)n-EKEK; ELEL-(D)n-ELEL; AABB-(D)n-BBAA; BBAA-(D)n-AABB; AAKK-(D)n-KKAA; AALL-(D)n-LLAA; EEBB-(D)n-BBEE; EEKK-(D)n-KKEE; EELL-(D)n-LLEE; AABB-(D)n-BBAA; AAKK-(D)n-KKAA; AALL-(D)n-LLAA; EEBB-(D)n-BBEE; EEKK-(D)n-KKEE; EELL-(D)n-LLEE; BBB-(D)n-BBA; KKK-(D)n-KKA; LLL-(D)n-LLA; BBB-(D)n-BBE; KKK-(D)n-KKE; LLL-(D)n-LLE; BBB-(D)n-BBA; KKK-(D)n-KKA; LLL-(D)n-LLA; BBB-(D)n-BBE; KKK-(D)n-KKE; LLL-(D)n-LLE; BBB-(D)n-BBA; KKK-(D)n-KKA; LLL-(D)n-LLA; BBB-(D)n-BBE; KKK-(D)n-KKE; LLL-(D)n-LLE; ABBB-(D)n-BBBA; AKKK-(D)n-KKKA; ALLL-(D)n-LLLA; EBBB-(D)n-BBBE; EKKK-(D)n-KKKE; ELLL-(D)n-LLLE; ABBB-(D)n-BBBA; AKKK-(D)n-KKKA; ALLL-(D)n-LLLA; EBBB-(D)n-BBBE; EKKK-(D)n-KKKE; ELLL-(D)n-LLLE; ABBB-(D)n-BBBAA; AKKK-(D)n-KKKAA; ALLL-(D)n-LLLAA; EBBB-(D)n-BBBEE; EKKK-(D)n-KKKEE; ELLL-(D)n-LLLEE; ABBB-(D)n-BBBAA; AKKK-(D)n-KKKAA; ALLL-(D)n-LLLAA; EBBB-(D)n-BBBEE; EKKK-(D)n-KKKEE; ELLL-(D)n-LLLEE; AABBB-(D)n-BBB; AAKKK-(D)n-KKK; AALLL-(D)n-LLL; EEBBB-(D)n-BBB; EEKKK-(D)n-KKK; EELLL-(D)n-LLL; AABBB-(D)n-BBB; AAKKK-(D)n-KKK; AALLL-(D)n-LLL; EEBBB-(D)n-BBB; EEKKK-(D)n-KKK; EELLL-(D)n-LLL; AABBB-(D)n-BBBA; AAKKK-(D)n-KKKA; AALLL-(D)n-LLLA; EEBBB-(D)n-BBBE; EEKKK-(D)n-KKKE; EELLL-(D)n-LLLE; AABBB-(D)n-BBBA; AAKKK-(D)n-KKKA; AALLL-(D)n-LLLA; EEBBB-(D)n-BBBE; EEKKK-(D)n-KKKE; EELLL-(D)n-LLLE; ABBAABB-(D)n-BB; AKKAAKK-(D)n-KK; ALLAALLL-(D)n-LL; EBBEEBB-(D)n-BB; EKKEEKK-(D)n-KK; ELLEELL-(D)n-LL; ABBAABB-(D)n-BB; AKKAAKK-(D)n-KK; ALLAALL-(D)n-LL; EBBEEBB-(D)n-BB; EKKEEKK-(D)n-KK; ELLEELL-(D)n-LL; ABBABB-(D)n-BBB; AKKAKK-(D)n-KKK; ALLALLL-(D)n-LLL; EBBEBB-(D)n-BBB; EKKEKK-(D)n-KKK; ELLELL-(D)n-LLL; ABBABB-(D)n-BBB; AKKAKK-(D)n-KKK; ALLALL-(D)n-LLL; EBBEBB-(D)n-BBB; EKKEKK-(D)n-KKK; ELLELL-(D)n-LLL; EEEK-(D)n-EEEEEEEE; EEK-(D)n-EEEEEEEEE; EK-(D)n-EEEEEEEEEE; EK-(D)n-EEEKK; K-(D)n-EEEKEKE; K-(D)n-EEEKEKEE; K-(D)n-EEKEK; EK-(D)n-EEEEKEKE; EK-(D)n-EEEKEK; EEK-(D)n-KEEKE; EK-(D)n-EEKEK; EK-(D)n-KEEK; EEK-(D)n-EEEKEK; EK-(D)n-KEEEKEE; EK-(D)n-EEKEKE; EK-(D)n-EEEKEKE; and EK-(D)n-EEEEKEK;. “A” nucleosides comprise a 2′-modified nucleoside; “B” represents a 2′-4′ bicyclic nucleoside; “K” represents a constrained ethyl nucleoside (cEt); “L” represents an LNA nucleoside; and “E” represents a 2′-MOE modified ribonucleoside; “D” represents a 2′-deoxyribonucleoside; “n” represents the length of the gap segment (Y in the 5′-X—Y-Z-3′ configuration) and is an integer between 1-20.

In some embodiments, any one of the gapmers described herein comprises one or more modified nucleoside linkages (e.g., a phosphorothioate linkage) in each of the X, Y, and Z regions. In some embodiments, each internucleoside linkage in the any one of the gapmers described herein is a phosphorothioate linkage. In some embodiments, each of the X, Y, and Z regions independently comprises a mix of phosphorothioate linkages and phosphodiester linkages. In some embodiments, each internucleoside linkage in the gap region Y is a phosphorothioate linkage, the 5′wing region X comprises a mix of phosphorothioate linkages and phosphodiester linkages, and the 3′wing region Z comprises a mix of phosphorothioate linkages and phosphodiester linkages.

### i. RNA Interference (RNAi)

In some embodiments, the oligonucleotides provided herein are small interfering RNAs (siRNA), also known as short interfering RNA or silencing RNA. SiRNA, is a class of double-stranded RNA molecules, typically about 20-25 base pairs in length that target nucleic acids (e.g., mRNAs) for degradation via the RNA interference (RNAi) pathway in cells. Specificity of siRNA molecules may be determined by the binding of the antisense strand of the molecule to its target RNA. Effective siRNA molecules are generally less than 30 to 35 base pairs in length to prevent the triggering of non-specific RNA interference pathways in the cell via the interferon response, although longer siRNA can also be effective. In some embodiments, the siRNA molecules are 7, 8, 9, 10, 11, 12, 13, 14, 15, 16, 17, 18, 19, 20, 21, 22, 23, 24, 25, 26, 27, 28, 29, 30, 35, 40, 45, 50, or more base pairs in length. In some embodiments, the siRNA molecules are 8 to 30 base pairs in length, 10 to 15 base pairs in length, 10 to 20 base pairs in length, 15 to 25 base pairs in length, 19 to 21 base pairs in length, 21 to 23 base pairs in length. In some embodiments, the siRNA molecules are 8 to 32 base pairs in length, 8 to 29 base pairs in length, 8 to 27 base pairs in length, 15 to 32 base pairs in length, 15 to 29 base pairs in length, 15 to 27 base pairs in length, 21 to 31 base pairs in length, 21 to 29 base pairs in length, 21 to 27 base pairs in length, 21-23 base pairs in length, 23 to 32 base pairs in length, 23 to 29 base pairs in length, or 23 to 27 base pairs in length.

Following selection of an appropriate target RNA sequence, siRNA molecules that comprise a nucleotide sequence complementary to all or a portion of the target sequence, i.e. an antisense sequence, can be designed and prepared using appropriate methods (see, e.g., PCT Publication Number WO 2004/016735; and U.S. Patent Publication Nos. 2004/0077574 and 2008/0081791).

The siRNA molecule can be double stranded (i.e. a dsRNA molecule comprising an antisense strand and a complementary sense strand) or single-stranded (i.e. a ssRNA molecule comprising just an antisense strand). The siRNA molecules can comprise a duplex, asymmetric duplex, hairpin or asymmetric hairpin secondary structure, having self-complementary sense and antisense strands. In some embodiments, the oligonucleotide described herein is an siRNA comprising an antisense strand and a sense strand.

In some embodiments, the antisense strand of the siRNA molecule is 7, 8, 9, 10, 11, 12, 13, 14, 15, 16, 17, 18, 19, 20, 21, 22, 23, 24, 25, 26, 27, 28, 29, 30, 35, 40, 45, 50, or more nucleotides in length. In some embodiments, the antisense strand is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 19 to 21 nucleotides in length, 21 to 23 nucleotides in lengths. In some embodiments, the antisense strand is 8 to 32 nucleotides in length, 8 to 29 nucleotides in length, 8 to 27 nucleotides in length, 15 to 32 nucleotides in length, 15 to 29 nucleotides in length, 15 to 27 nucleotides in length, 21 to 31 nucleotides in length, 21 to 29 nucleotides in length, 21 to 27 nucleotides in length, 21-23 nucleotides in length, 23 to 32 nucleotides in length, 23 to 29 nucleotides in length, or 23 to 27 nucleotides in length.

In some embodiments, the sense strand of the siRNA molecule is 7, 8, 9, 10, 11, 12, 13, 14, 15, 16, 17, 18, 19, 20, 21, 22, 23, 24, 25, 26, 27, 28, 29, 30, 35, 40, 45, 50, or more nucleotides in length. In some embodiments, the sense strand is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 19 to 21 nucleotides in length, 21 to 23 nucleotides in lengths. In some embodiments, the sense strand is 8 to 32 nucleotides in length, 8 to 29 nucleotides in length, 8 to 27 nucleotides in length, 15 to 32 nucleotides in length, 15 to 29 nucleotides in length, 15 to 27 nucleotides in length, 21 to 31 nucleotides in length, 21 to 29 nucleotides in length, 21 to 27 nucleotides in length, 21-23 nucleotides in length, 23 to 32 nucleotides in length, 23 to 29 nucleotides in length, or 23 to 27 nucleotides in length.

In some embodiments, siRNA molecules comprise an antisense strand comprising a region of complementarity to a target region in a DUX4 mRNA. In some embodiments, the region of complementarity is at least 80%, at least 85%, at least 90%, at least 91%, at least 92%, at least 93%, at least 94%, at least 95%, at least 96%, at least 97%, at least 98%, at least 99% or 100% complementary to a target region in a DUX4 mRNA. In some embodiments, the target region is a region of consecutive nucleotides in the DUX4 mRNA. In some embodiments, a complementary nucleotide sequence need not be 100% complementary to that of its target to be specifically hybridizable or specific for a target RNA sequence.

In some embodiments, siRNA molecules comprise an antisense strand that comprises a region of complementarity to a DUX4 mRNA sequence and the region of complementarity is in the range of 8 to 15, 8 to 30, 8 to 40, or 10 to 50, or 5 to 50, or 5 to 40 nucleotides in length. In some embodiments, a region of complementarity is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity is complementary with at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25 or more consecutive nucleotides of a DUX4 mRNA sequence. In some embodiments, the region of complementarity comprises a nucleotide sequence that contains no more than 1, 2, 3, 4, or 5 base mismatches compared to the complementary portion of a DUX4 mRNA sequence. In some embodiments, the region of complementarity comprises a nucleotide sequence that has up to 3 mismatches over 15 bases, or up to 2 mismatches over 10 bases.

In some embodiments, siRNA molecules comprise an antisense strand comprising a nucleotide sequence that is complementary (e.g., at least 85%, at least 90%, at least 95%, or 100%) to a target RNA sequence as set forth in any one of SEQ ID NOs: 174-235. In some embodiments, siRNA molecules comprise an antisense strand comprising a nucleotide sequence that is complementary (e.g., at least 85%, at least 90%, at least 95%, or 100%) to a target RNA sequence as set forth in any one of SEQ ID NOs: 186, 189-191, and 200. In some embodiments, siRNA molecules comprise an antisense strand of 18-25 nucleotides in length and comprising a region of complementarity of at least 15 nucleotides (e.g., at least 15 nucleotides, at least 16 nucleotides, at least 17 nucleotides, at least 18 nucleotides, or at least 19 nucleotides) to a target RNA sequence as set forth in any one of SEQ ID NOs: 174-235. In some embodiments, siRNA molecules comprise an antisense strand of 18-25 nucleotides in length and comprising a region of complementarity of at least 15 nucleotides (e.g., at least 15 nucleotides, at least 16 nucleotides, at least 17 nucleotides, at least 18 nucleotides, or at least 19 nucleotides) to a target RNA sequence as set forth in any one of SEQ ID NOs: 186, 189-191, and 200.

In some embodiments, siRNA molecules comprise an antisense strand comprising a nucleotide sequence that is at least 85%, at least 90%, at least 95%, or 100% identical to the oligonucleotides as set forth in any one of SEQ ID NOs: 236-266. In some embodiments, siRNA molecules comprise an antisense strand comprising a nucleotide sequence that is at least 85%, at least 90%, at least 95%, or 100% identical to the oligonucleotides as set forth in any one of SEQ ID NOs: 248, 251-253 and 262. In some embodiments, siRNA molecules comprise an antisense strand of 18-25 nucleotides in length and comprising at least 6, at least 7, at least 8, at least 9, at least 10, at least 11, at least 12, at least 13, at least 14, at least 15, at least 16, at least 17, at least 18, or at least 19 consecutive nucleotides of the oligonucleotides as set forth in any one of SEQ ID NOs: 236-266. In some embodiments, siRNA molecules comprise an antisense strand of 18-25 nucleotides in length and comprising at least 6, at least 7, at least 8, at least 9, at least 10, at least 11, at least 12, at least 13, at least 14, at least 15, at least 16, at least 17, at least 18, or at least 19 consecutive nucleotides of the oligonucleotides as set forth in any one of SEQ ID NOs: 248, 251-253 and 262.

Double-stranded siRNA may comprise sense and antisense RNA strands that are the same length or different lengths. Double-stranded siRNA molecules can also be assembled from a single oligonucleotide in a stem-loop structure, wherein self-complementary sense and antisense regions of the siRNA molecule are linked by means of a nucleic acid based or non-nucleic acid-based linker(s), as well as circular single-stranded RNA having two or more loop structures and a stem comprising self-complementary sense and antisense strands, wherein the circular RNA can be processed either in vivo or in vitro to generate an active siRNA molecule capable of mediating RNAi. Small hairpin RNA (shRNA) molecules thus are also contemplated herein. These molecules comprise a specific antisense sequence in addition to the reverse complement (sense) sequence, typically separated by a spacer or loop sequence. Cleavage of the spacer or loop provides a single-stranded RNA molecule and its reverse complement, such that they may anneal to form a dsRNA molecule (optionally with additional processing steps that may result in addition or removal of one, two, three or more nucleotides from the 3′ end and/or (e.g., and) the 5′ end of either or both strands). A spacer can be of a sufficient length to permit the antisense and sense sequences to anneal and form a double-stranded structure (or stem) prior to cleavage of the spacer (and, optionally, subsequent processing steps that may result in addition or removal of one, two, three, four, or more nucleotides from the 3′ end and/or (e.g., and) the 5′ end of either or both strands). A spacer sequence may be an unrelated nucleotide sequence that is situated between two complementary nucleotide sequence regions which, when annealed into a double-stranded nucleic acid, comprise a shRNA.

The overall length of the siRNA molecules can vary from about 14 to about 100 nucleotides depending on the type of siRNA molecule being designed. Generally between about 14 and about 50 of these nucleotides are complementary to the RNA target sequence, i.e. constitute the specific antisense sequence of the siRNA molecule. For example, when the siRNA is a double- or single-stranded siRNA, the length can vary from about 14 to about 50 nucleotides, whereas when the siRNA is a shRNA or circular molecule, the length can vary from about 40 nucleotides to about 100 nucleotides.

An siRNA molecule may comprise a 3′ overhang at one end of the molecule. The other end may be blunt-ended or have also an overhang (5′ or 3′). When the siRNA molecule comprises an overhang at both ends of the molecule, the length of the overhangs may be the same or different. In one embodiment, the siRNA molecule of the present disclosure comprises 3′ overhangs of about 1 to about 3 (e.g., 1, 2, 3) nucleotides on both ends of the molecule. In some embodiments, the siRNA molecule comprises 3′ overhangs of about 1 to about 3 nucleotides on the sense strand. In some embodiments, the siRNA molecule comprises 3′ overhangs of about 1 to about 3 (e.g., 1, 2, 3) nucleotides on the antisense strand. In some embodiments, the siRNA molecule comprises 3′ overhangs of about 1 to about 3 (e.g., 1, 2, 3) nucleotides on both the sense strand and the antisense strand.

In some embodiments, the siRNA molecule comprises one or more modified nucleotides (e.g. 1, 2, 3, 4, 5, 6, 7, 8, 9, 10 or more). In some embodiments, the siRNA molecule comprises one or more modified nucleotides and/or (e.g., and) one or more modified internucleoside linkages. In some embodiments, the modified nucleotide is a modified sugar moiety (e.g. a 2′ modified nucleotide). In some embodiments, the siRNA molecule comprises one or more 2′ modified nucleotides, e.g., a 2′-deoxy, 2′-fluoro (2′-F), 2′-O-methyl(2′—O-Me), 2′-O-methoxyethyl(2′-MOE), 2′-O-aminopropyl(2′—O-AP), 2′-O-dimethylaminoethyl (2′-O-DMAOE), 2′-O-dimethylaminopropyl(2′—O-DMAP), 2′-O-dimethylaminoethyloxyethyl(2′—O-DMAEOE), or 2′—O—N-methylacetamido (2′-O-NMA). In some embodiments, each nucleotide of the siRNA molecule is a modified nucleotide (e.g., a 2′-modified nucleotide). In some embodiments, the siRNA molecule comprises one or more 2′-O-methyl modified nucleotides. In some embodiments, the siRNA molecule comprises one or more 2′-F modified nucleotides. In some embodiments, the siRNA molecule comprises one or more 2′-O-methyl and 2′-F modified nucleotides. In some embodiments, the siRNA molecule comprises one or more modified nucleosides (e.g. 1, 2, 3, 4, 5, 6, 7, 8, 9, 10 or more). In some embodiments, the siRNA molecule comprises one or more modified nucleosides and/or (e.g., and) one or more modified internucleoside linkages. In some embodiments, the modified nucleoside is a modified sugar moiety (e.g. a 2′ modified nucleoside). In some embodiments, the siRNA molecule comprises one or more 2′ modified nucleosides, e.g., a 2′-deoxy, 2′-fluoro (2′-F), 2′-O-methyl(2′—O-Me), 2′-O-methoxyethyl(2′-MOE), 2′-O-aminopropyl(2′—O-AP), 2′-O-dimethylaminoethyl(2′—O-DMAOE), 2′-O-dimethylaminopropyl(2′—O-DMAP), 2′-O-dimethylaminoethyloxyethyl(2′—O-DMAEOE), or 2′—O—N-methylacetamido (2′-O-NMA). In some embodiments, each nucleoside of the siRNA molecule is a modified nucleotide (e.g., a 2′-modified nucleoside). In some embodiments, the siRNA molecule comprises one or more 2′-O-methyl modified nucleosides. In some embodiments, the siRNA molecule comprises one or more 2′-F modified nucleosides. In some embodiments, the siRNA molecule comprises one or more 2′-O-methyl and 2′-F modified nucleosides.

In some embodiments, the siRNA molecule contains a phosphorothioate or other modified internucleotide linkage. In some embodiments, the siRNA molecule contains a phosphorothioate or other modified internucleoside linkage. In some embodiments, the siRNA molecule comprises phosphorothioate internucleoside linkages. In some embodiments, the siRNA molecule comprises phosphorothioate internucleotide linkages. In some embodiments, the siRNA molecule comprises phosphorothioate internucleoside linkages between at least two nucleotides. In some embodiments, the siRNA molecule comprises phosphorothioate internucleoside linkages between at least two nucleosides. In some embodiments, the siRNA molecule comprises phosphorothioate internucleotide linkages between at least two nucleotides. In some embodiments, the siRNA molecule comprises phosphorothioate internucleoside linkages between all nucleotides. In some embodiments, the siRNA molecule comprises phosphorothioate internucleoside linkages between all nucleosides. For example, in some embodiments, the siRNA molecule comprises modified internucleoside linkages at the first, second, and/or (e.g., and) third internucleoside linkage at the 5′ or 3′ end of the siRNA molecule. In some embodiments, the siRNA molecule comprises phosphorothioate internucleotide linkages between all nucleotides. For example, in some embodiments, the siRNA molecule comprises modified internucleotide linkages at the first, second, and/or (e.g., and) third internucleotide linkage at the 5′ or 3′ end of the siRNA molecule. For example, in some embodiments, the siRNA molecule comprises modified internucleotide linkages at the first, second, and/or (e.g., and) third internucleoside linkage at the 5′ or 3′ end of the siRNA molecule.

In some embodiments, the modified internucleotide linkages are phosphorus-containing linkages. In some embodiments, the modified internucleoside linkages are phosphorus-containing linkages. In some embodiments, phosphorus-containing linkages that may be used include, but are not limited to, phosphorothioates, chiral phosphorothioates, phosphorodithioates, phosphotriesters, aminoalkylphosphotriesters, methyl and other alkyl phosphonates comprising 3′ alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Any of the modified chemistries or formats of siRNA molecules described herein can be combined with each other. For example, one, two, three, four, five, or more different types of modifications can be included within the same siRNA molecule.

In some embodiments, the antisense strand comprises one or more modified nucleotides (e.g. 1, 2, 3, 4, 5, 6, 7, 8, 9, 10 or more). In some embodiments, the antisense strand comprises one or more modified nucleotides and/or (e.g., and) one or more modified internucleotide linkages. In some embodiments, the modified nucleotide comprises a modified sugar moiety (e.g. a 2′ modified nucleotide). In some embodiments, the antisense strand comprises one or more 2′ modified nucleotides, e.g., a 2′-deoxy, 2′-fluoro (2′-F), 2′-O-methyl (2′-O-Me), 2′-O-methoxyethyl(2′-MOE), 2′-O-aminopropyl(2′—O-AP), 2′-O-dimethylaminoethyl(2′—O-DMAOE), 2′-O-dimethylaminopropyl(2′—O-DMAP), 2′-O-dimethylaminoethyloxyethyl(2′—O-DMAEOE), or 2′—O—N-methylacetamido (2′-O-NMA). In some embodiments, each nucleotide of the antisense strand is a modified nucleotide (e.g., a 2′-modified nucleotide). In some embodiments, the antisense strand comprises one or more 2′-O-methyl modified nucleotides. In some embodiments, the antisense strand comprises one or more 2′-F modified nucleotides. In some embodiments, the antisense strand comprises one or more 2′-O-methyl and 2′-F modified nucleotides. In some embodiments, the antisense strand comprises one or more modified nucleosides (e.g. 1, 2, 3, 4, 5, 6, 7, 8, 9, 10 or more). In some embodiments, the antisense strand comprises one or more modified nucleosides and/or (e.g., and) one or more modified internucleoside linkages. In some embodiments, the modified nucleoside comprises a modified sugar moiety (e.g. a 2′ modified nucleoside). In some embodiments, the antisense strand comprises one or more 2′ modified nucleosides, e.g., a 2′-deoxy, 2′-fluoro (2′-F), 2′-O-methyl(2′—O-Me), 2′-O-methoxyethyl(2′-MOE), 2′-O-aminopropyl(2′—O-AP), 2′-O-dimethylaminoethyl(2′—O-DMAOE), 2′-O-dimethylaminopropyl(2′—O-DMAP), 2′-O-dimethylaminoethyloxyethyl(2′—O-DMAEOE), or 2′—O—N-methylacetamido (2′-O-NMA). In some embodiments, each nucleoside of the antisense strand is a modified nucleoside (e.g., a 2′-modified nucleoside). In some embodiments, the antisense strand described herein comprises one or more 2′-F modified nucleoside. In some embodiments, the antisense strand described herein comprises at least two 2′-F modified nucleosides. In some embodiments, the antisense strand described herein comprises at least four 2′-F modified nucleosides. In some embodiments, the antisense strand described herein comprises at least six 2′-fluoro (2′-F) modified nucleosides. In some embodiments, the antisense strand described herein comprises one or more 2′-O-methyl modified nucleoside. In some embodiments, the antisense strand comprises one or more 2′-O-methyl and 2′-F modified nucleosides.

In some embodiments, antisense strand contains a phosphorothioate or other modified internucleotide linkage. In some embodiments, antisense strand contains a phosphorothioate or other modified internucleoside linkage. In some embodiments, the antisense strand comprises phosphorothioate internucleoside linkages. In some embodiments, the antisense strand comprises phosphorothioate internucleotide linkages. In some embodiments, the antisense strand comprises phosphorothioate internucleoside linkages between at least two nucleotides. In some embodiments, the antisense strand comprises phosphorothioate internucleoside linkages between at least two nucleosides. In some embodiments, the antisense strand comprises phosphorothioate internucleotide linkages between at least two nucleotides. In some embodiments, the antisense strand comprises phosphorothioate internucleoside linkages between all nucleotides. In some embodiments, the antisense strand comprises phosphorothioate internucleoside linkages between all nucleosides. For example, in some embodiments, the antisense strand comprises modified internucleoside linkages at the first, second, and/or (e.g., and) third internucleoside linkage at the 5′ or 3′ end of the siRNA molecule. In some embodiments, the two internucleoside linkages at the 3′ end of the antisense strands are phosphorothioate internucleoside linkages. In some embodiments, the antisense strand comprises phosphorothioate internucleotide linkages between all nucleotides. For example, in some embodiments, the antisense strand comprises modified internucleotide linkages at the first, second, and/or (e.g., and) third internucleotide linkage at the 5′ or 3′ end of the siRNA molecule. In some embodiments, the two internucleotide linkages at the 3′ end of the antisense strands are phosphorothioate internucleotide linkages. For example, in some embodiments, the antisense strand comprises modified internucleotide linkages at the first, second, and/or (e.g., and) third internucleoside linkage at the 5′ or 3′ end of the siRNA molecule. In some embodiments, the two internucleoside linkages at the 3′ end of the antisense strands are phosphorothioate internucleoside linkages.

In some embodiments, the modified internucleotide linkages are phosphorus-containing linkages. In some embodiments, the modified internucleoside linkages are phosphorus-containing linkages. In some embodiments, phosphorus-containing linkages that may be used include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Any of the modified chemistries or formats of the antisense strand described herein can be combined with each other. For example, one, two, three, four, five, or more different types of modifications can be included within the same antisense strand.

In some embodiments, the sense strand comprises one or more modified nucleotides (e.g., 1, 2, 3, 4, 5, 6, 7, 8, 9, 10 or more). In some embodiments, the sense strand comprises one or more modified nucleotides and/or (e.g., and) one or more modified internucleotide linkages. In some embodiments, the modified nucleotide comprises a modified sugar moiety (e.g. a 2′ modified nucleotide). In some embodiments, the sense strand comprises one or more 2′ modified nucleotides, e.g., a 2′-deoxy, 2′-fluoro (2′-F), 2′-O-methyl(2′—O-Me), 2′-O-methoxyethyl(2′-MOE), 2′-O-aminopropyl(2′—O-AP), 2′-O-dimethylaminoethyl (2′-O-DMAOE), 2′-O-dimethylaminopropyl(2′—O-DMAP), 2′-O-dimethylaminoethyloxyethyl(2′—O-DMAEOE), or 2′—O—N-methylacetamido (2′-O-NMA). In some embodiments, each nucleotide of the sense strand is a modified nucleotide (e.g., a 2′-modified nucleotide). In some embodiments, the sense strand comprises one or more modified nucleosides (e.g., 1, 2, 3, 4, 5, 6, 7, 8, 9, 10 or more). In some embodiments, the sense strand comprises one or more modified nucleotides and/or (e.g., and) one or more modified internucleoside linkages. In some embodiments, the modified nucleoside comprises a modified sugar moiety (e.g. a 2′ modified nucleoside). In some embodiments, the sense strand comprises one or more 2′ modified nucleosides, e.g., a 2′-deoxy, 2′-fluoro (2′-F), 2′-O-methyl(2′—O-Me), 2′-O-methoxyethyl(2′-MOE), 2′-O-aminopropyl(2′—O-AP), 2′-O-dimethylaminoethyl (2′-O-DMAOE), 2′-O-dimethylaminopropyl(2′—O-DMAP), 2′-O-dimethylaminoethyloxyethyl(2′—O-DMAEOE), or 2′—O—N-methylacetamido (2′-O-NMA). In some embodiments, each nucleoside of the sense strand is a modified nucleoside (e.g., a 2′-modified nucleoside). In some embodiments, the sense strand comprises one or more phosphorodiamidate morpholinos. In some embodiments, the sense strand is a phosphorodiamidate morpholino oligomer (PMO). In some embodiments, the sense strand comprises one or more 2′-O-methyl modified nucleotides. In some embodiments, the sense strand comprises one or more 2′-F modified nucleotides. In some embodiments, the sense strand comprises one or more 2′-O-methyl and 2′-F modified nucleotides.

In some embodiments, the sense strand described herein comprises one or more 2′-F modified nucleoside. In some embodiments, the sense strand described herein comprises at least two 2′-F modified nucleosides. In some embodiments, the sense strand described herein comprises at least four 2′-F modified nucleosides. In some embodiments, the sense strand described herein comprises at least six 2′-F modified nucleosides. In some embodiments, the sense strand described herein comprises one or more 2′-O-methyl modified nucleoside. In some embodiments, the sense strand comprises one or more 2′-O-methyl and 2′-F modified nucleosides.

In some embodiments, the sense strand contains a phosphorothioate or other modified internucleotide linkage. In some embodiments, the sense strand contains a phosphorothioate or other modified internucleoside linkage. In some embodiments, the sense strand comprises phosphorothioate internucleoside linkages. In some embodiments, the sense strand comprises phosphorothioate internucleotide linkages. In some embodiments, the sense strand comprises phosphorothioate internucleoside linkages between at least two nucleotides. In some embodiments, the sense strand comprises phosphorothioate internucleoside linkages between at least two nucleosides. In some embodiments, the sense strand comprises phosphorothioate internucleotide linkages between at least two nucleotides. In some embodiments, the sense strand comprises phosphorothioate internucleoside linkages between all nucleotides. In some embodiments, the sense strand comprises phosphorothioate internucleoside linkages between all nucleosides. For example, in some embodiments, the sense strand comprises modified internucleoside linkages at the first, second, and/or (e.g., and) third internucleoside linkage at the 5′ or 3′ end of the sense strand. In some embodiments, the sense strand comprises phosphorothioate internucleotide linkages between all nucleotides. For example, in some embodiments, the sense strand comprises modified internucleotide linkages at the first, second, and/or (e.g., and) third internucleotide linkage at the 5′ or 3′ end of the sense strand. For example, in some embodiments, the sense strand comprises modified internucleotide linkages at the first, second, and/or (e.g., and) third internucleoside linkage at the 5′ or 3′ end of the sense strand. In some embodiments, the sense strand comprises phosphodiester internucleoside linkage. In some embodiments, the sense strand does not comprise phosphorothioate internucleoside linkage. In some embodiments, the modified internucleotide linkages are phosphorus-containing linkages. In some embodiments, the modified internucleoside linkages are phosphorus-containing linkages. In some embodiments, phosphorus-containing linkages that may be used include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Any of the modified chemistries or formats of the sense strand described herein can be combined with each other. For example, one, two, three, four, five, or more different types of modifications can be included within the same sense strand.

In some embodiments, the antisense or sense strand of the siRNA molecule comprises modifications that enhance or reduce RNA-induced silencing complex (RISC) loading. In some embodiments, the antisense strand of the siRNA molecule comprises modifications that enhance RISC loading. In some embodiments, the sense strand of the siRNA molecule comprises modifications that reduce RISC loading and reduce off-target effects. In some embodiments, the antisense strand of the siRNA molecule comprises a 2′-O-methoxyethyl(2′-MOE) modification. The addition of the 2′-O-methoxyethyl(2′-MOE) group at the cleavage site improves both the specificity and silencing activity of siRNAs by facilitating the oriented RNA-induced silencing complex (RISC) loading of the modified strand, as described in Song et al., (2017) Mol Ther Nucleic Acids 9:242-250, incorporated herein by reference in its entirety. In some embodiments, the antisense strand of the siRNA molecule comprises a 2′-OMe-phosphorodithioate modification, which increases RISC loading as described in Wu et al., (2014) Nat Commun 5:3459, incorporated herein by reference in its entirety.

In some embodiments, the sense strand of the siRNA molecule comprises a 5′-morpholino, which reduces RISC loading of the sense strand and improves antisense strand selection and RNAi activity, as described in Kumar et al., (2019) Chem Commun (Camb) 55 (35): 5139-5142, incorporated herein by reference in its entirety. In some embodiments, the sense strand of the siRNA molecule is modified with a synthetic RNA-like high affinity nucleotide analogue, Locked Nucleic Acid (LNA), which reduces RISC loading of the sense strand and further enhances antisense strand incorporation into RISC, as described in Elman et al., (2005) Nucleic Acids Res. 33 (1): 439-447, incorporated herein by reference in its entirety. In some embodiments, the sense strand of the siRNA molecule comprises a 5′ unlocked nucleic acid (UNA) modification, which reduce RISC loading of the sense strand and improve silencing potency of the antisense strand, as described in Snead et al., (2013) Mol Ther Nucleic Acids 2 (7): e103, incorporated herein by reference in its entirety. In some embodiments, the sense strand of the siRNA molecule comprises a 5-nitroindole modification, which decreased the RNAi potency of the sense strand and reduces off-target effects as described in Zhang et al., (2012) Chembiochem 13 (13): 1940-1945, incorporated herein by reference in its entirety. In some embodiments, the sense strand comprises a 2′-O-methyl(2′—O-Me) modification, which reduces RISC loading and the off-target effects of the sense strand, as described in Zheng et al., FASEB (2013) 27 (10): 4017-4026, incorporated herein by reference in its entirety. In some embodiments, the sense strand of the siRNA molecule is fully substituted with morpholino, 2′-MOE or 2′-O-Me residues, and are not recognized by RISC as described in Kole et al., (2012) Nature reviews. Drug Discovery 11 (2): 125-140, incorporated herein by reference in its entirety. In some embodiments the antisense strand of the siRNA molecule comprises a 2′-MOE modification and the sense strand comprises a 2′-O-Me modification (see e.g., Song et al., (2017) Mol Ther Nucleic Acids 9:242-250). In some embodiments at least one (e.g., at least 2, at least 3, at least 4, at least 5, at least 10) siRNA molecule is linked (e.g., covalently) to a muscle-targeting agent. In some embodiments, the muscle-targeting agent may comprise, or consist of, a nucleic acid (e.g., DNA or RNA), a peptide (e.g., an antibody), a lipid (e.g., a microvesicle), or a sugar moiety (e.g., a polysaccharide). In some embodiments, the muscle-targeting agent is an antibody. In some embodiments, the muscle-targeting agent is an anti-transferrin receptor antibody (e.g., any one of the anti-TfR1 antibodies provided in Tables 2-7). In some embodiments, the muscle-targeting agent may be linked to the 5′ end of the sense strand of the siRNA molecule. In some embodiments, the muscle-targeting agent may be covalently linked to the 5′ end of the sense strand of the siRNA molecule. In some embodiments, the muscle-targeting agent may be linked to the 3′ end of the sense strand of the siRNA molecule. In some embodiments, the muscle-targeting agent may be covalently linked to the 3′ end of the sense strand of the siRNA molecule. In some embodiments, the muscle-targeting agent may be linked internally to the sense strand of the siRNA molecule. In some embodiments, the muscle-targeting agent may be covalently linked internally to the sense strand of the siRNA molecule. In some embodiments, the muscle-targeting agent may be linked to the 5′ end of the antisense strand of the siRNA molecule. In some embodiments, the muscle-targeting agent may be covalently linked to the 5′ end of the antisense strand of the siRNA molecule. In some embodiments, the muscle-targeting agent may be linked to the 3′ end of the antisense strand of the siRNA molecule. In some embodiments, the muscle-targeting agent may be covalently linked to the 3′ end of the antisense strand of the siRNA molecule. In some embodiments, the muscle-targeting agent may be linked internally to the antisense strand of the siRNA molecule. In some embodiments, the muscle-targeting agent may be covalently linked internally to the antisense strand of the siRNA molecule.

Non-limiting examples of siRNAs described herein are provided in Table 8.

TABLE 8
Non-limiting examples of siRNAs^{†‡}

| | Target | | | | | |
|---|---|---|---|---|---|---|
| | start | | | SEQ | | SEQ |
| siRNA | posi- | Target | Sense | ID | Antisense | ID |
| No. | iton^{∧} | Sequence | strand (5′-3′) | NO | strand (5′-3′) | NO |
| 1 | 192 | GATTTGGTT | Unmodified: | 205 | Unmodified: | 236 |
| | | TCAGAATG | GAUUUGGUUUCAGA | | UUCUCAUUCUGAAACC | |
| | | AGA (SEQ ID | AUGAGAA | | AAAUCUG | |
| | | NO: 174) | Modified (MS3-205): | | Modified (MAS3-236): | |
| | | | mG*mA*mUmUfUmGm | | fU*fU*mCmUmCmAmUm | |
| | | | GmUfUfUfCmAmGfAm | | UmCmUmGmAmAfAmCm | |
| | | | AmUmGmAfGmAmA | | CmAmAmAmUmC*mU*m | |
| | | | | | G | |
| 2 | 192 | GATTTGGTT | Unmodified: | 205 | Unmodified: | 236 |
| | | TCAGAATG | GAUUUGGUUUCAGA | | UUCUCAUUCUGAAACC | |
| | | AGA (SEQ ID | AUGAGAA | | AAAUCUG | |
| | | NO: 174) | Modified (MS4-205): | | Modified (MAS4-236): | |
| | | | mG*mA*mUmUmUmGf | | fU*fU*mCmUmCfAmUfUf | |
| | | | GmUfUfUfCmAmGmA | | CmUmGmAmAfAmCfCm | |
| | | | mAmUmGmAmGmAmA | | AmAmAmUmC*mU*mG | |
| 3 | 192 | GATTTGGTT | Unmodified: | 205 | Unmodified: | 236 |
| | | TCAGAATG | GAUUUGGUUUCAGA | | UUCUCAUUCUGAAACC | |
| | | AGA (SEQ ID | AUGAGAA | | AAAUCUG | |
| | | NO: 174) | Modified (MS6-205): | | Modified (MAS4-236): | |
| | | | mG*mA*mUmUmUmGf | | fU*fU*mCmUmCfAmUfUf | |
| | | | GmUfUfUfCmAmGmA | | CmUmGmAmAfAmCfCm | |
| | | | mAfUmGmAfGmAmA | | AmAmAmUmC*mU*mG | |
| 4 | 192 | GATTTGGTT | Unmodified: | 205 | Unmodified: | 236 |
| | | TCAGAATG | GAUUUGGUUUCAGA | | UUCUCAUUCUGAAACC | |
| | | AGA (SEQ ID | AUGAGAA | | AAAUCUG | |
| | | NO: 174) | Modified (MS5-205): | | Modified (MAS2-236): | |
| | | | mG*mA*mUmUmUmGf | | fU*fU*mCmUmCfAmUm | |
| | | | GmUfUfUfCmAmGmA | | UmCmUmGmAmAfAmCf | |
| | | | mAfUfGmAmGmAmA | | CmAmAmAmUmC*mU*m | |
| | | | | | G | |
| 5 | 192 | GATTTGGTT | Unmodified: | 205 | Unmodified: | 236 |
| | | TCAGAATG | GAUUUGGUUUCAGA | | UUCUCAUUCUGAAACC | |
| | | AGA (SEQ ID | AUGAGAA | | AAAUCUG | |
| | | NO: 174) | Modified (MS4-205): | | Modified (MAS2-236): | |
| | | | mG*mA*mUmUmUmGf | | fU*fU*mCmUmCfAmUm | |
| | | | GmUfUfUfCmAmGmA | | UmCmUmGmAmAfAmCf | |
| | | | mAmUmGmAmGmAmA | | CmAmAmAmUmC*mU*m | |
| | | | | | G | |
| 6 | 192 | GATTTGGTT | Unmodified: | 205 | Unmodified: | 236 |
| | | TCAGAATG | GAUUUGGUUUCAGA | | UUCUCAUUCUGAAACC | |
| | | AGA (SEQ ID | AUGAGAA | | AAAUCUG | |
| | | NO: 174) | Modified (MS1-205): | | Modified (MAS1-236): | |
| | | | mG*mA*fUmUfUmGfG | | fU*fU*mCfUmCfAmUfUm | |
| | | | mUfUmUfCmAfGmAfA | | CfUmGfAmAfAmCfCmAf | |
| | | | mUfGmAfGmAfA | | AmAfUmC*fU*mG | |
| 7 | 337 | TTTGAGAA | Unmodified: | 206 | Unmodified: | 237 |
| | | GGATCGCTT | UUUGAGAAGGAUCG | | UGGAAAGCGAUCCUUC | |
| | | TCC (SEQ ID | CUUUCCA | | UCAAAGG | |
| | | NO: 175) | Modified (MS3-206): | | Modified (MAS3-237): | |
| | | | mU*mU*mUmGfAmGm | | fU*fG*mGmAmAmAmGm | |
| | | | AmAfGfGfAmUmCfGm | | CmGmAmUmCmCfUmUm | |
| | | | CmUmUmUfCmCmA | | CmUmCmAmAmA*mG*m | |
| | | | | | G | |
| 8 | 337 | TTTGAGAA | Unmodified: | 206 | Unmodified: | 237 |
| | | GGATCGCTT | UUUGAGAAGGAUCG | | UGGAAAGCGAUCCUUC | |
| | | TCC (SEQ ID | CUUUCCA | | UCAAAGG | |
| | | NO: 175) | Modified (MS4-206): | | Modified (MAS4-237): | |
| | | | mU*mU*mUmGmAmGf | | fU*fG*mGmAmAfAmGfCf | |
| | | | AmAfGfGfAmUmCmG | | GmAmUmCmCfUmUfCm | |
| | | | mCmUmUmUmCmCmA | | UmCmAmAmA*mG*mG | |
| 9 | 337 | TTTGAGAA | Unmodified: | 206 | Unmodified: | 237 |
| | | GGATCGCTT | UUUGAGAAGGAUCG | | UGGAAAGCGAUCCUUC | |
| | | TCC (SEQ ID | CUUUCCA | | UCAAAGG | |
| | | NO: 175) | Modified (MS6-206): | | Modified (MAS4-237): | |
| | | | mU*mU*mUmGmAmGf | | fU*fG*mGmAmAfAmGfCf | |
| | | | AmAfGfGfAmUmCmG | | GmAmUmCmCfUmUfCm | |
| | | | mCfUmUmUfCmCmA | | UmCmAmAmA*mG*mG | |
| 10 | 337 | TTTGAGAA | Unmodified: | 206 | Unmodified: | 237 |
| | | GGATCGCTT | UUUGAGAAGGAUCG | | UGGAAAGCGAUCCUUC | |
| | | TCC (SEQ ID | CUUUCCA | | UCAAAGG | |
| | | NO: 175) | Modified (MS5-206): | | Modified (MAS2-237): | |
| | | | mU*mU*mUmGmAmGf | | fU*fG*mGmAmAfAmGm | |
| | | | AmAfGfGfAmUmCmG | | CmGmAmUmCmCfUmUf | |
| | | | mCfUfUmUmCmCmA | | CmUmCmAmAmA*mG*m | |
| | | | | | G | |
| 11 | 337 | TTTGAGAA | Unmodified: | 206 | Unmodified: | 237 |
| | | GGATCGCTT | UUUGAGAAGGAUCG | | UGGAAAGCGAUCCUUC | |
| | | TCC (SEQ ID | CUUUCCA | | UCAAAGG | |
| | | NO: 175) | Modified (MS4-206): | | Modified (MAS2-237): | |
| | | | mU*mU*mUmGmAmGf | | fU*fG*mGmAmAfAmGm | |
| | | | AmAfGfGfAmUmCmG | | CmGmAmUmCmCfUmUf | |
| | | | mCmUmUmUmCmCmA | | CmUmCmAmAmA*mG*m | |
| | | | | | G | |
| 12 | 337 | TTTGAGAA | Unmodified: | 206 | Unmodified: | 237 |
| | | GGATCGCTT | UUUGAGAAGGAUCG | | UGGAAAGCGAUCCUUC | |
| | | TCC (SEQ ID | CUUUCCA | | UCAAAGG | |
| | | NO: 175) | Modified (MS1-206): | | Modified (MAS1-237): | |
| | | | mU*mU*fUmGfAmGfA | | fU*fG*mGfAmAfAmGfCm | |
| | | | mAfGmGfAmUfCmGfC | | GfAmUfCmCfUmUfCmUf | |
| | | | mUfUmUfCmCfA | | CmAfAmA*fG*mG | |
| 13 | 1234 | CTCAGCGA | Unmodified: | 207 | Unmodified: | 238 |
| | | GGAAGAAT | CUCAGCGAGGAAGA | | UCGGUAUUCUUCCUCG | |
| | | ACCG (SEQ | AUACCGA | | CUGAGGG | |
| | | ID NO: 176) | Modified (MS3-207): | | Modified (MAS3-238): | |
| | | | mC*mU*mCmAfGmCm | | fU*fC*mGmGmUmAmUm | |
| | | | GmAfGfGfAmAmGfAm | | UmCmUmUmCmCfUmCm | |
| | | | AmUmAmCfCmGmA | | GmCmUmGmAmG*mG*m | |
| | | | | | G | |
| 14 | 1234 | CTCAGCGA | Unmodified: | 207 | Unmodified: | 238 |
| | | GGAAGAAT | CUCAGCGAGGAAGA | | UCGGUAUUCUUCCUCG | |
| | | ACCG (SEQ | AUACCGA | | CUGAGGG | |
| | | ID NO: 176) | Modified (MS4-207): | | Modified (MAS4-238): | |
| | | | mC*mU*mCmAmGmCf | | fU*fC*mGmGmUfAmUfUf | |
| | | | GmAfGfGfAmAmGmA | | CmUmUmCmCfUmCfGm | |
| | | | mAmUmAmCmCmGmA | | CmUmGmAmG*mG*mG | |
| 15 | 1234 | CTCAGCGA | Unmodified: | 207 | Unmodified: | 238 |
| | | GGAAGAAT | CUCAGCGAGGAAGA | | UCGGUAUUCUUCCUCG | |
| | | ACCG (SEQ | AUACCGA | | CUGAGGG | |
| | | ID NO: 176) | Modified (MS6-207): | | Modified (MAS 4-238): | |
| | | | mC*mU*mCmAmGmCf | | fU*fC*mGmGmUfAmUfUf | |
| | | | GmAfGfGfAmAmGmA | | CmUmUmCmCfUmCfGm | |
| | | | mAfUmAmCfCmGmA | | CmUmGmAmG*mG*mG | |
| 16 | 1234 | CTCAGCGA | Unmodified: | 207 | Unmodified: | 238 |
| | | GGAAGAAT | CUCAGCGAGGAAGA | | UCGGUAUUCUUCCUCG | |
| | | ACCG (SEQ | AUACCGA | | CUGAGGG | |
| | | ID NO: 176) | Modified (MS5-207): | | Modified (MAS2-238): | |
| | | | mC*mU*mCmAmGmCf | | fU*fC*mGmGmUfAmUm | |
| | | | GmAfGfGfAmAmGmA | | UmCmUmUmCmCfUmCf | |
| | | | mAfUfAmCmCmGmA | | GmCmUmGmAmG*mG*m | |
| | | | | | G | |
| 17 | 1234 | CTCAGCGA | Unmodified: | 207 | Unmodified: | 238 |
| | | GGAAGAAT | CUCAGCGAGGAAGA | | UCGGUAUUCUUCCUCG | |
| | | ACCG (SEQ | AUACCGA | | CUGAGGG | |
| | | ID NO: 176) | Modified (MS4-207): | | Modified (MAS2-238): | |
| | | | mC*mU*mCmAmGmCf | | fU*fC*mGmGmUfAmUm | |
| | | | GmAfGfGfAmAmGmA | | UmCmUmUmCmCfUmCf | |
| | | | mAmUmAmCmCmGmA | | GmCmUmGmAmG*mG*m | |
| | | | | | G | |
| 18 | 1234 | CTCAGCGA | Unmodified: | 207 | Unmodified: | 238 |
| | | GGAAGAAT | CUCAGCGAGGAAGA | | UCGGUAUUCUUCCUCG | |
| | | ACCG (SEQ | AUACCGA | | CUGAGGG | |
| | | ID NO: 176) | Modified (MS1-207): | | Modified (MAS1-238): | |
| | | | mC*mU*fCmAfGmCfG | | fU*fC*mGfGmUfAmUfUm | |
| | | | mAfGmGfAmAfGmAfA | | CfUmUfCmCfUmCfGmCf | |
| | | | mUfAmCfCmGfA | | UmGfAmG*fG*mG | |
| 19 | 1568 | TGCCCTTGT | Unmodified: | 208 | Unmodified: | 239 |
| | | TCTTCCGTG | UGCCCUUGUUCUUCC | | UUUCACGGAAGAACAA | |
| | | AA (SEQ ID | GUGAAA | | GGGCACA | |
| | | NO: 177) | Modified (MS3-208): | | Modified (MAS3-239): | |
| | | | mU*mG*mCmCfCmUm | | fU*fU*mUmCmAmCmGm | |
| | | | UmGfUfUfCmUmUfCm | | GmAmAmGmAmAfCmAm | |
| | | | CmGmUmGfAmAmA | | AmGmGmGmCmA*mC*m | |
| | | | | | A | |
| 20 | 1568 | TGCCCTTGT | Unmodified: | 208 | Unmodified: | 239 |
| | | TCTTCCGTG | UGCCCUUGUUCUUCC | | UUUCACGGAAGAACAA | |
| | | AA (SEQ ID | GUGAAA | | GGGCACA | |
| | | NO: 177) | Modified (MS4-208): | | Modified (MAS4-239): | |
| | | | mU*mG*mCmCmCmUf | | fU*fU*mUmCmAfCmGfGf | |
| | | | UmGfUfUfCmUmUmC | | AmAmGmAmAfCmAfAm | |
| | | | mCmGmUmGmAmAmA | | GmGmGmCmA*mC*mA | |
| 21 | 1568 | TGCCCTTGT | Unmodified: | 208 | Unmodified: | 239 |
| | | TCTTCCGTG | UGCCCUUGUUCUUCC | | UUUCACGGAAGAACAA | |
| | | AA (SEQ ID | GUGAAA | | GGGCACA | |
| | | NO: 177) | Modified (MS6-208): | | Modified (MAS4-239): | |
| | | | mU*mG*mCmCmCmUf | | fU*fU*mUmCmAfCmGfGf | |
| | | | UmGfUfUfCmUmUmC | | AmAmGmAmAfCmAfAm | |
| | | | mCfGmUmGfAmAmA | | GmGmGmCmA*mC*mA | |
| 22 | 1568 | TGCCCTTGT | Unmodified: | 208 | Unmodified: | 239 |
| | | TCTTCCGTG | UGCCCUUGUUCUUCC | | UUUCACGGAAGAACAA | |
| | | AA (SEQ ID | GUGAAA | | GGGCACA | |
| | | NO: 177) | Modified (MS5-208): | | Modified (MAS2-239): | |
| | | | mU*mG*mCmCmCmUf | | fU*fU*mUmCmAfCmGm | |
| | | | UmGfUfUfCmUmUmC | | GmAmAmGmAmAfCmAf | |
| | | | mCfGfUmGmAmAmA | | AmGmGmGmCmA*mC*m | |
| | | | | | A | |
| 23 | 1568 | TGCCCTTGT | Unmodified: | 208 | Unmodified: | 239 |
| | | TCTTCCGTG | UGCCCUUGUUCUUCC | | UUUCACGGAAGAACAA | |
| | | AA (SEQ ID | GUGAAA | | GGGCACA | |
| | | NO: 177) | Modified (MS4-208): | | Modified (MAS2-239): | |
| | | | mU*mG*mCmCmCmUf | | fU*fU*mUmCmAfCmGm | |
| | | | UmGfUfUfCmUmUmC | | GmAmAmGmAmAfCmAf | |
| | | | mCmGmUmGmAmAmA | | AmGmGmGmCmA*mC*m | |
| | | | | | A | |
| 24 | 1568 | TGCCCTTGT | Unmodified: | 208 | Unmodified: | 239 |
| | | TCTTCCGTG | UGCCCUUGUUCUUCC | | UUUCACGGAAGAACAA | |
| | | AA (SEQ ID | GUGAAA | | GGGCACA | |
| | | NO: 177) | Modified (MS2-208): | | Modified (MAS4-239): | |
| | | | mU*mG*mCmCfCmUfU | | fU*fU*mUmCmAfCmGfGf | |
| | | | mGfUfUfCmUmUmCmC | | AmAmGmAmAfCmAfAm | |
| | | | fGmUmGmAmAmA | | GmGmGmCmA*mC*mA | |
| 25 | 1654 | TCCTGGATG | Unmodified: | 209 | Unmodified: | 240 |
| | | ATTAGTTCA | UCCUGGAUGAUUAG | | UUCUGAACUAAUCAUC | |
| | | GA (SEQ ID | UUCAGAA | | CAGGAGA | |
| | | NO: 178) | Modified (MS3-209): | | Modified (MAS3-240): | |
| | | | mU*mC*mCmUfGmGm | | fU*fU*mCmUmGmAmAm | |
| | | | AmUfGfAfUmUmAfGm | | CmUmAmAmUmCfAmUm | |
| | | | UmUmCmAfGmAmA | | CmCmAmGmGmA*mG*m | |
| | | | | | A | |
| 26 | 1654 | TCCTGGATG | Unmodified: | 209 | Unmodified: | 240 |
| | | ATTAGTTCA | UCCUGGAUGAUUAG | | UUCUGAACUAAUCAUC | |
| | | GA (SEQ ID | UUCAGAA | | CAGGAGA | |
| | | NO: 178) | Modified (MS4-209): | | Modified (MAS4-240): | |
| | | | mU*mC*mCmUmGmGf | | fU*fU*mCmUmGfAmAfCf | |
| | | | AmUfGfAfUmUmAmG | | UmAmAmUmCfAmUfCm | |
| | | | mUmUmCmAmGmAmA | | CmAmGmGmA*mG*mA | |
| 27 | 1654 | TCCTGGATG | Unmodified: | 209 | Unmodified: | 240 |
| | | ATTAGTTCA | UCCUGGAUGAUUAG | | UUCUGAACUAAUCAUC | |
| | | GA (SEQ ID | UUCAGAA | | CAGGAGA | |
| | | NO: 178) | Modified (MS6-209): | | Modified (MAS4-240): | |
| | | | mU*mC*mCmUmGmGf | | fU*fU*mCmUmGfAmAfCf | |
| | | | AmUfGfAfUmUmAmG | | UmAmAmUmCfAmUfCm | |
| | | | mUfUmCmAfGmAmA | | CmAmGmGmA*mG*mA | |
| 28 | 1654 | TCCTGGATG | Unmodified: | 209 | Unmodified: | 240 |
| | | ATTAGTTCA | UCCUGGAUGAUUAG | | UUCUGAACUAAUCAUC | |
| | | GA (SEQ ID | UUCAGAA | | CAGGAGA | |
| | | NO: 178) | Modified (MS5-209): | | Modified (MAS2-240): | |
| | | | mU*mC*mCmUmGmGf | | fU*fU*mCmUmGfAmAm | |
| | | | AmUfGfAfUmUmAmG | | CmUmAmAmUmCfAmUf | |
| | | | mUfUfCmAmGmAmA | | CmCmAmGmGmA*mG*m | |
| | | | | | A | |
| 29 | 1654 | TCCTGGATG | Unmodified: | 209 | Unmodified: | 240 |
| | | ATTAGTTCA | UCCUGGAUGAUUAG | | UUCUGAACUAAUCAUC | |
| | | GA (SEQ ID | UUCAGAA | | CAGGAGA | |
| | | NO: 178) | Modified (MS4-209): | | Modified (MAS2-240): | |
| | | | mU*mC*mCmUmGmGf | | fU*fU*mCmUmGfAmAm | |
| | | | AmUfGfAfUmUmAmG | | CmUmAmAmUmCfAmUf | |
| | | | mUmUmCmAmGmAmA | | CmCmAmGmGmA*mG*m | |
| | | | | | A | |
| 30 | 1654 | TCCTGGATG | Unmodified: | 209 | Unmodified: | 240 |
| | | ATTAGTTCA | UCCUGGAUGAUUAG | | UUCUGAACUAAUCAUC | |
| | | GA (SEQ ID | UUCAGAA | | CAGGAGA | |
| | | NO: 178) | Modified (MS2-209): | | Modified (MAS4-240): | |
| | | | mU*mC*mCmUfGmGfA | | fU*fU*mCmUmGfAmAfCf | |
| | | | mUfGfAfUmUmAmGm | | UmAmAmUmCfAmUfCm | |
| | | | UfUmCmAmGmAmA | | CmAmGmGmA*mG*mA | |
| 31 | 1624 | GTCTAGGC | Unmodified: | 210 | Unmodified: | 241 |
| | | AAACCTGG | GUCUAGGCAAACCU | | UUAAUCCAGGUUUGCC | |
| | | ATTA (SEQ | GGAUUAA | | UAGACAG | |
| | | ID NO: 179) | Modified (MS3-210): | | Modified (MAS3-241): | |
| | | | mG*mU*mCmUfAmGm | | fU*fU*mAmAmUmCmCm | |
| | | | GmCfAfAfAmCmCfUm | | AmGmGmUmUmUfGmCm | |
| | | | GmGmAmUfUmAmA | | CmUmAmGmAmC*mA*m | |
| | | | | | G | |
| 32 | 1624 | GTCTAGGC | Unmodified: | 210 | Unmodified: | 241 |
| | | AAACCTGG | GUCUAGGCAAACCU | | UUAAUCCAGGUUUGCC | |
| | | ATTA (SEQ | GGAUUAA | | UAGACAG | |
| | | ID NO: 179) | Modified (MS4-210): | | Modified (MAS4-241): | |
| | | | mG*mU*mCmUmAmGf | | fU*fU*mAmAmUfCmCfAf | |
| | | | GmCfAfAfAmCmCmUm | | GmGmUmUmUfGmCfCm | |
| | | | GmGmAmUmUmAmA | | UmAmGmAmC*mA*mG | |
| 33 | 1624 | GTCTAGGC | Unmodified: | 210 | Unmodified: | 241 |
| | | AAACCTGG | GUCUAGGCAAACCU | | UUAAUCCAGGUUUGCC | |
| | | ATTA (SEQ | GGAUUAA | | UAGACAG | |
| | | ID NO: 179) | Modified (MS6-210): | | Modified (MAS4-241): | |
| | | | mG*mU*mCmUmAmGf | | fU*fU*mAmAmUfCmCfAf | |
| | | | GmCfAfAfAmCmCmUm | | GmGmUmUmUfGmCfCm | |
| | | | GfGmAmUfUmAmA | | UmAmGmAmC*mA*mG | |
| 34 | 1624 | GTCTAGGC | Unmodified: | 210 | Unmodified: | 241 |
| | | AAACCTGG | GUCUAGGCAAACCU | | UUAAUCCAGGUUUGCC | |
| | | ATTA (SEQ | GGAUUAA | | UAGACAG | |
| | | ID NO: 179) | Modified (MS5-210): | | Modified (MAS2-241): | |
| | | | mG*mU*mCmUmAmGf | | fU*fU*mAmAmUfCmCm | |
| | | | GmCfAfAfAmCmCmUm | | AmGmGmUmUmUfGmCf | |
| | | | GfGfAmUmUmAmA | | CmUmAmGmAmC*mA*m | |
| | | | | | G | |
| 35 | 1624 | GTCTAGGC | Unmodified: | 210 | Unmodified: | 241 |
| | | AAACCTGG | GUCUAGGCAAACCU | | UUAAUCCAGGUUUGCC | |
| | | ATTA (SEQ | GGAUUAA | | UAGACAG | |
| | | ID NO: 179) | Modified (MS4-210): | | Modified (MAS2-241): | |
| | | | mG*mU*mCmUmAmGf | | fU*fU*mAmAmUfCmCm | |
| | | | GmCfAfAfAmCmCmUm | | AmGmGmUmUmUfGmCf | |
| | | | GmGmAmUmUmAmA | | CmUmAmGmAmC*mA*m | |
| | | | | | G | |
| 36 | 1624 | GTCTAGGC | Unmodified: | 210 | Unmodified: | 241 |
| | | AAACCTGG | GUCUAGGCAAACCU | | UUAAUCCAGGUUUGCC | |
| | | ATTA (SEQ | GGAUUAA | | UAGACAG | |
| | | ID NO: 179) | Modified (MS2-210): | | Modified (MAS4-241): | |
| | | | mG*mU*mCmUfAmGf | | fU*fU*mAmAmUfCmCfAf | |
| | | | GmCfAfAfAmCmCmUm | | GmGmUmUmUfGmCfCm | |
| | | | GfGmAmUmUmAmA | | UmAmGmAmC*mA*mG | |
| 37 | 1619 | ACGCTGTCT | Unmodified: | 211 | Unmodified: | 242 |
| | | AGGCAAAC | ACGCUGUCUAGGCA | | UCAGGUUUGCCUAGAC | |
| | | CTG (SEQ ID | AACCUGA | | AGCGUCG | |
| | | NO: 180) | Modified (MS3-211): | | Modified (MAS3-242): | |
| | | | mA*mC*mGmCfUmGm | | fU*fC*mAmGmGmUmUm | |
| | | | UmCfUfAfGmGmCfAm | | UmGmCmCmUmAfGmAm | |
| | | | AmAmCmCfUmGmA | | CmAmGmCmGmU*mC*m | |
| | | | | | G | |
| 38 | 1619 | ACGCTGTCT | Unmodified: | 211 | Unmodified: | 242 |
| | | AGGCAAAC | ACGCUGUCUAGGCA | | UCAGGUUUGCCUAGAC | |
| | | CTG (SEQ ID | AACCUGA | | AGCGUCG | |
| | | NO: 180) | Modified (MS4-211): | | Modified (MAS4-242): | |
| | | | mA*mC*mGmCmUmGf | | fU*fC*mAmGmGfUmUfUf | |
| | | | UmCfUfAfGmGmCmA | | GmCmCmUmAfGmAfCm | |
| | | | mAmAmCmCmUmGmA | | AmGmCmGmU*mC*mG | |
| 39 | 1619 | ACGCTGTCT | Unmodified: | 211 | Unmodified: | 242 |
| | | AGGCAAAC | ACGCUGUCUAGGCA | | UCAGGUUUGCCUAGAC | |
| | | CTG (SEQ ID | AACCUGA | | AGCGUCG | |
| | | NO: 180) | Modified (MS6-211): | | Modified (MAS4-242): | |
| | | | mA*mC*mGmCmUmGf | | fU*fC*mAmGmGfUmUfUf | |
| | | | UmCfUfAfGmGmCmA | | GmCmCmUmAfGmAfCm | |
| | | | mAfAmCmCfUmGmA | | AmGmCmGmU*mC*mG | |
| 40 | 1619 | ACGCTGTCT | Unmodified: | 211 | Unmodified: | 242 |
| | | AGGCAAAC | ACGCUGUCUAGGCA | | UCAGGUUUGCCUAGAC | |
| | | CTG (SEQ ID | AACCUGA | | AGCGUCG | |
| | | NO: 180) | Modified (MS5-211): | | Modified (MAS2-242): | |
| | | | mA*mC*mGmCmUmGf | | fU*fC*mAmGmGfUmUm | |
| | | | UmCfUfAfGmGmCmA | | UmGmCmCmUmAfGmAf | |
| | | | mAfAfCmCmUmGmA | | CmAmGmCmGmU*mC*m | |
| | | | | | G | |
| 41 | 1619 | ACGCTGTCT | Unmodified: | 211 | Unmodified: | 242 |
| | | AGGCAAAC | ACGCUGUCUAGGCA | | UCAGGUUUGCCUAGAC | |
| | | CTG (SEQ ID | AACCUGA | | AGCGUCG | |
| | | NO: 180) | Modified (MS4-211): | | Modified (MAS2-242): | |
| | | | mA*mC*mGmCmUmGf | | fU*fC*mAmGmGfUmUm | |
| | | | UmCfUfAfGmGmCmA | | UmGmCmCmUmAfGmAf | |
| | | | mAmAmCmCmUmGmA | | CmAmGmCmGmU*mC*m | |
| | | | | | G | |
| 42 | 1619 | ACGCTGTCT | Unmodified: | 211 | Unmodified: | 242 |
| | | AGGCAAAC | ACGCUGUCUAGGCA | | UCAGGUUUGCCUAGAC | |
| | | CTG (SEQ ID | AACCUGA | | AGCGUCG | |
| | | NO: 180) | Modified (MS2-211): | | Modified (MAS4-242): | |
| | | | mA*mC*mGmCfUmGfU | | fU*fC*mAmGmGfUmUfUf | |
| | | | mCfUfAfGmGmCmAm | | GmCmCmUmAfGmAfCm | |
| | | | AfAmCmCmUmGmA | | AmGmCmGmU*mC*mG | |
| 43 | 1651 | ATCTCCTGG | Unmodified: | 212 | Unmodified: | 243 |
| | | ATGATTAGT | AUCUCCUGGAUGAU | | UGAACUAAUCAUCCAG | |
| | | TC (SEQ ID | UAGUUCA | | GAGAUGU | |
| | | NO: 181) | Modified (MS3-212): | | Modified (MAS3-243): | |
| | | | mA*mU*mCmUfCmCm | | fU*fG*mAmAmCmUmAm | |
| | | | UmGfGfAfUmGmAfUm | | AmUmCmAmUmCfCmAm | |
| | | | UmAmGmUfUmCmA | | GmGmAmGmAmU*mG*m | |
| | | | | | U | |
| 44 | 1651 | ATCTCCTGG | Unmodified: | 212 | Unmodified: | 243 |
| | | ATGATTAGT | AUCUCCUGGAUGAU | | UGAACUAAUCAUCCAG | |
| | | TC (SEQ ID | UAGUUCA | | GAGAUGU | |
| | | NO: 181) | Modified (MS4-212): | | Modified (MAS4-243): | |
| | | | mA*mU*mCmUmCmCf | | fU*fG*mAmAmCfUmAfAf | |
| | | | UmGfGfAfUmGmAmU | | UmCmAmUmCfCmAfGm | |
| | | | mUmAmGmUmUmCmA | | GmAmGmAmU*mG*mU | |
| 45 | 1651 | ATCTCCTGG | Unmodified: | 212 | Unmodified: | 243 |
| | | ATGATTAGT | AUCUCCUGGAUGAU | | UGAACUAAUCAUCCAG | |
| | | TC (SEQ ID | UAGUUCA | | GAGAUGU | |
| | | NO: 181) | Modified (MS6-212): | | Modified (MAS4-243): | |
| | | | mA*mU*mCmUmCmCf | | fU*fG*mAmAmCfUmAfAf | |
| | | | UmGfGfAfUmGmAmU | | UmCmAmUmCfCmAfGm | |
| | | | mUfAmGmUfUmCmA | | GmAmGmAmU*mG*mU | |
| 46 | 1651 | ATCTCCTGG | Unmodified: | 212 | Unmodified: | 243 |
| | | ATGATTAGT | AUCUCCUGGAUGAU | | UGAACUAAUCAUCCAG | |
| | | TC (SEQ ID | UAGUUCA | | GAGAUGU | |
| | | NO: 181) | Modified (MS5-212): | | Modified (MAS2-243): | |
| | | | mA*mU*mCmUmCmCf | | fU*fG*mAmAmCfUmAm | |
| | | | UmGfGfAfUmGmAmU | | AmUmCmAmUmCfCmAf | |
| | | | mUfAfGmUmUmCmA | | GmGmAmGmAmU*mG*m | |
| | | | | | U | |
| 47 | 1651 | ATCTCCTGG | Unmodified: | 212 | Unmodified: | 243 |
| | | ATGATTAGT | AUCUCCUGGAUGAU | | UGAACUAAUCAUCCAG | |
| | | TC (SEQ ID | UAGUUCA | | GAGAUGU | |
| | | NO: 181) | Modified (MS4-212): | | Modified (MAS2-243): | |
| | | | mA*mU*mCmUmCmCf | | fU*fG*mAmAmCfUmAm | |
| | | | UmGfGfAfUmGmAmU | | AmUmCmAmUmCfCmAf | |
| | | | mUmAmGmUmUmCmA | | GmGmAmGmAmU*mG*m | |
| | | | | | U | |
| 48 | 1651 | ATCTCCTGG | Unmodified: | 212 | Unmodified: | 243 |
| | | ATGATTAGT | AUCUCCUGGAUGAU | | UGAACUAAUCAUCCAG | |
| | | TC (SEQ ID | UAGUUCA | | GAGAUGU | |
| | | NO: 181) | Modified (MS2-212): | | Modified (MAS4-243): | |
| | | | mA*mU*mCmUfCmCfU | | fU*fG*mAmAmCfUmAfAf | |
| | | | mGfGfAfUmGmAmUm | | UmCmAmUmCfCmAfGm | |
| | | | UfAmGmUmUmCmA | | GmAmGmAmU*mG*mU | |
| 49 | 1565 | CTGTGCCCT | Unmodified: | 213 | Unmodified: | 244 |
| | | TGTTCTTCC | CUGUGCCCUUGUUC | | UACGGAAGAACAAGGG | |
| | | GT (SEQ ID | UUCCGUA | | CACAGAG | |
| | | NO: 182) | Modified (MS3-213): | | Modified (MAS3-244): | |
| | | | mC*mU*mGmUfGmCm | | fU*fA*mCmGmGmAmAm | |
| | | | CmCfUfUfGmUmUfCm | | GmAmAmCmAmAfGmGm | |
| | | | UmUmCmCfGmUmA | | GmCmAmCmAmG*mA*m | |
| | | | | | G | |
| 50 | 1565 | CTGTGCCCT | Unmodified: | 213 | Unmodified: | 244 |
| | | TGTTCTTCC | CUGUGCCCUUGUUC | | UACGGAAGAACAAGGG | |
| | | GT (SEQ ID | UUCCGUA | | CACAGAG | |
| | | NO: 182) | Modified (MS4-213): | | Modified (MAS4-244): | |
| | | | mC*mU*mGmUmGmCf | | fU*fA*mCmGmGfAmAfGf | |
| | | | CmCfUfUfGmUmUmCm | | AmAmCmAmAfGmGfGm | |
| | | | UmUmCmCmGmUmA | | CmAmCmAmG*mA*mG | |
| 51 | 1565 | CTGTGCCCT | Unmodified: | 213 | Unmodified: | 244 |
| | | TGTTCTTCC | CUGUGCCCUUGUUC | | UACGGAAGAACAAGGG | |
| | | GT (SEQ ID | UUCCGUA | | CACAGAG | |
| | | NO: 182) | Modified (MS6-213): | | Modified (MAS4-244): | |
| | | | mC*mU*mGmUmGmCf | | fU*fA*mCmGmGfAmAfGf | |
| | | | CmCfUfUfGmUmUmCm | | AmAmCmAmAfGmGfGm | |
| | | | UfUmCmCfGmUmA | | CmAmCmAmG*mA*mG | |
| 52 | 1565 | CTGTGCCCT | Unmodified: | 213 | Unmodified: | 244 |
| | | TGTTCTTCC | CUGUGCCCUUGUUC | | UACGGAAGAACAAGGG | |
| | | GT (SEQ ID | UUCCGUA | | CACAGAG | |
| | | NO: 182) | Modified (MS5-213): | | Modified (MAS2-244): | |
| | | | mC*mU*mGmUmGmCf | | fU*fA*mCmGmGfAmAm | |
| | | | CmCfUfUfGmUmUmCm | | GmAmAmCmAmAfGmGf | |
| | | | UfUfCmCmGmUmA | | GmCmAmCmAmG*mA*m | |
| | | | | | G | |
| 53 | 1565 | CTGTGCCCT | Unmodified: | 213 | Unmodified: | 244 |
| | | TGTTCTTCC | CUGUGCCCUUGUUC | | UACGGAAGAACAAGGG | |
| | | GT (SEQ ID | UUCCGUA | | CACAGAG | |
| | | NO: 182) | Modified (MS4-213): | | Modified (MAS2-244): | |
| | | | mC*mU*mGmUmGmCf | | fU*fA*mCmGmGfAmAm | |
| | | | CmCfUfUfGmUmUmCm | | GmAmAmCmAmAfGmGf | |
| | | | UmUmCmCmGmUmA | | GmCmAmCmAmG*mA*m | |
| | | | | | G | |
| 54 | 1565 | CTGTGCCCT | Unmodified: | 213 | Unmodified: | 244 |
| | | TGTTCTTCC | CUGUGCCCUUGUUC | | UACGGAAGAACAAGGG | |
| | | GT (SEQ ID | UUCCGUA | | CACAGAG | |
| | | NO: 182) | Modified (MS2-213): | | Modified (MAS4-244): | |
| | | | mC*mU*mGmUfGmCfC | | fU*fA*mCmGmGfAmAfGf | |
| | | | mCfUfUfGmUmUmCm | | AmAmCmAmAfGmGfGm | |
| | | | UfUmCmCmGmUmA | | CmAmCmAmG*mA*mG | |
| 55 | 1656 | CTGGATGAT | Unmodified: | 214 | Unmodified: | 245 |
| | | TAGTTCAGA | CUGGAUGAUUAGUU | | UUCUCUGAACUAAUCA | |
| | | GA (SEQ ID | CAGAGAA | | UCCAGGA | |
| | | NO: 183) | Modified (MS3-214): | | Modified (MAS3-245): | |
| | | | mC*mU*mGmGfAmUm | | fU*fU*mCmUmCmUmGm | |
| | | | GmAfUfUfAmGmUfUm | | AmAmCmUmAmAfUmCm | |
| | | | CmAmGmAfGmAmA | | AmUmCmCmAmG*mG*m | |
| | | | | | A | |
| 56 | 1656 | CTGGATGAT | Unmodified: | 214 | Unmodified: | 245 |
| | | TAGTTCAGA | CUGGAUGAUUAGUU | | UUCUCUGAACUAAUCA | |
| | | GA (SEQ ID | CAGAGAA | | UCCAGGA | |
| | | NO: 183) | Modified (MS4-214): | | Modified (MAS4-245): | |
| | | | mC*mU*mGmGmAmUf | | fU*fU*mCmUmCfUmGfAf | |
| | | | GmAfUfUfAmGmUmU | | AmCmUmAmAfUmCfAm | |
| | | | mCmAmGmAmGmAmA | | UmCmCmAmG*mG*mA | |
| 57 | 1656 | CTGGATGAT | Unmodified: | 214 | Unmodified: | 245 |
| | | TAGTTCAGA | CUGGAUGAUUAGUU | | UUCUCUGAACUAAUCA | |
| | | GA (SEQ ID | CAGAGAA | | UCCAGGA | |
| | | NO: 183) | Modified (MS6-214): | | Modified (MAS4-245): | |
| | | | mC*mU*mGmGmAmUf | | fU*fU*mCmUmCfUmGfAf | |
| | | | GmAfUfUfAmGmUmU | | AmCmUmAmAfUmCfAm | |
| | | | mCfAmGmAfGmAmA | | UmCmCmAmG*mG*mA | |
| 58 | 1656 | CTGGATGAT | Unmodified: | 214 | Unmodified: | 245 |
| | | TAGTTCAGA | CUGGAUGAUUAGUU | | UUCUCUGAACUAAUCA | |
| | | GA (SEQ ID | CAGAGAA | | UCCAGGA | |
| | | NO: 183) | Modified (MS5-214): | | Modified (MAS2-245): | |
| | | | mC*mU*mGmGmAmUf | | fU*fU*mCmUmCfUmGm | |
| | | | GmAfUfUfAmGmUmU | | AmAmCmUmAmAfUmCf | |
| | | | mCfAfGmAmGmAmA | | AmUmCmCmAmG*mG*m | |
| | | | | | A | |
| 59 | 1656 | CTGGATGAT | Unmodified: | 214 | Unmodified: | 245 |
| | | TAGTTCAGA | CUGGAUGAUUAGUU | | UUCUCUGAACUAAUCA | |
| | | GA (SEQ ID | CAGAGAA | | UCCAGGA | |
| | | NO: 183) | Modified (MS4-214): | | Modified (MAS2-245): | |
| | | | mC*mU*mGmGmAmUf | | fU*fU*mCmUmCfUmGm | |
| | | | GmAfUfUfAmGmUmU | | AmAmCmUmAmAfUmCf | |
| | | | mCmAmGmAmGmAmA | | AmUmCmCmAmG*mG*m | |
| | | | | | A | |
| 60 | 1656 | CTGGATGAT | Unmodified: | 214 | Unmodified: | 245 |
| | | TAGTTCAGA | CUGGAUGAUUAGUU | | UUCUCUGAACUAAUCA | |
| | | GA (SEQ ID | CAGAGAA | | UCCAGGA | |
| | | NO: 183) | Modified (MS2-214): | | Modified (MAS4-245): | |
| | | | mC*mU*mGmGfAmUf | | fU*fU*mCmUmCfUmGfAf | |
| | | | GmAfUfUfAmGmUmU | | AmCmUmAmAfUmCfAm | |
| | | | mCfAmGmAmGmAmA | | UmCmCmAmG*mG*mA | |
| 61 | 1570 | CCCTTGTTC | Unmodified: | 215 | Unmodified: | 246 |
| | | TTCCGTGAA | CCCUUGUUCUUCCGU | | UAUUUCACGGAAGAAC | |
| | | AT (SEQ ID | GAAAUA | | AAGGGCA | |
| | | NO: 184) | Modified (MS3-215): | | Modified (MAS3-246): | |
| | | | mC*mC*mCmUfUmGm | | fU*fA*mUmUmUmCmAm | |
| | | | UmUfCfUfUmCmCfGm | | CmGmGmAmAmGfAmAm | |
| | | | UmGmAmAfAmUmA | | CmAmAmGmGmG*mC*m | |
| | | | | | A | |
| 62 | 1570 | CCCTTGTTC | Unmodified: | 215 | Unmodified: | 246 |
| | | TTCCGTGAA | CCCUUGUUCUUCCGU | | UAUUUCACGGAAGAAC | |
| | | AT (SEQ ID | GAAAUA | | AAGGGCA | |
| | | NO: 184) | Modified (MS4-215): | | Modified (MAS4-246): | |
| | | | mC*mC*mCmUmUmGf | | fU*fA*mUmUmUfCmAfCf | |
| | | | UmUfCfUfUmCmCmGm | | GmGmAmAmGfAmAfCm | |
| | | | UmGmAmAmAmUmA | | AmAmGmGmG*mC*mA | |
| 63 | 1570 | CCCTTGTTC | Unmodified: | 215 | Unmodified: | 246 |
| | | TTCCGTGAA | CCCUUGUUCUUCCGU | | UAUUUCACGGAAGAAC | |
| | | AT (SEQ ID | GAAAUA | | AAGGGCA | |
| | | NO: 184) | Modified (MS6-215): | | Modified (MAS4-246): | |
| | | | mC*mC*mCmUmUmGf | | fU*fA*mUmUmUfCmAfCf | |
| | | | UmUfCfUfUmCmCmGm | | GmGmAmAmGfAmAfCm | |
| | | | UfGmAmAfAmUmA | | AmAmGmGmG*mC*mA | |
| 64 | 1570 | CCCTTGTTC | Unmodified: | 215 | Unmodified: | 246 |
| | | TTCCGTGAA | CCCUUGUUCUUCCGU | | UAUUUCACGGAAGAAC | |
| | | AT (SEQ ID | GAAAUA | | AAGGGCA | |
| | | NO: 184) | Modified (MS5-215): | | Modified (MAS2-246): | |
| | | | mC*mC*mCmUmUmGf | | fU*fA*mUmUmUfCmAm | |
| | | | UmUfCfUfUmCmCmGm | | CmGmGmAmAmGfAmAf | |
| | | | UfGfAmAmAmUmA | | CmAmAmGmGmG*mC*m | |
| | | | | | A | |
| 65 | 1570 | CCCTTGTTC | Unmodified: | 215 | Unmodified: | 246 |
| | | TTCCGTGAA | CCCUUGUUCUUCCGU | | UAUUUCACGGAAGAAC | |
| | | AT (SEQ ID | GAAAUA | | AAGGGCA | |
| | | NO: 184) | Modified (MS4-215): | | Modified (MAS2-246): | |
| | | | mC*mC*mCmUmUmGf | | fU*fA*mUmUmUfCmAm | |
| | | | UmUfCfUfUmCmCmGm | | CmGmGmAmAmGfAmAf | |
| | | | UmGmAmAmAmUmA | | CmAmAmGmGmG*mC*m | |
| | | | | | A | |
| 66 | 1570 | CCCTTGTTC | Unmodified: | 215 | Unmodified: | 246 |
| | | TTCCGTGAA | CCCUUGUUCUUCCGU | | UAUUUCACGGAAGAAC | |
| | | AT (SEQ ID | GAAAUA | | AAGGGCA | |
| | | NO: 184) | Modified: (MS2-215) | | Modified (MAS4-246): | |
| | | | mC*mC*mCmUfUmGfU | | fU*fA*mUmUmUfCmAfCf | |
| | | | mUfCfUfUmCmCmGmU | | GmGmAmAmGfAmAfCm | |
| | | | fGmAmAmAmUmA | | AmAmGmGmG*mC*mA | |
| 67 | 1629 | GGCAAACC | Unmodified: | 216 | Unmodified: | 247 |
| | | TGGATTAG | GGCAAACCUGGAUU | | UAACUCUAAUCCAGGU | |
| | | AGTT (SEQ | AGAGUUA | | UUGCCUA | |
| | | ID NO: 185) | Modified (MS3-216): | | Modified (MAS3-247): | |
| | | | mG*mG*mCmAfAmAm | | fU*fA*mAmCmUmCmUm | |
| | | | CmCfUfGfGmAmUfUm | | AmAmUmCmCmAfGmGm | |
| | | | AmGmAmGfUmUmA | | UmUmUmGmCmC*mU*m | |
| | | | | | A | |
| 68 | 1629 | GGCAAACC | Unmodified: | 216 | Unmodified: | 247 |
| | | TGGATTAG | GGCAAACCUGGAUU | | UAACUCUAAUCCAGGU | |
| | | AGTT (SEQ | AGAGUUA | | UUGCCUA | |
| | | ID NO: 185) | Modified (MS4-216): | | Modified (MAS4-247): | |
| | | | mG*mG*mCmAmAmAf | | fU*fA*mAmCmUfCmUfAf | |
| | | | CmCfUfGfGmAmUmU | | AmUmCmCmAfGmGfUm | |
| | | | mAmGmAmGmUmUmA | | UmUmGmCmC*mU*mA | |
| 69 | 1629 | GGCAAACC | Unmodified: | 216 | Unmodified: | 247 |
| | | TGGATTAG | GGCAAACCUGGAUU | | UAACUCUAAUCCAGGU | |
| | | AGTT (SEQ | AGAGUUA | | UUGCCUA | |
| | | ID NO: 185) | Modified (MS6-216): | | Modified (MAS4-247): | |
| | | | mG*mG*mCmAmAmAf | | fU*fA*mAmCmUfCmUfAf | |
| | | | CmCfUfGfGmAmUmU | | AmUmCmCmAfGmGfUm | |
| | | | mAfGmAmGfUmUmA | | UmUmGmCmC*mU*mA | |
| 70 | 1629 | GGCAAACC | Unmodified: | 216 | Unmodified: | 247 |
| | | TGGATTAG | GGCAAACCUGGAUU | | UAACUCUAAUCCAGGU | |
| | | AGTT (SEQ | AGAGUUA | | UUGCCUA | |
| | | ID NO: 185) | Modified (MS5-216): | | Modified (MAS2-247): | |
| | | | mG*mG*mCmAmAmAf | | fU*fA*mAmCmUfCmUm | |
| | | | CmCfUfGfGmAmUmU | | AmAmUmCmCmAfGmGf | |
| | | | mAfGfAmGmUmUmA | | UmUmUmGmCmC*mU*m | |
| | | | | | A | |
| 71 | 1629 | GGCAAACC | Unmodified: | 216 | Unmodified: | 247 |
| | | TGGATTAG | GGCAAACCUGGAUU | | UAACUCUAAUCCAGGU | |
| | | AGTT (SEQ | AGAGUUA | | UUGCCUA | |
| | | ID NO: 185) | Modified (MS4-216): | | Modified (MAS2-247): | |
| | | | mG*mG*mCmAmAmAf | | fU*fA*mAmCmUfCmUm | |
| | | | CmCfUfGfGmAmUmU | | AmAmUmCmCmAfGmGf | |
| | | | mAmGmAmGmUmUmA | | UmUmUmGmCmC*mU*m | |
| | | | | | A | |
| 72 | 1629 | GGCAAACC | Unmodified: | 216 | Unmodified: | 247 |
| | | TGGATTAG | GGCAAACCUGGAUU | | UAACUCUAAUCCAGGU | |
| | | AGTT (SEQ | AGAGUUA | | UUGCCUA | |
| | | ID NO: 185) | Modified (MS2-216): | | Modified (MAS4-247): | |
| | | | mG*mG*mCmAfAmAfC | | fU*fA*mAmCmUfCmUfAf | |
| | | | mCfUfGfGmAmUmUm | | AmUmCmCmAfGmGfUm | |
| | | | AfGmAmGmUmUmA | | UmUmGmCmC*mU*mA | |
| 73 | 1627 | TAGGCAAA | Unmodified: | 217 | Unmodified: | 248 |
| | | CCTGGATTA | UAGGCAAACCUGGA | | UCUCUAAUCCAGGUUU | |
| | | GAG (SEQ ID | UUAGAGA | | GCCUAGA | |
| | | NO: 186) | Modified: (MS3-217) | | Modified (MAS3-248): | |
| | | | mU*mA*mGmGfCmAm | | fU*fC*mUmCmUmAmAm | |
| | | | AmAfCfCfUmGmGfAm | | UmCmCmAmGmGfUmUm | |
| | | | UmUmAmGfAmGmA | | UmGmCmCmUmA*mG*m | |
| | | | | | A | |
| 74 | 1627 | TAGGCAAA | Unmodified: | 217 | Unmodified: | 248 |
| | | CCTGGATTA | UAGGCAAACCUGGA | | UCUCUAAUCCAGGUUU | |
| | | GAG (SEQ ID | UUAGAGA | | GCCUAGA | |
| | | NO: 186) | Modified (MS4-217): | | Modified (MAS4-248): | |
| | | | mU*mA*mGmGmCmAf | | fU*fC*mUmCmUfAmAfUf | |
| | | | AmAfCfCfUmGmGmA | | CmCmAmGmGfUmUfUm | |
| | | | mUmUmAmGmAmGmA | | GmCmCmUmA*mG*mA | |
| 75 | 1627 | TAGGCAAA | Unmodified: | 217 | Unmodified: | 248 |
| | | CCTGGATTA | UAGGCAAACCUGGA | | UCUCUAAUCCAGGUUU | |
| | | GAG (SEQ ID | UUAGAGA | | GCCUAGA | |
| | | NO: 186) | Modified (MS6-217): | | Modified (MAS4-248): | |
| | | | mU*mA*mGmGmCmAf | | fU*fC*mUmCmUfAmAfUf | |
| | | | AmAfCfCfUmGmGmA | | CmCmAmGmGfUmUfUm | |
| | | | mUfUmAmGfAmGmA | | GmCmCmUmA*mG*mA | |
| 76 | 1627 | TAGGCAAA | Unmodified: | 217 | Unmodified: | 248 |
| | | CCTGGATTA | UAGGCAAACCUGGA | | UCUCUAAUCCAGGUUU | |
| | | GAG (SEQ ID | UUAGAGA | | GCCUAGA | |
| | | NO: 186) | Modified (MS5-217): | | Modified (MAS2-248): | |
| | | | mU*mA*mGmGmCmAf | | fU*fC*mUmCmUfAmAm | |
| | | | AmAfCfCfUmGmGmA | | UmCmCmAmGmGfUmUf | |
| | | | mUfUfAmGmAmGmA | | UmGmCmCmUmA*mG*m | |
| | | | | | A | |
| 77 | 1627 | TAGGCAAA | Unmodified: | 217 | Unmodified: | 248 |
| | | CCTGGATTA | UAGGCAAACCUGGA | | UCUCUAAUCCAGGUUU | |
| | | GAG (SEQ ID | UUAGAGA | | GCCUAGA | |
| | | NO: 186) | Modified (MS4-217): | | Modified (MAS2-248): | |
| | | | mU*mA*mGmGmCmAf | | fU*fC*mUmCmUfAmAm | |
| | | | AmAfCfCfUmGmGmA | | UmCmCmAmGmGfUmUf | |
| | | | mUmUmAmGmAmGmA | | UmGmCmCmUmA*mG*m | |
| | | | | | A | |
| 78 | 1627 | TAGGCAAA | Unmodified: | 217 | Unmodified: | 248 |
| | | CCTGGATTA | UAGGCAAACCUGGA | | UCUCUAAUCCAGGUUU | |
| | | GAG (SEQ ID | UUAGAGA | | GCCUAGA | |
| | | NO: 186) | Modified (MS2-217): | | Modified (MAS4-248): | |
| | | | mU*mA*mGmGfCmAf | | fU*fC*mUmCmUfAmAfUf | |
| | | | AmAfCfCfUmGmGmA | | CmCmAmGmGfUmUfUm | |
| | | | mUfUmAmGmAmGmA | | GmCmCmUmA*mG*mA | |
| 79 | 1628 | AGGCAAAC | Unmodified: | 218 | Unmodified: | 249 |
| | | CTGGATTAG | AGGCAAACCUGGAU | | UACUCUAAUCCAGGUU | |
| | | AGT (SEQ ID | UAGAGUA | | UGCCUAG | |
| | | NO: 187) | Modified (MS3-218): | | Modified (MAS3-249): | |
| | | | mA*mG*mGmCfAmAm | | fU*fA*mCmUmCmUmAm | |
| | | | AmCfCfUfGmGmAfUm | | AmUmCmCmAmGfGmUm | |
| | | | UmAmGmAfGmUmA | | UmUmGmCmCmU*mA*m | |
| | | | | | G | |
| 80 | 1628 | AGGCAAAC | Unmodified: | 218 | Unmodified | 249 |
| | | CTGGATTAG | AGGCAAACCUGGAU | | UACUCUAAUCCAGGUU | |
| | | AGT (SEQ ID | UAGAGUA | | UGCCUAG | |
| | | NO: 187) | Modified (MS4-218): | | Modified (MAS4-249): | |
| | | | mA*mG*mGmCmAmAf | | fU*fA*mCmUmCfUmAfAf | |
| | | | AmCfCfUfGmGmAmU | | UmCmCmAmGfGmUfUm | |
| | | | mUmAmGmAmGmUmA | | UmGmCmCmU*mA*mG | |
| 81 | 1628 | AGGCAAAC | Unmodified: | 218 | Unmodified: | 249 |
| | | CTGGATTAG | AGGCAAACCUGGAU | | UACUCUAAUCCAGGUU | |
| | | AGT (SEQ ID | UAGAGUA | | UGCCUAG | |
| | | NO: 187) | Modified (MS6-218): | | Modified (MAS4-249): | |
| | | | mA*mG*mGmCmAmAf | | fU*fA*mCmUmCfUmAfAf | |
| | | | AmCfCfUfGmGmAmU | | UmCmCmAmGfGmUfUm | |
| | | | mUfAmGmAfGmUmA | | UmGmCmCmU*mA*mG | |
| 82 | 1628 | AGGCAAAC | Unmodified: | 218 | Unmodified: | 249 |
| | | CTGGATTAG | AGGCAAACCUGGAU | | UACUCUAAUCCAGGUU | |
| | | AGT (SEQ ID | UAGAGUA | | UGCCUAG | |
| | | NO: 187) | Modified (MS5-218): | | Modified (MAS2-249): | |
| | | | mA*mG*mGmCmAmAf | | fU*fA*mCmUmCfUmAm | |
| | | | AmCfCfUfGmGmAmU | | AmUmCmCmAmGfGmUf | |
| | | | mUfAfGmAmGmUmA | | UmUmGmCmCmU*mA*m | |
| | | | | | G | |
| 83 | 1628 | AGGCAAAC | Unmodified: | 218 | Unmodified: | 249 |
| | | CTGGATTAG | AGGCAAACCUGGAU | | UACUCUAAUCCAGGUU | |
| | | AGT (SEQ ID | UAGAGUA | | UGCCUAG | |
| | | NO: 187) | Modified (MS4-218): | | Modified (MAS2-249): | |
| | | | mA*mG*mGmCmAmAf | | fU*fA*mCmUmCfUmAm | |
| | | | AmCfCfUfGmGmAmU | | AmUmCmCmAmGfGmUf | |
| | | | mUmAmGmAmGmUmA | | UmUmGmCmCmU*mA*m | |
| | | | | | G | |
| 84 | 1628 | AGGCAAAC | Unmodified: | 218 | Unmodified: | 249 |
| | | CTGGATTAG | AGGCAAACCUGGAU | | UACUCUAAUCCAGGUU | |
| | | AGT (SEQ ID | UAGAGUA | | UGCCUAG | |
| | | NO: 187) | Modified (MS2-218): | | Modified (MAS4-249): | |
| | | | mA*mG*mGmCfAmAf | | fU*fA*mCmUmCfUmAfAf | |
| | | | AmCfCfUfGmGmAmU | | UmCmCmAmGfGmUfUm | |
| | | | mUfAmGmAmGmUmA | | UmGmCmCmU*mA*mG | |
| 85 | 1634 | ACCTGGATT | Unmodified: | 219 | Unmodified: | 250 |
| | | AGAGTTAC | ACCUGGAUUAGAGU | | UGAUGUAACUCUAAUC | |
| | | ATC (SEQ ID | UACAUCA | | CAGGUUU | |
| | | NO: 188) | Modified (MS3-219): | | Modified (MAS3-250): | |
| | | | mA*mC*mCmUfGmGm | | fU*fG*mAmUmGmUmAm | |
| | | | AmUfUfAfGmAmGfUm | | AmCmUmCmUmAfAmUm | |
| | | | UmAmCmAfUmCmA | | CmCmAmGmGmU*mU*m | |
| | | | | | U | |
| 86 | 1634 | ACCTGGATT | Unmodified: | 219 | Unmodified: | 250 |
| | | AGAGTTAC | ACCUGGAUUAGAGU | | UGAUGUAACUCUAAUC | |
| | | ATC (SEQ ID | UACAUCA | | CAGGUUU | |
| | | NO: 188) | Modified (MS4-219): | | Modified (MAS4-250): | |
| | | | mA*mC*mCmUmGmGf | | fU*fG*mAmUmGfUmAfA | |
| | | | AmUfUfAfGmAmGmU | | fCmUmCmUmAfAmUfCm | |
| | | | mUmAmCmAmUmCmA | | CmAmGmGmU*mU*mU | |
| 87 | 1634 | ACCTGGATT | Unmodified: | 219 | Unmodified: | 250 |
| | | AGAGTTAC | ACCUGGAUUAGAGU | | UGAUGUAACUCUAAUC | |
| | | ATC (SEQ ID | UACAUCA | | CAGGUUU | |
| | | NO: 188) | Modified (MS6-219): | | Modified (MAS4-250): | |
| | | | mA*mC*mCmUmGmGf | | fU*fG*mAmUmGfUmAfA | |
| | | | AmUfUfAfGmAmGmU | | fCmUmCmUmAfAmUfCm | |
| | | | mUfAmCmAfUmCmA | | CmAmGmGmU*mU*mU | |
| 88 | 1634 | ACCTGGATT | Unmodified: | 219 | Unmodified: | 250 |
| | | AGAGTTAC | ACCUGGAUUAGAGU | | UGAUGUAACUCUAAUC | |
| | | ATC (SEQ ID | UACAUCA | | CAGGUUU | |
| | | NO: 188) | Modified (MS5-219): | | Modified (MAS2-250): | |
| | | | mA*mC*mCmUmGmGf | | fU*fG*mAmUmGfUmAm | |
| | | | AmUfUfAfGmAmGmU | | AmCmUmCmUmAfAmUf | |
| | | | mUfAfCmAmUmCmA | | CmCmAmGmGmU*mU*m | |
| | | | | | U | |
| 89 | 1634 | ACCTGGATT | Unmodified: | 219 | Unmodified: | 250 |
| | | AGAGTTAC | ACCUGGAUUAGAGU | | UGAUGUAACUCUAAUC | |
| | | ATC (SEQ ID | UACAUCA | | CAGGUUU | |
| | | NO: 188) | Modified (MS4-219): | | Modified (MAS2-250): | |
| | | | mA*mC*mCmUmGmGf | | fU*fG*mAmUmGfUmAm | |
| | | | AmUfUfAfGmAmGmU | | AmCmUmCmUmAfAmUf | |
| | | | mUmAmCmAmUmCmA | | CmCmAmGmGmU*mU*m | |
| | | | | | U | |
| 90 | 1634 | ACCTGGATT | Unmodified: | 219 | Unmodified: | 250 |
| | | AGAGTTAC | ACCUGGAUUAGAGU | | UGAUGUAACUCUAAUC | |
| | | ATC (SEQ ID | UACAUCA | | CAGGUUU | |
| | | NO: 188) | Modified (MS2-219): | | Modified (MAS4-250): | |
| | | | mA*mC*mCmUfGmGfA | | fU*fG*mAmUmGfUmAfA | |
| | | | mUfUfAfGmAmGmUm | | fCmUmCmUmAfAmUfCm | |
| | | | UfAmCmAmUmCmA | | CmAmGmGmU*mU*mU | |
| 91 | 1659 | GATGATTA | Unmodified: | 220 | Unmodified: | 251 |
| | | GTTCAGAG | GAUGAUUAGUUCAG | | UAUAUCUCUGAACUAA | |
| | | ATAT (SEQ | AGAUAUA | | UCAUCCA | |
| | | ID NO: 189) | Modified (MS3-220): | | Modified (MAS3-251): | |
| | | | mG*mA*mUmGfAmUm | | fU*fA*mUmAmUmCmUm | |
| | | | UmAfGfUfUmCmAfGm | | CmUmGmAmAmCfUmAm | |
| | | | AmGmAmUfAmUmA | | AmUmCmAmUmC*mC*m | |
| | | | | | A | |
| 92 | 1659 | GATGATTA | Unmodified: | 220 | Unmodified: | 251 |
| | | GTTCAGAG | GAUGAUUAGUUCAG | | UAUAUCUCUGAACUAA | |
| | | ATAT (SEQ | AGAUAUA | | UCAUCCA | |
| | | ID NO: 189) | Modified (MS4-220): | | Modified (MAS4-251): | |
| | | | mG*mA*mUmGmAmUf | | fU*fA*mUmAmUfCmUfCf | |
| | | | UmAfGfUfUmCmAmG | | UmGmAmAmCfUmAfAm | |
| | | | mAmGmAmUmAmUmA | | UmCmAmUmC*mC*mA | |
| 93 | 1659 | GATGATTA | Unmodified: | 220 | Unmodified: | 251 |
| | | GTTCAGAG | GAUGAUUAGUUCAG | | UAUAUCUCUGAACUAA | |
| | | ATAT (SEQ | AGAUAUA | | UCAUCCA | |
| | | ID NO: 189) | Modified (MS6-220): | | Modified (MAS4-251): | |
| | | | mG*mA*mUmGmAmUf | | fU*fA*mUmAmUfCmUfCf | |
| | | | UmAfGfUfUmCmAmG | | UmGmAmAmCfUmAfAm | |
| | | | mAfGmAmUfAmUmA | | UmCmAmUmC*mC*mA | |
| 94 | 1659 | GATGATTA | Unmodified: | 220 | Unmodified: | 251 |
| | | GTTCAGAG | GAUGAUUAGUUCAG | | UAUAUCUCUGAACUAA | |
| | | ATAT (SEQ | AGAUAUA | | UCAUCCA | |
| | | ID NO: 189) | Modified (MS5-220): | | Modified (MAS2-251): | |
| | | | mG*mA*mUmGmAmUf | | fU*fA*mUmAmUfCmUm | |
| | | | UmAfGfUfUmCmAmG | | CmUmGmAmAmCfUmAf | |
| | | | mAfGfAmUmAmUmA | | AmUmCmAmUmC*mC*m | |
| | | | | | A | |
| 95 | 1659 | GATGATTA | Unmodified: | 220 | Unmodified: | 251 |
| | | GTTCAGAG | GAUGAUUAGUUCAG | | UAUAUCUCUGAACUAA | |
| | | ATAT (SEQ | AGAUAUA | | UCAUCCA | |
| | | ID NO: 189) | Modified (MS4-220): | | Modified (MAS2-251): | |
| | | | mG*mA*mUmGmAmUf | | fU*fA*mUmAmUfCmUm | |
| | | | UmAfGfUfUmCmAmG | | CmUmGmAmAmCfUmAf | |
| | | | mAmGmAmUmAmUmA | | AmUmCmAmUmC*mC*m | |
| | | | | | A | |
| 96 | 1659 | GATGATTA | Unmodified: | 220 | Unmodified: | 251 |
| | | GTTCAGAG | GAUGAUUAGUUCAG | | UAUAUCUCUGAACUAA | |
| | | ATAT (SEQ | AGAUAUA | | UCAUCCA | |
| | | ID NO: 189) | Modified (MS2-220): | | Modified (MAS4-251): | |
| | | | mG*mA*mUmGfAmUf | | fU*fA*mUmAmUfCmUfCf | |
| | | | UmAfGfUfUmCmAmG | | UmGmAmAmCfUmAfAm | |
| | | | mAfGmAmUmAmUmA | | UmCmAmUmC*mC*mA | |
| 97 | 1641 | TTAGAGTTA | Unmodified: | 221 | Unmodified: | 252 |
| | | CATCTCCTG | UUAGAGUUACAUCU | | UUCCAGGAGAUGUAAC | |
| | | GA (SEQ ID | CCUGGAA | | UCUAAUC | |
| | | NO: 190) | Modified (MS3-221): | | Modified (MAS3-252): | |
| | | | mU*mU*mAmGfAmGm | | fU*fU*mCmCmAmGmGm | |
| | | | UmUfAfCfAmUmCfUm | | AmGmAmUmGmUfAmA | |
| | | | CmCmUmGfGmAmA | | mCmUmCmUmAmA*mU* | |
| | | | | | mC | |
| 98 | 1641 | TTAGAGTTA | Unmodified: | 221 | Unmodified: | 252 |
| | | CATCTCCTG | UUAGAGUUACAUCU | | UUCCAGGAGAUGUAAC | |
| | | GA (SEQ ID | CCUGGAA | | UCUAAUC | |
| | | NO: 190) | Modified (MS4-221): | | Modified (MAS4-252): | |
| | | | mU*mU*mAmGmAmGf | | fU*fU*mCmCmAfGmGfAf | |
| | | | UmUfAfCfAmUmCmU | | GmAmUmGmUfAmAfCm | |
| | | | mCmCmUmGmGmAmA | | UmCmUmAmA*mU*mC | |
| 99 | 1641 | TTAGAGTTA | Unmodified: | 221 | Unmodified: | 252 |
| | | CATCTCCTG | UUAGAGUUACAUCU | | UUCCAGGAGAUGUAAC | |
| | | GA (SEQ ID | CCUGGAA | | UCUAAUC | |
| | | NO: 190) | Modified (MS6-221): | | Modified (MAS4-252): | |
| | | | mU*mU*mAmGmAmGf | | fU*fU*mCmCmAfGmGfAf | |
| | | | UmUfAfCfAmUmCmU | | GmAmUmGmUfAmAfCm | |
| | | | mCfCmUmGfGmAmA | | UmCmUmAmA*mU*mC | |
| 100 | 1641 | TTAGAGTTA | Unmodified: | 221 | Unmodified: | 252 |
| | | CATCTCCTG | UUAGAGUUACAUCU | | UUCCAGGAGAUGUAAC | |
| | | GA (SEQ ID | CCUGGAA | | UCUAAUC | |
| | | NO: 190) | Modified (MS5-221): | | Modified (MAS2-252): | |
| | | | mU*mU*mAmGmAmGf | | fU*fU*mCmCmAfGmGm | |
| | | | UmUfAfCfAmUmCmU | | AmGmAmUmGmUfAmAf | |
| | | | mCfCfUmGmGmAmA | | CmUmCmUmAmA*mU*m | |
| | | | | | C | |
| 101 | 1641 | TTAGAGTTA | Unmodified: | 221 | Unmodified: | 252 |
| | | CATCTCCTG | UUAGAGUUACAUCU | | UUCCAGGAGAUGUAAC | |
| | | GA (SEQ ID | CCUGGAA | | UCUAAUC | |
| | | NO: 190) | Modified (MS4-221): | | Modified (MAS2-252): | |
| | | | mU*mU*mAmGmAmGf | | fU*fU*mCmCmAfGmGm | |
| | | | UmUfAfCfAmUmCmU | | AmGmAmUmGmUfAmAf | |
| | | | mCmCmUmGmGmAmA | | CmUmCmUmAmA*mU*m | |
| | | | | | C | |
| 102 | 1641 | TTAGAGTTA | Unmodified: | 221 | Unmodified: | 252 |
| | | CATCTCCTG | UUAGAGUUACAUCU | | UUCCAGGAGAUGUAAC | |
| | | GA (SEQ ID | CCUGGAA | | UCUAAUC | |
| | | NO: 190) | Modified (MS2-221): | | Modified (MAS4-252): | |
| | | | mU*mU*mAmGfAmGf | | fU*fU*mCmCmAfGmGfAf | |
| | | | UmUfAfCfAmUmCmU | | GmAmUmGmUfAmAfCm | |
| | | | mCfCmUmGmGmAmA | | UmCmUmAmA*mU*mC | |
| 103 | 1631 | CAAACCTG | Unmodified: | 222 | Unmodified: | 253 |
| | | GATTAGAG | CAAACCUGGAUUAG | | UGUAACUCUAAUCCAG | |
| | | TTAC (SEQ | AGUUACA | | GUUUGCC | |
| | | ID NO: 191) | Modified (MS3-222): | | Modified (MAS3-253): | |
| | | | mC*mA*mAmAfCmCm | | fU*fG*mUmAmAmCmUm | |
| | | | UmGfGfAfUmUmAfGm | | CmUmAmAmUmCfCmAm | |
| | | | AmGmUmUfAmCmA | | GmGmUmUmUmG*mC*m | |
| | | | | | C | |
| 104 | 1631 | CAAACCTG | Unmodified: | 222 | Unmodified: | 253 |
| | | GATTAGAG | CAAACCUGGAUUAG | | UGUAACUCUAAUCCAG | |
| | | TTAC (SEQ | AGUUACA | | GUUUGCC | |
| | | ID NO: 191) | Modified (MS4-222): | | Modified (MAS4-253): | |
| | | | mC*mA*mAmAmCmCf | | fU*fG*mUmAmAfCmUfCf | |
| | | | UmGfGfAfUmUmAmG | | UmAmAmUmCfCmAfGm | |
| | | | mAmGmUmUmAmCmA | | GmUmUmUmG*mC*mC | |
| 105 | 1631 | CAAACCTG | Unmodified: | 222 | Unmodified: | 253 |
| | | GATTAGAG | CAAACCUGGAUUAG | | UGUAACUCUAAUCCAG | |
| | | TTAC (SEQ | AGUUACA | | GUUUGCC | |
| | | ID NO: 191) | Modified (MS6-222): | | Modified (MAS4-253): | |
| | | | mC*mA*mAmAmCmCf | | fU*fG*mUmAmAfCmUfCf | |
| | | | UmGfGfAfUmUmAmG | | UmAmAmUmCfCmAfGm | |
| | | | mAfGmUmUfAmCmA | | GmUmUmUmG*mC*mC | |
| 106 | 1631 | CAAACCTG | Unmodified: | 222 | Unmodified: | 253 |
| | | GATTAGAG | CAAACCUGGAUUAG | | UGUAACUCUAAUCCAG | |
| | | TTAC (SEQ | AGUUACA | | GUUUGCC | |
| | | ID NO: 191) | Modified (MS5-222): | | Modified (MAS2-253): | |
| | | | mC*mA*mAmAmCmCf | | fU*fG*mUmAmAfCmUm | |
| | | | UmGfGfAfUmUmAmG | | CmUmAmAmUmCfCmAf | |
| | | | mAfGfUmUmAmCmA | | GmGmUmUmUmG*mC*m | |
| | | | | | C | |
| 107 | 1631 | CAAACCTG | Unmodified: | 222 | Unmodified: | 253 |
| | | GATTAGAG | CAAACCUGGAUUAG | | UGUAACUCUAAUCCAG | |
| | | TTAC (SEQ | AGUUACA | | GUUUGCC | |
| | | ID NO: 191) | Modified (MS4-222): | | Modified (MAS2-253): | |
| | | | mC*mA*mAmAmCmCf | | fU*fG*mUmAmAfCmUm | |
| | | | UmGfGfAfUmUmAmG | | CmUmAmAmUmCfCmAf | |
| | | | mAmGmUmUmAmCmA | | GmGmUmUmUmG*mC*m | |
| | | | | | C | |
| 108 | 1631 | CAAACCTG | Unmodified: | 222 | Unmodified: | 253 |
| | | GATTAGAG | CAAACCUGGAUUAG | | UGUAACUCUAAUCCAG | |
| | | TTAC (SEQ | AGUUACA | | GUUUGCC | |
| | | ID NO: 191) | Modified (MS2-222): | | Modified (MAS4-253): | |
| | | | mC*mA*mAmAfCmCfU | | fU*fG*mUmAmAfCmUfCf | |
| | | | mGfGfAfUmUmAmGm | | UmAmAmUmCfCmAfGm | |
| | | | AfGmUmUmAmCmA | | GmUmUmUmG*mC*mC | |
| 109 | 1571 | CCTTGTTCT | Unmodified: | 223 | Unmodified: | 254 |
| | | TCCGTGAA | CCUUGUUCUUCCGU | | UAAUUUCACGGAAGAA | |
| | | ATT (SEQ ID | GAAAUUA | | CAAGGGC | |
| | | NO: 192) | Modified (MS3-223): | | Modified (MAS3-254): | |
| | | | mC*mC*mUmUfGmUm | | fU*fA*mAmUmUmUmCm | |
| | | | UmCfUfUfCmCmGfUm | | AmCmGmGmAmAfGmAm | |
| | | | GmAmAmAfUmUmA | | AmCmAmAmGmG*mG*m | |
| | | | | | C | |
| 110 | 1571 | CCTTGTTCT | Unmodified: | 223 | Unmodified: | 254 |
| | | TCCGTGAA | CCUUGUUCUUCCGU | | UAAUUUCACGGAAGAA | |
| | | ATT (SEQ ID | GAAAUUA | | CAAGGGC | |
| | | NO: 192) | Modified (MS4-223): | | Modified (MAS4-254): | |
| | | | mC*mC*mUmUmGmUf | | fU*fA*mAmUmUfUmCfAf | |
| | | | UmCfUfUfCmCmGmUm | | CmGmGmAmAfGmAfAm | |
| | | | GmAmAmAmUmUmA | | CmAmAmGmG*mG*mC | |
| 111 | 1571 | CCTTGTTCT | Unmodified: | 223 | Unmodified: | 254 |
| | | TCCGTGAA | CCUUGUUCUUCCGU | | UAAUUUCACGGAAGAA | |
| | | ATT (SEQ ID | GAAAUUA | | CAAGGGC | |
| | | NO: 192) | Modified (MS6-223): | | Modified (MAS4-254): | |
| | | | mC*mC*mUmUmGmUf | | fU*fA*mAmUmUfUmCfAf | |
| | | | UmCfUfUfCmCmGmUm | | CmGmGmAmAfGmAfAm | |
| | | | GfAmAmAfUmUmA | | CmAmAmGmG*mG*mC | |
| 112 | 1571 | CCTTGTTCT | Unmodified: | 223 | Unmodified: | 254 |
| | | TCCGTGAA | CCUUGUUCUUCCGU | | UAAUUUCACGGAAGAA | |
| | | ATT (SEQ ID | GAAAUUA | | CAAGGGC | |
| | | NO: 192) | Modified (MS5-223): | | Modified (MAS2-254): | |
| | | | mC*mC*mUmUmGmUf | | fU*fA*mAmUmUfUmCm | |
| | | | UmCfUfUfCmCmGmUm | | AmCmGmGmAmAfGmAf | |
| | | | GfAfAmAmUmUmA | | AmCmAmAmGmG*mG*m | |
| | | | | | C | |
| 113 | 1571 | CCTTGTTCT | Unmodified: | 223 | Unmodified: | 254 |
| | | TCCGTGAA | CCUUGUUCUUCCGU | | UAAUUUCACGGAAGAA | |
| | | ATT (SEQ ID | GAAAUUA | | CAAGGGC | |
| | | NO: 192) | Modified (MS4-223): | | Modified (MAS2-254): | |
| | | | mC*mC*mUmUmGmUf | | fU*fA*mAmUmUfUmCm | |
| | | | UmCfUfUfCmCmGmUm | | AmCmGmGmAmAfGmAf | |
| | | | GmAmAmAmUmUmA | | AmCmAmAmGmG*mG*m | |
| | | | | | C | |
| 114 | 1571 | CCTTGTTCT | Unmodified: | 223 | Unmodified: | 254 |
| | | TCCGTGAA | CCUUGUUCUUCCGU | | UAAUUUCACGGAAGAA | |
| | | ATT (SEQ ID | GAAAUUA | | CAAGGGC | |
| | | NO: 192) | Modified (MS2-223): | | Modified (MAS4-254): | |
| | | | mC*mC*mUmUfGmUfU | | fU*fA*mAmUmUfUmCfAf | |
| | | | mCfUfUfCmCmGmUmG | | CmGmGmAmAfGmAfAm | |
| | | | fAmAmAmUmUmA | | CmAmAmGmG*mG*mC | |
| 115 | 1648 | TACATCTCC | Unmodified: | 224 | Unmodified: | 255 |
| | | TGGATGATT | UACAUCUCCUGGAU | | UCUAAUCAUCCAGGAG | |
| | | AG (SEQ ID | GAUUAGA | | AUGUAAC | |
| | | NO: 193) | Modified (MS3-224): | | Modified (MAS3-255): | |
| | | | mU*mA*mCmAfUmCm | | fU*fC*mUmAmAmUmCm | |
| | | | UmCfCfUfGmGmAfUm | | AmUmCmCmAmGfGmAm | |
| | | | GmAmUmUfAmGmA | | GmAmUmGmUmA*mA*m | |
| | | | | | C | |
| 116 | 1648 | TACATCTCC | Unmodified: | 224 | Unmodified: | 255 |
| | | TGGATGATT | UACAUCUCCUGGAU | | UCUAAUCAUCCAGGAG | |
| | | AG (SEQ ID | GAUUAGA | | AUGUAAC | |
| | | NO: 193) | Modified (MS4-224): | | Modified (MAS4-255): | |
| | | | mU*mA*mCmAmUmCf | | fU*fC*mUmAmAfUmCfAf | |
| | | | UmCfCfUfGmGmAmU | | UmCmCmAmGfGmAfGm | |
| | | | mGmAmUmUmAmGmA | | AmUmGmUmA*mA*mC | |
| 117 | 1648 | TACATCTCC | Unmodified: | 224 | Unmodified: | 255 |
| | | TGGATGATT | UACAUCUCCUGGAU | | UCUAAUCAUCCAGGAG | |
| | | AG (SEQ ID | GAUUAGA | | AUGUAAC | |
| | | NO: 193) | Modified (MS6-224): | | Modified (MAS4-255): | |
| | | | mU*mA*mCmAmUmCf | | fU*fC*mUmAmAfUmCfAf | |
| | | | UmCfCfUfGmGmAmU | | UmCmCmAmGfGmAfGm | |
| | | | mGfAmUmUfAmGmA | | AmUmGmUmA*mA*mC | |
| 118 | 1648 | TACATCTCC | Unmodified: | 224 | Unmodified: | 255 |
| | | TGGATGATT | UACAUCUCCUGGAU | | UCUAAUCAUCCAGGAG | |
| | | AG (SEQ ID | GAUUAGA | | AUGUAAC | |
| | | NO: 193) | Modified (MS5-224): | | Modified (MAS2-255): | |
| | | | mU*mA*mCmAmUmCf | | fU*fC*mUmAmAfUmCm | |
| | | | UmCfCfUfGmGmAmU | | AmUmCmCmAmGfGmAf | |
| | | | mGfAfUmUmAmGmA | | GmAmUmGmUmA*mA*m | |
| | | | | | C | |
| 119 | 1648 | TACATCTCC | Unmodified: | 224 | Unmodified: | 255 |
| | | TGGATGATT | UACAUCUCCUGGAU | | UCUAAUCAUCCAGGAG | |
| | | AG (SEQ ID | GAUUAGA | | AUGUAAC | |
| | | NO: 193) | Modified (MS4-224): | | Modified (MAS2-255): | |
| | | | mU*mA*mCmAmUmCf | | fU*fC*mUmAmAfUmCm | |
| | | | UmCfCfUfGmGmAmU | | AmUmCmCmAmGfGmAf | |
| | | | mGmAmUmUmAmGmA | | GmAmUmGmUmA*mA*m | |
| | | | | | C | |
| 120 | 1648 | TACATCTCC | Unmodified: | 224 | Unmodified: | 255 |
| | | TGGATGATT | UACAUCUCCUGGAU | | UCUAAUCAUCCAGGAG | |
| | | AG (SEQ ID | GAUUAGA | | AUGUAAC | |
| | | NO: 193) | Modified (MS2-224): | | Modified (MAS4-255): | |
| | | | mU*mA*mCmAfUmCfU | | fU*fC*mUmAmAfUmCfAf | |
| | | | mCfCfUfGmGmAmUm | | UmCmCmAmGfGmAfGm | |
| | | | GfAmUmUmAmGmA | | AmUmGmUmA*mA*mC | |
| 121 | 1503 | TACCAGCA | Unmodified: | 225 | Unmodified: | 256 |
| | | GACCTGCG | UACCAGCAGACCUGC | | UUGCGCGCAGGUCUGC | |
| | | CGCA (SEQ | GCGCAA | | UGGUACC | |
| | | ID NO: 194) | Modified (MS3-225): | | Modified (MAS3-256): | |
| | | | mU*mA*mCmCfAmGm | | fU*fU*mGmCmGmCmGm | |
| | | | CmAfGfAfCmCmUfGm | | CmAmGmGmUmCfUmGm | |
| | | | CmGmCmGfCmAmA | | CmUmGmGmUmA*mC*m | |
| | | | | | C | |
| 122 | 1503 | TACCAGCA | Unmodified: | 225 | Unmodified: | 256 |
| | | GACCTGCG | UACCAGCAGACCUGC | | UUGCGCGCAGGUCUGC | |
| | | CGCA (SEQ | GCGCAA | | UGGUACC | |
| | | ID NO: 194) | Modified (MS4-225): | | Modified (MAS4-256): | |
| | | | mU*mA*mCmCmAmGf | | fU*fU*mGmCmGfCmGfCf | |
| | | | CmAfGfAfCmCmUmGm | | AmGmGmUmCfUmGfCm | |
| | | | CmGmCmGmCmAmA | | UmGmGmUmA*mC*mC | |
| 123 | 1503 | TACCAGCA | Unmodified: | 225 | Unmodified: | 256 |
| | | GACCTGCG | UACCAGCAGACCUGC | | UUGCGCGCAGGUCUGC | |
| | | CGCA (SEQ | GCGCAA | | UGGUACC | |
| | | ID NO: 194) | Modified (MS6-225): | | Modified (MAS4-256): | |
| | | | mU*mA*mCmCmAmGf | | fU*fU*mGmCmGfCmGfCf | |
| | | | CmAfGfAfCmCmUmGm | | AmGmGmUmCfUmGfCm | |
| | | | CfGmCmGfCmAmA | | UmGmGmUmA*mC*mC | |
| 124 | 1503 | TACCAGCA | Unmodified: | 225 | Unmodified: | 256 |
| | | GACCTGCG | UACCAGCAGACCUGC | | UUGCGCGCAGGUCUGC | |
| | | CGCA (SEQ | GCGCAA | | UGGUACC | |
| | | ID NO: 194) | Modified (MS5-225): | | Modified (MAS2-256): | |
| | | | mU*mA*mCmCmAmGf | | fU*fU*mGmCmGfCmGmC | |
| | | | CmAfGfAfCmCmUmGm | | mAmGmGmUmCfUmGfC | |
| | | | CfGfCmGmCmAmA | | mUmGmGmUmA*mC*mC | |
| 125 | 1503 | TACCAGCA | Unmodified: | 225 | Unmodified: | 256 |
| | | GACCTGCG | UACCAGCAGACCUGC | | UUGCGCGCAGGUCUGC | |
| | | CGCA (SEQ | GCGCAA | | UGGUACC | |
| | | ID NO: 194) | Modified (MS4-225): | | Modified (MAS2-256): | |
| | | | mU*mA*mCmCmAmGf | | fU*fU*mGmCmGfCmGmC | |
| | | | CmAfGfAfCmCmUmGm | | mAmGmGmUmCfUmGfC | |
| | | | CmGmCmGmCmAmA | | mUmGmGmUmA*mC*mC | |
| 126 | 1503 | TACCAGCA | Unmodified: | 225 | Unmodified: | 256 |
| | | GACCTGCG | UACCAGCAGACCUGC | | UUGCGCGCAGGUCUGC | |
| | | CGCA (SEQ | GCGCAA | | UGGUACC | |
| | | ID NO: 194) | Modified (MS2-225): | | Modified (MAS4-256): | |
| | | | mU*mA*mCmCfAmGfC | | fU*fU*mGmCmGfCmGfCf | |
| | | | mAfGfAfCmCmUmGmC | | AmGmGmUmCfUmGfCm | |
| | | | fGmCmGmCmAmA | | UmGmGmUmA*mC*mC | |
| 127 | 1643 | AGAGTTAC | Unmodified: | 226 | Unmodified: | 257 |
| | | ATCTCCTGG | AGAGUUACAUCUCC | | UCAUCCAGGAGAUGUA | |
| | | ATG (SEQ ID | UGGAUGA | | ACUCUAA | |
| | | NO: 195) | Modified (MS3-226): | | Modified (MAS3-257): | |
| | | | mA*mG*mAmGfUmUm | | fU*fC*mAmUmCmCmAm | |
| | | | AmCfAfUfCmUmCfCm | | GmGmAmGmAmUfGmU | |
| | | | UmGmGmAfUmGmA | | mAmAmCmUmCmU*mA* | |
| | | | | | mA | |
| 128 | 1643 | AGAGTTAC | Unmodified: | 226 | Unmodified: | 257 |
| | | ATCTCCTGG | AGAGUUACAUCUCC | | UCAUCCAGGAGAUGUA | |
| | | ATG (SEQ ID | UGGAUGA | | ACUCUAA | |
| | | NO: 195) | Modified (MS4-226): | | Modified (MAS4-257): | |
| | | | mA*mG*mAmGmUmUf | | fU*fC*mAmUmCfCmAfGf | |
| | | | AmCfAfUfCmUmCmCm | | GmAmGmAmUfGmUfAm | |
| | | | UmGmGmAmUmGmA | | AmCmUmCmU*mA*mA | |
| 129 | 1643 | AGAGTTAC | Unmodified: | 226 | Unmodified: | 257 |
| | | ATCTCCTGG | AGAGUUACAUCUCC | | UCAUCCAGGAGAUGUA | |
| | | ATG (SEQ ID | UGGAUGA | | ACUCUAA | |
| | | NO: 195) | Modified (MS6-226): | | Modified (MAS4-257): | |
| | | | mA*mG*mAmGmUmUf | | fU*fC*mAmUmCfCmAfGf | |
| | | | AmCfAfUfCmUmCmCm | | GmAmGmAmUfGmUfAm | |
| | | | UfGmGmAfUmGmA | | AmCmUmCmU*mA*mA | |
| 130 | 1643 | AGAGTTAC | Unmodified: | 226 | Unmodified: | 257 |
| | | ATCTCCTGG | AGAGUUACAUCUCC | | UCAUCCAGGAGAUGUA | |
| | | ATG (SEQ ID | UGGAUGA | | ACUCUAA | |
| | | NO: 195) | Modified (MS5-226): | | Modified (MAS2-257): | |
| | | | mA*mG*mAmGmUmUf | | fU*fC*mAmUmCfCmAmG | |
| | | | AmCfAfUfCmUmCmCm | | mGmAmGmAmUfGmUfA | |
| | | | UfGfGmAmUmGmA | | mAmCmUmCmU*mA*mA | |
| 131 | 1643 | AGAGTTAC | Unmodified: | 226 | Unmodified: | 257 |
| | | ATCTCCTGG | AGAGUUACAUCUCC | | UCAUCCAGGAGAUGUA | |
| | | ATG (SEQ ID | UGGAUGA | | ACUCUAA | |
| | | NO: 195) | Modified (MS4-226): | | Modified (MAS2-257): | |
| | | | mA*mG*mAmGmUmUf | | fU*fC*mAmUmCfCmAmG | |
| | | | AmCfAfUfCmUmCmCm | | mGmAmGmAmUfGmUfA | |
| | | | UmGmGmAmUmGmA | | mAmCmUmCmU*mA*mA | |
| 132 | 1643 | AGAGTTAC | Unmodified: | 226 | Unmodified: | 257 |
| | | ATCTCCTGG | AGAGUUACAUCUCC | | UCAUCCAGGAGAUGUA | |
| | | ATG (SEQ ID | UGGAUGA | | ACUCUAA | |
| | | NO: 195) | Modified (MS2-226): | | Modified (MAS4-257): | |
| | | | mA*mG*mAmGfUmUf | | fU*fC*mAmUmCfCmAfGf | |
| | | | AmCfAfUfCmUmCmCm | | GmAmGmAmUfGmUfAm | |
| | | | UfGmGmAmUmGmA | | AmCmUmCmU*mA*mA | |
| 133 | 1635 | CCTGGATTA | Unmodified: | 227 | Unmodified: | 258 |
| | | GAGTTACAT | CCUGGAUUAGAGUU | | UAGAUGUAACUCUAAU | |
| | | CT (SEQ ID | ACAUCUA | | CCAGGUU | |
| | | NO: 196) | Modified (MS3-227): | | Modified (MAS3-258): | |
| | | | mC*mC*mUmGfGmAm | | fU*fA*mGmAmUmGmUm | |
| | | | UmUfAfGfAmGmUfUm | | AmAmCmUmCmUfAmAm | |
| | | | AmCmAmUfCmUmA | | UmCmCmAmGmG*mU*m | |
| | | | | | U | |
| 134 | 1650 | CATCTCCTG | Unmodified: | 228 | Unmodified: | 259 |
| | | GATGATTA | CAUCUCCUGGAUGA | | UAACUAAUCAUCCAGG | |
| | | GTT (SEQ ID | UUAGUUA | | AGAUGUA | |
| | | NO: 197) | Modified (MS3-228): | | Modified (MAS3-259): | |
| | | | mC*mA*mUmCfUmCm | | fU*fA*mAmCmUmAmAm | |
| | | | CmUfGfGfAmUmGfAm | | UmCmAmUmCmCfAmGm | |
| | | | UmUmAmGfUmUmA | | GmAmGmAmUmG*mU*m | |
| | | | | | A | |
| 135 | 1638 | GGATTAGA | Unmodified: | 229 | Unmodified: | 260 |
| | | GTTACATCT | GGAUUAGAGUUACA | | JAGGAGAUGUAACUCU | |
| | | CCT (SEQ ID | UCUCCUA | | AAUCCAG | |
| | | NO: 198) | Modified (MS3-229): | | Modified (MAS3-260): | |
| | | | mG*mG*mAmUfUmAm | | fU*fA*mGmGmAmGmAm | |
| | | | GmAfGfUfUmAmCfAm | | UmGmUmAmAmCfUmCm | |
| | | | UmCmUmCfCmUmA | | UmAmAmUmCmC*mA*m | |
| | | | | | G | |
| 136 | 1566 | TGTGCCCTT | Unmodified: | 230 | Unmodified: | 261 |
| | | GTTCTTCCG | UGUGCCCUUGUUCU | | UCACGGAAGAACAAGG | |
| | | TG (SEQ ID | UCCGUGA | | GCACAGA | |
| | | NO: 199) | Modified (MS3-230): | | Modified (MAS3-261): | |
| | | | mU*mG*mUmGfCmCm | | fU*fC*mAmCmGmGmAm | |
| | | | CmUfUfGfUmUmCfUm | | AmGmAmAmCmAfAmGm | |
| | | | UmCmCmGfUmGmA | | GmGmCmAmCmA*mG*m | |
| | | | | | A | |
| 137 | 1576 | TTCTTCCGT | Unmodified: | 231 | Unmodified: | 262 |
| | | GAAATTCTG | UUCUUCCGUGAAAU | | UGCCAGAAUUUCACGG | |
| | | GC (SEQ ID | UCUGGCA | | AAGAACA | |
| | | NO: 200) | Modified (MS3-231): | | Modified (MAS3-262): | |
| | | | mU*mU*mCmUfUmCm | | fU*fG*mCmCmAmGmAm | |
| | | | CmGfUfGfAmAmAfUm | | AmUmUmUmCmAfCmGm | |
| | | | UmCmUmGfGmCmA | | GmAmAmGmAmA*mC*m | |
| | | | | | A | |
| 138 | 1621 | GCTGTCTAG | Unmodified: | 232 | Unmodified: | 263 |
| | | GCAAACCT | GCUGUCUAGGCAAA | | UUCCAGGUUUGCCUAG | |
| | | GGA (SEQ ID | CCUGGAA | | ACAGCGU | |
| | | NO: 201) | Modified (MS3-232): | | Modified (MAS3-263): | |
| | | | mG*mC*mUmGfUmCm | | fU*fU*mCmCmAmGmGm | |
| | | | UmAfGfGfCmAmAfAm | | UmUmUmGmCmCfUmAm | |
| | | | CmCmUmGfGmAmA | | GmAmCmAmGmC*mG*m | |
| | | | | | U | |
| 139 | 1649 | ACATCTCCT | Unmodified: | 233 | Unmodified: | 264 |
| | | GGATGATT | ACAUCUCCUGGAUG | | UACUAAUCAUCCAGGA | |
| | | AGT (SEQ ID | AUUAGUA | | GAUGUAA | |
| | | NO: 202) | Modified (MS3-233): | | Modified (MAS3-264): | |
| | | | mA*mC*mAmUfCmUm | | fU*fA*mCmUmAmAmUm | |
| | | | CmCfUfGfGmAmUfGm | | CmAmUmCmCmAfGmGm | |
| | | | AmUmUmAfGmUmA | | AmGmAmUmGmU*mA*m | |
| | | | | | A | |
| 140 | 1639 | GATTAGAG | Unmodified: | 234 | Unmodified: | 265 |
| | | TTACATCTC | GAUUAGAGUUACAU | | UCAGGAGAUGUAACUC | |
| | | CTG (SEQ ID | CUCCUGA | | UAAUCCA | |
| | | NO: 203) | Modified (MS3-234): | | Modified (MAS3-265): | |
| | | | mG*mA*mUmUfAmGm | | fU*fC*mAmGmGmAmGm | |
| | | | AmGfUfUfAmCmAfUm | | AmUmGmUmAmAfCmUm | |
| | | | CmUmCmCfUmGmA | | CmUmAmAmUmC*mC*m | |
| | | | | | A | |
| 141 | 1588 | ATTCTGGCT | Unmodified: | 235 | Unmodified: | 266 |
| | | GAATGTCTC | AUUCUGGCUGAAUG | | UGGGAGACAUUCAGCC | |
| | | CC (SEQ ID | UCUCCCA | | AGAAUUU | |
| | | NO: 204) | Modified (MS3-235): | | Modified (MAS3-266): | |
| | | | mA*mU*mUmCfUmGm | | fU*fG*mGmGmAmGmAm | |
| | | | GmCfUfGfAmAmUfGm | | CmAmUmUmCmAfGmCm | |
| | | | UmCmUmCfCmCmA | | CmAmGmAmAmU*mU*m | |

“m” indicates a 2′-O-methyl (2′-O-Me) modified nucleoside; “f” indicates a 2′-fluoro (2′-F) modified nucleoside; “*” indicates phosphorothioate internucleoside linkage; and the absence of “*” between two nucleosides indicate phosphodiester internucleoside linkage.
^{†}Each uracil base (U) in any one of the oligonucleotides and/or target sequences provided in Table 8 may independently and optionally be replaced with a thymine base (T), and/or each T may independently and optionally be replaced with a U. Target sequences listed in Table 8 contain T′s, but binding of an oligonucleotide described herein to RNA and/or DNA is contemplated.
^{∧}Target sequence start position is in NM_001306068.3 (SEQ ID NO: 161)
^{‡}siRNA oligonucleotides provided in Table 8 that have sense and antisense strands with the same nucleobase sequences as indicated by the SEQ ID NOs can have different modification patterns. The modified sense and antisense strands are indicated by an “MS#” or “MAS#,” respectively. siRNA oligonucleotides provided in Table 8 comprise an antisense strand without a 5′-(E)-Vinylphosphonate modification. However, siRNA oligonucleotides comprising an antisense strand with a 5′-(E)-Vinylphosphonate modification are also contemplated and are represented herein by the same MAS# with a “VP” before the MAS# (i.e., VP-MAS#). siRNA oligonucleotides provided in Table 8 may be conjugated to a compound of the formula NH₂-(CH₂)₆- at the 5′ or 3′ nucleoside of the sense strand.

TABLE 9
Additional non-limiting examples of siRNAs^{†‡}

| | Target | | | | | |
|---|---|---|---|---|---|---|
| | start | | | SEQ | | SEQ |
| siRNA | posi- | Target | Sense | ID | Antisense | ID |
| No. | tion^{∧} | Sequence | strand (5′-3′) | NO | strand (5′-3′) | NO |
| 142 | 1659 | GATGATTA | Unmodified: | 220 | Unmodified: | 251 |
| | | GTTCAGAG | GAUGAUUAGUUCAG | | UAUAUCUCUGAACUAA | |
| | | ATAT (SEQ | AGAUAUA | | UCAUCCA | |
| | | ID NO: 189) | Modified (MS7-220): | | Modified (VP-MAS6-251): | |
| | | | mG*mA*mUmGmAmUf | | VP- | |
| | | | UmAfGfUfUmCmAmG | | mU*fA*mUmAmUfCmUf | |
| | | | mAmGmAmUmA*mU* | | CfUmGmAmAmCfUmAfA | |
| | | | mA | | mUmCmAmUmC*mC*mA | |
| 143 | 1641 | TTAGAGTTA | Unmodified: | 221 | Unmodified: | 252 |
| | | CATCTCCTG | UUAGAGUUACAUCU | | UUCCAGGAGAUGUAAC | |
| | | GA (SEQ ID | CCUGGAA | | UCUAAUC | |
| | | NO: 190) | Modified (MS8-221): | | Modified (VP-MAS7-252): | |
| | | | mU*mU*mAmGfAmGm | | VP- | |
| | | | UmUfAfCfAmUmCfUm | | mU*fU*mCmCmAmGmG | |
| | | | CmCmUmGfG*mA*mA | | mAmGmAmUmGmUfAm | |
| | | | | | AmCmUmCmUmAmA*m | |
| | | | | | U*mC | |
| 144 | 1631 | CAAACCTG | Unmodified: | 222 | Unmodified: | 253 |
| | | GATTAGAG | CAAACCUGGAUUAG | | UGUAACUCUAAUCCAG | |
| | | TTAC (SEQ | AGUUACA | | GUUUGCC | |
| | | ID NO: 191) | Modified (MS7-222): | | Modified (VP-MAS6-253): | |
| | | | mC*mA*mAmAmCmCf | | VP- | |
| | | | UmGfGfAfUmUmAmG | | mU*fG*mUmAmAfCmUf | |
| | | | mAmGmUmUmA*mC* | | CfUmAmAmUmCfCmAfG | |
| | | | mA | | mGmUmUmUmG*mC*mC | |
| 145 | 1576 | TTCTTCCGT | Unmodified: | 231 | Unmodified: | 262 |
| | | GAAATTCTG | UUCUUCCGUGAAAU | | UGCCAGAAUUUCACGG | |
| | | GC (SEQ ID | UCUGGCA | | AAGAACA | |
| | | NO: 200) | Modified (MS8-231): | | Modified (VP-MAS7-262): | |
| | | | mU*mU*mCmUfUmCm | | VP- | |
| | | | CmGfUfGfAmAmAfUm | | mU*fG*mCmCmAmGmA | |
| | | | UmCmUmGfG*mC*mA | | mAmUmUmUmCmAfCmG | |
| | | | | | mGmAmAmGmAmA*mC* | |
| | | | | | mA | |
| 146 | 1627 | TAGGCAAA | Unmodified: | 217 | Unmodified: | 248 |
| | | CCTGGATTA | UAGGCAAACCUGGA | | UCUCUAAUCCAGGUUU | |
| | | GAG (SEQ ID | UUAGAGA | | GCCUAGA | |
| | | NO: 186) | Modified (MS9-217): | | Modified (VP-MAS6-248): | |
| | | | mU*mA*mGmGfCmAf | | VP- | |
| | | | AmAfCfCfUmGmGmA | | mU*fC*mUmCmUfAmAf | |
| | | | mUfUmAmGmA*mG*m | | UfCmCmAmGmGfUmUfU | |
| | | | A | | mGmCmCmUmA*mG*mA | |
| 147 | 1627 | TAGGCAAA | Unmodified: | 217 | Unmodified: | 248 |
| | | CCTGGATTA | UAGGCAAACCUGGA | | UCUCUAAUCCAGGUUU | |
| | | GAG (SEQ ID | UUAGAGA | | GCCUAGA | |
| | | NO: 186) | Modified (MS7-217): | | Modified (VP-MAS6-248): | |
| | | | mU*mA*mGmGmCmAf | | VP- | |
| | | | AmAfCfCfUmGmGmA | | mU*fC*mUmCmUfAmAf | |
| | | | mUmUmAmGmA*mG* | | UfCmCmAmGmGfUmUfU | |
| | | | mA | | mGmCmCmUmA*mG*mA | |
| 148 | 1627 | TAGGCAAA | Unmodified: | 217 | Unmodified: | 248 |
| | | CCTGGATTA | UAGGCAAACCUGGA | | UCUCUAAUCCAGGUUU | |
| | | GAG (SEQ ID | UUAGAGA | | GCCUAGA | |
| | | NO: 186) | Modified (MS7-217): | | Modified (VP-MAS5-248): | |
| | | | mU*mA*mGmGmCmAf | | VP- | |
| | | | AmAfCfCfUmGmGmA | | mU*fC*mUmCmUfAmAm | |
| | | | mUmUmAmGmA*mG* | | UmCmCmAmGmGfUmUf | |
| | | | mA | | UmGmCmCmUmA*mG*m | |
| | | | | | A | |

“m” indicates a 2′-O-methyl (2′-O-Me) modified nucleoside; “f” indicates a 2′-fluoro (2′-F) modified nucleoside; “*” indicates phosphorothioate internucleoside linkage; and the absence of “*” between two nucleosides indicate phosphodiester internucleoside linkage. “VP” indicates 5′-(E)-Vinylphosphonate.
^{†}Each uracil base (U) in any one of the oligonucleotides and/or target sequences provided in Table 9 may independently and optionally be replaced with a thymine base (T), and/or each T may independently and optionally be replaced with a U. Target sequences listed in Table 9 contain T′s, but binding of an oligonucleotide described herein to RNA and/or DNA is contemplated.
^{∧}Target sequence start position is in NM_001306068.3 (SEQ ID NO: 161)
^{‡}siRNA oligonucleotides provided in Table 9 that have sense and antisense strands with the same nucleobase sequences as indicated by the SEQ ID NOs can have different modification patterns. The modified sense and antisense strands are indicated by an “MS#” or “MAS#,” respectively. siRNA oligonucleotides provided in Table 9 comprise an antisense strand comprising a 5′-(E)-Vinylphosphonate modification, represented by “VP” before the MAS# (i.e., VP-MAS#). siRNA oligonucleotides comprising an antisense strand without a 5′-(E)-Vinylphosphonate modification are also contemplated and are represented herein by the same MAS# without the “VP”. siRNA oligonucleotides provided in Table 9 may be conjugated to a compound of the formula NH₂-(CH₂)₆- at the 5′ or 3′ (e.g., 5′) nucleoside of the sense strand.

In some embodiments, an oligonucleotide described herein comprises an antisense strand that is 18-25 nucleosides (e.g., 18, 19, 20, 21, 22, 23, 24, or 25 nucleosides) in length and comprises a region of complementarity to a target sequence as set forth in any one of SEQ ID NOs: 174-235, wherein the region of complementarity is at least 16 nucleotides (e.g., 16, 17, 18, or 19 nucleotides) in length. In some embodiments, the antisense strand is 23 nucleotides in length and comprises a region of complementarity to a target sequence as set forth in any one of SEQ ID NOs: 174-235, wherein the region of complementarity is 20 nucleotides in length. In some embodiments, the region of complementarity is fully complementarity with all or a portion of its target sequence. In some embodiments, the region of complementarity includes 1, 2, 3 or more mismatches.

In some embodiments, an oligonucleotide described herein comprises an antisense strand that comprises at least 15 consecutive nucleosides of (e.g., at least 15, at least 16, at least 17, at least 18, at least 19, or at least 20) the nucleotide sequence of any one of SEQ ID NOs: 236-266. In some embodiments, an oligonucleotide described herein further comprises a sense strand that comprises at least 15 consecutive nucleosides of (e.g., at least 15, at least 16, at least 17, at least 18, at least 19, or at least 20) the nucleotide sequence of any one of SEQ ID NOs: 205-235.

In some embodiments, an oligonucleotide described herein comprises an antisense strand that comprises the nucleotide sequence of any one of SEQ ID NOs: 236-266. In some embodiments, an oligonucleotide described herein further comprises a sense strand that comprises the nucleotide sequence of any one of SEQ ID NOs: 205-235.

In some embodiments, an oligonucleotide described herein is a double stranded oligonucleotide (e.g., an siRNA) comprising an antisense strand that comprises the nucleotide sequence of any one of SEQ ID NOs: 236-266 and a sense strand that hybridizes to the antisense strand and comprises the nucleotide sequence of any one of SEQ ID NOs: 205-235, wherein the antisense strand and/or (e.g., and) comprises one or more modified nucleosides (e.g., 2′-modified nucleosides). In some embodiment, the one or more modified nucleosides are selected from 2′-O-Me and 2′-F modified nucleosides.

In some embodiments, an oligonucleotide described herein is a double stranded oligonucleotide (e.g., an siRNA) comprising an antisense strand that comprises the nucleotide sequence of any one of SEQ ID NOs: 236-266 and a sense strand that hybridizes to the antisense strand and comprises the nucleotide sequence of any one of SEQ ID NOs: 205-235, wherein each nucleoside in the antisense strand and/or (e.g., and) each nucleoside in the sense strand is a 2′-modified nucleoside selected from 2′-O-Me and 2′-F modified nucleosides.

In some embodiments, an oligonucleotide described herein is a double stranded oligonucleotide (e.g., an siRNA) comprising an antisense strand that comprises the nucleotide sequence of any one of SEQ ID NOs: 236-266 and a sense strand that hybridizes to the antisense strand and comprises the nucleotide sequence of any one of SEQ ID NOs: 205-235, wherein each nucleoside in the antisense strand and each nucleoside in the sense strand is a 2′-modified nucleoside selected from 2′-O-Me and 2′-F modified nucleosides, and wherein the antisense strand and/or (e.g., and) the sense strand each comprises one or more phosphorothioate internucleoside linkages. In some embodiments, the sense strand does not comprise any phosphorothioate internucleoside linkages (all the internucleoside linkages in the sense strand are phosphodiester internucleoside linkages), and the antisense strand comprises 1, 2, or 3 phosphorothioate internucleoside linkages. In some embodiments, the sense strand comprises 2 phosphorothioate internucleoside linkages, and the antisense strand comprises 4 phosphorothioate internucleoside linkages. In some embodiments, the sense strand comprises 4 phosphorothioate internucleoside linkages, and the antisense strand comprises 4 phosphorothioate internucleoside linkages.

In some embodiments, the antisense strand comprises 2 phosphorothioate internucleoside linkages, optionally wherein the two internucleoside linkages at the 3′ end of the antisense strand are phosphorothioate internucleoside linkages and the rest of the internucleoside linkages in the antisense strand are phosphodiester internucleoside linkages. In some embodiments, the antisense strand comprises 4 phosphorothioate internucleoside linkages, optionally wherein the two internucleoside linkages at the 3′ end and the two internucleoside linkage at the 5′ end of the antisense strand are phosphorothioate internucleoside linkages and the rest of the internucleoside linkages in the antisense strand are phosphodiester internucleoside linkages. In some embodiments, the sense strand comprises 2 phosphorothioate internucleoside linkages, optionally wherein the two internucleoside linkages at the 5′ end of the sense strand are phosphorothioate internucleoside linkages and the rest of the internucleoside linkages in the sense strand are phosphodiester internucleoside linkages. In some embodiments, the sense strand comprises 4 phosphorothioate internucleoside linkages. In some embodiments, the two internucleoside linkages at the 5′ end and the two internucleoside linkages at the 3′ end of the sense strand are phosphorothioate internucleoside linkages, and the rest of the internucleoside linkages in the sense strand are phosphodiester internucleoside linkages.

In some embodiments, the antisense strand of the oligonucleotide described herein comprises a structure of (5′ to 3′; referred to herein as “MAS1”): fN*fN*mNfNmNfNmNfNmNfNmNfNmNfNmNfNmNfNmNfNmN*fN*mN, wherein “mN” indicates 2′-O-methyl(2′—O-Me) modified nucleosides; “IN” indicates 2′-fluoro (2′-F) modified nucleosides; “*” indicates phosphorothioate internucleoside linkage; and the absence of “*” between two nucleosides indicate phosphodiester internucleoside linkage. For example, in Table 8, antisense strands MAS1-236, MAS1-237, and MAS1-238 comprise this structure.

In some embodiments, the antisense strand of the oligonucleotide described herein comprises a structure of (5′ to 3′; referred to herein as “MAS2”): fN*fN*mNmNmNfNmNmNmNmNmNmNmNfNmNfNmNmNmNmNmN*mN*mN wherein “mN” indicates 2′-O-methyl(2′—O-Me) modified nucleosides; “fN” indicates 2′-fluoro (2′-F) modified nucleosides; “*” indicates phosphorothioate internucleoside linkage; and the absence of “*” between two nucleosides indicate phosphodiester internucleoside linkage. For example, in Table 8, antisense strands MAS2-236, MAS2-237, MAS2-238, MAS2-239, MAS2-240, MAS2-241, MAS2-242, MAS2-243, MAS2-244, MAS2-245, MAS2-246, MAS2-247, MAS2-248, MAS2-249, MAS2-250, MAS2-251, MAS2-252, MAS2-253, MAS2-254, MAS2-255, MAS2-256, and MAS2-257 comprise this structure.

In some embodiments, the antisense strand of the oligonucleotide described herein comprises a structure of (5′ to 3′; referred to herein as “MAS3”): fN*fN*mNmNmNmNmNmNmNmNmNmNmNfNmNmNmNmNmNmNmN*mN*mN, wherein “mN” indicates 2′-O-methyl(2′—O-Me) modified nucleosides; “IN” indicates 2′-fluoro (2′-F) modified nucleosides; “*” indicates phosphorothioate internucleoside linkage; and the absence of “*” between two nucleosides indicate phosphodiester internucleoside linkage. For example, in Table 8, antisense strands MAS3-236, MAS3-237, MAS3-238, MAS3-239, MAS3-240, MAS3-241, MAS3-242, MAS3-243, MAS3-244, MAS3-245, MAS3-246, MAS3-247, MAS3-248, MAS3-249, MAS3-250, MAS3-251, MAS3-252, MAS3-253, MAS3-254, MAS3-255, MAS3-256, MAS3-257, MAS3-258, MAS3-259, MAS3-260, MAS3-261, MAS3-262, MAS3-263, MAS3-264, MAS3-265, and MAS3-266 comprise this structure.

In some embodiments, the antisense strand of the oligonucleotide described herein comprises a structure of (5′ to 3′; referred to herein as “MAS4”): fN*fN*mNmNmNfNmNfNfNmNmNmNmNfNmNfNmNmNmNmNmN*mN*mN wherein “mN” indicates 2′-O-methyl(2′—O-Me) modified nucleosides; “IN” indicates 2′-fluoro (2′-F) modified nucleosides; “*” indicates phosphorothioate internucleoside linkage; and the absence of “*” between two nucleosides indicate phosphodiester internucleoside linkage. For example, in Table 8, antisense strands MAS4-236, MAS4-237, MAS4-238, MAS4-239, MAS4-240, MAS4-241, MAS4-242, MAS4-243, MAS4-244, MAS4-245, MAS4-246, MAS4-247, MAS4-248, MAS4-249, MAS4-250, MAS4-251, MAS4-252, MAS4-253, MAS4-254, MAS4-255, MAS4-256, and MAS4-257 comprise this structure.

In some embodiments, the antisense strand of the oligonucleotide described herein comprises a structure of (5′ to 3′; referred to herein as “MAS5”):
mN*fN*mNmNmNfNmNmNmNmNmNmNmNfNmNfNmNmNmNmNmN*mN*mN wherein “mN” indicates 2′-O-methyl(2′—O-Me) modified nucleosides; “fN” indicates 2′-fluoro (2′-F) modified nucleosides; “*” indicates phosphorothioate internucleoside linkage; and the absence of “*” between two nucleosides indicate phosphodiester internucleoside linkage.

In some embodiments, the MAS5 structure further comprises a 5′ vinylphosphonate (e.g., 5′-(E)-vinylphosphonate) modification (5′ to 3′; referred to herein as “VP-MAS5”): VP-mN*fN*mNmNmNfNmNmNmNmNmNmNmNfNmNfNmNmNmNmNmN*mN*mN, wherein “mN” indicates 2′-O-methyl(2′—O-Me) modified nucleosides; “IN” indicates 2′-fluoro (2′-F) modified nucleosides; “*” indicates phosphorothioate internucleoside linkage; the absence of “*” between two nucleosides indicate phosphodiester internucleoside linkage; and VP indicates 5′-(E)-vinylphosphonate. For example, in Table 9, antisense strand VP-MAS5-248 comprises this structure.

In some embodiments, the antisense strand of the oligonucleotide described herein comprises a structure of (5′ to 3′; referred to herein as “MAS6”):
mN*fN*mNmNmNfNmNfNfNmNmNmNmNfNmNfNmNmNmNmNmN*mN*mN wherein “mN” indicates 2′-O-methyl(2′—O-Me) modified nucleosides; “fN” indicates 2′-fluoro (2′-F) modified nucleosides; “*” indicates phosphorothioate internucleoside linkage; and the absence of “*” between two nucleosides indicate phosphodiester internucleoside linkage.

In some embodiments, the MAS6 structure further comprises a 5′ vinylphosphonate (e.g., 5′-(E)-vinylphosphonate) modification (5′ to 3′; referred to herein as “VP-MAS6”): VP-mN*fN*mNmNmNfNmNfNfNmNmNmNmNfNmNfNmNmNmNmNmN*mN*mN, wherein “mN” indicates 2′-O-methyl(2′—O-Me) modified nucleosides; “IN” indicates 2′-fluoro (2′-F) modified nucleosides; “*” indicates phosphorothioate internucleoside linkage; the absence of “*” between two nucleosides indicate phosphodiester internucleoside linkage; and VP indicates 5′-(E)-vinylphosphonate. For example, in Table 9, antisense strands VP-MAS6-251, VP-MAS6-253, and VP-MAS6-248 comprise this structure.

In some embodiments, the antisense strand of the oligonucleotide described herein comprises a structure of (5′ to 3′; referred to herein as “MAS7”):
mN*fN*mNmNmNmNmNmNmNmNmNmNmNfNmNmNmNmNmNmNmN*mN*mN, wherein “mN” indicates 2′-O-methyl(2′—O-Me) modified nucleosides; “IN” indicates 2′-fluoro (2′-F) modified nucleosides; “*” indicates phosphorothioate internucleoside linkage; and the absence of “*” between two nucleosides indicate phosphodiester internucleoside linkage.

In some embodiments, the MAS7 structure further comprises a 5′ vinylphosphonate (e.g., 5′-(E)-vinylphosphonate) modification (5′ to 3′; referred to herein as “VP-MAS7”): VP-mN*fN*mNmNmNmNmNmNmNmNmNmNmNfNmNmNmNmNmNmNmN*mN*mN, wherein “mN” indicates 2′-O-methyl(2′—O-Me) modified nucleosides; “IN” indicates 2′-fluoro (2′-F) modified nucleosides; “*” indicates phosphorothioate internucleoside linkage; the absence of “*” between two nucleosides indicate phosphodiester internucleoside linkage; and VP indicates 5′-(E)-vinylphosphonate. For example, in Table 9, antisense strands VP-MAS7-252 and VP-MAS7-262 comprise this structure.

In some embodiments, the sense strand of the oligonucleotide described herein comprises a structure of (5′ to 3′; referred to herein as “MS1”):
mN*mN*fNmNfNmNfNmNfNmNfNmNfNmNfNmNfNmNfNmNfN, wherein “mN” indicates 2′-O-methyl(2′—O-Me) modified nucleosides; “IN” indicates 2′-fluoro (2′-F) modified nucleosides; “*” indicates phosphorothioate internucleoside linkage; and the absence of “*” between two nucleosides indicate phosphodiester internucleoside linkage. For example, in Table 8, sense strands MS1-205, MS1-206, and MS1-207 comprise this structure.

In some embodiments, the sense strand of the oligonucleotide described herein comprises a structure of (5′ to 3′; referred to herein as “MS2”):
mN*mN*mNmNfNmNfNmNfNfNfNmNmNmNmNfNmNmNmNmNmN, wherein “mN” indicates 2′-O-methyl(2′—O-Me) modified nucleosides; “fN” indicates 2′-fluoro (2′-F) modified nucleosides; “*” indicates phosphorothioate internucleoside linkage; and the absence of “*” between two nucleosides indicate phosphodiester internucleoside linkage. For example, in Table 8, sense strands MS2-208, MS2-209, MS2-210, MS2-211, MS2-212, MS2-213, MS2-214, MS2-215, MS2-216, MS2-217, MS2-218, MS2-219, MS2-220, MS2-221, MS2-222, MS2-223, MS2-224, MS2-225, and MS2-226 comprise this structure.

In some embodiments, the sense strand of the oligonucleotide described herein comprises a structure of (5′ to 3′; referred to herein as “MS3”):
mN*mN*mNmNfNmNmNmNfNfNfNmNmNfNmNmNmNmNfNmNmN, wherein “mN” indicates 2′-O-methyl(2′—O-Me) modified nucleosides; “IN” indicates 2′-fluoro (2′-F) modified nucleosides; “*” indicates phosphorothioate internucleoside linkage; and the absence of “*” between two nucleosides indicate phosphodiester internucleoside linkage. For example, in Table 8, sense strands MS3-205, MS3-206, MS3-207, MS3-208, MS3-209, MS3-210, MS3-211, MS3-212, MS3-213, MS3-214, MS3-215, MS3-216, MS3-217, MS3-218, MS3-219, MS3-220, MS3-221, MS3-222, MS3-223, MS3-224, MS3-225, MS3-226, MS3-227, MS3-228, MS3-229, MS3-230, MS3-231, MS3-232, MS3-233, MS3-234, and MS3-235 comprise this structure.

In some embodiments, the sense strand of the oligonucleotide described herein comprises a structure of (5′ to 3′; referred to herein as “MS4”):
mN*mN*mNmNmNmNfNmNfNfNfNmNmNmNmNmNmNmNmNmNmN, wherein “mN” indicates 2′-O-methyl(2′—O-Me) modified nucleosides; “IN” indicates 2′-fluoro (2′-F) modified nucleosides; “*” indicates phosphorothioate internucleoside linkage; and the absence of “*” between two nucleosides indicate phosphodiester internucleoside linkage. For example, in Table 8, sense strands MS4-205, MS4-206, MS4-207, MS4-208, MS4-209, MS4-210, MS4-211, MS4-212, MS4-213, MS4-214, MS4-215, MS4-216, MS4-217, MS4-218, MS4-219, MS4-220, MS4-221, MS4-222, MS4-223, MS4-224, MS4-225, and MS4-226 comprise this structure.

In some embodiments, the sense strand of the oligonucleotide described herein comprises a structure of (5′ to 3′; referred to herein as “MS5”):
mN*mN*mNmNmNmNfNmNfNfNfNmNmNmNmNfNfNmNmNmNmN, wherein “mN” indicates 2′-O-methyl(2′—O-Me) modified nucleosides; “IN” indicates 2′-fluoro (2′-F) modified nucleosides; “*” indicates phosphorothioate internucleoside linkage; and the absence of “*” between two nucleosides indicate phosphodiester internucleoside linkage. For example, in Table 8, sense strands MS5-205, MS5-206, MS5-207, MS5-208, MS5-209, MS5-210, MS5-211, MS5-212, MS5-213, MS5-214, MS5-215, MS5-216, MS5-217, MS5-218, MS5-219, MS5-220, MS5-221, MS5-222, MS5-223, MS5-224, MS5-225, and MS5-226 comprise this structure.

In some embodiments, the sense strand of the oligonucleotide described herein comprises a structure of (5′ to 3′; referred to herein as “MS6”):
mN*mN*mNmNmNmNfNmNfNfNfNmNmNmNmNfNmNmNfNmNmN, wherein “mN” indicates 2′-O-methyl(2′—O-Me) modified nucleosides; “IN” indicates 2′-fluoro (2′-F) modified nucleosides; “*” indicates phosphorothioate internucleoside linkage; and the absence of “*” between two nucleosides indicate phosphodiester internucleoside linkage. For example, in Table 8, sense strands MS6-205, MS6-206, MS6-207, MS6-208, MS6-209, MS6-210, MS6-211, MS6-212, MS6-213, MS6-214, MS6-215, MS6-216, MS6-217, MS6-218, MS6-219, MS6-220, MS6-221, MS6-222, MS6-223, MS6-224, MS6-225, and MS6-226 comprise this structure.

In some embodiments, the sense strand of the oligonucleotide described herein comprises a structure of (5′ to 3′; referred to herein as “MS7”):
mN*mN*mNmNmNmNfNmNfNfNfNmNmNmNmNmNmNmNmN*mN*mN, wherein “mN” indicates 2′-O-methyl(2′—O-Me) modified nucleosides; “IN” indicates 2′-fluoro (2′-F) modified nucleosides; “*” indicates phosphorothioate internucleoside linkage; and the absence of “*” between two nucleosides indicate phosphodiester internucleoside linkage. For example, in Table 9, sense strands MS7-220, MS7-222, and MS7-217 comprise this structure.

In some embodiments, the sense strand of the oligonucleotide described herein comprises a structure of (5′ to 3′; referred to herein as “MS8”):
mN*mN*mNmNfNmNmNmNfNfNfNmNmNfNmNmNmNmNfN*mN*mN, wherein “mN” indicates 2′-O-methyl(2′—O-Me) modified nucleosides; “IN” indicates 2′-fluoro (2′-F) modified nucleosides; “*” indicates phosphorothioate internucleoside linkage; and the absence of “*” between two nucleosides indicate phosphodiester internucleoside linkage. For example, in Table 9, sense strands MS8-221 and MS8-231 comprise this structure.

In some embodiments, the sense strand of the oligonucleotide described herein comprises a structure of (5′ to 3′; referred to herein as “MS9”):
mN*mN*mNmNfNmNfNmNfNfNfNmNmNmNmNfNmNmNmN*mN*mN, wherein “mN” indicates 2′-O-methyl(2′—O-Me) modified nucleosides; “IN” indicates 2′-fluoro (2′-F) modified nucleosides; “*” indicates phosphorothioate internucleoside linkage; and the absence of “*” between two nucleosides indicate phosphodiester internucleoside linkage. For example, in Table 9, sense strand MS9-217 comprises this structure.

In some embodiments, the antisense strand of the oligonucleotide described herein is selected from the modified versions (e.g., MAS1-MAS4) of SEQ ID NOs: 236-266 listed in Table 8. For example, in some embodiments, the antisense strand is selected from MAS1-236, MAS1-237, MAS1-238, MAS2-236, MAS2-237, MAS2-238, MAS2-239, MAS2-240, MAS2-241, MAS2-242, MAS2-243, MAS2-244, MAS2-245, MAS2-246, MAS2-247, MAS2-248, MAS2-249, MAS2-250, MAS2-251, MAS2-252, MAS2-253, MAS2-254, MAS2-255, MAS2-256, MAS2-257, MAS3-236, MAS3-237, MAS3-238, MAS3-239, MAS3-240, MAS3-241, MAS3-242, MAS3-243, MAS3-244, MAS3-245, MAS3-246, MAS3-247, MAS3-248, MAS3-249, MAS3-250, MAS3-251, MAS3-252, MAS3-253, MAS3-254, MAS3-255, MAS3-256, MAS3-257, MAS3-258, MAS3-259, MAS3-260, MAS3-261, MAS3-262, MAS3-263, MAS3-264, MAS3-265, MAS3-266, MAS4-236, MAS4-237, MAS4-238, MAS4-239, MAS4-240, MAS4-241, MAS4-242, MAS4-243, MAS4-244, MAS4-245, MAS4-246, MAS4-247, MAS4-248, MAS4-249, MAS4-250, MAS4-251, MAS4-252, MAS4-253, MAS4-254, MAS4-255, MAS4-256, and MAS4-257.

In some embodiments, the antisense strand of the oligonucleotide described herein is selected from the modified versions (e.g., MAS5-MAS7) of SEQ ID NOs: 236-266 listed in Table 8. In some embodiments, the antisense strand of the oligonucleotide described herein is selected from the modified versions (e.g., MAS5, MAS6, MAS7, VP-MAS5, VP-MAS6, and VP-MAS7) of SEQ ID NOs: 251, 253, 262, 248, and 252 listed in Table 9. For example, in some embodiments, the antisense strand is selected from VP-MAS6-251, VP-MAS7-252, VP-MAS6-253, VP-MAS7-262, VP-MAS6-248, and VP-MAS5-248.

In some embodiments, the sense strand of the oligonucleotide described herein is selected from the modified versions (e.g., MS1-MS6) of SEQ ID NOs: 205-235 listed in Table 8. For example, in some embodiments, the sense strand is selected from MS1-205, MS1-206, MS1-207, MS2-208, MS2-209, MS2-210, MS2-211, MS2-212, MS2-213, MS2-214, MS2-215, MS2-216, MS2-217, MS2-218, MS2-219, MS2-220, MS2-221, MS2-222, MS2-223, MS2-224, MS2-225, MS2-226, MS3-205, MS3-206, MS3-207, MS3-208, MS3-209, MS3-210, MS3-211, MS3-212, MS3-213, MS3-214, MS3-215, MS3-216, MS3-217, MS3-218, MS3-219, MS3-220, MS3-221, MS3-222, MS3-223, MS3-224, MS3-225, MS3-226, MS3-227, MS3-228, MS3-229, MS3-230, MS3-231, MS3-232, MS3-233, MS3-234, MS3-235, MS4-205, MS4-206, MS4-207, MS4-208, MS4-209, MS4-210, MS4-211, MS4-212, MS4-213, MS4-214, MS4-215, MS4-216, MS4-217, MS4-218, MS4-219, MS4-220, MS4-221, MS4-222, MS4-223, MS4-224, MS4-225, MS4-226, MS5-205, MS5-206, MS5-207, MS5-208, MS5-209, MS5-210, MS5-211, MS5-212, MS5-213, MS5-214, MS5-215, MS5-216, MS5-217, MS5-218, MS5-219, MS5-220, MS5-221, MS5-222, MS5-223, MS5-224, MS5-225, MS5-226, MS6-205, MS6-206, MS6-207, MS6-208, MS6-209, MS6-210, MS6-211, MS6-212, MS6-213, MS6-214, MS6-215, MS6-216, MS6-217, MS6-218, MS6-219, MS6-220, MS6-221, MS6-222, MS6-223, MS6-224, MS6-225, and MS6-226.

In some embodiments, the sense strand of the oligonucleotide described herein is selected from the modified versions (e.g., MS7-MS9) of SEQ ID NOs: 205-235 listed in Table 8. In some embodiments, the sense strand of the oligonucleotide described herein is selected from the modified versions (e.g., MS7-MS9) of SEQ ID NOs: 220, 222, 231, 217, and 221 listed in Table 9. For example, in some embodiments, the sense strand is selected from MS7-220, MS8-221, MS7-222, MS8-231, MS9-217, and MS7-217.

In some embodiments, an oligonucleotide described herein may comprise an antisense strand comprising any one of the antisense strand structures described herein (e.g., MAS1-MAS4, irrespective to its nucleobase sequence), and a sense strand comprising any one of the sense strand structures described herein (e.g., MS1-MS6, irrespective to its nucleobase sequence). In some embodiments, an oligonucleotide described herein may comprise an antisense strand comprising any one of the antisense strand structures described herein (e.g., MAS5, MAS6, MAS7, VP-MAS5, VP-MAS6, and VP-MAS7, irrespective to its nucleobase sequence), and a sense strand comprising any one of the sense strand structures described herein (e.g., MS7-MS9, irrespective to its nucleobase sequence).

In some embodiments, the oligonucleotide described herein comprises an antisense strand comprising a structure of (5′ to 3′; MAS3):
fN*fN*mNmNmNmNmNmNmNmNmNmNmNfNmNmNmNmNmNmNmN*mN*mN, and a sense strand comprising a structure of (5′ to 3′; MS3):
mN*mN*mNmNfNmNmNmNfNfNfNmNmNfNmNmNmNmNfNmNmN, wherein “mN” indicates 2′-O-methyl(2′—O-Me) modified nucleosides; “IN” indicates 2′-fluoro (2′-F) modified nucleosides; “*” indicates phosphorothioate internucleoside linkage; and the absence of “*” between two nucleosides indicate phosphodiester internucleoside linkage.

In some embodiments, the oligonucleotide described herein comprises an antisense strand comprising a structure of (5′ to 3′; MAS4):
fN*fN*mNmNmNfNmNfNfNmNmNmNmNfNmNfNmNmNmNmNmN*mN*mN, and a sense strand comprising a structure of (5′ to 3′; MS4):
mN*mN*mNmNmNmNfNmNfNfNfNmNmNmNmNmNmNmNmNmNmN, wherein “mN” indicates 2′-O-methyl(2′—O-Me) modified nucleosides; “IN” indicates 2′-fluoro (2′-F) modified nucleosides; “*” indicates phosphorothioate internucleoside linkage; and the absence of “*” between two nucleosides indicate phosphodiester internucleoside linkage.

In some embodiments, the oligonucleotide described herein comprises an antisense strand comprising a structure of (5′ to 3′; MAS4):
fN*fN*mNmNmNfNmNfNfNmNmNmNmNfNmNfNmNmNmNmNmN*mN*mN, and a sense strand comprising a structure of (5′ to 3′; MS6):
mN*mN*mNmNmNmNfNmNfNfNfNmNmNmNmNfNmNmNfNmNmN, wherein “mN” indicates 2′-O-methyl(2′—O-Me) modified nucleosides; “IN” indicates 2′-fluoro (2′-F) modified nucleosides; “*” indicates phosphorothioate internucleoside linkage; and the absence of “*” between two nucleosides indicate phosphodiester internucleoside linkage.

In some embodiments, the oligonucleotide described herein comprises an antisense strand comprising a structure of (5′ to 3′; MAS2):
fN*fN*mNmNmNfNmNmNmNmNmNmNmNfNmNfNmNmNmNmNmN*mN*mN, and a sense strand comprising a structure of (5′ to 3′; MS5):
mN*mN*mNmNmNmNfNmNfNfNfNmNmNmNmNfNfNmNmNmNmN, wherein “mN” indicates 2′-O-methyl(2′—O-Me) modified nucleosides; “IN” indicates 2′-fluoro (2′-F) modified nucleosides; “*” indicates phosphorothioate internucleoside linkage; and the absence of “*” between two nucleosides indicate phosphodiester internucleoside linkage.

In some embodiments, the oligonucleotide described herein comprises an antisense strand comprising a structure of (5′ to 3′; MAS2):
fNfN*mNmNmNfNmNmNmNmNmNmNmNfNmNfNmNmNmNmNmN*mN*mN, and a sense strand comprising a structure of (5′ to 3′; MS4):
mN*mN*mNmNmNmNfNmNfNfNfNmNmNmNmNmNmNmNmNmNmN, wherein “mN” indicates 2′-O-methyl(2′—O-Me) modified nucleosides; “IN” indicates 2′-fluoro (2′-F) modified nucleosides; “*” indicates phosphorothioate internucleoside linkage; and the absence of “*” between two nucleosides indicate phosphodiester internucleoside linkage.

In some embodiments, the oligonucleotide described herein comprises an antisense strand comprising a structure of (5′ to 3′; MAS1):
fN*fN*mNfNmNfNmNfNmNfNmNfNmNfNmNfNmNfNmNfNmN*fN*mN, and a sense strand comprising a structure of (5′ to 3′; MS1):
mN*mN*fNmNfNmNfNmNfNmNfNmNfNmNfNmNfNmNfNmNfN, wherein “mN” indicates 2′-O-methyl(2′—O-Me) modified nucleosides; “IN” indicates 2′-fluoro (2′-F) modified nucleosides; “*” indicates phosphorothioate internucleoside linkage; and the absence of “*” between two nucleosides indicate phosphodiester internucleoside linkage.

In some embodiments, the oligonucleotide described herein comprises an antisense strand comprising a structure of (5′ to 3′; MAS4):
fN*fN*mNmNmNfNmNfNfNmNmNmNmNfNmNfNmNmNmNmNmN*mN*mN, and a sense strand comprising a structure of (5′ to 3′; MS2):
mN*mN*mNmNfNmNfNmNfNfNfNmNmNmNmNfNmNmNmNmNmN, wherein “mN” indicates 2′-O-methyl(2′—O-Me) modified nucleosides; “IN” indicates 2′-fluoro (2′-F) modified nucleosides; “*” indicates phosphorothioate internucleoside linkage; and the absence of “*” between two nucleosides indicate phosphodiester internucleoside linkage.

In some embodiments, the oligonucleotide described herein comprises an antisense strand comprising a structure of (5′ to 3′; MAS5):
mN*fN*mNmNmNfNmNmNmNmNmNmNmNfNmNfNmNmNmNmNmN*mN*mN, and a sense strand comprising a structure of (5′ to 3′; MS7):
mN*mN*mNmNmNmNfNmNfNfNfNmNmNmNmNmNmNmNmN*mN*mN, wherein “mN” indicates 2′-O-methyl(2′—O-Me) modified nucleosides; “IN” indicates 2′-fluoro (2′-F) modified nucleosides; “*” indicates phosphorothioate internucleoside linkage; and the absence of “*” between two nucleosides indicate phosphodiester internucleoside linkage.

In some embodiments, the oligonucleotide described herein comprises an antisense strand comprising a structure of (5′ to 3′; MAS6):
mN*fN*mNmNmNfNmNfNfNmNmNmNmNfNmNfNmNmNmNmNmN*mN*mN, and a sense strand comprising a structure of (5′ to 3′; MS7):
mN*mN*mNmNmNmNfNmNfNfNfNmNmNmNmNmNmNmNmN*mN*mN, wherein “mN” indicates 2′-O-methyl(2′—O-Me) modified nucleosides; “IN” indicates 2′-fluoro (2′-F) modified nucleosides; “*” indicates phosphorothioate internucleoside linkage; and the absence of “*” between two nucleosides indicate phosphodiester internucleoside linkage.

In some embodiments, the oligonucleotide described herein comprises an antisense strand comprising a structure of (5′ to 3′; MAS7):
mN*fN*mNmNmNmNmNmNmNmNmNmNmNfNmNmNmNmNmNmNmN*mN*mN, and a sense strand comprising a structure of (5′ to 3′; MS8):
mN*mN*mNmNfNmNmNmNfNfNfNmNmNfNmNmNmNmNfN*mN*mN, wherein “mN” indicates 2′-O-methyl(2′—O-Me) modified nucleosides; “IN” indicates 2′-fluoro (2′-F) modified nucleosides; “*” indicates phosphorothioate internucleoside linkage; and the absence of “*” between two nucleosides indicate phosphodiester internucleoside linkage.

In some embodiments, the oligonucleotide described herein comprises an antisense strand comprising a structure of (5′ to 3′; MAS6):
mN*fN*mNmNmNfNmNfNfNmNmNmNmNfNmNfNmNmNmNmNmN*mN*mN, and a sense strand comprising a structure of (5′ to 3′; MS9):
mN*mN*mNmNfNmNfNmNfNfNfNmNmNmNmNfNmNmNmN*mN*mN, wherein “mN” indicates 2′-O-methyl(2′—O-Me) modified nucleosides; “IN” indicates 2′-fluoro (2′-F) modified nucleosides; “*” indicates phosphorothioate internucleoside linkage; and the absence of “*” between two nucleosides indicate phosphodiester internucleoside linkage. In some embodiments, the oligonucleotide described herein comprises an antisense strand comprising a structure of (5′ to 3′; VP-MAS5):
VP-mN*fN*mNmNmNfNmNmNmNmNmNmNmNfNmNfNmNmNmNmNmN*mN*mN, and a sense strand comprising a structure of (5′ to 3′; MS7):
mN*mN*mNmNmNmNfNmNfNfNfNmNmNmNmNmNmNmNmN*mN*mN, wherein “mN” indicates 2′-O-methyl(2′—O-Me) modified nucleosides; “IN” indicates 2′-fluoro (2′-F) modified nucleosides; “*” indicates phosphorothioate internucleoside linkage; and the absence of “*” between two nucleosides indicate phosphodiester internucleoside linkage.

In some embodiments, the oligonucleotide described herein comprises an antisense strand comprising a structure of (5′ to 3′; VP-MAS6):
VP-mN*fN*mNmNmNfNmNfNfNmNmNmNmNfNmNfNmNmNmNmNmN*mN*mN, and a sense strand comprising a structure of (5′ to 3′; MS7):
mN*mN*mNmNmNmNfNmNfNfNfNmNmNmNmNmNmNmNmN*mN*mN, wherein “mN” indicates 2′-O-methyl(2′—O-Me) modified nucleosides; “IN” indicates 2′-fluoro (2′-F) modified nucleosides; “*” indicates phosphorothioate internucleoside linkage; and the absence of “*” between two nucleosides indicate phosphodiester internucleoside linkage.

In some embodiments, the oligonucleotide described herein comprises an antisense strand comprising a structure of (5′ to 3′; VP-MAS7): VP-mN*fN*mNmNmNmNmNmNmNmNmNmNmNfNmNmNmNmNmNmNmN*mN*mN, and a sense strand comprising a structure of (5′ to 3′; MS8):
mN*mN*mNmNfNmNmNmNfNfNfNmNmNfNmNmNmNmNfN*mN*mN, wherein “mN” indicates 2′-O-methyl(2′—O-Me) modified nucleosides; “IN” indicates 2′-fluoro (2′-F) modified nucleosides; “*” indicates phosphorothioate internucleoside linkage; and the absence of “*” between two nucleosides indicate phosphodiester internucleoside linkage.

In some embodiments, the oligonucleotide described herein comprises an antisense strand comprising a structure of (5′ to 3′; VP-MAS6):
VP-mN*fN*mNmNmNfNmNfNfNmNmNmNmNfNmNfNmNmNmNmNmN*mN*mN, and a sense strand comprising a structure of (5′ to 3′; MS9):
mN*mN*mNmNfNmNfNmNfNfNfNmNmNmNmNfNmNmNmN*mN*mN, wherein “mN” indicates 2′-O-methyl(2′—O-Me) modified nucleosides; “IN” indicates 2′-fluoro (2′-F) modified nucleosides; “*” indicates phosphorothioate internucleoside linkage; and the absence of “*” between two nucleosides indicate phosphodiester internucleoside linkage. In some embodiments, the oligonucleotide described herein is an siRNA selected from the siRNAs listed in Table 8. In some embodiments, the oligonucleotide described herein is an siRNA selected from the siRNAs listed in Table 9.

In some embodiments, any one of the oligonucleotides described herein (e.g., siRNAs selected from the siRNAs in Table 8) can be in salt form, e.g., as sodium, potassium, magnesium salts. In some embodiments, any one of the oligonucleotides described herein (e.g., siRNAs selected from the siRNAs in Table 9) can be in salt form, e.g., as sodium, potassium, magnesium salts.

In some embodiments, the 5′ or 3′ nucleoside (e.g., terminal nucleoside) of any one of the oligonucleotides described herein (e.g., the oligonucleotides listed in Table 8) is conjugated to an amine group, optionally via a spacer. In some embodiments, the 5′ or 3′ nucleoside (e.g., terminal nucleoside) of any one of the oligonucleotides described herein (e.g., the oligonucleotides listed in Table 9) is conjugated to an amine group, optionally via a spacer. In some embodiments, the spacer comprises an aliphatic moiety. In some embodiments, the spacer comprises a polyethylene glycol moiety. In some embodiments, a phosphodiester linkage is present between the spacer and the 5′ or 3′ nucleoside of the oligonucleotide. In some embodiments, the 5′ or 3′ nucleoside (e.g., terminal nucleoside) of any of the oligonucleotides described herein (e.g., the oligonucleotides listed in Table 8) is conjugated to a spacer that is a substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof; each R^{A} is independently hydrogen or substituted or unsubstituted alkyl. In some embodiments, the 5′ or 3′ nucleoside (e.g., terminal nucleoside) of any of the oligonucleotides described herein (e.g., the oligonucleotides listed in Table 9) is conjugated to a spacer that is a substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof; each R^{A} is independently hydrogen or substituted or unsubstituted alkyl.

In some embodiments, the 5′ or 3′ nucleoside of any one of the oligonucleotides described herein (e.g., the oligonucleotides listed in Table 8, sense or antisense strand) is conjugated to a compound of the formula —NH₂—(CH₂)ₙ—, wherein n is an integer from 1 to 12. In some embodiments, the 5′ or 3′ nucleoside of any one of the oligonucleotides described herein (e.g., the oligonucleotides listed in Table 9, sense or antisense strand) is conjugated to a compound of the formula —NH₂—(CH₂)ₙ—, wherein n is an integer from 1 to 12. In some embodiments, n is 6, 7, 8, 9, 10, 11, or 12. In particular embodiments, n is 6. In some embodiments, a phosphodiester linkage is present between the compound of the formula NH₂—(CH₂)ₙ- and the 5′ or 3′ nucleoside of the oligonucleotide (e.g., the oligonucleotides listed in Table 8, sense or antisense strand), wherein n is an integer from 1 to 12. In some embodiments, n is 6, 7, 8, 9, 10, 11, or 12. In particular embodiments, n is 6.

In some embodiments, the 5′ nucleoside of the sense strand of any one of the oligonucleotides described herein (e.g., the oligonucleotides listed in Table 8) is conjugated to a compound of the formula —NH₂—(CH₂)ₙ—, wherein n is an integer from 1 to 12 (e.g., 6).

In some embodiments, the 5′ nucleoside of the sense strand of any one of the oligonucleotides described herein (e.g., the oligonucleotides listed in Table 9) is conjugated to a compound of the formula —NH₂—(CH₂)ₙ—, wherein n is an integer from 1 to 12 (e.g., 6).

In some embodiments, the 3′ nucleoside of the sense strand of any one of the oligonucleotides described herein (e.g., the oligonucleotides listed in Table 8) is conjugated to a compound of the formula —NH₂—(CH₂)ₙ—, wherein n is an integer from 1 to 12 (e.g., 6).

In some embodiments, the 3′ nucleoside of the sense strand of any one of the oligonucleotides described herein (e.g., the oligonucleotides listed in Table 9) is conjugated to a compound of the formula —NH₂—(CH₂)ₙ—, wherein n is an integer from 1 to 12 (e.g., 6).

In some embodiments, a compound of the formula NH₂—(CH₂) 6- is conjugated to the oligonucleotide via a reaction between 6-amino-1-hexanol (NH₂—(CH₂) 6—OH) and the 5′ phosphate of the oligonucleotide (e.g., 5′ phosphate of the sense or antisense strand). In some embodiments, a compound of the formula NH₂—(CH₂) 6- is conjugated to the oligonucleotide via a reaction between 6-amino-1-hexanol (NH₂—(CH₂) 6—OH) and the 5′ phosphate of the sense strand of an oligonucleotide (e.g., an oligonucleotide listed in Table 8). In some embodiments, a compound of the formula NH₂—(CH₂) 6- is conjugated to the oligonucleotide via a reaction between 6-amino-1-hexanol (NH₂—(CH₂) 6—OH) and the 5′ phosphate of the sense strand of an oligonucleotide (e.g., an oligonucleotide listed in Table 9). In some embodiments, a compound of the formula NH₂—(CH₂) 6- is conjugated to the oligonucleotide via a reaction between 6-amino-1-hexanol (NH₂—(CH₂) 6—OH) and the 5′ phosphate of the antisense strand of an oligonucleotide (e.g., an oligonucleotide listed in Table 8). In some embodiments, a compound of the formula NH₂—(CH₂) 6- is conjugated to the oligonucleotide via a reaction between 6-amino-1-hexanol (NH₂—(CH₂) 6—OH) and the 5′ phosphate of the antisense strand of an oligonucleotide (e.g., an oligonucleotide listed in Table 9). In some embodiments, a compound of the formula NH₂—(CH₂) 6- is conjugated to the oligonucleotide via a reaction between 6-amino-1-hexanol (NH₂—(CH₂) 6—OH) and the 3′ phosphate of the oligonucleotide (e.g., 3′ phosphate of the sense or antisense strand). In some embodiments, a compound of the formula NH₂—(CH₂) 6- is conjugated to the oligonucleotide via a reaction between 6-amino-1-hexanol (NH₂—(CH₂) 6—OH) and the 3′ phosphate of the sense strand of an oligonucleotide (e.g., an oligonucleotide listed in Table 8). In some embodiments, a compound of the formula NH₂—(CH₂) 6- is conjugated to the oligonucleotide via a reaction between 6-amino-1-hexanol (NH₂—(CH₂) 6—OH) and the 3′ phosphate of the sense strand of an oligonucleotide (e.g., an oligonucleotide listed in Table 9). In some embodiments, a compound of the formula NH₂—(CH₂) 6- is conjugated to the oligonucleotide via a reaction between 6-amino-1-hexanol (NH₂—(CH₂) 6—OH) and the 3′ phosphate of the antisense strand of an oligonucleotide (e.g., an oligonucleotide listed in Table 8). In some embodiments, a compound of the formula NH₂—(CH₂) 6- is conjugated to the oligonucleotide via a reaction between 6-amino-1-hexanol (NH₂—(CH₂) 6—OH) and the 3′ phosphate of the antisense strand of an oligonucleotide (e.g., an oligonucleotide listed in Table 9). In some embodiments, the oligonucleotide is conjugated to a targeting agent, e.g., a muscle targeting agent such as an anti-TfR1 antibody, e.g., via the amine group.

In some embodiments, an oligonucleotide described herein (e.g., an siRNA molecule listed in Table 8 or Table 9) has reduced off-target effects. e.g., compared to other known DUX4-targeting siRNAs.

### C. Linkers

Complexes described herein generally comprise a linker that covalently links any one of the anti-TfR1 antibodies described herein to a molecular payload. A linker comprises at least one covalent bond. In some embodiments, a linker may be a single bond, e.g., a disulfide bond or disulfide bridge, that covalently links an anti-TfR1 antibody to a molecular payload. However, in some embodiments, a linker may covalently link any one of the anti-TfR1 antibodies described herein to a molecular payload through multiple covalent bonds. In some embodiments, a linker may be a cleavable linker. However, in some embodiments, a linker may be a non-cleavable linker. A linker is typically stable in vitro and in vivo, and may be stable in certain cellular environments. Additionally, typically a linker does not negatively impact the functional properties of either the anti-TfR1 antibody or the molecular payload. Examples and methods of synthesis of linkers are known in the art (see, e.g. Kline, T. et al. “Methods to Make Homogenous Antibody Drug Conjugates.” Pharmaceutical Research, 2015, 32:11, 3480-3493.; Jain, N. et al. “Current ADC Linker Chemistry” Pharm Res. 2015, 32:11, 3526-3540.; McCombs, J. R. and Owen, S. C. “Antibody Drug Conjugates: Design and Selection of Linker, Payload and Conjugation Chemistry” AAPS J. 2015, 17:2, 339-351.).

A linker typically will contain two different reactive species that allow for attachment to both the anti-TfR1 antibody and a molecular payload. In some embodiments, the two different reactive species may be a nucleophile and/or an electrophile. In some embodiments, a linker contains two different electrophiles or nucleophiles that are specific for two different nucleophiles or electrophiles. In some embodiments, a linker is covalently linked to an anti-TfR1 antibody via conjugation to a lysine residue or a cysteine residue of the anti-TfR1 antibody. In some embodiments, a linker is covalently linked to a cysteine residue of an anti-TfR1 antibody via a maleimide-containing linker, wherein optionally the maleimide-containing linker comprises a maleimidocaproyl or maleimidomethyl cyclohexane-1-carboxylate group. In some embodiments, a linker is covalently linked to a cysteine residue of an anti-TfR1 antibody or thiol functionalized molecular payload via a 3-arylpropionitrile functional group. In some embodiments, a linker is covalently linked to a lysine residue of an anti-TfR1 antibody. In some embodiments, a linker is covalently linked to an anti-TfR1 antibody and/or (e.g., and) a molecular payload, independently, via an amide bond, a carbamate bond, a hydrazide, a triazole, a thioether, and/or a disulfide bond.

### i. Cleavable Linkers

A cleavable linker may be a protease-sensitive linker, a pH-sensitive linker, or a glutathione-sensitive linker. These linkers are typically cleavable only intracellularly and are preferably stable in extracellular environments, e.g., extracellular to a muscle cell.

Protease-sensitive linkers are cleavable by protease enzymatic activity. These linkers typically comprise peptide sequences and may be 2-10 amino acids, about 2-5 amino acids, about 5-10 amino acids, about 10 amino acids, about 5 amino acids, about 3 amino acids, or about 2 amino acids in length. In some embodiments, a peptide sequence may comprise naturally-occurring amino acids, e.g. cysteine, alanine, or non-naturally-occurring or modified amino acids. Non-naturally occurring amino acids include β-amino acids, homo-amino acids, proline derivatives, 3-substituted alanine derivatives, linear core amino acids, N-methyl amino acids, and others known in the art. In some embodiments, a protease-sensitive linker comprises a valine-citrulline or alanine-citrulline sequence. In some embodiments, a protease-sensitive linker can be cleaved by a lysosomal protease, e.g. cathepsin B, and/or (e.g., and) an endosomal protease.

A pH-sensitive linker is a covalent linkage that readily degrades in high or low pH environments. In some embodiments, a pH-sensitive linker may be cleaved at a pH in a range of 4 to 6. In some embodiments, a pH-sensitive linker comprises a hydrazone or cyclic acetal. In some embodiments, a pH-sensitive linker is cleaved within an endosome or a lysosome.

In some embodiments, a glutathione-sensitive linker comprises a disulfide moiety. In some embodiments, a glutathione-sensitive linker is cleaved by a disulfide exchange reaction with a glutathione species inside a cell. In some embodiments, the disulfide moiety further comprises at least one amino acid, e.g., a cysteine residue.

In some embodiments, a linker comprises a valine-citrulline sequence (e.g., as described in U.S. Pat. No. 6,214,345, incorporated herein by reference). In some embodiments, before conjugation, a linker comprises a structure of:

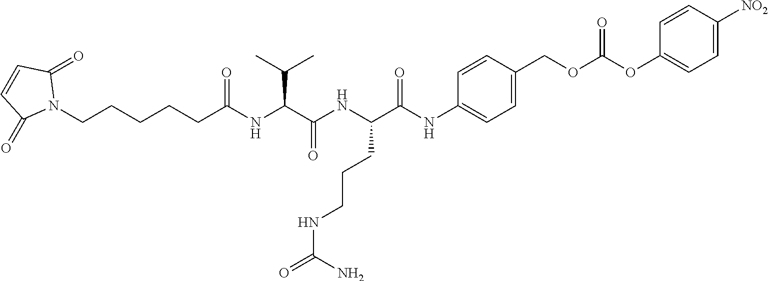 [SMILES: CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1]

In some embodiments, after conjugation, a linker comprises a structure of:

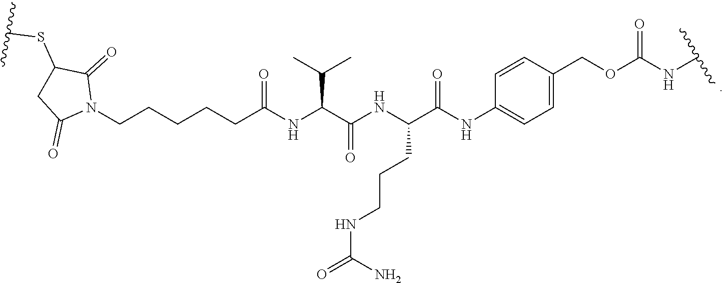 [SMILES: *NC(=O)OCc1ccc(NC(=O)[C@H](CCCNC(N)=O)NC(=O)[C@@H](NC(=O)CCCCCN2C(=O)CC(S*)C2=O)C(C)C)cc1]

In some embodiments, before conjugation, a linker comprises a structure of formula (A):

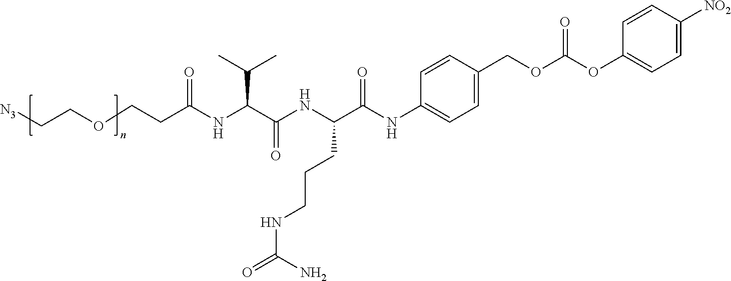

wherein n is any number from 0-10. In some embodiments, n is 3.

In some embodiments, a linker comprises a structure of formula (H):

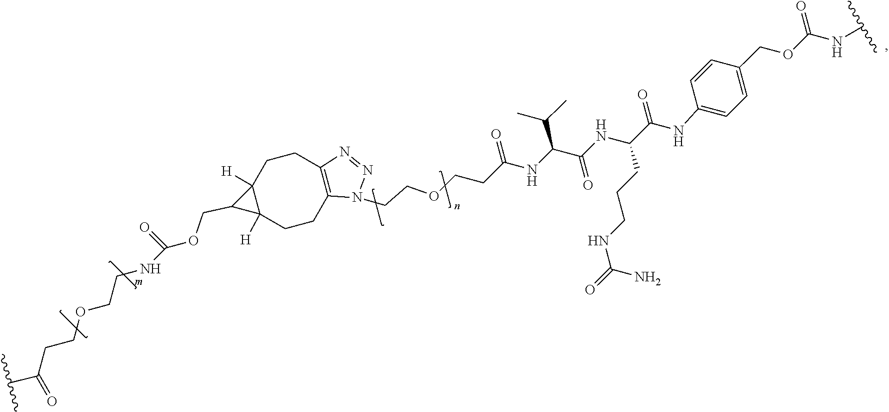

wherein n is any number from 0-10, wherein m is any number from 0-10. In some embodiments, n is 3 and/or (e.g., and) m is 4.

In some embodiments, a linker comprises a structure of formula (I):

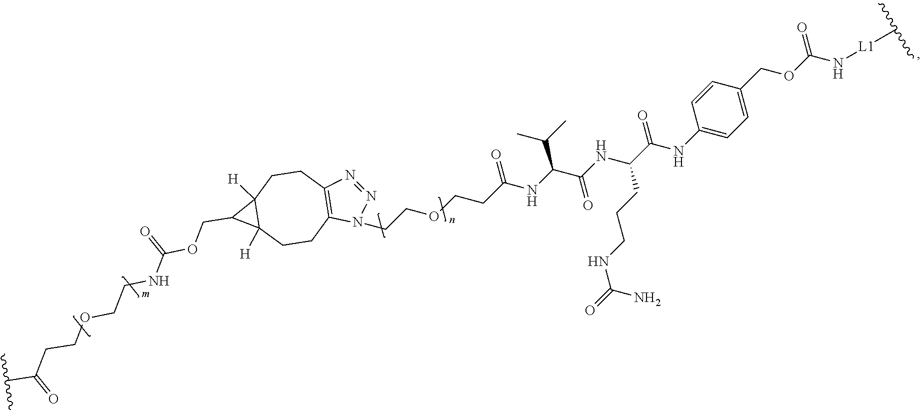

wherein n is any number from 0-10, wherein m is any number from 0-10. In some embodiments, n is 3 and/or (e.g., and) m is 4.

### ii. Non-Cleavable Linkers

In some embodiments, non-cleavable linkers may be used. Generally, a non-cleavable linker cannot be readily degraded in a cellular or physiological environment. In some embodiments, a non-cleavable linker comprises an optionally substituted alkyl group, wherein the substitutions may include halogens, hydroxyl groups, oxygen species, and other common substitutions. In some embodiments, a linker may comprise an optionally substituted alkyl, an optionally substituted alkylene, an optionally substituted arylene, a heteroarylene, a peptide sequence comprising at least one non-natural amino acid, a truncated glycan, a sugar or sugars that cannot be enzymatically degraded, an azide, an alkyne-azide, a peptide sequence comprising a LPXT sequence, a thioether, a biotin, a biphenyl, repeating units of polyethylene glycol or equivalent compounds, acid esters, acid amides, sulfamides, and/or an alkoxy-amine linker. In some embodiments, sortase-mediated ligation can be utilized to covalently link an anti-TfR1 antibody comprising a LPXT sequence to a molecular payload comprising a (G) n sequence (see, e.g. Proft T. Sortase-mediated protein ligation: an emerging biotechnology tool for protein modification and immobilization. Biotechnol Lett. 2010, 32 (1): 1-10.).

In some embodiments, a linker may comprise a substituted alkylene, an optionally substituted alkenylene, an optionally substituted alkynylene, an optionally substituted cycloalkylene, an optionally substituted cycloalkenylene, an optionally substituted arylene, an optionally substituted heteroarylene further comprising at least one heteroatom selected from N, O, and S,; an optionally substituted heterocyclylene further comprising at least one heteroatom selected from N, O, and S, an imino, an optionally substituted nitrogen species, an optionally substituted oxygen species O, an optionally substituted sulfur species, or a poly (alkylene oxide), e.g. polyethylene oxide or polypropylene oxide. In some embodiments, a linker may be a non-cleavable N-gamma-maleimidobutyryl-oxysuccinimide ester (GMBS) linker.

### iii. Linker conjugation

In some embodiments, a linker is covalently linked to an anti-TfR1 antibody and/or (e.g., and) molecular payload via a phosphate, thioether, ether, carbon-carbon, carbamate, or amide bond. In some embodiments, a linker is covalently linked to an oligonucleotide through a phosphate or phosphorothioate group, e.g. a terminal phosphate of an oligonucleotide backbone. In some embodiments, a linker is covalently linked to an anti-TfR1 antibody, through a lysine or cysteine residue present on the anti-TfR1 antibody.

In some embodiments, a linker, or a portion thereof is covalently linked to an anti-TfR1 antibody and/or (e.g., and) molecular payload by a cycloaddition reaction between an azide and an alkyne to form a triazole, wherein the azide or the alkyne may be located on the anti-TfR1 antibody, molecular payload, or the linker. In some embodiments, an alkyne may be a cyclic alkyne, e.g., a cyclooctyne. In some embodiments, an alkyne may be bicyclononyne (also known as bicyclo[6.1.0]nonyne or BCN) or substituted bicyclononyne. In some embodiments, a cyclooctyne is as described in International Patent Application Publication WO2011136645, published on Nov. 3, 2011, entitled, “Fused Cyclooctyne Compounds And Their Use In Metal-free Click Reactions”. In some embodiments, an azide may be a sugar or carbohydrate molecule that comprises an azide. In some embodiments, an azide may be 6-azido-6-deoxygalactose or 6-azido-N-acetylgalactosamine. In some embodiments, a sugar or carbohydrate molecule that comprises an azide is as described in International Patent Application Publication WO2016170186, published on Oct. 27, 2016, entitled, “Process For The Modification Of A Glycoprotein Using A Glycosyltransferase That Is Or Is Derived From A β (1,4)-N-Acetylgalactosaminyltransferase”. In some embodiments, a cycloaddition reaction between an azide and an alkyne to form a triazole, wherein the azide or the alkyne may be located on the anti-TfR1 antibody, molecular payload, or the linker is as described in International Patent Application Publication WO2014065661, published on May 1, 2014, entitled, “Modified antibody, antibody-conjugate and process for the preparation thereof′; or International Patent Application Publication WO2016170186, published on Oct. 27, 2016, entitled, “Process For The Modification Of A Glycoprotein Using A Glycosyltransferase That Is Or Is Derived From A β (1,4)-N-Acetylgalactosaminyltransferase”.

In some embodiments, a linker comprises a spacer, e.g., a polyethylene glycol spacer or an acyl/carbomoyl sulfamide spacer, e.g., a HydraSpace™ spacer. In some embodiments, a spacer is as described in Verkade, J. M. M. et al., “A Polar Sulfamide Spacer Significantly Enhances the Manufacturability, Stability, and Therapeutic Index of Antibody-Drug Conjugates”, Antibodies, 2018, 7, 12.

In some embodiments, a linker is covalently linked to an anti-TfR1 antibody and/or (e.g., and) molecular payload by the Diels-Alder reaction between a dienophile and a diene/hetero-diene, wherein the dienophile or the diene/hetero-diene may be located on the anti-TfR1 antibody, molecular payload, or the linker. In some embodiments a linker is covalently linked to an anti-TfR1 antibody and/or (e.g., and) molecular payload by other pericyclic reactions such as an ene reaction. In some embodiments, a linker is covalently linked to an anti-TfR1 antibody and/or (e.g., and) molecular payload by an amide, thioamide, or sulfonamide bond reaction. In some embodiments, a linker is covalently linked to an anti-TfR1 antibody and/or (e.g., and) molecular payload by a condensation reaction to form an oxime, hydrazone, or semicarbazide group existing between the linker and the anti-TfR1 antibody and/or (e.g., and) molecular payload.

In some embodiments, a linker is covalently linked to an anti-TfR1 antibody and/or (e.g., and) molecular payload by a conjugate addition reactions between a nucleophile, e.g. an amine or a hydroxyl group, and an electrophile, e.g. a carboxylic acid, carbonate, or an aldehyde. In some embodiments, a nucleophile may exist on a linker and an electrophile may exist on an anti-TfR1 antibody or molecular payload prior to a reaction between a linker and an anti-TfR1 antibody or molecular payload. In some embodiments, an electrophile may exist on a linker and a nucleophile may exist on an anti-TfR1 antibody or molecular payload prior to a reaction between a linker and an anti-TfR1 antibody or molecular payload. In some embodiments, an electrophile may be an azide, pentafluorophenyl, a silicon centers, a carbonyl, a carboxylic acid, an anhydride, an isocyanate, a thioisocyanate, a succinimidyl ester, a sulfosuccinimidyl ester, a maleimide, an alkyl halide, an alkyl pseudohalide, an epoxide, an episulfide, an aziridine, an aryl, an activated phosphorus center, and/or an activated sulfur center. In some embodiments, a nucleophile may be an optionally substituted alkene, an optionally substituted alkyne, an optionally substituted aryl, an optionally substituted heterocyclyl, a hydroxyl group, an amino group, an alkylamino group, an anilido group, and/or a thiol group.

In some embodiments, a linker comprises a valine-citrulline sequence covalently linked to a reactive chemical moiety (e.g., an azide moiety or a BCN moiety for click chemistry). In some embodiments, a linker comprising a valine-citrulline sequence covalently linked to a reactive chemical moiety (e.g., an azide moiety for click chemistry) comprises a structure of formula (A):

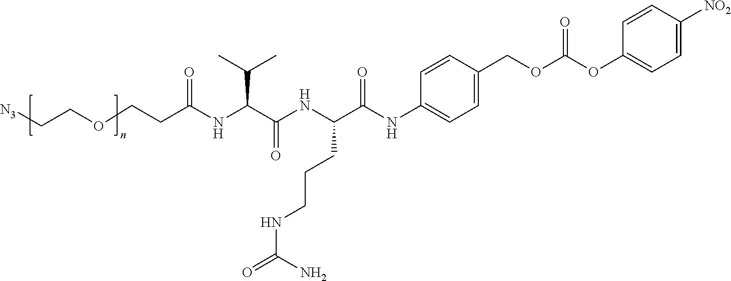

wherein n is any number from 0-10. In some embodiments, n is 3.

In some embodiments, a linker comprising the structure of Formula (A) is covalently linked (e.g., optionally via additional chemical moieties) to a molecular payload (e.g., an oligonucleotide). In some embodiments, the molecular payload is a double stranded siRNA oligonucleotide, and a linker comprising the structure of Formula (A) is covalently linked (e.g., optionally via additional chemical moieties) to the sense strand (e.g., at the 5′ end) of the siRNA oligonucleotide. In some embodiments, the molecular payload is a double stranded siRNA oligonucleotide, and a linker comprising the structure of Formula (A) is covalently linked (e.g., optionally via additional chemical moieties) to the sense strand (e.g., at the 3′ end) of the siRNA oligonucleotide. In some embodiments, a linker comprising the structure of Formula (A) is covalently linked to an oligonucleotide, e.g., through a nucleophilic substitution with amine-L1-oligonucleotides forming a carbamate bond, yielding a compound comprising a structure of formula (B):

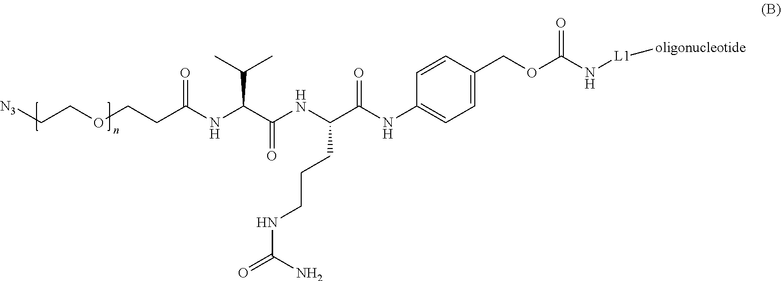

wherein n is any number from 0-10. In some embodiments, n is 3. In some embodiments, the oligonucleotide of a compound comprising a structure of formula (B) comprises the sense strand of an siRNA oligonucleotide. In some embodiments, an antisense strand is annealed to (e.g., at an annealing temperature of 25-105 °C) the sense strand of the siRNA oligonucleotide. In some embodiments, the annealing step is performed at an annealing temperature of 25 °C-150 °C (e.g., 25 °C-150 °C, 25 °C-100 °C, 25 °C-75 °C, 25 °C-50 °C, 25 °C-40 °C, 25 °C-30 °C, 30 °C-150° °C, 30 °C-100 °C, 30 °C-75 °C, 30 °C-50 °C, 30 °C-40 °C, 75 °C-150 °C, 75 °C-125 °C, or 75 °C-100 °C). In some embodiments, the annealing step is performed at an annealing temperature of 100 °C. For example, the annealing reaction mixture maybe incubated at 100 °C (e.g., for 3, 4, 5, 6, 7, 8, 9, 10 minutes, or longer) and allowed to cool down at room temperature for the sense strand and antisense strand to anneal. In some embodiments, the annealing step is performed at an annealing temperature of 20 °C, 21 °C, 22 °C, 23 °C, 24 °C, 25 °C, 26 °C, 27 °C, 28 °C, 29 °C, 30 °C, 31 °C, 32 °C, 33 °C, 34 °C, 35 °C, 36 °C, 37 °C, 38 °C, 39 °C, 40 °C. In some embodiments, the annealing step is performed at an annealing temperature of 30 °C. For example, the annealing reaction mixture maybe incubated at 30 °C (e.g., for 1-24 hours such as 1-24, 5-20, 5-15, 5-10, 1-5, 10-24, 10-20, 10-15, 15-24, 15-20, or 20-24 hours, or overnight) for the sense strand and antisense strand to anneal. In some embodiments, the oligonucleotide of a compound comprising a structure of formula (B) comprises an siRNA oligonucleotide comprising a sense strand and an antisense strand, in which the 5′ end of the sense strand is covalently linked to L1. In some embodiments, the oligonucleotide of formula (B) comprises an siRNA oligonucleotide comprising a sense strand and an antisense strand, in which the 3′ end of the sense strand is covalently linked to L1.

In some embodiments, the compound of Formula (B) is further covalently linked via a triazole to additional moieties, wherein the triazole is formed by a click reaction between the azide of Formula (A) or Formula (B) and an alkyne provided on a bicyclononyne. In some embodiments, a compound comprising a bicyclononyne comprises a structure of formula (C):

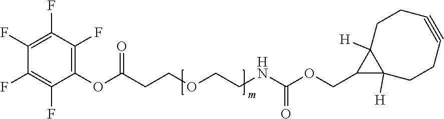

wherein m is any number from 0-10. In some embodiments, m is 4. In some embodiments, in a click reaction between a compound comprising the structure of Formula (B) and a compound comprising the structure of formula (C), the compound of formula (C) is in molar excess (e.g., at a molar ratio of (C) to (B) of 3:1 or 4:1). In some embodiments, in a click reaction between a compound comprising the structure of Formula (B) and a compound comprising the structure of formula (C), the compound of formula (B) is in molar excess (e.g., at a molar ratio of (C) to (B) of 0.9:1 or 0.85:1).

In some embodiments, the azide of the compound of structure (B) forms a triazole via a click reaction with the alkyne of the compound of structure (C), forming a compound comprising a structure of formula (D):

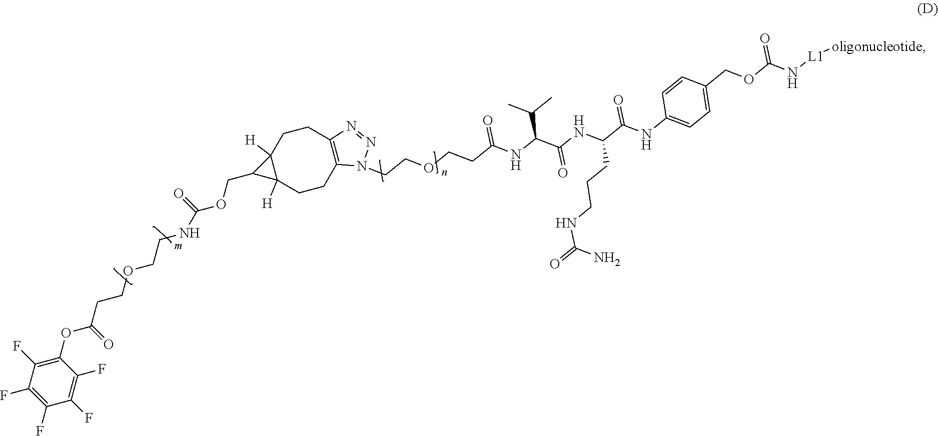

wherein n is any number from 0-10, and wherein m is any number from 0-10. In some embodiments, n is 3 and m is 4. In some embodiments, the oligonucleotide of formula (D) comprises an siRNA oligonucleotide comprising a sense strand and an antisense strand, in which the 5′ end of the sense strand is covalently linked to L1. In some embodiments, the oligonucleotide of formula (D) comprises an siRNA oligonucleotide comprising a sense strand and an antisense strand, in which the 3′ end of the sense strand is covalently linked to L1. In some embodiments, the compound comprising a structure of formula (D) is isolated prior to further covalently linking to a lysine of an anti-TfR1 antibody. In some embodiments, when the compound comprising a structure of formula (D) is isolated prior to further covalently linking to a lysine of an anti-TfR1 antibody, in a click reaction between a compound comprising the structure of Formula (B) and a compound comprising the structure of formula (C), the molar ratio between the compound comprising the structure of Formula (C) and the compound comprising the structure of Formula (B) is more than 1, e.g., 3:1 or 4:1. In some embodiments, the compound comprising a structure of formula (D) is not isolated prior to further covalently linking to a lysine of an anti-TfR1 antibody. In some embodiments, when the compound comprising a structure of formula (D) is not isolated prior to further covalently linking to a lysine of an anti-TfR1 antibody, in a click reaction between a compound comprising the structure of Formula (B) and a compound comprising the structure of formula (C), the molar ratio between the compound comprising the structure of Formula (C) and the compound comprising the structure of Formula (B) is less than 1, e.g., 0.9:1 or 0.85:1.

In some embodiments, the compound of structure (D) is further covalently linked to a lysine of the anti-TfR1 antibody, forming a complex comprising a structure of formula (E):

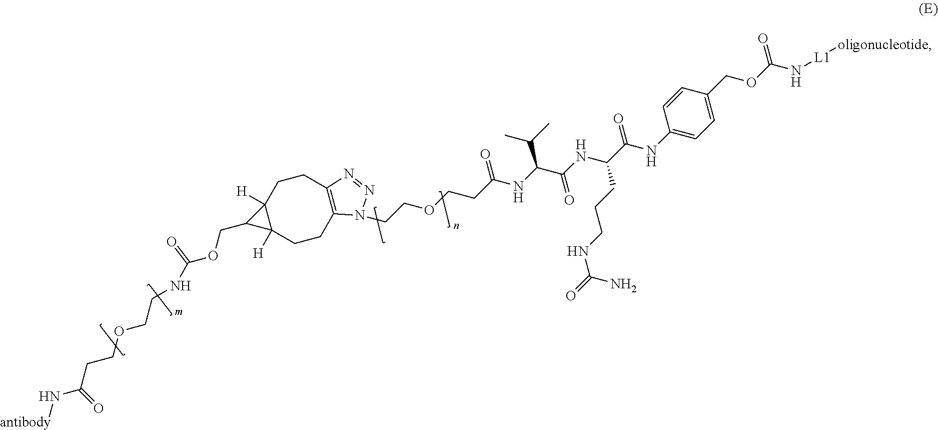

wherein n is any number from 0-10, wherein m is any number from 0-10. In some embodiments, n is 3 and/or (e.g., and) m is 4. It should be understood that the amide shown adjacent the anti-TfR1 antibody in Formula (E) results from a reaction with an amine of the anti-TfR1 antibody, such as a lysine epsilon amine. In some embodiments, the oligonucleotide of formula (E) comprises an siRNA oligonucleotide comprising a sense strand and an antisense strand, in which the 5′ end of the sense strand is covalently linked to L1. In some embodiments, the oligonucleotide of formula (E) comprises an siRNA oligonucleotide comprising a sense strand and an antisense strand, in which the 3′ end of the sense strand is covalently linked to L1.

In some embodiments, the compound of Formula (C) is further covalently linked to a lysine of the anti-TfR1 antibody, forming a compound comprising a structure of formula (F):

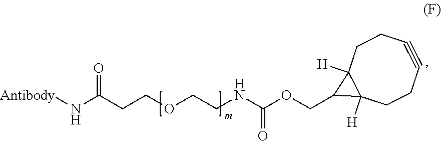

wherein m is 0-15 (e.g., 4). It should be understood that the amide shown adjacent the anti-TfR1 antibody in Formula (F) results from a reaction with an amine of the anti-TfR1 antibody, such as a lysine epsilon amine.

In some embodiments, the azide of the compound of structure (B) forms a triazole via a click reaction with the alkyne of the compound of structure (F), forming a complex comprising a structure of formula (E):

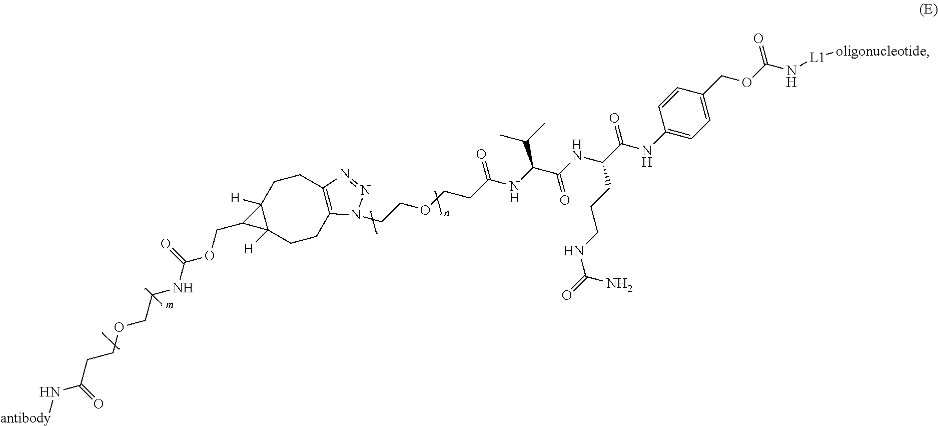

wherein n is any number from 0-10, wherein m is any number from 0-10. In some embodiments, n is 3 and/or (e.g., and) m is 4. It should be understood that the amide shown adjacent the anti-TfR1 antibody in Formula (E) results from a reaction with an amine of the anti-TfR1 antibody, such as a lysine epsilon amine. In some embodiments, the oligonucleotide of formula (E) comprises an siRNA oligonucleotide comprising a sense strand and an antisense strand, in which the 5′ end of the sense strand is covalently linked to L1. In some embodiments, the oligonucleotide of formula (E) comprises an siRNA oligonucleotide comprising a sense strand and an antisense strand, in which the 3′ end of the sense strand is covalently linked to L1.

In some embodiments, the azide of the compound of structure (A) forms a triazole via a click reaction with the alkyne of the compound of structure (F), forming a compound comprising a structure of formula (G):

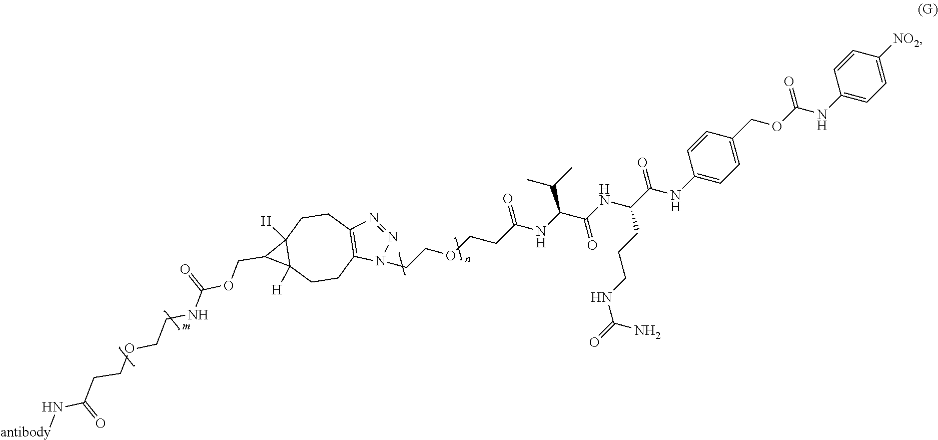

wherein n is any number from 0-10, wherein m is any number from 0-10. In some embodiments, n is 3 and/or (e.g., and) m is 4. In some embodiments, an oligonucleotide is covalently linked to a compound comprising a structure of formula (G), thereby forming a complex comprising a structure of formula (E). It should be understood that the amide shown adjacent the anti-TfR1 antibody in Formula (G) results from a reaction with an amine of the anti-TfR1 antibody, such as a lysine epsilon amine.

In some embodiments, in any one of the complexes described herein, the anti-TfR1 antibody is covalently linked via a lysine of the anti-TfR1 antibody to a molecular payload (e.g., an oligonucleotide) via a linker comprising a structure of formula (H):

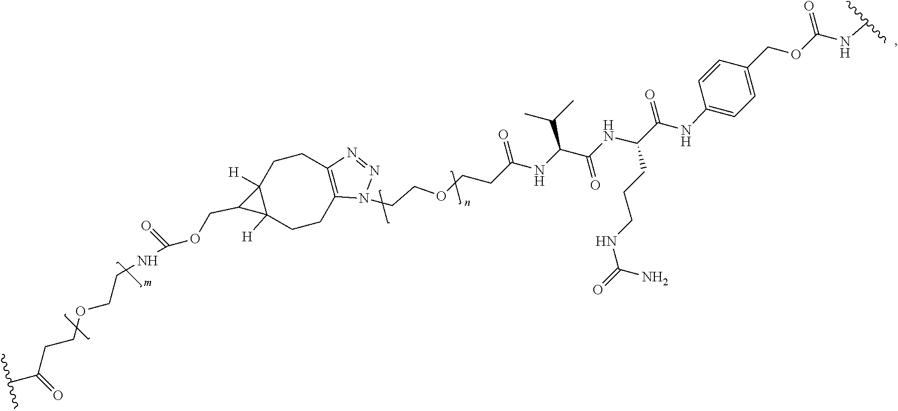

wherein n is any number from 0-10, wherein m is any number from 0-10. In some embodiments, n is 3 and/or (e.g., and) m is 4.

In some embodiments, in any one of the complexes described herein, the anti-TfR1 antibody is covalently linked via a lysine of the anti-TfR1 antibody to a molecular payload (e.g., an oligonucleotide) via a linker comprising a structure of formula (I):

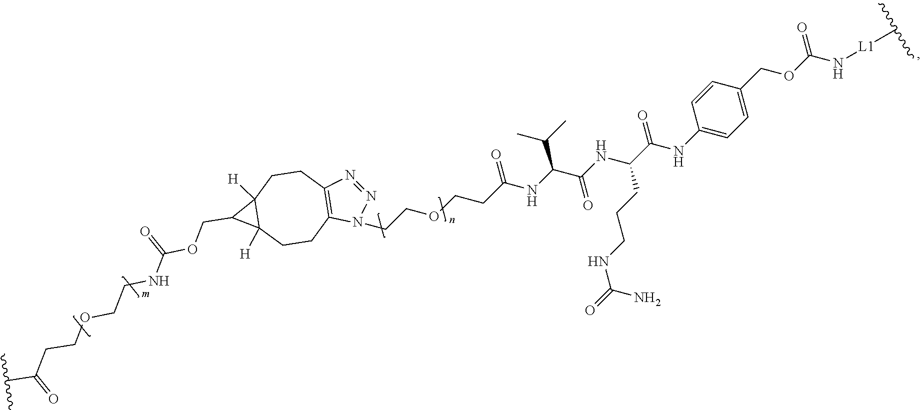

wherein n is any number from 0-10, wherein m is any number from 0-10. In some embodiments, n is 3 and/or (e.g., and) m is 4.

In some embodiments, in formulae (B), (D), (E), and (I), L1 is a spacer that is a substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof, wherein each R^{A} is independently hydrogen or substituted or unsubstituted alkyl. In some embodiments, L1 is

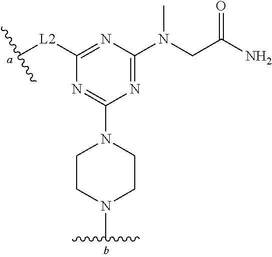

wherein L2 is

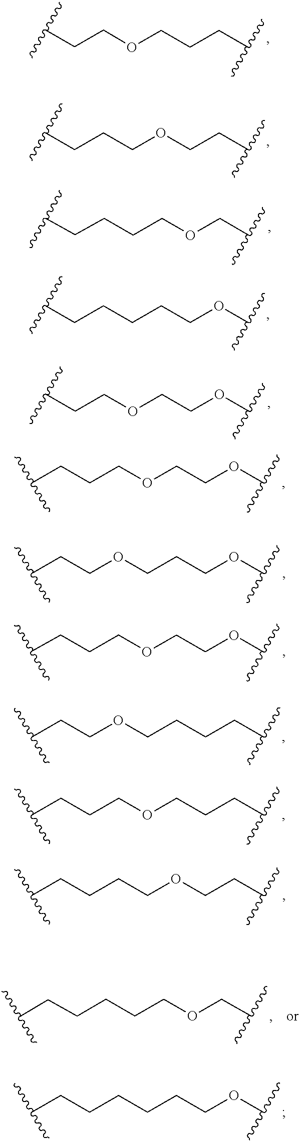

wherein a labels the site directly linked to the carbamate moiety of formulae (B), (D), (E), and (I); and b labels the site covalently linked (directly or via additional chemical moieties) to the oligonucleotide.

In some embodiments, L1 is:

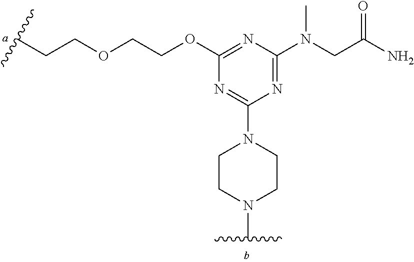 [SMILES: *CCOCCOc1nc(N(C)CC(N)=O)nc(N2CCN(*)CC2)n1]

wherein a labels the site directly linked to the carbamate moiety of formulae (B), (D), (E), and (I); and b labels the site covalently linked (directly or via additional chemical moieties) to the oligonucleotide.

In some embodiments, L1 is

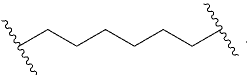 [SMILES: *CCCCCC*]

In some embodiments, L1 is linked to a 5′ phosphate of the oligonucleotide. In some embodiments, the linkage of L1 to a 5′ phosphate of the oligonucleotide forms a phosphodiester bond between L1 and the oligonucleotide.

In some embodiments, L1 is linked to a 3′ phosphate of the oligonucleotide. In some embodiments, the linkage of L1 to a 3′ phosphate of the oligonucleotide forms a phosphodiester bond between L1 and the oligonucleotide.

In some embodiments, L1 is optional (e.g., need not be present).

In some embodiments, any one of the complexes described herein has a structure of formula (J):

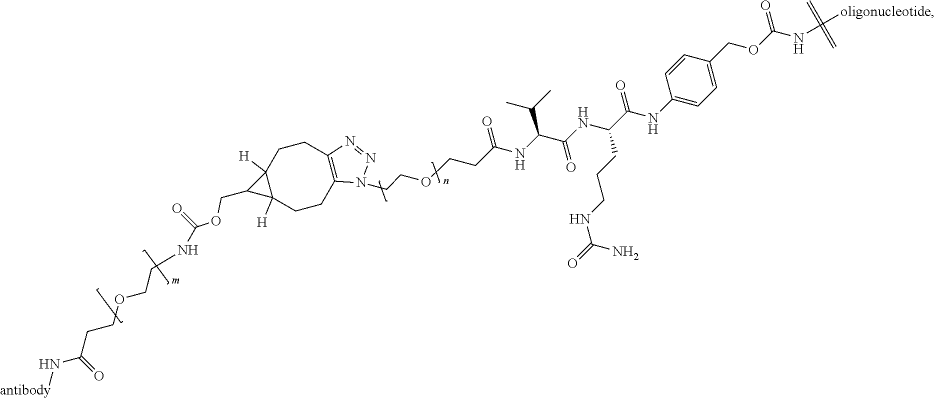

wherein n is 0-15 (e.g., 3) and m is 0-15 (e.g., 4). It should be understood that the amide shown adjacent the anti-TfR1 antibody in Formula (J) results from a reaction with an amine of the anti-TfR1 antibody, such as a lysine epsilon amine.

In some embodiments, any one of the complexes described herein has a structure of formula (K):

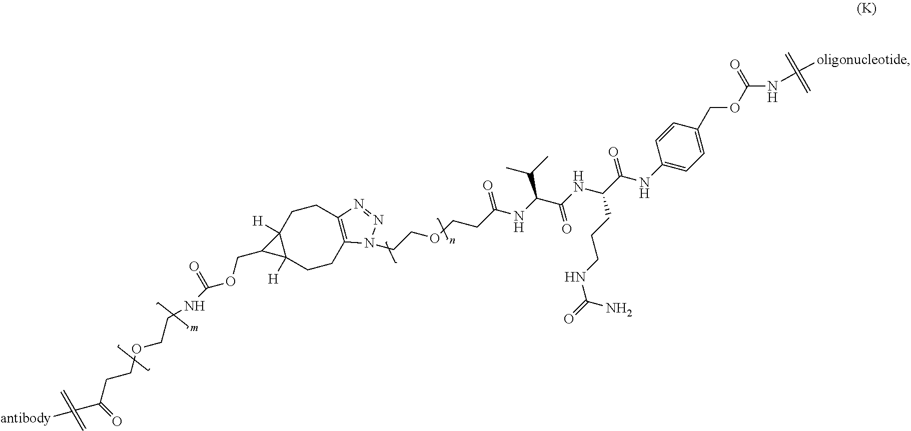

wherein n is 0-15 (e.g., 3) and m is 0-15 (e.g., 4).

In some embodiments, the oligonucleotide is modified to comprise an amine group at the 5′ end, the 3′ end, or internally (e.g., as an amine functionalized nucleobase), prior to linking to a compound, e.g., a compound of formula (A) or formula (G).

Although linker conjugation is described in the context of anti-TfR1 antibodies and oligonucleotide molecular payloads, it should be understood that use of such linker conjugation on other muscle-targeting agents, such as other muscle-targeting antibodies, and/or on other molecular payloads is contemplated.

### D. Examples of Antibody-Molecular Payload Complexes

Further provided herein are non-limiting examples of complexes comprising any one the anti-TfR1 antibodies described herein covalently linked to any of the molecular payloads (e.g., an oligonucleotide) described herein. In some embodiments, the anti-TfR1 antibody (e.g., any one of the anti-TfR1 antibodies provided in Tables 2-7) is covalently linked to a molecular payload (e.g., an oligonucleotide comprising at least 12 (e.g., at least 12, at least 13, at least 14, at least 15, at least 16, at least 17, at least 18, or at least 19) consecutive nucleotides of the nucleotide sequence of any one of SEQ ID NOs: 174-266) via a linker. In some embodiments, the anti-TfR1 antibody (e.g., any one of the anti-TfR1 antibodies provided in Tables 2-7) is covalently linked to a molecular payload (e.g., an oligonucleotide such as the oligonucleotides provided in Table 8) via a linker. In some embodiments, the anti-TfR1 antibody (e.g., any one of the anti-TfR1 antibodies provided in Tables 2-7) is covalently linked to a molecular payload (e.g., an oligonucleotide such as the oligonucleotides provided in Table 9) via a linker. Any of the linkers described herein may be used. In some embodiments, if the molecular payload is an oligonucleotide, the linker is linked to the 5′ end, the 3′ end, or internally of the sense strand or antisense strand. In some embodiments, the molecular payload is an siRNA, the linker is linked to the 5′ end of the sense strand. In some embodiments, the molecular payload is an siRNA, the linker is linked to the 3′ end of the sense strand. In some embodiments, the linker is linked to the anti-TfR1 antibody via a thiol-reactive linkage (e.g., via a cysteine in the anti-TfR1 antibody). In some embodiments, the linker (e.g., a linker comprising a valine-citrulline sequence) is linked to the antibody (e.g., an anti-TfR1 antibody described herein) via an amine group (e.g., via a lysine in the antibody). In some embodiments, the molecular payload is an oligonucleotide (e.g., an oligonucleotide listed in Table 8). In some embodiments, the molecular payload is an oligonucleotide (e.g., an oligonucleotide listed in Table 9). In some embodiments, the molecular payload is the sense strand of an siRNA. In some embodiments, the molecular payload is the antisense strand of an siRNA. In some embodiments, the molecular payload is an siRNA comprising a sense strand and an antisense strand.

An example of a structure of a complex comprising an anti-TfR1 antibody covalently linked to a molecular payload via a linker is provided below:

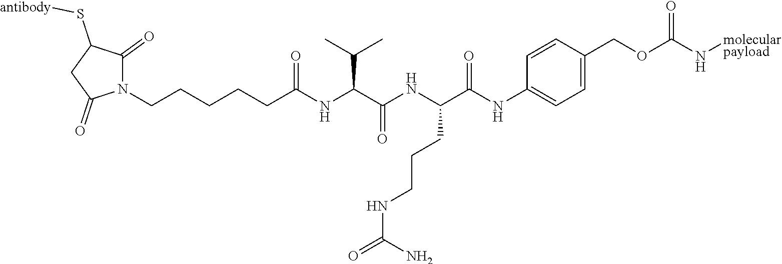

wherein the linker is linked to the antibody via a thiol-reactive linkage (e.g., via a cysteine in the antibody). In some embodiments, the molecular payload is an oligonucleotide comprising at least 12 (e.g., at least 12, at least 13, at least 14, at least 15, at least 16, at least 17, at least 18, or at least 19) consecutive nucleotides of the nucleotide sequence of any one of SEQ ID NOS: 174-266. In some embodiments, the molecular payload is an oligonucleotide comprising the nucleotide sequence of any one of SEQ ID NOs: 174-266. In some embodiments, the molecular payload is an oligonucleotide comprising an antisense strand comprising a region of complementarity of at least 16 nucleotides to a target sequence in DUX4 mRNA (e.g., a target sequence listed in Table 8), optionally wherein the antisense strand comprising at least 16 consecutive nucleotides of any one of the antisense sequences listed in Table 8, optionally wherein the oligonucleotide further comprises a sense strand that hybridizes to the antisense strand. In some embodiments, the molecular payload is an oligonucleotide comprising an antisense strand comprising a region of complementarity of at least 16 nucleotides to a target sequence in DUX4 mRNA (e.g., a target sequence listed in Table 9), optionally wherein the antisense strand comprising at least 16 consecutive nucleotides of any one of the antisense sequences listed in Table 9, optionally wherein the oligonucleotide further comprises a sense strand that hybridizes to the antisense strand.

Another example of a structure of a complex comprising an anti-TfR1 antibody covalently linked to a molecular payload via a linker is provided below:

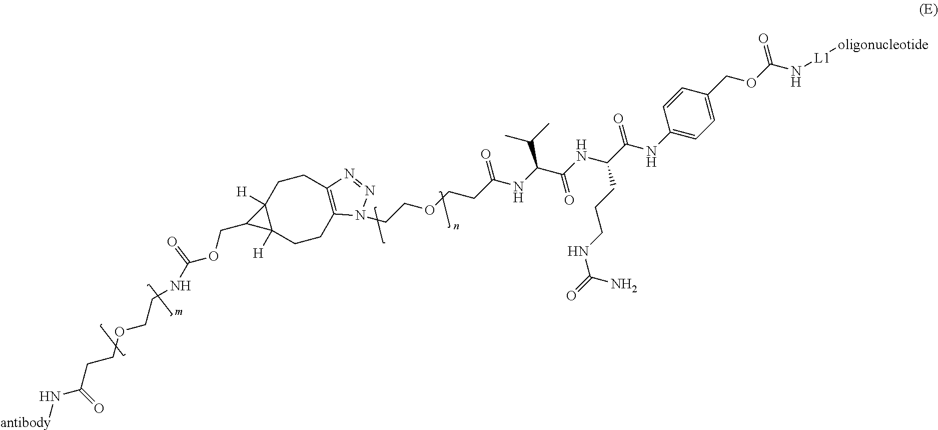

wherein n is a number between 0-10, wherein m is a number between 0-10, wherein the linker is linked to the antibody via an amine group (e.g., on a lysine residue), and/or (e.g., and) wherein the linker is linked to the sense strand or the antisense strand (e.g., at the 5′ end, 3′ end, or internally). In some embodiments, the linker is linked to the 5′ end of the sense strand. In some embodiments, the linker is linked to the 3′ end of the sense strand. In some embodiments, the linker is linked to the antibody via a lysine, the linker is linked to the oligonucleotide at the 5′ end, n is 3, and m is 4. In some embodiments, the molecular payload is an oligonucleotide comprising at least 12 (e.g., at least 12, at least 13, at least 14, at least 15, at least 16, at least 17, at least 18, or at least 19) nucleotides of the nucleotide sequence of any one of SEQ ID NOs: 174-266. In some embodiments, the molecular payload is an oligonucleotide comprising the nucleotide sequence of any one of SEQ ID NOs: 174-266. In some embodiments, the molecular payload is an oligonucleotide comprising an antisense strand comprising a region of complementarity of at least 16 nucleotides to a target sequence in DUX4 mRNA (e.g., a target sequence listed in Table 8), optionally wherein the antisense strand comprising at least 16 consecutive nucleotides of any one of the antisense sequences listed in Table 8, optionally wherein the oligonucleotide further comprises a sense strand that hybridizes to the antisense strand. In some embodiments, the molecular payload is an oligonucleotide comprising an antisense strand comprising a region of complementarity of at least 16 nucleotides to a target sequence in DUX4 mRNA (e.g., a target sequence listed in Table 9), optionally wherein the antisense strand comprising at least 16 consecutive nucleotides of any one of the antisense sequences listed in Table 9, optionally wherein the oligonucleotide further comprises a sense strand that hybridizes to the antisense strand. In some embodiments, L1 is any one of the spacers described herein. It should be understood that the amide shown adjacent the anti-TfR1 antibody in Formula (E) results from a reaction with an amine of the anti-TfR1 antibody, such as a lysine epsilon amine.

It should be appreciated that antibodies can be linked to molecular payloads with different stoichiometries, a property that may be referred to as a drug to antibody ratios (DAR) with the “drug” being the molecular payload. In some embodiments, one molecular payload is linked to an antibody (DAR=1). In some embodiments, two molecular payloads are linked to an antibody (DAR=2). In some embodiments, three molecular payloads are linked to an antibody (DAR=3). In some embodiments, four molecular payloads are linked to an antibody (DAR=4). In some embodiments, a mixture of different complexes, each having a different DAR, is provided. In some embodiments, an average DAR of complexes in such a mixture may be in a range of 1 to 3, 1 to 4, 1 to 5 or more. DAR may be increased by conjugating molecular payloads to different sites on an antibody and/or (e.g., and) by conjugating multimers to one or more sites on antibody. For example, a DAR of 2 may be achieved by conjugating a single molecular payload to two different sites on an antibody or by conjugating a dimer molecular payload to a single site of an antibody.

In some embodiments, the complex described herein comprises an anti-TfR1 antibody described herein (e.g., the antibodies provided in Tables 2-7) covalently linked to a molecular payload. In some embodiments, the complex described herein comprises an anti-TfR1 antibody described herein (e.g., the antibodies provided in Tables 2-7) covalently linked to molecular payload via a linker (e.g., a linker comprising a valine-citrulline sequence). In some embodiments, the linker (e.g., a linker comprising a valine-citrulline sequence) is linked to the antibody (e.g., an anti-TfR1 antibody described herein) via a thiol-reactive linkage (e.g., via a cysteine in the antibody). In some embodiments, the linker (e.g., a linker comprising a valine-citrulline sequence) is linked to the antibody (e.g., an anti-TfR1 antibody described herein) via an amine group (e.g., via a lysine in the antibody). In some embodiments, the molecular payload is an oligonucleotide comprising at least 12 (e.g., at least 12, at least 13, at least 14, at least 15, at least 16, at least 17, at least 18, or at least 19) nucleotides of the nucleotide sequence of any one of SEQ ID NOs: 174-266. In some embodiments, the molecular payload is an oligonucleotide comprising the nucleotide sequence of any one of SEQ ID NOs: 174-266. In some embodiments, the molecular payload is an oligonucleotide comprising an antisense strand comprising a region of complementarity of at least 16 nucleotides to a target sequence in DUX4 mRNA (e.g., a target sequence listed in Table 8), optionally wherein the antisense strand comprising at least 16 consecutive nucleotides of any one of the antisense sequences listed in Table 8, optionally wherein the oligonucleotide further comprises a sense strand that hybridizes to the antisense strand. In some embodiments, the molecular payload is an oligonucleotide comprising an antisense strand comprising a region of complementarity of at least 16 nucleotides to a target sequence in DUX4 mRNA (e.g., a target sequence listed in Table 9), optionally wherein the antisense strand comprising at least 16 consecutive nucleotides of any one of the antisense sequences listed in Table 9, optionally wherein the oligonucleotide further comprises a sense strand that hybridizes to the antisense strand.

In some embodiments, the complex described herein comprises an anti-TfR1 antibody covalently linked to a molecular payload, wherein the anti-TfR1 antibody comprises a CDR-H1, a CDR-H2, a CDR-H3, a CDR-L1, a CDR-L2, and a CDR-L3 of any one of the antibodies listed in Table 2. In some embodiments, the molecular payload is an oligonucleotide comprising an antisense strand comprising a region of complementarity of at least 16 nucleotides to a target sequence in DUX4 mRNA (e.g., a target sequence listed in Table 8), optionally wherein the antisense strand comprising at least 16 consecutive nucleotides of any one of the antisense sequences listed in Table 8, optionally wherein the oligonucleotide further comprises a sense strand that hybridizes to the antisense strand. In some embodiments, the molecular payload is an oligonucleotide comprising an antisense strand comprising a region of complementarity of at least 16 nucleotides to a target sequence in DUX4 mRNA (e.g., a target sequence listed in Table 9), optionally wherein the antisense strand comprising at least 16 consecutive nucleotides of any one of the antisense sequences listed in Table 9, optionally wherein the oligonucleotide further comprises a sense strand that hybridizes to the antisense strand.

In some embodiments, the complex described herein comprises an anti-TfR1 antibody covalently linked to a molecular payload, wherein the anti-TfR1 antibody comprises a VH comprising the amino acid sequence of SEQ ID NO: 69, SEQ ID NO: 71, or SEQ ID NO: 72, and a VL comprising the amino acid sequence of SEQ ID NO: 70. In some embodiments, the molecular payload is an oligonucleotide comprising an antisense strand comprising a region of complementarity of at least 16 nucleotides to a target sequence in DUX4 mRNA (e.g., a target sequence listed in Table 8), optionally wherein the antisense strand comprising at least 16 consecutive nucleotides of any one of the antisense sequences listed in Table 8, optionally wherein the oligonucleotide further comprises a sense strand that hybridizes to the antisense strand. In some embodiments, the molecular payload is an oligonucleotide comprising an antisense strand comprising a region of complementarity of at least 16 nucleotides to a target sequence in DUX4 mRNA (e.g., a target sequence listed in Table 9), optionally wherein the antisense strand comprising at least 16 consecutive nucleotides of any one of the antisense sequences listed in Table 9, optionally wherein the oligonucleotide further comprises a sense strand that hybridizes to the antisense strand.

In some embodiments, the complex described herein comprises an anti-TfR1 antibody covalently linked to a molecular payload, wherein the anti-TfR1 antibody comprises a VH comprising the amino acid sequence of SEQ ID NO: 73 or SEQ ID NO: 76, and a VL comprising the amino acid sequence of SEQ ID NO: 74. In some embodiments, the molecular payload is an oligonucleotide comprising an antisense strand comprising a region of complementarity of at least 16 nucleotides to a target sequence in DUX4 mRNA (e.g., a target sequence listed in Table 8), optionally wherein the antisense strand comprising at least 16 consecutive nucleotides of any one of the antisense sequences listed in Table 8, optionally wherein the oligonucleotide further comprises a sense strand that hybridizes to the antisense strand. In some embodiments, the molecular payload is an oligonucleotide comprising an antisense strand comprising a region of complementarity of at least 16 nucleotides to a target sequence in DUX4 mRNA (e.g., a target sequence listed in Table 9), optionally wherein the antisense strand comprising at least 16 consecutive nucleotides of any one of the antisense sequences listed in Table 9, optionally wherein the oligonucleotide further comprises a sense strand that hybridizes to the antisense strand.

In some embodiments, the complex described herein comprises an anti-TfR1 antibody covalently linked to a molecular payload, wherein the anti-TfR1 antibody comprises a VH comprising the amino acid sequence of SEQ ID NO: 73 or SEQ ID NO: 76, and a VL comprising the amino acid sequence of SEQ ID NO: 75. In some embodiments, the molecular payload is an oligonucleotide comprising an antisense strand comprising a region of complementarity of at least 16 nucleotides to a target sequence in DUX4 mRNA (e.g., a target sequence listed in Table 8), optionally wherein the antisense strand comprising at least 16 consecutive nucleotides of any one of the antisense sequences listed in Table 8, optionally wherein the oligonucleotide further comprises a sense strand that hybridizes to the antisense strand. In some embodiments, the molecular payload is an oligonucleotide comprising an antisense strand comprising a region of complementarity of at least 16 nucleotides to a target sequence in DUX4 mRNA (e.g., a target sequence listed in Table 9), optionally wherein the antisense strand comprising at least 16 consecutive nucleotides of any one of the antisense sequences listed in Table 9, optionally wherein the oligonucleotide further comprises a sense strand that hybridizes to the antisense strand.

In some embodiments, the complex described herein comprises an anti-TfR1 antibody covalently linked to a molecular payload, wherein the anti-TfR1 antibody comprises a VH comprising the amino acid sequence of SEQ ID NO: 77, and a VL comprising the amino acid sequence of SEQ ID NO: 78. In some embodiments, the molecular payload is an oligonucleotide comprising an antisense strand comprising a region of complementarity of at least 16 nucleotides to a target sequence in DUX4 mRNA (e.g., a target sequence listed in Table 8), optionally wherein the antisense strand comprising at least 16 consecutive nucleotides of any one of the antisense sequences listed in Table 8, optionally wherein the oligonucleotide further comprises a sense strand that hybridizes to the antisense strand. In some embodiments, the molecular payload is an oligonucleotide comprising an antisense strand comprising a region of complementarity of at least 16 nucleotides to a target sequence in DUX4 mRNA (e.g., a target sequence listed in Table 9), optionally wherein the antisense strand comprising at least 16 consecutive nucleotides of any one of the antisense sequences listed in Table 9, optionally wherein the oligonucleotide further comprises a sense strand that hybridizes to the antisense strand.

In some embodiments, the complex described herein comprises an anti-TfR1 antibody covalently linked to a molecular payload, wherein the anti-TfR1 antibody comprises a VH comprising the amino acid sequence of SEQ ID NO: 77 or SEQ ID NO: 79, and a VL comprising the amino acid sequence of SEQ ID NO: 80. In some embodiments, the molecular payload is an oligonucleotide comprising an antisense strand comprising a region of complementarity of at least 16 nucleotides to a target sequence in DUX4 mRNA (e.g., a target sequence listed in Table 8), optionally wherein the antisense strand comprising at least 16 consecutive nucleotides of any one of the antisense sequences listed in Table 8, optionally wherein the oligonucleotide further comprises a sense strand that hybridizes to the antisense strand. In some embodiments, the molecular payload is an oligonucleotide comprising an antisense strand comprising a region of complementarity of at least 16 nucleotides to a target sequence in DUX4 mRNA (e.g., a target sequence listed in Table 9), optionally wherein the antisense strand comprising at least 16 consecutive nucleotides of any one of the antisense sequences listed in Table 9, optionally wherein the oligonucleotide further comprises a sense strand that hybridizes to the antisense strand.

In some embodiments, the complex described herein comprises an anti-TfR1 antibody covalently linked to a molecular payload, wherein the anti-TfR1 antibody comprises a VH comprising the amino acid sequence of SEQ ID NO: 154, and a VL comprising the amino acid sequence of SEQ ID NO: 155. In some embodiments, the molecular payload is an oligonucleotide comprising an antisense strand comprising a region of complementarity of at least 16 nucleotides to a target sequence in DUX4 mRNA (e.g., a target sequence listed in Table 8), optionally wherein the antisense strand comprising at least 16 consecutive nucleotides of any one of the antisense sequences listed in Table 8, optionally wherein the oligonucleotide further comprises a sense strand that hybridizes to the antisense strand. In some embodiments, the molecular payload is an oligonucleotide comprising an antisense strand comprising a region of complementarity of at least 16 nucleotides to a target sequence in DUX4 mRNA (e.g., a target sequence listed in Table 9), optionally wherein the antisense strand comprising at least 16 consecutive nucleotides of any one of the antisense sequences listed in Table 9, optionally wherein the oligonucleotide further comprises a sense strand that hybridizes to the antisense strand.

In some embodiments, the molecular payload is an oligonucleotide (e.g., an oligonucleotide listed in Table 8). In some embodiments, the molecular payload is an oligonucleotide (e.g., an oligonucleotide listed in Table 9).

In some embodiments, the complex described herein comprises an anti-TfR1 antibody covalently linked to a molecular payload, wherein the anti-TfR1 antibody comprises a heavy chain comprising the amino acid sequence of SEQ ID NO: 84, SEQ ID NO: 86 or SEQ ID NO: 87 and a light chain comprising the amino acid sequence of SEQ ID NO: 85. In some embodiments, the molecular payload is an oligonucleotide comprising an antisense strand comprising a region of complementarity of at least 16 nucleotides to a target sequence in DUX4 mRNA (e.g., a target sequence listed in Table 8), optionally wherein the antisense strand comprising at least 16 consecutive nucleotides of any one of the antisense sequences listed in Table 8, optionally wherein the oligonucleotide further comprises a sense strand that hybridizes to the antisense strand. In some embodiments, the molecular payload is an oligonucleotide comprising an antisense strand comprising a region of complementarity of at least 16 nucleotides to a target sequence in DUX4 mRNA (e.g., a target sequence listed in Table 9), optionally wherein the antisense strand comprising at least 16 consecutive nucleotides of any one of the antisense sequences listed in Table 9, optionally wherein the oligonucleotide further comprises a sense strand that hybridizes to the antisense strand.

In some embodiments, the complex described herein comprises an anti-TfR1 antibody covalently linked to a molecular payload, wherein the anti-TfR1 antibody comprises a heavy chain comprising the amino acid sequence of SEQ ID NO: 88 or SEQ ID NO: 91, and a light chain comprising the amino acid sequence of SEQ ID NO: 89. In some embodiments, the molecular payload is an oligonucleotide comprising an antisense strand comprising a region of complementarity of at least 16 nucleotides to a target sequence in DUX4 mRNA (e.g., a target sequence listed in Table 8), optionally wherein the antisense strand comprising at least 16 consecutive nucleotides of any one of the antisense sequences listed in Table 8, optionally wherein the oligonucleotide further comprises a sense strand that hybridizes to the antisense strand. In some embodiments, the molecular payload is an oligonucleotide comprising an antisense strand comprising a region of complementarity of at least 16 nucleotides to a target sequence in DUX4 mRNA (e.g., a target sequence listed in Table 9), optionally wherein the antisense strand comprising at least 16 consecutive nucleotides of any one of the antisense sequences listed in Table 9, optionally wherein the oligonucleotide further comprises a sense strand that hybridizes to the antisense strand.

In some embodiments, the complex described herein comprises an anti-TfR1 antibody covalently linked to a molecular payload, wherein the anti-TfR1 antibody comprises a heavy chain comprising the amino acid sequence of SEQ ID NO: 88 or SEQ ID NO: 91, and a light chain comprising the amino acid sequence of SEQ ID NO: 90. In some embodiments, the molecular payload is an oligonucleotide comprising an antisense strand comprising a region of complementarity of at least 16 nucleotides to a target sequence in DUX4 mRNA (e.g., a target sequence listed in Table 8), optionally wherein the antisense strand comprising at least 16 consecutive nucleotides of any one of the antisense sequences listed in Table 8, optionally wherein the oligonucleotide further comprises a sense strand that hybridizes to the antisense strand. In some embodiments, the molecular payload is an oligonucleotide comprising an antisense strand comprising a region of complementarity of at least 16 nucleotides to a target sequence in DUX4 mRNA (e.g., a target sequence listed in Table 9), optionally wherein the antisense strand comprising at least 16 consecutive nucleotides of any one of the antisense sequences listed in Table 9, optionally wherein the oligonucleotide further comprises a sense strand that hybridizes to the antisense strand.

In some embodiments, the complex described herein comprises an anti-TfR1 antibody covalently linked to a molecular payload, wherein the anti-TfR1 antibody comprises a heavy chain comprising the amino acid sequence of SEQ ID NO: 92 or SEQ ID NO: 94, and a light chain comprising the amino acid sequence of SEQ ID NO: 95. In some embodiments, the molecular payload is an oligonucleotide comprising an antisense strand comprising a region of complementarity of at least 16 nucleotides to a target sequence in DUX4 mRNA (e.g., a target sequence listed in Table 8), optionally wherein the antisense strand comprising at least 16 consecutive nucleotides of any one of the antisense sequences listed in Table 8, optionally wherein the oligonucleotide further comprises a sense strand that hybridizes to the antisense strand. In some embodiments, the molecular payload is an oligonucleotide comprising an antisense strand comprising a region of complementarity of at least 16 nucleotides to a target sequence in DUX4 mRNA (e.g., a target sequence listed in Table 9), optionally wherein the antisense strand comprising at least 16 consecutive nucleotides of any one of the antisense sequences listed in Table 9, optionally wherein the oligonucleotide further comprises a sense strand that hybridizes to the antisense strand.

In some embodiments, the complex described herein comprises an anti-TfR1 antibody covalently linked to a molecular payload, wherein the anti-TfR1 antibody comprises a heavy chain comprising the amino acid sequence of SEQ ID NO: 92, and a light chain comprising the amino acid sequence of SEQ ID NO: 93. In some embodiments, the molecular payload is an oligonucleotide comprising an antisense strand comprising a region of complementarity of at least 16 nucleotides to a target sequence in DUX4 mRNA (e.g., a target sequence listed in Table 8), optionally wherein the antisense strand comprising at least 16 consecutive nucleotides of any one of the antisense sequences listed in Table 8, optionally wherein the oligonucleotide further comprises a sense strand that hybridizes to the antisense strand. In some embodiments, the molecular payload is an oligonucleotide comprising an antisense strand comprising a region of complementarity of at least 16 nucleotides to a target sequence in DUX4 mRNA (e.g., a target sequence listed in Table 9), optionally wherein the antisense strand comprising at least 16 consecutive nucleotides of any one of the antisense sequences listed in Table 9, optionally wherein the oligonucleotide further comprises a sense strand that hybridizes to the antisense strand.

In some embodiments, the complex described herein comprises an anti-TfR1 antibody covalently linked to a molecular payload, wherein the anti-TfR1 antibody comprises a heavy chain comprising the amino acid sequence of SEQ ID NO: 156, and a light chain comprising the amino acid sequence of SEQ ID NO: 157. In some embodiments, the molecular payload is an oligonucleotide comprising an antisense strand comprising a region of complementarity of at least 16 nucleotides to a target sequence in DUX4 mRNA (e.g., a target sequence listed in Table 8), optionally wherein the antisense strand comprising at least 16 consecutive nucleotides of any one of the antisense sequences listed in Table 8, optionally wherein the oligonucleotide further comprises a sense strand that hybridizes to the antisense strand. In some embodiments, the molecular payload is an oligonucleotide comprising an antisense strand comprising a region of complementarity of at least 16 nucleotides to a target sequence in DUX4 mRNA (e.g., a target sequence listed in Table 9), optionally wherein the antisense strand comprising at least 16 consecutive nucleotides of any one of the antisense sequences listed in Table 9, optionally wherein the oligonucleotide further comprises a sense strand that hybridizes to the antisense strand.

In some embodiments, the complex described herein comprises an anti-TfR1 antibody covalently linked to a molecular payload,, wherein the anti-TfR1 antibody comprises a heavy chain comprising the amino acid sequence of SEQ ID NO: 97, SEQ ID NO: 98, or SEQ ID NO: 99 and a VL comprising the amino acid sequence of SEQ ID NO: 85. In some embodiments, the molecular payload is an oligonucleotide comprising an antisense strand comprising a region of complementarity of at least 16 nucleotides to a target sequence in DUX4 mRNA (e.g., a target sequence listed in Table 8), optionally wherein the antisense strand comprising at least 16 consecutive nucleotides of any one of the antisense sequences listed in Table 8, optionally wherein the oligonucleotide further comprises a sense strand that hybridizes to the antisense strand. In some embodiments, the molecular payload is an oligonucleotide comprising an antisense strand comprising a region of complementarity of at least 16 nucleotides to a target sequence in DUX4 mRNA (e.g., a target sequence listed in Table 9), optionally wherein the antisense strand comprising at least 16 consecutive nucleotides of any one of the antisense sequences listed in Table 9, optionally wherein the oligonucleotide further comprises a sense strand that hybridizes to the antisense strand.

In some embodiments, the complex described herein comprises an anti-TfR1 antibody covalently linked to a molecular payload, wherein the anti-TfR1 antibody comprises a heavy chain comprising the amino acid sequence of SEQ ID NO: 100 or SEQ ID NO: 101 and a light chain comprising the amino acid sequence of SEQ ID NO: 89. In some embodiments, the molecular payload is an oligonucleotide comprising an antisense strand comprising a region of complementarity of at least 16 nucleotides to a target sequence in DUX4 mRNA (e.g., a target sequence listed in Table 8), optionally wherein the antisense strand comprising at least 16 consecutive nucleotides of any one of the antisense sequences listed in Table 8, optionally wherein the oligonucleotide further comprises a sense strand that hybridizes to the antisense strand. In some embodiments, the molecular payload is an oligonucleotide comprising an antisense strand comprising a region of complementarity of at least 16 nucleotides to a target sequence in DUX4 mRNA (e.g., a target sequence listed in Table 9), optionally wherein the antisense strand comprising at least 16 consecutive nucleotides of any one of the antisense sequences listed in Table 9, optionally wherein the oligonucleotide further comprises a sense strand that hybridizes to the antisense strand.

In some embodiments, the complex described herein comprises an anti-TfR1 antibody covalently linked to a molecular payload, wherein the anti-TfR1 antibody comprises a heavy chain comprising the amino acid sequence of SEQ ID NO: 100 or SEQ ID NO: 101 and a light chain comprising the amino acid sequence of SEQ ID NO: 90. In some embodiments, the molecular payload is an oligonucleotide comprising an antisense strand comprising a region of complementarity of at least 16 nucleotides to a target sequence in DUX4 mRNA (e.g., a target sequence listed in Table 8), optionally wherein the antisense strand comprising at least 16 consecutive nucleotides of any one of the antisense sequences listed in Table 8, optionally wherein the oligonucleotide further comprises a sense strand that hybridizes to the antisense strand. In some embodiments, the molecular payload is an oligonucleotide comprising an antisense strand comprising a region of complementarity of at least 16 nucleotides to a target sequence in DUX4 mRNA (e.g., a target sequence listed in Table 9), optionally wherein the antisense strand comprising at least 16 consecutive nucleotides of any one of the antisense sequences listed in Table 9, optionally wherein the oligonucleotide further comprises a sense strand that hybridizes to the antisense strand.

In some embodiments, the complex described herein comprises an anti-TfR1 antibody covalently linked to a molecular payload, wherein the anti-TfR1 antibody comprises a heavy chain comprising the amino acid sequence of SEQ ID NO: 102 and a light chain comprising the amino acid sequence of SEQ ID NO: 93. In some embodiments, the molecular payload is an oligonucleotide comprising an antisense strand comprising a region of complementarity of at least 16 nucleotides to a target sequence in DUX4 mRNA (e.g., a target sequence listed in Table 8), optionally wherein the antisense strand comprising at least 16 consecutive nucleotides of any one of the antisense sequences listed in Table 8, optionally wherein the oligonucleotide further comprises a sense strand that hybridizes to the antisense strand. In some embodiments, the molecular payload is an oligonucleotide comprising an antisense strand comprising a region of complementarity of at least 16 nucleotides to a target sequence in DUX4 mRNA (e.g., a target sequence listed in Table 9), optionally wherein the antisense strand comprising at least 16 consecutive nucleotides of any one of the antisense sequences listed in Table 9, optionally wherein the oligonucleotide further comprises a sense strand that hybridizes to the antisense strand.

In some embodiments, the complex described herein comprises an anti-TfR1 antibody covalently linked to a molecular payload, wherein the anti-TfR1 antibody comprises a heavy chain comprising the amino acid sequence of SEQ ID NO: 102 or SEQ ID NO: 103 and a light chain comprising the amino acid sequence of SEQ ID NO: 95. In some embodiments, the molecular payload is an oligonucleotide comprising an antisense strand comprising a region of complementarity of at least 16 nucleotides to a target sequence in DUX4 mRNA (e.g., a target sequence listed in Table 8), optionally wherein the antisense strand comprising at least 16 consecutive nucleotides of any one of the antisense sequences listed in Table 8, optionally wherein the oligonucleotide further comprises a sense strand that hybridizes to the antisense strand. In some embodiments, the molecular payload is an oligonucleotide comprising an antisense strand comprising a region of complementarity of at least 16 nucleotides to a target sequence in DUX4 mRNA (e.g., a target sequence listed in Table 9), optionally wherein the antisense strand comprising at least 16 consecutive nucleotides of any one of the antisense sequences listed in Table 9, optionally wherein the oligonucleotide further comprises a sense strand that hybridizes to the antisense strand.

In some embodiments, the complex described herein comprises an anti-TfR1 antibody covalently linked to a molecular payload, wherein the anti-TfR1 antibody comprises a heavy chain comprising the amino acid sequence of SEQ ID NO: 158 or SEQ ID NO: 159 and a light chain comprising the amino acid sequence of SEQ ID NO: 157. In some embodiments, the molecular payload is an oligonucleotide comprising an antisense strand comprising a region of complementarity of at least 16 nucleotides to a target sequence in DUX4 mRNA (e.g., a target sequence listed in Table 8), optionally wherein the antisense strand comprising at least 16 consecutive nucleotides of any one of the antisense sequences listed in Table 8, optionally wherein the oligonucleotide further comprises a sense strand that hybridizes to the antisense strand. In some embodiments, the molecular payload is an oligonucleotide comprising an antisense strand comprising a region of complementarity of at least 16 nucleotides to a target sequence in DUX4 mRNA (e.g., a target sequence listed in Table 9), optionally wherein the antisense strand comprising at least 16 consecutive nucleotides of any one of the antisense sequences listed in Table 9, optionally wherein the oligonucleotide further comprises a sense strand that hybridizes to the antisense strand.

In some embodiments, the molecular payload is an oligonucleotide (e.g., an oligonucleotide listed in Table 8). In some embodiments, the molecular payload is an oligonucleotide (e.g., an oligonucleotide listed in Table 9).

In any of the example complexes described herein, in some embodiments, the anti-TfR1 antibody is covalently linked to the molecular payload via a linker comprising a structure of formula (I):

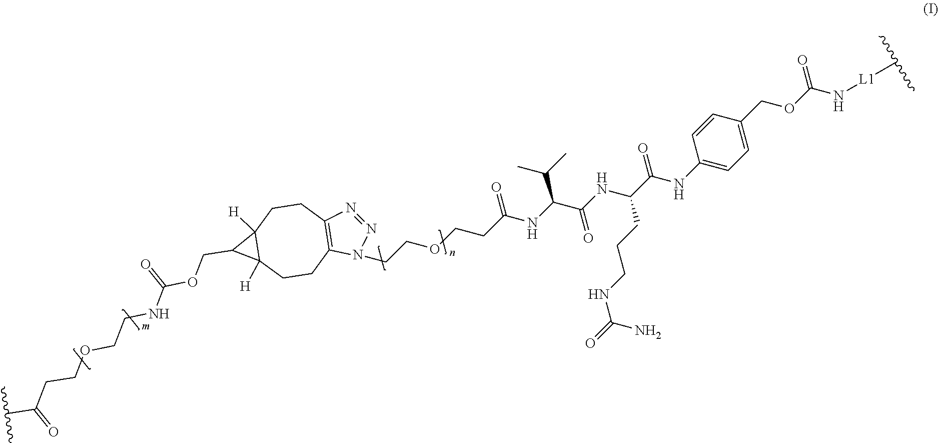

wherein n is 3, m is 4.

In some embodiments, the complex described herein comprises an anti-TfR1 antibody covalently linked to the 3′ or 5′ end of an oligonucleotide described herein (e.g., the sense or antisense strand of an oligonucleotide listed in Table 8) via a lysine in the anti-TfR1 antibody, wherein the anti-TfR1 antibody comprises a CDR-H1, a CDR-H2, a CDR-H3, a CDR-L1, a CDR-L2, and a CDR-L3 of any one of the antibodies listed in Table 2, wherein the complex has a structure of formula (E):

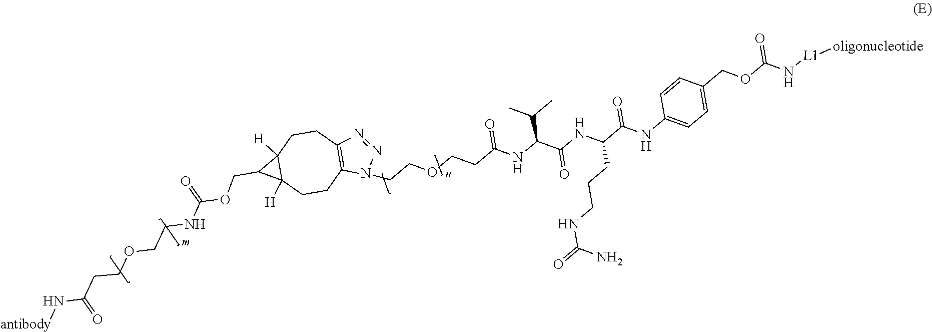

wherein n is 3 and m is 4. It should be understood that the amide shown adjacent the anti-TfR1 antibody in Formula (E) results from a reaction with an amine of the anti-TfR1 antibody, such as a lysine epsilon amine. In some embodiments, the anti-TfR1 antibody is covalently linked to the 5′ end of the sense strand of an oligonucleotide described herein. In some embodiments, the anti-TfR1 antibody is covalently linked to the 3′ end of the sense strand of an oligonucleotide described herein.

In some embodiments, the complex described herein comprises an anti-TfR1 antibody covalently linked to the 3′ or 5′ end of an oligonucleotide described herein (e.g., the sense or antisense strand of an oligonucleotide listed in Table 9) via a lysine in the anti-TfR1 antibody, wherein the anti-TfR1 antibody comprises a CDR-H1, a CDR-H2, a CDR-H3, a CDR-L1, a CDR-L2, and a CDR-L3 of any one of the antibodies listed in Table 2, wherein the complex has a structure of formula (E):

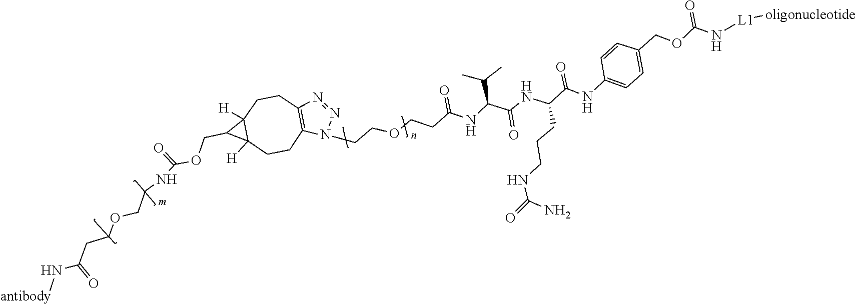

wherein n is 3 and m is 4. It should be understood that the amide shown adjacent the anti-TfR1 antibody in Formula (E) results from a reaction with an amine of the anti-TfR1 antibody, such as a lysine epsilon amine. In some embodiments, the anti-TfR1 antibody is covalently linked to the 5′ end or 3′ end of the sense strand of an oligonucleotide described herein. In some embodiments, the anti-TfR1 antibody is covalently linked to the 5′ end of the sense strand of an oligonucleotide described herein. In some embodiments, the anti-TfR1 antibody is covalently linked to the 3′ end of the sense strand of an oligonucleotide described herein.

In some embodiments, the complex described herein comprises an anti-TfR1 antibody covalently linked to the 3′ or 5′ end of an oligonucleotide described herein (e.g., the sense or antisense strand of an oligonucleotide listed in Table 8) via a lysine in the anti-TfR1 antibody, wherein the anti-TfR1 antibody comprises a VH and VL of any one of the antibodies listed in Table 3, wherein the complex has a structure of formula (E):

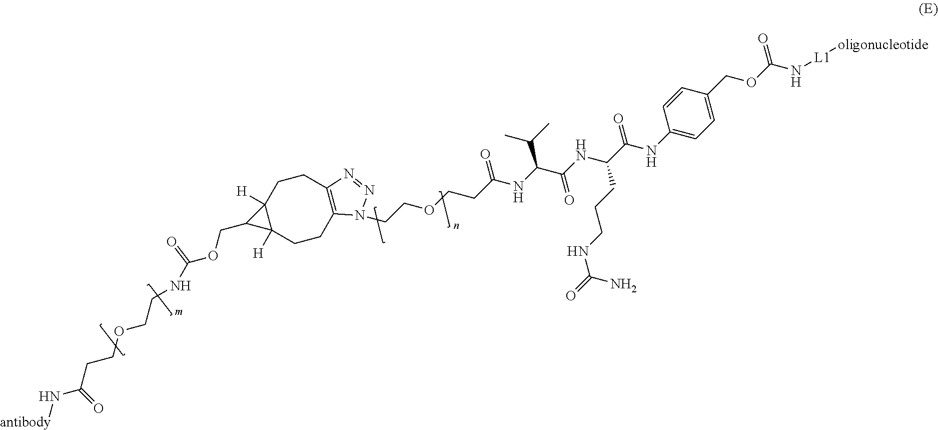

wherein n is 3 and m is 4. It should be understood that the amide shown adjacent the anti-TfR1 antibody in Formula (E) results from a reaction with an amine of the anti-TfR1 antibody, such as a lysine epsilon amine. In some embodiments, the anti-TfR1 antibody is covalently linked to the 5′ end of the sense strand of an oligonucleotide described herein. In some embodiments, the anti-TfR1 antibody is covalently linked to the 3′ end of the sense strand of an oligonucleotide described herein.

In some embodiments, the complex described herein comprises an anti-TfR1 antibody covalently linked to the 3′ or 5′ end of an oligonucleotide described herein (e.g., the sense or antisense strand of an oligonucleotide listed in Table 9) via a lysine in the anti-TfR1 antibody, wherein the anti-TfR1 antibody comprises a VH and VL of any one of the antibodies listed in Table 3, wherein the complex has a structure of formula (E):

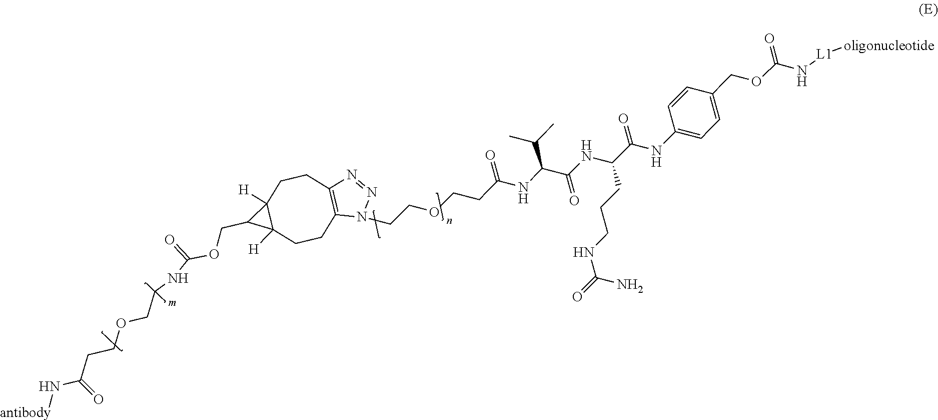

wherein n is 3 and m is 4. It should be understood that the amide shown adjacent the anti-TfR1 antibody in Formula (E) results from a reaction with an amine of the anti-TfR1 antibody, such as a lysine epsilon amine. In some embodiments, the anti-TfR1 antibody is covalently linked to the 5′ end or 3′ end of the sense strand of an oligonucleotide described herein. In some embodiments, the anti-TfR1 antibody is covalently linked to the 5′ end of the sense strand of an oligonucleotide described herein. In some embodiments, the anti-TfR1 antibody is covalently linked to the 3′ end of the sense strand of an oligonucleotide described herein. In some embodiments, the complex described herein comprises an anti-TfR1 antibody covalently linked to the 3′ or 5′ end of an oligonucleotide described herein (e.g., the sense or antisense strand of an oligonucleotide listed in Table 8) via a lysine in the anti-TfR1 antibody, wherein the anti-TfR1 antibody comprises a heavy chain and light chain of any one of the antibodies listed in Table 4, wherein the complex has a structure of formula (E):

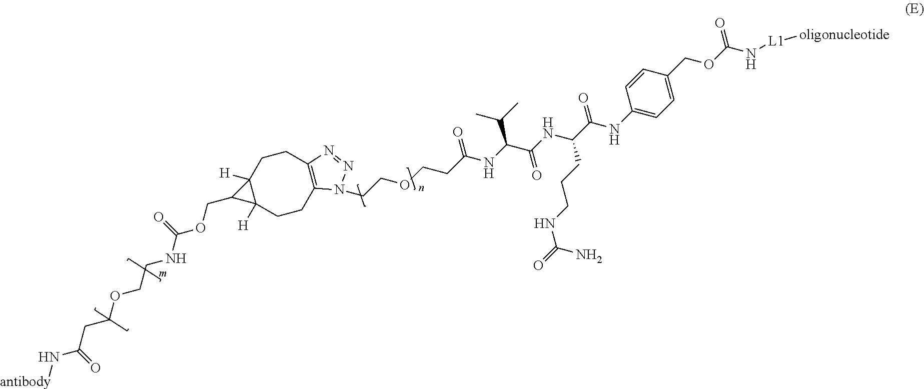

wherein n is 3 and m is 4. It should be understood that the amide shown adjacent the anti-TfR1 antibody in Formula (E) results from a reaction with an amine of the anti-TfR1 antibody, such as a lysine epsilon amine. In some embodiments, the anti-TfR1 antibody is covalently linked to the 5′ end of the sense strand of an oligonucleotide described herein. In some embodiments, the anti-TfR1 antibody is covalently linked to the 3′ end of the sense strand of an oligonucleotide described herein.

In some embodiments, the complex described herein comprises an anti-TfR1 antibody covalently linked to the 3′ or 5′ end of an oligonucleotide described herein (e.g., the sense or antisense strand of an oligonucleotide listed in Table 9) via a lysine in the anti-TfR1 antibody, wherein the anti-TfR1 antibody comprises a heavy chain and light chain of any one of the antibodies listed in Table 4, wherein the complex has a structure of formula (E):

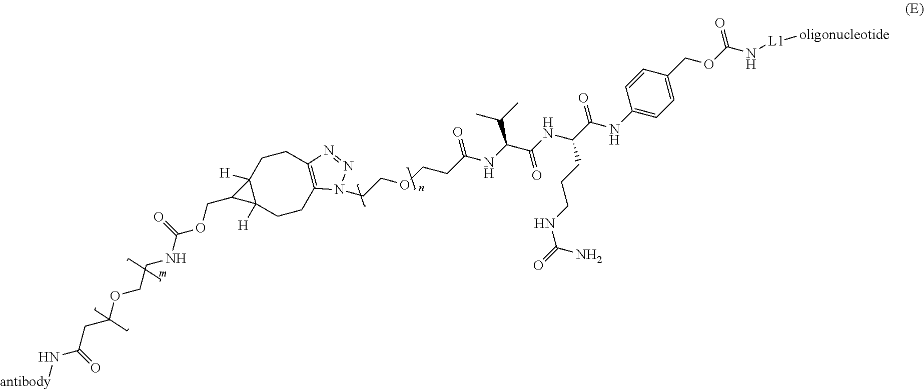

wherein n is 3 and m is 4. It should be understood that the amide shown adjacent the anti-TfR1 antibody in Formula (E) results from a reaction with an amine of the anti-TfR1 antibody, such as a lysine epsilon amine. In some embodiments, the anti-TfR1 antibody is covalently linked to the 5′ end or 3′ end of the sense strand of an oligonucleotide described herein. In some embodiments, the anti-TfR1 antibody is covalently linked to the 5′ end of the sense strand of an oligonucleotide described herein. In some embodiments, the anti-TfR1 antibody is covalently linked to the 3′ end of the sense strand of an oligonucleotide described herein.

In some embodiments, the complex described herein comprises an anti-TfR1 antibody that is a Fab covalently linked to the 3′ or 5′ end of an oligonucleotide described herein (e.g., the sense or antisense strand of an oligonucleotide listed in Table 8) via a lysine in the anti-TfR1 Fab, wherein the anti-TfR1 Fab comprises a heavy chain and light chain of any one of the antibodies listed in Table 5, wherein the complex has a structure of formula (E):

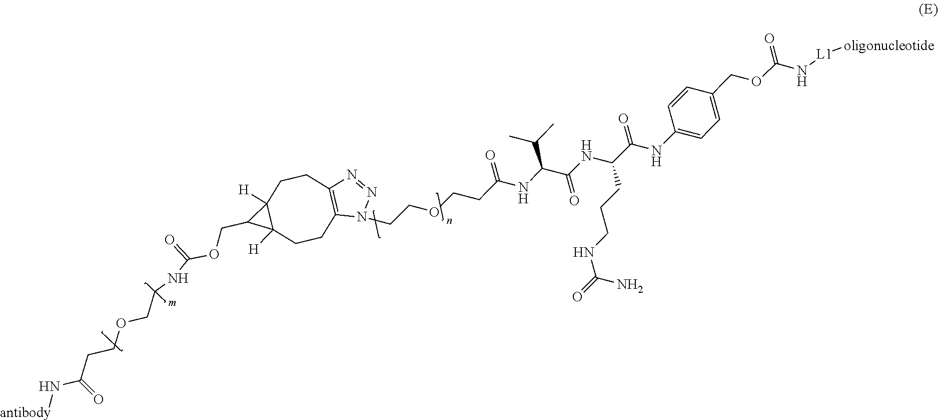

wherein n is 3 and m is 4. It should be understood that the amide shown adjacent the anti-TfR1 antibody in Formula (E) results from a reaction with an amine of the anti-TfR1 antibody, such as a lysine epsilon amine. In some embodiments, the anti-TfR1 antibody is covalently linked to the 5′ end of the sense strand of an oligonucleotide described herein. In some embodiments, the anti-TfR1 antibody is covalently linked to the 3′ end of the sense strand of an oligonucleotide described herein.

In some embodiments, the complex described herein comprises an anti-TfR1 antibody that is a Fab covalently linked to the 3′ or 5′ end of an oligonucleotide described herein (e.g., the sense or antisense strand of an oligonucleotide listed in Table 9) via a lysine in the anti-TfR1 Fab, wherein the anti-TfR1 Fab comprises a heavy chain and light chain of any one of the antibodies listed in Table 5, wherein the complex has a structure of formula (E):

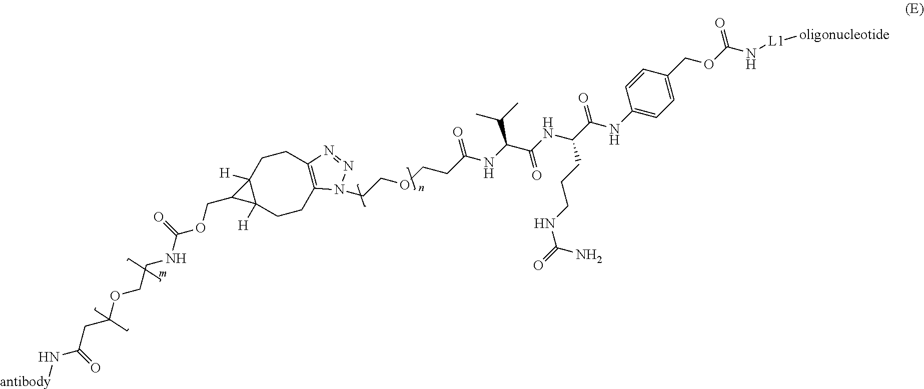

wherein n is 3 and m is 4. It should be understood that the amide shown adjacent the anti-TfR1 antibody in Formula (E) results from a reaction with an amine of the anti-TfR1 antibody, such as a lysine epsilon amine. In some embodiments, the anti-TfR1 antibody is covalently linked to the 5′ end or 3′ end of the sense strand of an oligonucleotide described herein. In some embodiments, the anti-TfR1 antibody is covalently linked to the 5′ end of the sense strand of an oligonucleotide described herein. In some embodiments, the anti-TfR1 antibody is covalently linked to the 3′ end of the sense strand of an oligonucleotide described herein.

In some embodiments, the complex described herein comprises an anti-TfR1 antibody covalently linked to the 3′ or 5′ end of an oligonucleotide described herein (e.g., the sense or antisense strand of an oligonucleotide listed in Table 8) via a lysine in the anti-TfR1 antibody, wherein the anti-TfR1 antibody comprises:
(i) a CDR-H1 comprising the amino acid sequence of SEQ ID NO: 27, a CDR-H2 comprising the amino acid sequence of SEQ ID NO: 28, a CDR-H3 comprising the amino acid sequence of SEQ ID NO: 29, a CDR-L1 comprising the amino acid sequence of SEQ ID NO: 30, a CDR-L2 comprising the amino acid sequence of SEQ ID NO: 31, and a CDR-L3 comprising the amino acid sequence of SEQ ID NO: 32;
(ii) a CDR-H1 comprising the amino acid sequence of SEQ ID NO: 33, a CDR-H2 comprising the amino acid sequence of SEQ ID NO: 34, a CDR-H3 comprising the amino acid sequence of SEQ ID NO: 35, a CDR-L1 comprising the amino acid sequence of SEQ ID NO: 36, a CDR-L2 comprising the amino acid sequence of SEQ ID NO: 37, and a CDR-L3 comprising the amino acid sequence of SEQ ID NO: 32; or
(ii) a CDR-H1 comprising the amino acid sequence of SEQ ID NO: 38, a CDR-H2 comprising the amino acid sequence of SEQ ID NO: 39, a CDR-H3 comprising the amino acid sequence of SEQ ID NO: 40, a CDR-L1 comprising the amino acid sequence of SEQ ID NO: 41, a CDR-L2 comprising the amino acid sequence of SEQ ID NO: 31, and a CDR-L3 comprising the amino acid sequence of SEQ ID NO: 42,
wherein the complex has a structure of formula (E):

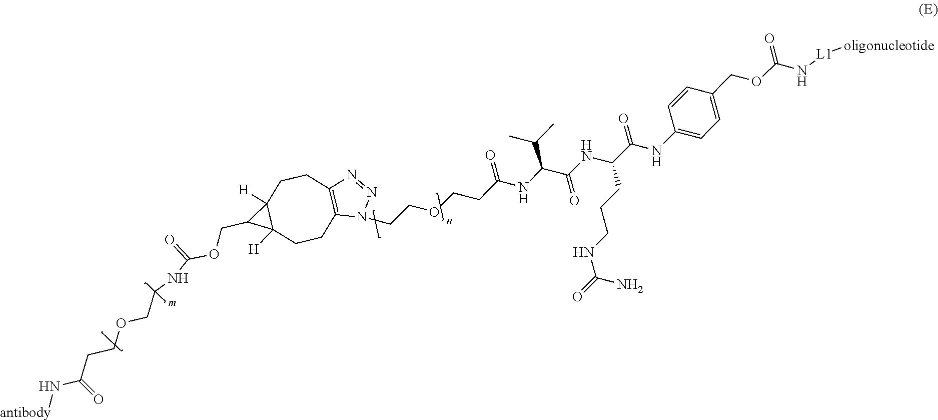

wherein n is 3 and m is 4. It should be understood that the amide shown adjacent the anti-TfR1 antibody in Formula (E) results from a reaction with an amine of the anti-TfR1 antibody, such as a lysine epsilon amine. In some embodiments, the anti-TfR1 antibody is covalently linked to the 5′ end of the sense strand of an oligonucleotide described herein. In some embodiments, the anti-TfR1 antibody is covalently linked to the 3′ end of the sense strand of an oligonucleotide described herein.

In some embodiments, the complex described herein comprises an anti-TfR1 antibody covalently linked to the 3′ or 5′ end of an oligonucleotide described herein (e.g., the sense or antisense strand of an oligonucleotide listed in Table 9) via a lysine in the anti-TfR1 antibody, wherein the anti-TfR1 antibody comprises:
(i) a CDR-H1 comprising the amino acid sequence of SEQ ID NO: 27, a CDR-H2 comprising the amino acid sequence of SEQ ID NO: 28, a CDR-H3 comprising the amino acid sequence of SEQ ID NO: 29, a CDR-L1 comprising the amino acid sequence of SEQ ID NO: 30, a CDR-L2 comprising the amino acid sequence of SEQ ID NO: 31, and a CDR-L3 comprising the amino acid sequence of SEQ ID NO: 32;
(ii) a CDR-H1 comprising the amino acid sequence of SEQ ID NO: 33, a CDR-H2 comprising the amino acid sequence of SEQ ID NO: 34, a CDR-H3 comprising the amino acid sequence of SEQ ID NO: 35, a CDR-L1 comprising the amino acid sequence of SEQ ID NO: 36, a CDR-L2 comprising the amino acid sequence of SEQ ID NO: 37, and a CDR-L3 comprising the amino acid sequence of SEQ ID NO: 32; or
(ii) a CDR-H1 comprising the amino acid sequence of SEQ ID NO: 38, a CDR-H2 comprising the amino acid sequence of SEQ ID NO: 39, a CDR-H3 comprising the amino acid sequence of SEQ ID NO: 40, a CDR-L1 comprising the amino acid sequence of SEQ ID NO: 41, a CDR-L2 comprising the amino acid sequence of SEQ ID NO: 31, and a CDR-L3 comprising the amino acid sequence of SEQ ID NO: 42,
wherein the complex has a structure of formula (E):

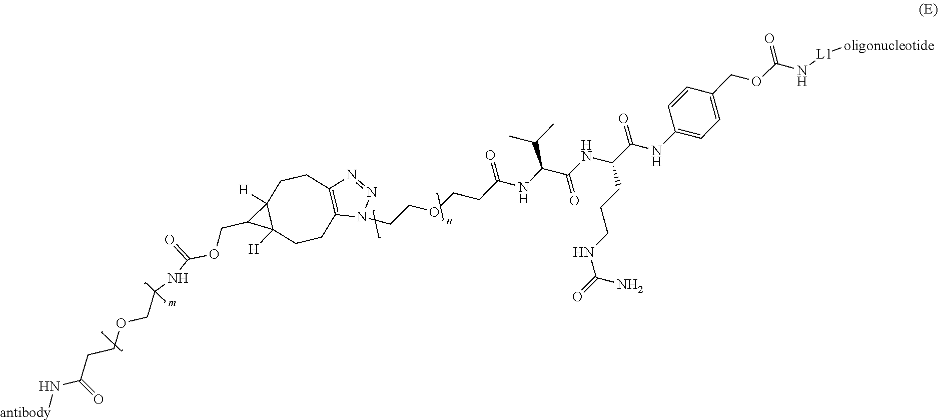

wherein n is 3 and m is 4. It should be understood that the amide shown adjacent the anti-TfR1 antibody in Formula (E) results from a reaction with an amine of the anti-TfR1 antibody, such as a lysine epsilon amine. In some embodiments, the anti-TfR1 antibody is covalently linked to the 5′ end or 3′ end of the sense strand of an oligonucleotide described herein. In some embodiments, the anti-TfR1 antibody is covalently linked to the 5′ end of the sense strand of an oligonucleotide described herein. In some embodiments, the anti-TfR1 antibody is covalently linked to the 3′ end of the sense strand of an oligonucleotide described herein.

In some embodiments, the complex described herein comprises an anti-TfR1 antibody covalently linked to the 3′ or 5′ end of an oligonucleotide described herein (e.g., the sense or antisense strand of an oligonucleotide listed in Table 8) via a lysine in the anti-TfR1 antibody, wherein the anti-TfR1 antibody a VH comprising the amino acid sequence of SEQ ID NO: 76, and a VL comprising the amino acid sequence of SEQ ID NO: 75, wherein the complex has a structure of formula (E):

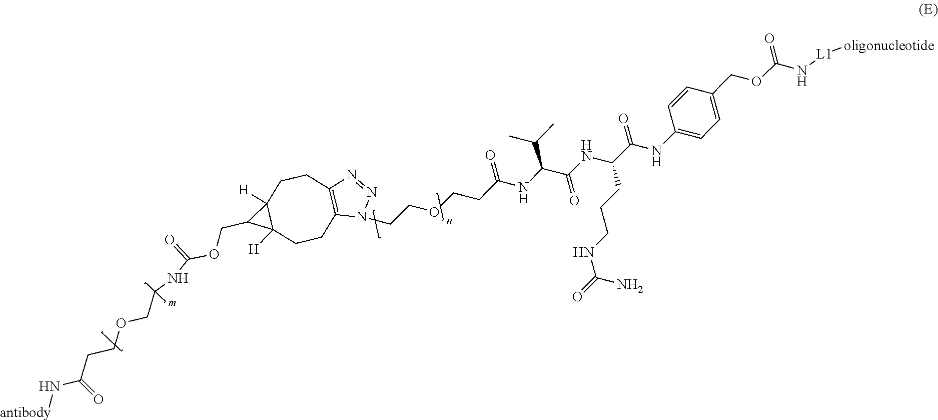

wherein n is 3 and m is 4. It should be understood that the amide shown adjacent the anti-TfR1 antibody in Formula (E) results from a reaction with an amine of the anti-TfR1 antibody, such as a lysine epsilon amine. In some embodiments, the anti-TfR1 antibody is covalently linked to the 5′ end of the sense strand of an oligonucleotide described herein. In some embodiments, the anti-TfR1 antibody is covalently linked to the 3′ end of the sense strand of an oligonucleotide described herein.

In some embodiments, the complex described herein comprises an anti-TfR1 antibody covalently linked to the 3′ or 5′ end of an oligonucleotide described herein (e.g., the sense or antisense strand of an oligonucleotide listed in Table 9) via a lysine in the anti-TfR1 antibody, wherein the anti-TfR1 antibody a VH comprising the amino acid sequence of SEQ ID NO: 76, and a VL comprising the amino acid sequence of SEQ ID NO: 75, wherein the complex has a structure of formula (E):

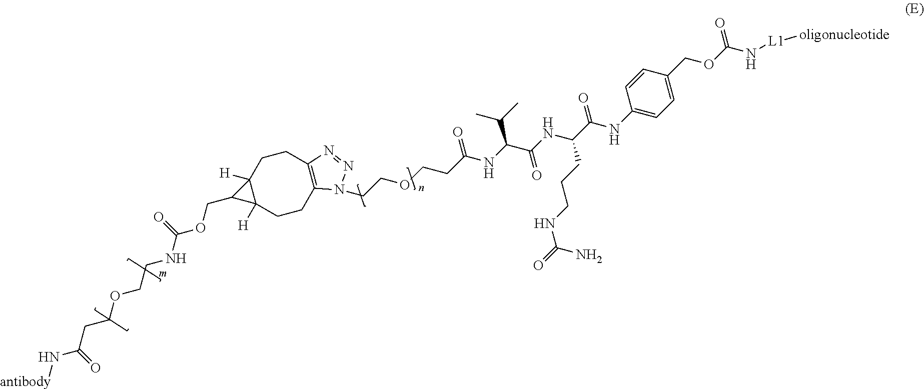

wherein n is 3 and m is 4. It should be understood that the amide shown adjacent the anti-TfR1 antibody in Formula (E) results from a reaction with an amine of the anti-TfR1 antibody, such as a lysine epsilon amine. In some embodiments, the anti-TfR1 antibody is covalently linked to the 5′ end or 3′ end of the sense strand of an oligonucleotide described herein. In some embodiments, the anti-TfR1 antibody is covalently linked to the 5′ end of the sense strand of an oligonucleotide described herein. In some embodiments, the anti-TfR1 antibody is covalently linked to the 3′ end of the sense strand of an oligonucleotide described herein.

In some embodiments, the complex described herein comprises an anti-TfR1 antibody that is a Fab covalently linked to the 3′ or 5′ end of an oligonucleotide described herein (e.g., the sense or antisense strand of an oligonucleotide listed in Table 8) via a lysine in the anti-TfR1 Fab, wherein the anti-TfR1 Fab comprises a heavy chain comprising the amino acid sequence of SEQ ID NO: 101 and a light chain comprising the amino acid sequence of SEQ ID NO: 90, wherein the complex has a structure of formula (E):

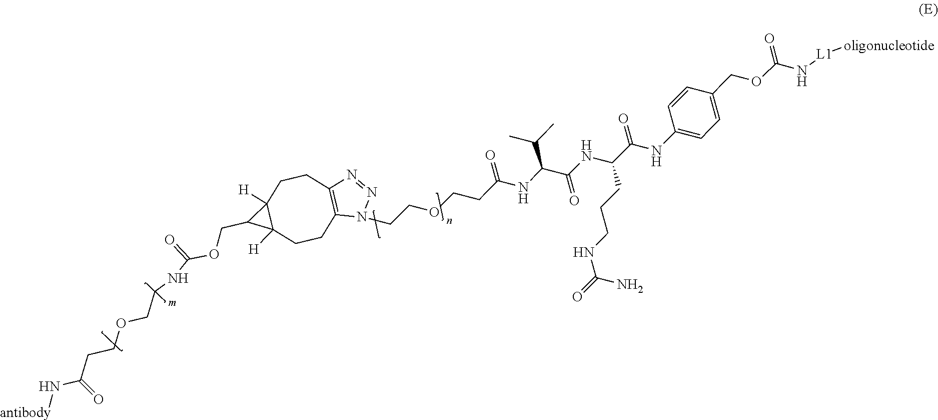

wherein n is 3 and m is 4. It should be understood that the amide shown adjacent the anti-TfR1 antibody in Formula (E) results from a reaction with an amine of the anti-TfR1 antibody, such as a lysine epsilon amine. In some embodiments, the anti-TfR1 antibody is covalently linked to the 5′ end of the sense strand of an oligonucleotide described herein. In some embodiments, the anti-TfR1 antibody is covalently linked to the 3′ end of the sense strand of an oligonucleotide described herein.

In some embodiments, the complex described herein comprises an anti-TfR1 antibody that is a Fab covalently linked to the 3′ or 5′ end of an oligonucleotide described herein (e.g., the sense or antisense strand of an oligonucleotide listed in Table 9) via a lysine in the anti-TfR1 Fab, wherein the anti-TfR1 Fab comprises a heavy chain comprising the amino acid sequence of SEQ ID NO: 101 and a light chain comprising the amino acid sequence of SEQ ID NO: 90, wherein the complex has a structure of formula (E):

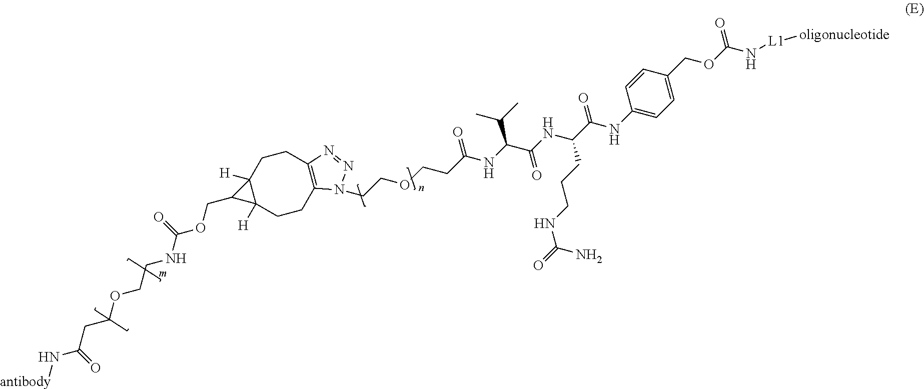

wherein n is 3 and m is 4. It should be understood that the amide shown adjacent the anti-TfR1 antibody in Formula (E) results from a reaction with an amine of the anti-TfR1 antibody, such as a lysine epsilon amine. In some embodiments, the anti-TfR1 antibody is covalently linked to the 5′ end or 3′ end of the sense strand of an oligonucleotide described herein. In some embodiments, the anti-TfR1 antibody is covalently linked to the 5′ end of the sense strand of an oligonucleotide described herein. In some embodiments, the anti-TfR1 antibody is covalently linked to the 3′ end of the sense strand of an oligonucleotide described herein.

In some embodiments, in any one of the examples of complexes described herein, L1 is:

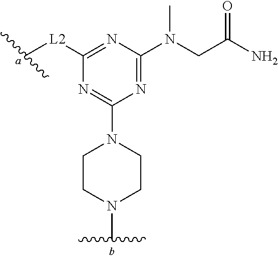

wherein L2 is

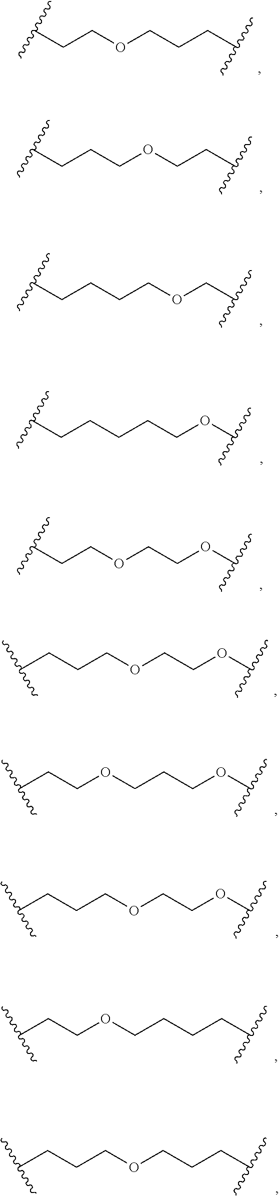

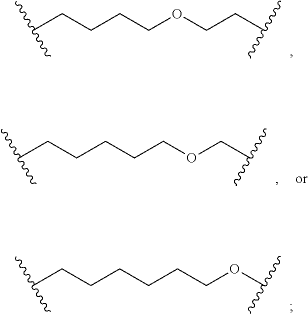 [SMILES: *CCCCOCC* ; *CCCCCOC* ; *CCCCCCO*]

wherein a labels the site directly linked to the carbamate moiety of formulae (B), (D), (E), and (I); and b labels the site covalently linked (directly or via additional chemical moieties) to the oligonucleotide.

In some embodiments. L1 is:

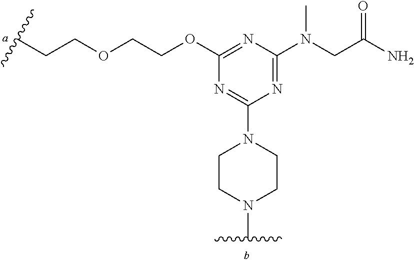 [SMILES: *CCOCCOc1nc(N(C)CC(N)=O)nc(N2CCN(*)CC2)n1]

wherein a labels the site directly linked to the carbamate moiety of formulae (B), (D), (E), and (I); and b labels the site covalently linked (directly or via additional chemical moieties) to the oligonucleotide.

In some embodiments, L1 is

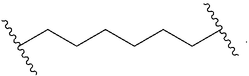 [SMILES: *CCCCCC*]

In some embodiments, L1 is linked to a 5′ phosphate of the oligonucleotide. In some embodiments, the linkage of L1 to a 5′ phosphate of the oligonucleotide forms a phosphodiester bond between L1 and the oligonucleotide.

In some embodiments, L1 is linked to a 3′ phosphate of the oligonucleotide. In some embodiments, the linkage of L1 to a 3′ phosphate of the oligonucleotide forms a phosphodiester bond between L1 and the oligonucleotide.

In some embodiments, L1 is optional (e.g., need not be present).

### III. Formulations

Complexes provided herein may be formulated in any suitable manner. Generally, complexes provided herein are formulated in a manner suitable for pharmaceutical use. For example, complexes can be delivered to a subject using a formulation that minimizes degradation, facilitates delivery and/or (e.g., and) uptake, or provides another beneficial property to the complexes in the formulation. In some embodiments, provided herein are compositions comprising complexes and pharmaceutically acceptable carriers. Such compositions can be suitably formulated such that when administered to a subject, either into the immediate environment of a target cell or systemically, a sufficient amount of the complexes enter target muscle cells. In some embodiments, complexes are formulated in buffer solutions such as phosphate-buffered saline solutions, liposomes, micellar structures, and capsids.

It should be appreciated that, in some embodiments, compositions may include separately one or more components of complexes provided herein (e.g., muscle-targeting agents, linkers, molecular payloads, or precursor molecules of any one of them).

In some embodiments, complexes are formulated in water or in an aqueous solution (e.g., water with pH adjustments). In some embodiments, complexes are formulated in basic buffered aqueous solutions (e.g., PBS). In some embodiments, formulations as disclosed herein comprise an excipient. In some embodiments, an excipient confers to a composition improved stability, improved absorption, improved solubility and/or (e.g., and) therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethyl sulfoxide, or mineral oil).

In some embodiments, a complex or component thereof (e.g., oligonucleotide or antibody) is lyophilized for extending its shelf-life and then made into a solution before use (e.g., administration to a subject). Accordingly, an excipient in a composition comprising a complex, or component thereof, described herein may be a lyoprotectant (e.g., mannitol, lactose, polyethylene glycol, or polyvinyl pyrolidone), or a collapse temperature modifier (e.g., dextran, ficoll, or gelatin).

In some embodiments, a pharmaceutical composition is formulated to be compatible with its intended route of administration. Examples of routes of administration include parenteral, e.g., intravenous, intradermal, subcutaneous, administration. Typically, the route of administration is intravenous or subcutaneous.

Pharmaceutical compositions suitable for injectable use include sterile aqueous solutions (where water soluble) or dispersions and sterile powders for the extemporaneous preparation of sterile injectable solutions or dispersions. The carrier can be a solvent or dispersion medium containing, for example, water, ethanol, polyol (for example, glycerol, propylene glycol, and liquid polyethylene glycol, and the like), and suitable mixtures thereof. In some embodiments, formulations include isotonic agents, for example, sugars, polyalcohols such as mannitol, sorbitol, and sodium chloride in the composition. Sterile injectable solutions can be prepared by incorporating the complexes in a required amount in a selected solvent with one or a combination of ingredients enumerated above, as required, followed by filtered sterilization.

In some embodiments, a composition may contain at least about 0.1% of the complex, or component thereof, or more, although the percentage of the active ingredient(s) may be between about 1% and about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

### IV. Methods of Use/Treatment

Complexes comprising a muscle-targeting agent covalently linked to a molecular payload as described herein are effective in treating FSHD. In some embodiments, complexes are effective in treating Type 1 FSHD. In some embodiments, complexes are effective in treating Type 2 FSHD. In some embodiments, FSHD is associated with deletions in D4Z4 repeat regions on chromosome 4 which contain the DUX4 gene. In some embodiments, FSHD is associated with mutations in the SMCHD1 gene. In some embodiments, FSHD is associated with mutations in the DNMT3B gene. In some embodiments, FSHD is associated with mutations in the LRIF1 gene. In some embodiments, a subject may be a human subject, a non-human primate subject, a rodent subject, or any suitable mammalian subject. In some embodiments, a subject may have myotonic dystrophy. In some embodiments, a subject has elevated expression of the DUX4 gene outside of fetal development and the testes. In some embodiments, the subject has facioscapulohumeral muscular dystrophy of Type 1 or Type 2. In some embodiments, the subject having FSHD has mutations in the SMCHD1 gene. In some embodiments, the subject having FSHD has mutations in the DNMT3B gene. In some embodiments, the subject having FSHD has mutations in the LRIF1 gene. In some embodiments, the subject having FSHD has deletion mutations in D4Z4 repeat regions on chromosome 4.

An aspect of the disclosure includes methods involving administering to a subject an effective amount of a complex as described herein. In some embodiments, an effective amount of a pharmaceutical composition that comprises a complex comprising a muscle-targeting agent covalently linked to a molecular payload can be administered to a subject in need of treatment. In some embodiments, a pharmaceutical composition comprising a complex as described herein may be administered by a suitable route, which may include intravenous administration, e.g., as a bolus or by continuous infusion over a period of time. In some embodiments, intravenous administration may be performed by intramuscular, intraperitoneal, intracerebrospinal, subcutaneous, intra-articular, intrasynovial, or intrathecal routes. In some embodiments, a pharmaceutical composition may be in solid form, aqueous form, or a liquid form. In some embodiments, an aqueous or liquid form may be nebulized or lyophilized. In some embodiments, a nebulized or lyophilized form may be reconstituted with an aqueous or liquid solution.

Compositions for intravenous administration may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In some embodiments, a pharmaceutical composition that comprises a complex comprising a muscle-targeting agent covalently linked to a molecular payload is administered via site-specific or local delivery techniques. Examples of these techniques include implantable depot sources of the complex, local delivery catheters, site specific carriers, direct injection, or direct application.

In some embodiments, a pharmaceutical composition that comprises a complex comprising a muscle-targeting agent covalently linked to a molecular payload is administered at an effective concentration that confers therapeutic effect on a subject. Effective amounts vary, as recognized by those skilled in the art, depending on the severity of the disease, unique characteristics of the subject being treated, e.g., age, physical conditions, health, or weight, the duration of the treatment, the nature of any concurrent therapies, the route of administration and related factors. These related factors are known to those in the art and may be addressed with no more than routine experimentation. In some embodiments, an effective concentration is the maximum dose that is considered to be safe for the patient. In some embodiments, an effective concentration will be the lowest possible concentration that provides maximum efficacy.

Empirical considerations, e.g., the half-life of the complex in a subject, generally will contribute to determination of the concentration of pharmaceutical composition that is used for treatment. The frequency of administration may be empirically determined and adjusted to maximize the efficacy of the treatment.

Generally, for administration of any of the complexes described herein, an initial candidate dosage may be about 1 to 100 mg/kg, or more, depending on the factors described above, e.g., safety or efficacy. In some embodiments, a treatment will be administered once. In some embodiments, a treatment will be administered daily, biweekly, weekly, bimonthly, monthly, or at any time interval that provide maximum efficacy while minimizing safety risks to the subject. Generally, the efficacy and the treatment and safety risks may be monitored throughout the course of treatment.

The efficacy of treatment may be assessed using any suitable methods. In some embodiments, the efficacy of treatment may be assessed by evaluation of observation of symptoms associated with FSHD including muscle mass loss and muscle atrophy, primarily in the muscles of the face, shoulder blades, and upper arms.

In some embodiments, a pharmaceutical composition that comprises a complex comprising a muscle-targeting agent covalently linked to a molecular payload described herein is administered to a subject at an effective concentration sufficient to inhibit activity or expression of a target gene by at least 10%, at least 20%, at least 30%, at least 40%, at least 50%, at least 60%, at least 70%, at least 80%, at least 90% or at least 95% relative to a control, e.g. baseline level of gene expression prior to treatment.

In some embodiments, a single dose or administration of a pharmaceutical composition that comprises a complex comprising a muscle-targeting agent covalently linked to a molecular payload described herein to a subject is sufficient to inhibit activity or expression of a target gene for at least 1-5, 1-10, 5-15, 10-20, 15-30, 20-40, 25-50, or more days. In some embodiments, a single dose or administration of a pharmaceutical composition that comprises a complex comprising a muscle-targeting agent covalently linked to a molecular payload described herein to a subject is sufficient to inhibit activity or expression of a target gene for at least 1, 2, 3, 4, 5, 6, 7, 8, 9, 10, 11, or 12 weeks. In some embodiments, a single dose or administration of a pharmaceutical composition that comprises a complex comprising a muscle-targeting agent covalently linked to a molecular payload described herein to a subject is sufficient to inhibit activity or expression of a target gene for at least 1, 2, 3, 4, 5, or 6 months.

In some embodiments, a pharmaceutical composition may comprise more than one complex comprising a muscle-targeting agent covalently linked to a molecular payload. In some embodiments, a pharmaceutical composition may further comprise any other suitable therapeutic agent for treatment of a subject, e.g. a human subject having FSHD. In some embodiments, the other therapeutic agents may enhance or supplement the effectiveness of the complexes described herein. In some embodiments, the other therapeutic agents may function to treat a different symptom or disease than the complexes described herein.

### EXAMPLES

### Example 1: Targeting gene expression with transfected antisense oligonucleotides

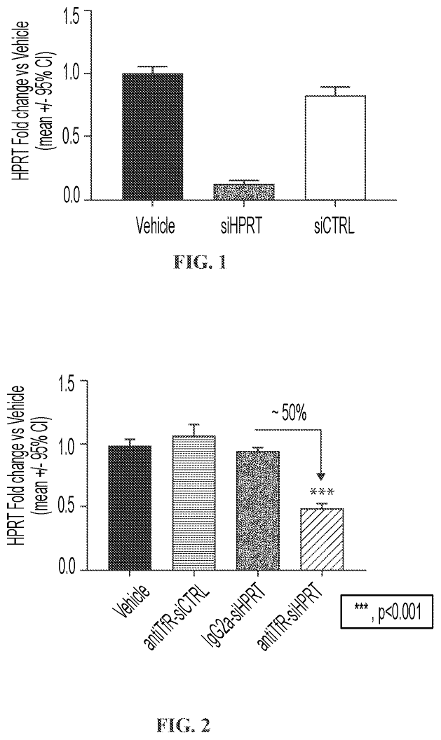

An siRNA that targets hypoxanthine phosphoribosyltransferase (HPRT) was tested in vitro for its ability to reduce expression levels of HPRT in an immortalized cell line. Briefly, Hepa 1-6 cells were transfected with either a control siRNA (siCTRL; 100 nM) or the siRNA that targets HPRT (siHPRT; 100 nM), formulated with lipofectamine 2000. HPRT expression levels were evaluated 48 hours following transfection. A control experiment was also performed in which vehicle (phosphate-buffered saline) was delivered to Hepa 1-6 cells in culture and the cells were maintained for 48 hours. As shown in FIG. 1, it was found that the HPRT siRNA reduced HPRT expression levels by about 90% compared with controls. Sequences of the siRNAs used are provided in Table 10. HPRT expression levels are indicated by HPRT mRNA expression levels.

TABLE 10
Sequences of siHPRT and siCTRL

| | Sequence | SEQ ID NO: |
|---|---|---|
| siHPRT sense strand | 5′-UcCuAuGaCuGuAgAuUuUaU-(CH₂)₆NH2-3′ | 163 |
| siHPRT antisense strand | 5′-aUaAaAuCuAcAgUcAuAgGasAsu-3′ | 164 |
| siCTRL sense strand | 5′-UgUaAuAaCcAuAuCuAcCuU-(CH₂)₆NH2-3′ | 165 |
| siCTRL antisense strand | 5′-aAgGuAgAuAuGgUuAuUaCasAsa-3′ | 166 |

*Lower case-2′ O-Me ribonucleosides′; Capital letter-2′ Fluoro ribonucleosides; s-phosphorothioate linkage

### Example 2: Targeting HPRT with a Muscle-Targeting Complex

A muscle-targeting complex was generated comprising the HPRT siRNA used in Example 1 (siHPRT) covalently linked, via a non-cleavable N-gamma-maleimidobutyryl-oxysuccinimide ester (GMBS) linker, to DTX-A-002, an anti-transferrin receptor antibody. DTX-A-002 is RI7 217 anti-TfR1 Fab.

Briefly, the GMBS linker was dissolved in dry DMSO and coupled to the 3′ end of the sense strand of siHPRT through amide bond formation under aqueous conditions. Completion of the reaction was verified by Kaiser test. Excess linker and organic solvents were removed by gel permeation chromatography. The purified, maleimide functionalized sense strand of siHPRT was then coupled to DTX-A-002 antibody using a Michael addition reaction.

The product of the antibody coupling reaction was then subjected to hydrophobic interaction chromatography (HIC-HPLC). antiTfR1-siHPRT complexes comprising one or two siHPRT molecules covalently attached to DTX-A-002 antibody were purified. Densitometry confirmed that the purified sample of complexes had an average siHPRT to antibody ratio of 1.46. SDS-PAGE analysis demonstrated that >90% of the purified sample of complexes comprised DTX-A-002 linked to either one or two siHPRT molecules.

Using the same methods as described above, a control IgG2a-siHPRT complex was generated comprising the HPRT siRNA used in Example 1 (siHPRT) covalently linked via the GMBS linker to an IgG2a (Fab) antibody (DTX-A-003). Densitometry confirmed that DTX-C-001 (the IgG2a-siHPRT complex) had an average siHPRT to antibody ratio of 1.46 and SDS-PAGE demonstrated that >90% of the purified sample of control complexes comprised DTX-A-003 linked to either one or two siHPRT molecules.

The antiTfR1-siHPRT complex was then tested for cellular internalization and inhibition of HPRT in cellulo. Hepa 1-6 cells, which have relatively high expression levels of transferrin receptor, were incubated in the presence of vehicle (phosphate-buffered saline), IgG2a-siHPRT (100 nM), antiTfR1-siCTRL (100 nM), or antiTfR1-siHPRT (100 nM), for 72 hours. After the 72 hour incubation, the cells were isolated and assayed for expression levels of HPRT (FIG. 2). Cells treated with the antiTfR1-siHPRT demonstrated a reduction in HPRT expression by ~50% relative to the cells treated with the vehicle control and to those treated with the IgG2a-siHPRT complex. Meanwhile, cells treated with either of the IgG2a-siHPRT or antiTfR1-siCTRL had HPRT expression levels comparable to the vehicle control (no reduction in HPRT expression). These data indicate that the anti-transferrin receptor antibody of the antiTfR1-siHPRT enabled cellular internalization of the complex, thereby allowing the siHPRT to inhibit expression of HPRT. HPRT expression levels are indicated by HPRT mRNA expression levels.

### Example 3: Targeting HPRT in Mouse Muscle Tissues with a Muscle-Targeting Complex

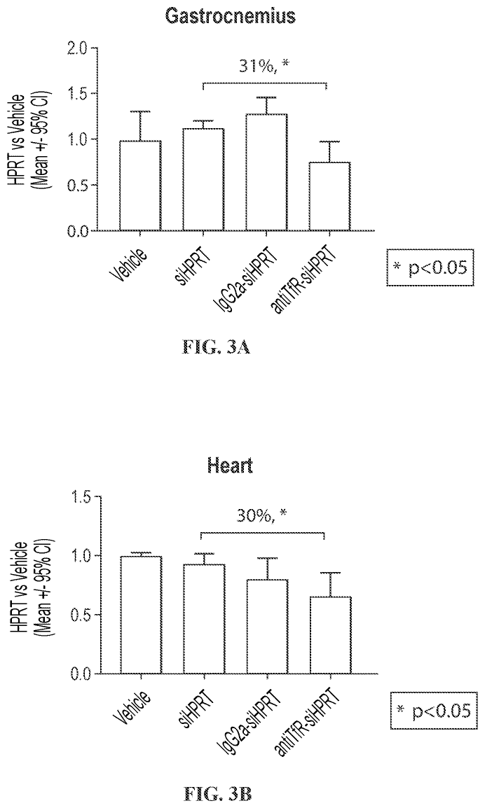

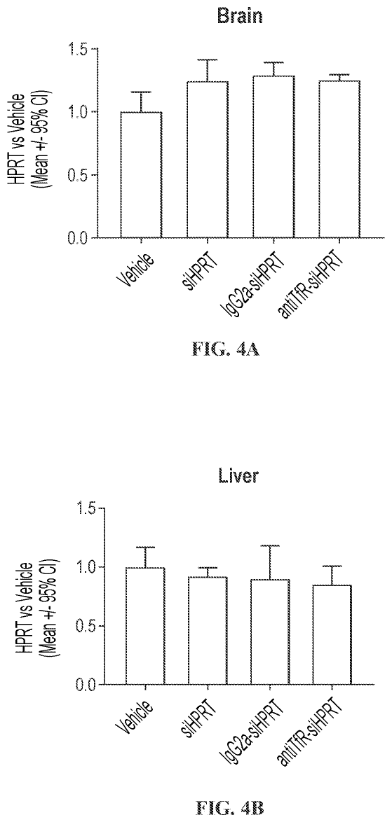

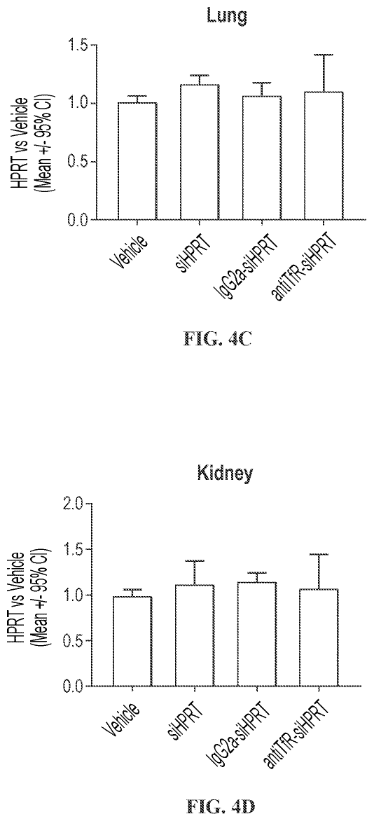

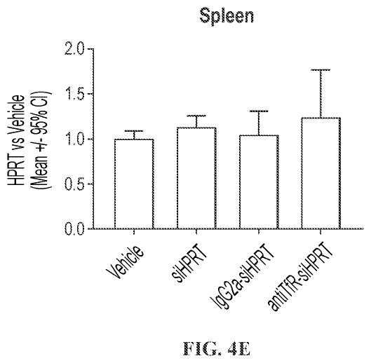

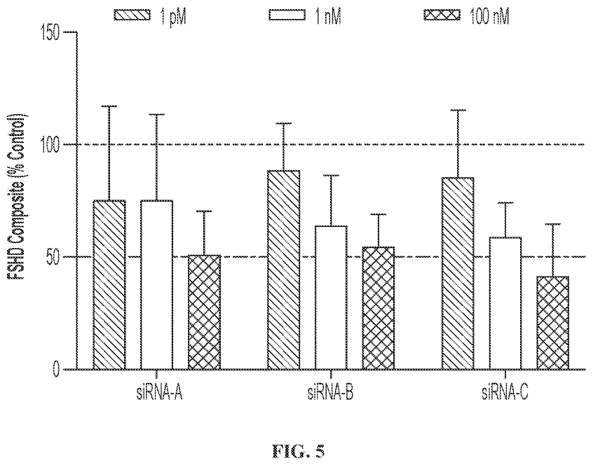

The muscle-targeting complex described in Example 2, antiTfR1-siHPRT, was tested for inhibition of HPRT in mouse tissues. C57BL/6 wild-type mice were intravenously injected with a single dose of a vehicle control (phosphate-buffered saline); siHPRT (2 mg/kg of siRNA); IgG2a-siHPRT (2 mg/kg of siRNA, corresponding to 9 mg/kg antibody complex); or antiTfR1-siHPRT (2 mg/kg of siRNA, corresponding to 9 mg/kg antibody complex. Each experimental condition was replicated in four individual C57BL/6 wild-type mice. Following a three-day period after injection, the mice were euthanized and segmented into isolated tissue types. Individual tissue samples were subsequently assayed for expression levels of HPRT (FIGS. 3A-3B and 4A-4E).

Mice treated with the antiTfR1-siHPRT complex demonstrated a reduction in HPRT expression in gastrocnemius (31% reduction; p<0.05) and heart (30% reduction; p<0.05), relative to the mice treated with the siHPRT control (FIGS. 3A-3B). Meanwhile, mice treated with the IgG2a-siHPRT complex had HPRT expression levels comparable to the siHPRT control (little or no reduction in HPRT expression) for all assayed muscle tissue types.

Mice treated with the antiTfR1-siHPRT complex demonstrated no change in HPRT expression in non-muscle tissues such as brain, liver, lung, kidney, and spleen tissues (FIGS. 4A-4E).

These data indicate that the anti-transferrin receptor antibody of the antiTfR1-siHPRT complex enabled cellular internalization of the complex into muscle-specific tissues in an in vivo mouse model, thereby allowing the siHIPRT to inhibit expression of HPRT. These data further demonstrate that the antiTfR1-oligonucleotide complexes of the current disclosure are capable of specifically targeting muscle tissues. Inhibition of HPRT is indicated by reduction in HPRT mRNA.

### Example 4: Activities of DUX4-Targeting siRNA Conjugates in FSHD Patient Myotubes

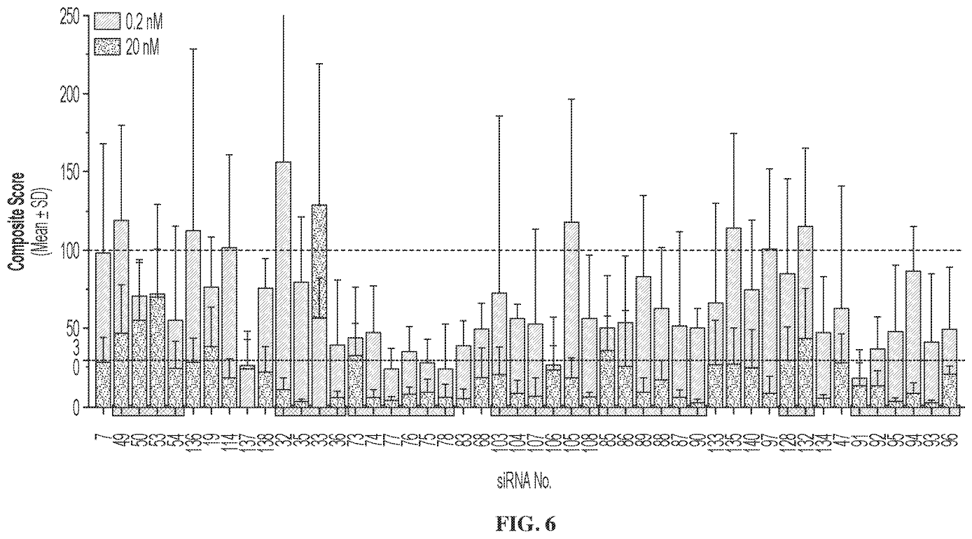

Activities of conjugates containing an anti-TfR1 Fab 3M12 VH4/VK3 covalently linked to an DUX4-targeting siRNA were tested in AB1080 immortalized FSHD patient-derived myotubes. In the conjugates, the anti-TfR1 Fab is covalently linked to the 3′ end of the sense strand of each siRNA via a linker, and the corresponding antisense strand is annealed to the sense strand. The siRNAs tested are siRNA-A (having a region of complementarity to SEQ ID NO 174), siRNA-B (having a region of complementarity to SEQ ID NO: 175), or siRNA-C(having a region of complementarity to SEQ ID NO: 176). AB1080 (C6) immortalized FSHD patient-derived myotubes were treated with the siRNA conjugates at a concentration equivalent to 1 μM, 1 nM, or 100 nM of siRNA for 10 days. cDNA was obtained from cells with the TaqMan Fast Advanced Cells-to-Ct Kit (Thermo Fisher Scientific), and levels of three DUX4 transcriptome markers MBD3L2 (Hs00544743_m1), TRIM43 (Hs00299174_m1), ZSCAN4 (Hs00537549_m1), and RPL13A (Hs04194366_g1) were analyzed via qPCR with specific TaqMan assays (Thermo Fisher Scientific). Two-step amplification reactions and fluorescence measurements for determination of cycle threshold (Ct) were conducted on a QuantStudio 7 instrument (Thermo Fisher Scientific). FSHD composite scores in FSHD1 cells were calculated using the three DUX4 transcriptome markers (MBD3L2, TRIM43, and ZSCAN4) where ΔCt=(Average Ct of 3 DUX4 markers)-(Average Ct of RPL13A), ΔΔCt=ΔCt (Treated)-ΔCt (Vehicle), FSHD Composite=2-ΔΔCT*100(%) (see FIG. 5). Results show that siRNA conjugates achieved reduction of the tested DUX4 transcriptome markers in FSHD patient-derived myotubes, and treatment with conjugate equivalent to 100 nM of siRNA achieved about 50% reduction of the tested DUX4 transcriptome markers.

### siRNA and Antibody Conjugation Protocol for Example 4

The following protocol was used to make the siRNA conjugates tested in Example 4. Conjugates containing siRNA-A, siRNA-B, siRNA-C covalently linked to an anti-TfR1 Fab 3M12 VH4/VK3 were generated. The conjugates may be generated by the reaction shown below.

### Materials:

Azide-PEG3-vc-PABC-PFP linker: MW=773.79
Azido-PEG8-PFP ester linker: MW=633.6
BCN-PEG4-PFP linker: MW=607.6
Anti-TfR1 Fab 3M12 VH4/VK3: MW=47968
siRNA-A, siRNA-B, and siRNA-C

The Anti-TfR1 Fab 3M12 VH4/VK3 was buffer exchanged into 50 mM EPPS pH of 8.0 and concentrated to a concentration of about 10 mg/ml.

The sense strand of each tested siRNA was dissolved in water to a concentration of 50 mg/ml (concentration confirmed with UV absorbance). 4x azide-linker (20 mg/ml in DMA), 50× Tributylamine (4.2M), and 70% DMA were added into the sense strand solution and incubated at room temperature overnight to generate the azido-sense strand intermediate. After incubation, 1/10 volume of 3M NaCl and 3 volume of cold isopropanol alcohol (IPA) were added into the reaction mixtures. The reaction mixtures were placed in a −80 °C freezer for 30 minutes, followed by spinning at 4500 rpm for 25 minutes. Pellets containing the azido-sense strand intermediate were washed two times with 70% ethanol (pellets were lifted with pipette tips during washing), dissolved in PBS to a final concentration of about 40 mg/ml (concentration confirmed with UV absorbance).

To generate the azide-siRNA duplex intermediate, the antisense strand of each tested siRNA was dissolved in PBS to a concentration of 50 mg/ml (concentration confirmed with UV absorbance. The azide-sense strand intermediate and the corresponding antisense strand were mixed at a 1:1 ratio. For annealing, 300-500 ml of water were heated to boiling in a glass beaker and the tubes containing the azide-sense strand intermediate and antisense strand mixture were placed into the boiling water bath for 5 minutes, and left in the water bath as it cooled down on the bench to room temperature. The annealing efficiencies for each siRNA were measured with an UPLC SEC column.

To generate the BCN-azide-siRNA duplex intermediate, azide-siRNA duplex intermediate solution in 30 mM MES, pH 5.0 was slowly added into the same volume of DMA on ice. Then 4x BCN-PEG4-PFP linker (20 mg/ml in DMA) was slowly added into the azide-siRNA duplex intermediate/DMA mixture and incubated at room temperature for 3-3.5 hours to generate the BCN-azide-siRNA duplex intermediate. Alternatively, 3x BCN-PEG4-PFP linker (20 mg/ml in DMA) may be used in this step. A UPLC C18 column was used to check the completion of reaction.

The crude reaction mixture was then purified prior to the conjugation reaction with anti-TfR1 Fab 3M12 VH4/VK3. To isolate the BCN-azide-siRNA duplex intermediate, 1/10 volume of 3M NaCl and 3 volume of cold IPA was added into the reaction mixture and placed in a −80 °C freezer for 30 minutes, followed by spinning at 4500 rpm for 25 minutes. The pellets containing the BCN-azide-siRNA duplex intermediate were washed two times with 70% ethanol (pellets were lifted with pipette tips during washing), dissolved in 20 mM MES, pH 5.0, to a concentration of about 20 mg/ml (concentration confirmed by UV absorbance).

Alternatively, the crude reaction mixture can be carried forward to the conjugation reaction immediately without any purification. In this case, a reaction mixture of 2-3 mM of azide-siRNA duplex intermediate solution in 25 mM MES, pH5.0, BCN-PEG4-PFP linker and 40% of DMA is set up and incubated in 30 °C water bath for 3-4 hours to generate the BCN-azide-siRNA duplex intermediate. The molar ratio between the azide-siRNA duplex and BCN-PEG4-PFP linker is 1:0.85.

Lastly, the anti-TfR1 Fab 3M12 VH4/VK3 was mixed with 1x (targeting DAR1) of the BCN-azide-siRNA duplex intermediate and incubated at room temperature overnight to generate the anti-TfR1 Fab-siRNA conjugates.

An alternative conjugation method can also be used. The Anti-TfR1 Fab 3M12 VH4/VK3 is buffer exchanged into 50 mM EPPS pH of 8.0 and concentrated to a concentration of about 10 mg/ml. The sense strand of each tested siRNA is dissolved in water to a concentration of 100 mg/ml (concentration confirmed with UV absorbance). 4x azide-linker (20 mg/ml in DMA), 100x Tributylamine (4.2M), and 70% DMA are added into the sense strand solution and incubated at room temperature overnight to generate the azido-sense strand intermediate. After incubation, 1/10 volume of 3M NaCl and 5 volume of cold isopropanol alcohol (IPA) are added into the reaction mixtures. The reaction mixtures are placed in a −80 °C freezer for 30 minutes, followed by spinning at 4500 rpm for 25 minutes. Pellets containing the azido-sense strand intermediate are washed two times with 80% ethanol, dissolved in water to a final concentration of about 40 mg/ml (concentration confirmed with UV absorbance).

To generate the azide-siRNA duplex intermediate, the antisense strand of each tested siRNA are dissolved in water to a concentration of 50 mg/ml. The azide-sense strand intermediate and the corresponding antisense strand are mixed at a 1:1 ratio and incubated in 30 °C water bath for more than 4 hours for annealing.

To generate the BCN-azide-siRNA duplex intermediate, a reaction mixture of 2-3 mM of azide-siRNA duplex intermediate solution in 25 mM MES, pH 5.0, 4 molar excess of BCN-PEG4-PFP linker, and 40% of DMA is set up and incubated in 30 °C water bath for 2-3 hours to generate the BCN-azide-siRNA duplex intermediate. Alternatively, 3x BCN-PEG4-PFP linker (20 mg/ml in DMA) may be used in this step. A UPLC C18 column is used to check the completion of reaction.

The crude reaction mixture was then purified prior to the conjugation reaction with anti-TfR1 Fab 3M12 VH4/VK3. To isolate the BCN-azide-siRNA duplex intermediate, 1/10 volume of 3M NaCl and 3 volume of cold IPA was added into the reaction mixture and placed in a −80 °C freezer for 30 minutes, followed by spinning at 4500 rpm for 25 minutes. The pellets containing the BCN-azide-siRNA duplex intermediate were washed two times with 80% ethanol, dissolved in water to a concentration of about 20 mg/ml.

Alternatively, the crude reaction mixture can be carried forward to the conjugation reaction immediately without any purification. In this case, a reaction mixture of 2-3 mM of azide-siRNA duplex intermediate solution in 25 mM MES, pH5.0, molar excess of BCN-PEG4-PFP linker and 40% of DMA is set up and incubated in 30 °C water bath for 3-4 hours to generate the BCN-azide-siRNA duplex intermediate. The molar ratio between the azide-siRNA duplex and BCN-PEG4-PFP linker is 1:0.85.

Lastly, the anti-TfR1 Fab 3M12 VH4/VK3 at 5-10 mg/ml in 50 mM of EPPS, pH8.5 is mixed with 1x (targeting DAR1) of the BCN-azide-siRNA duplex intermediate and incubated at room temperature overnight to generate the anti-TfR1 Fab-siRNA conjugates.

An alternative conjugation method can also be used and was used to make the siRNA conjugates tested in Examples 5-7 below. In the alternative method, anti-TfR1 Fab 3M12 VH4/VK3 in PBS (5-6 mg/ml), 1xBCN (20 mg/ml in DMA, 32.9 mM), and 10% DMA are mixed and incubated at room temperature for at least 5 hours to generate the anti-TfR1 Fab 3M12 VH4/VK3-BCN intermediate. The antibody-BCN intermediate is then purified using TFF and buffer exchanged into 50 mM EPPS, pH 8.0.

The azide-siRNA duplex intermediate is generated as described above.

The anti-TfR1 Fab 3M12 VH4/VK3-BCN intermediate (at a concentration of 6-7 mg/ml in EPPS, pH 8.0) is mixed with 1.0 molar excess of the azide-siRNA duplex intermediate, and incubated at room temperature overnight to generate the anti-TfR1 Fab-siRNA conjugates.

Purification by TSKgel superQ-5 PW Column:

The anti-TfR1 Fab-siRNA conjugates generated using either the two-step reaction or the one-step reaction were purified using the methods below.

The crude conjugation reaction products in 50 mM EPPS, pH of 8.0 were diluted with 5 column volume (cv) of 10 mM Tris, pH of 8.0. The sample was loaded at 0.5 ml/minute onto a 1 ml TSKgel superQ-5 PW column at less than 10 mg of conjugate per ml of resin. The column was washed with Buffer A (20 mM Tris, pH8.0) for 5-6 cv at 1 ml/minute. The conjugates were then eluted with 15-20 cv of an elution buffer containing 79% of Buffer A (20 mM Tris, pH8.0) and 21% Buffer B (20 mM Tris, pH8.0+1.5M NaCl) at 1 ml/minute. The eluted conjugates were then buffer exchanged into PBS and concentrated to a concentration of >5 mg/ml.

In an alternative purification method, the crude conjugation reaction products are diluted with 1 volume of 10 mM Tris, pH of 8.0, and loaded onto a 5 ml TSKgel superQ-5 PW column. The column is eluted with a gradient of 15% Buffer B (Buffer B: 20 mM Tris, pH 8.0+1.5M NaCl; Buffer A: 20 mM Tris, pH 8.0)) to 40% Buffer B over 25 column volume (CV) at 1.5 ml/minute. The DAR 1 conjugate fractions were collected, buffer exchanged into PBS and concentrated to a concentration of >5 mg/ml.

### Example 5: Activities of DUX4-Targeting siRNA In Vitro

48 DUX4-targeting siRNAs were tested for their activities in knocking down DUX4 transcriptome in FSHD patient-derived C6 myotubes at two siRNA concentrations (0.2 nM and 20 nM). The siRNAs tested were siRNAs 49, 50, 53, 54, 136, 19, 114, 137, 138, 32, 35, 33, 36, 73, 74, 77, 76, 75, 78, 83, 68, 103, 104, 107, 106, 105, 108, 85, 86, 89, 88, 87, 90, 133, 135, 140, 97, 128, 132, 134, 47, 91, 92, 95, 94, 93, and 96. An exon 1 targeting siRNA, siRNA 7, was used as a positive control. The siRNA numbers correspond to the siRNA numbers in Table 8. Composite scores calculated using the average mRNA level of three DUX4 transcriptome markers MBD3L2, TRIM43, and ZSCAN4 in FSHD patient-derived C6 myotubes are shown in FIG. 6.

Results show that the majority of the tested siRNAs achieved a comparable level of knockdown of the tested DUX4 transcriptome markers in FSHD patient-derived C6 myotubes compared to the positive control. Some of the siRNAs had more significant knockdown compared to the positive control.

Additionally, 16 of the 48 siRNAs (siRNA 136, 137, 74, 77, 78, 104, 107, 106, 86, 89, 133, 97, 128, 92, 95, and 94) were also tested in Hepa1-6 cells at concentration of 20 nM. A DualGlo reporter-plasmid was designed for screening the siRNAs. The plasmid contains coding sequence for the human DUX4 mRNA in the 3′-UTR of a reporter luciferase. Hepa1-6 cells were transfected with the DualGlo reporter plasmid and 20 nM of the 16 DUX targeting siRNAs. siRNA 7 was also used as a positive control in Hepa1-6 cells. After transfection, cells were maintained for 1 day. The DUX4 knockdown activity of each of the 16 siRNAs is shown in Table 10, along with the DUX4 transcriptome knockdown activity of the same 16 siRNAs tested above in FSHD patient-derived C6 myotubes.

Results show that the tested siRNAs achieved a comparable or more significant level of knockdown of the DUX4 in Hepa1-6 cells and DUX4 transcriptome in FSHD patient-derived C6 myotubes compared to the positive control, indicating robust potency in multiple in vitro FSHD models.

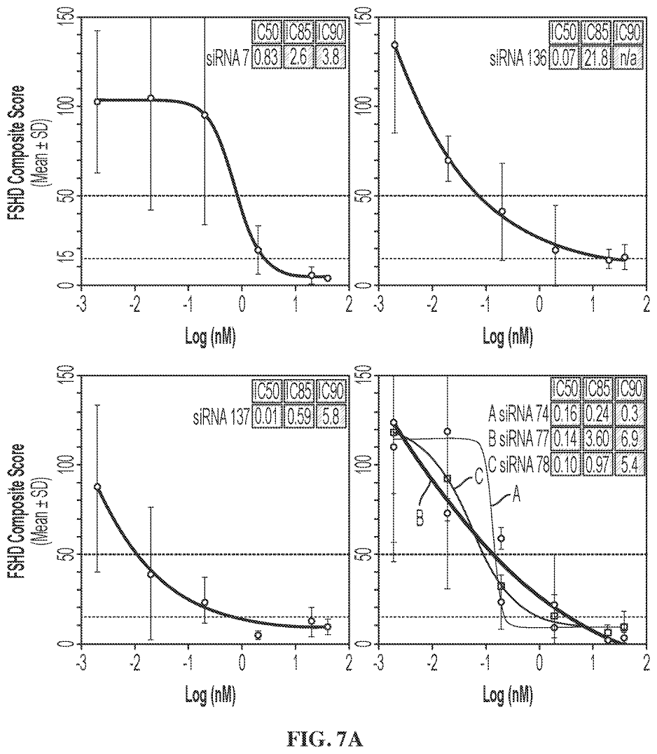

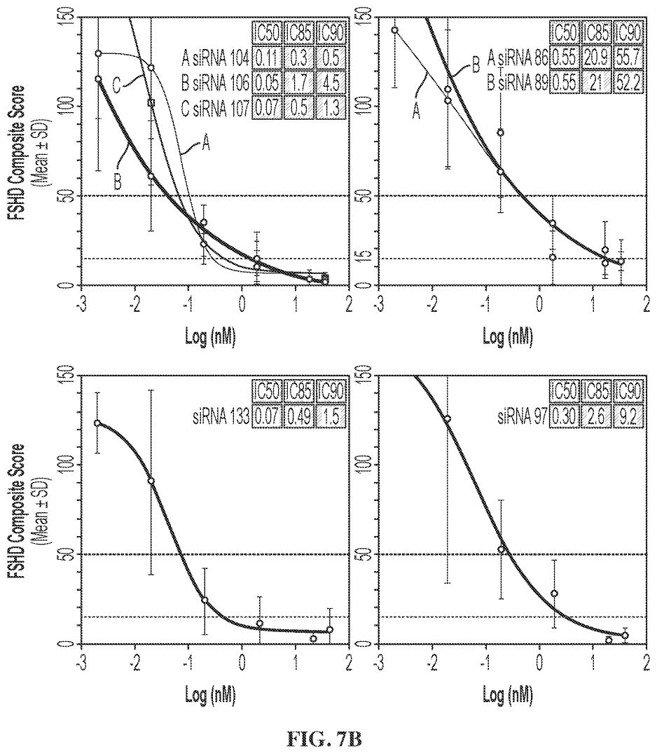

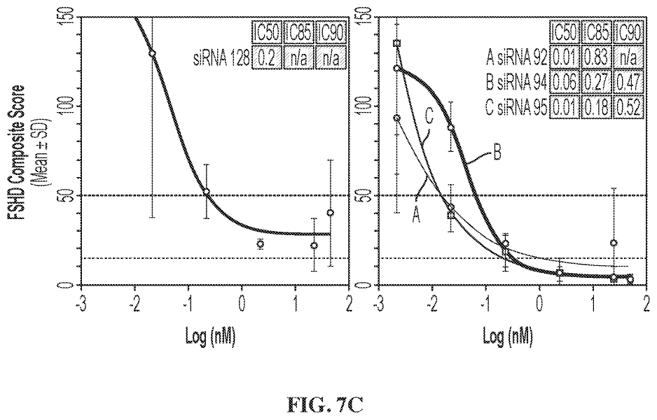

Dose response curves were also generated using composite scores calculated from the average mRNA levels of human MBD3L2, TRIM43, and ZSCAN4 markers for the 16 siRNAs tested and positive control over a range of concentrations (2 μM to 40 nM) and show knockdown of the DUX4 transcriptome markers (see FIGS. 7A-7C).

TABLE 10
Activity of DUX-4 targeting siRNA in vitro

| | Hepa1-6 (20 nM) | | FSHD C6 (20 nM) | |
|---|---|---|---|---|
| | DUX4 | | FSHD composite | |
| | (% control) | | (% control) | |
| SiRNA | AVERAGE | SD | AVERAGE | SD |
| 7 | 8.0 | 1.3 | 29.0 | 17.6 |
| 136 | 15.0 | 2.8 | 28.5 | 18.7 |
| 137 | 6.5 | 1.2 | 26.3 | 21.9 |
| 74 | 13.0 | 1.6 | 6.8 | 5.9 |
| 77 | 10.8 | 0.7 | 4.5 | 3.1 |
| 78 | 12.8 | 2.3 | 6.5 | 0.6 |
| 104 | 10.8 | 0.2 | 9.3 | 9.1 |
| 107 | 8.8 | 1.7 | 7.3 | 13.3 |
| 106 | 10.6 | 1.5 | 24.8 | 38.3 |
| 86 | 9.6 | 1.4 | 26.0 | 38.7 |
| 89 | 8.4 | 1.2 | 10.0 | 5.0 |
| 133 | 10.8 | 2.0 | 27.0 | 34.0 |
| 97 | 7.3 | 1.1 | 8.8 | 12.3 |
| 128 | 15.8 | 2.5 | 30.5 | 18.0 |
| 92 | 6.7 | 0.3 | 10.5 | 4.0 |
| 95 | 5.7 | 0.6 | 9.0 | 7.8 |
| 94 | 6.4 | 0.8 | 3.8 | 0.6 |

### Example 5: Activities of DUX4-Targeting siRNA Complexes in FSHD Patient Myotubes

Activities of seven complexes containing an anti-TfR1 Fab 3M12 VH4/VK3 covalently linked via a linker to a DUX4-targeting siRNA were tested in AB1080 immortalized FSHD patient-derived myotubes. For each siRNA complex, two versions were tested, one in which the anti-TfR1 Fab was covalently linked to the 3′ end of the sense strand of the siRNA and one in which the anti-TfR1 Fab was covalently linked to the 5′ end of the sense strand of each siRNA. The siRNAs tested were siRNAs 142-148. The siRNA numbers correspond to the siRNA numbers in Table 9. An exon 1 targeting siRNA was used as a positive control.

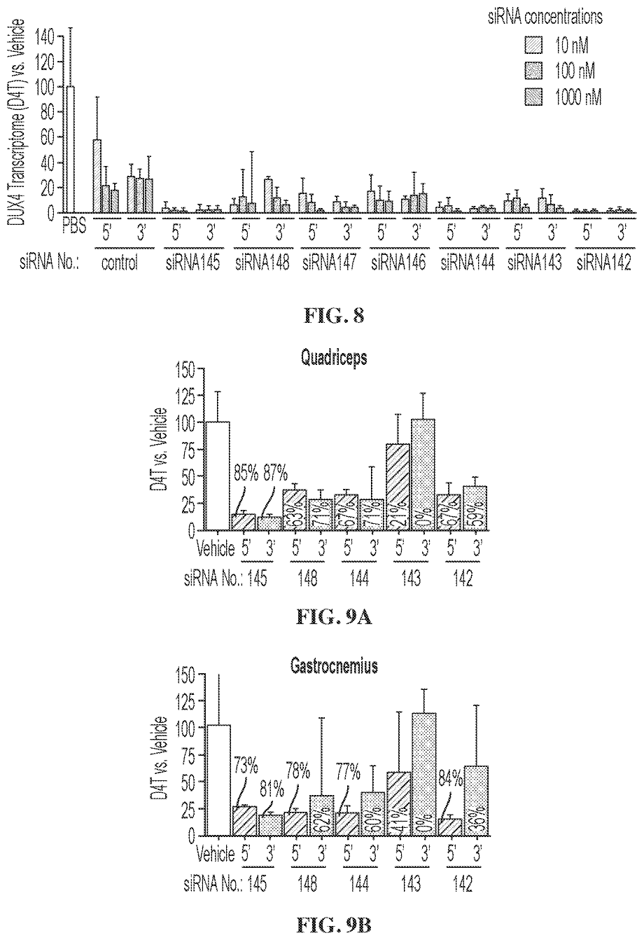

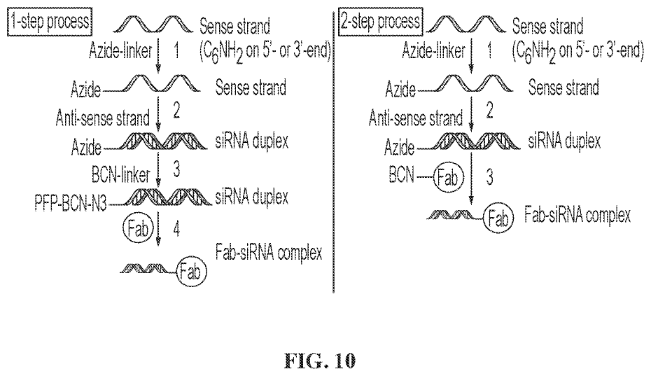

AB1080 (C6) immortalized FSHD patient-derived myotubes were treated with the siRNA complexes at a concentration equivalent to 10 nM, 100 nM, or 1000 nM for 10 days. cDNA was obtained from cells with the TaqMan Fast Advanced Cells-to-Ct Kit (Thermo Fisher Scientific), and levels of three DUX4 transcriptome markers MBD3L2 (Hs00544743_m1), TRIM43 (Hs00299174_m1), ZSCAN4 (Hs00537549_m1), and RPL13A (Hs04194366_g1) were analyzed via qPCR with specific TaqMan assays (Thermo Fisher Scientific). Two-step amplification reactions and fluorescence measurements for determination of cycle threshold (Ct) were conducted on a QuantStudio 7 instrument (Thermo Fisher Scientific). Composite scores calculated using the average mRNA level of three DUX4 transcriptome markers MBD3L2, TRIM43, and ZSCAN4 in FSHD patient-derived C6 myotubes are shown in FIG. 8.

Results show that the tested siRNAs achieved significant level of knockdown of the tested DUX4 transcriptome markers in FSHD patient-derived C6 myotubes compared to the positive control.

### Example 6: Activities of DUX4-targeting siRNA Complexes in FSHD Patient-Derived Primary Cells

Activities of complexes containing an anti-TfR1 Fab 3M12 VH4/VK3 covalently linked to an DUX4-targeting siRNA were tested in FSHD1 primary cells (MB010, MB015, MB016, MB018 and MB020). In the complexes, the anti-TfR1 Fab is covalently linked to the 5′ end of the sense strand of each siRNA via a linker, and the corresponding antisense strand is annealed to the sense strand. The siRNA tested was siRNA 145. The siRNA number corresponds to the siRNA number in Table 9. The primary cells were treated with the siRNA complexes at a concentration equivalent of 100 nM of siRNA for 7 days. cDNA was obtained from cells with the TaqMan Fast Advanced Cells-to-Ct Kit (Thermo Fisher Scientific), and levels of three DUX4 transcriptome markers MBD3L2 (Hs00544743_m1), TRIM43 (Hs00299174_m1), ZSCAN4 (Hs00537549_m1), and RPL13A (Hs04194366_g1) were analyzed via qPCR with specific TaqMan assays (Thermo Fisher Scientific). Two-step amplification reactions and fluorescence measurements for determination of cycle threshold (Ct) were conducted on a QuantStudio 7 instrument (Thermo Fisher Scientific).

Results (see Table 11) show that the siRNA achieved significant reduction of the tested DUX4 transcriptome markers in FSHD patient-derived primary cells.

TABLE 11

| | DUX4 transcriptomics | |
|---|---|---|
| | (% control) | |
| FSHD cells | Average | SD |
| MB010 | 60.3 | 20.7 |
| MB015 | 33.7 | 12.0 |
| MB016 | 37.6 | 10.9 |
| MB018 | 34.5 | 13.4 |
| MB020 | 2.7 | 1.2 |

### Example 7: Activities of DUX4-Targeting siRNA Complexes in Mouse Muscle Tissues

Activities of complexes containing an anti-TfR1 Fab 3M12 VH4/VK3 covalently linked via a linker to a DUX4-targeting siRNA were tested in eight- to nine-week-old hTfR1/FLExDUX4/ΔCTA mice. For each siRNA complex, two versions were tested, one in which the anti-TfR1 Fab was covalently linked to the 3′ end of the sense strand of the siRNA and one in which the anti-TfR1 Fab was covalently linked to the 5′ end of the sense strand of each siRNA. The siRNAs tested were siRNA145, siRNA148, siRNA144, siRNA143, and siRNA142. The siRNA numbers correspond to the siRNA numbers in Table 9.

The hTfR1/FLExDUX4/ΔCTA mice were intravenously injected with a single dose of a vehicle control (phosphate-buffered saline) or an siRNA complex at a concentration equivalent to 10 mg/kg of siRNA. Each experimental condition was replicated in three to fifteen individual hTfR1/FLExDUX4/ΔCTA mice. Following twenty-eight days after injection, the mice were euthanized. Gastrocnemius and quadricep muscles were collected and snap frozen in RNAlater™ Stabilization Solution (Thermo Fisher Scientific). Total miRNA was isolated from gastrocnemius and quadricep muscles with the Maxwell® RSC miRNA tissue kit (Promega) and real-time quantitative RT-PCR was performed.

Three murine downstream DUX4 transcriptome markers Wfdc3 (Mm01243777_m1), Sord (Mm00455377_g1), Serpinb6c (Mm00655242_m1), and Rpl37A (Mm00782745_s1) were analyzed via qPCR with specific TaqMan assays (Thermo Fisher Scientific). Two-step amplification reactions and fluorescence measurements for determination of cycle threshold (Ct) were conducted on a QuantStudio 7 instrument (Thermo Fisher Scientific). FSHD composite scores were calculated using the three DUX4 transcriptome markers (Wfdc3, Sord, Serpinb6c) where ΔCt=(Average Ct of 3 DUX4 markers)-(Average Ct of Rpl37A), ΔΔCt=ΔCt (Treated)-ΔCt (Vehicle), FSHD Composite=2-ΔΔCT*100(%) (see Table 12, FIGS. 9A and 9B).

Results show that many of the siRNA complexes achieved significant reduction of the tested DUX4 mouse transcriptome markers in the gastrocnemius and quadriceps of hTfR1/FLExDUX4/ΔCTA mice.

TABLE 12

| | DUX4 transcriptome knockdown | |
|---|---|---|
| | (% control) (geometric mean) | |
| antiTfR1-siRNA | Quadriceps | Gastrocnemius |
| siRNA 145-5′ | 85 | 73 |
| siRNA 145-3′ | 87 | 81 |
| siRNA 148-5′ | 63 | 78 |
| siRNA 148-3′ | 71 | 62 |
| siRNA 144-5′ | 67 | 77 |
| siRNA 144-3′ | 71 | 60 |
| siRNA 143-5′ | 21 | 41 |
| siRNA 143-3′ | 0 | 0 |
| siRNA 142-5′ | 67 | 84 |
| siRNA 142-3′ | 59 | 36 |

### Additional Embodiments

1. A complex comprising a muscle-targeting agent covalently linked to an oligonucleotide targeting a double homeobox 4 (DUX4) RNA, wherein the oligonucleotide comprises an antisense strand of 18-25 nucleotides in length and comprises a region of complementarity to a target sequence as set forth in SEQ ID NOs: 174-235, and wherein the region of complementarity is at least 16 consecutive nucleosides in length.

2. The complex of embodiment 1, wherein the muscle-targeting agent is an anti-transferrin receptor 1 (TfR1) antibody.

3. The complex of embodiment 1 or embodiment 2, wherein the oligonucleotide is an RNAi oligonucleotide.

4. The complex of embodiment 1 or embodiment 2, wherein the antisense strand comprises the nucleotide sequence of any one of SEQ ID NOs: 236-266.

5. The complex of any one of embodiments 1-4, wherein the oligonucleotide further comprises a sense strand which comprises at least 18 consecutive nucleosides complementary to the antisense strand.

6. The complex of embodiment 5, wherein the sense strand comprises the nucleotide sequence of any one of SEQ ID NOs: 205-235.

7. The complex of any one of embodiments 1-6, wherein the oligonucleotide comprises one or more modified nucleosides.

8. The complex of embodiment 7, wherein the one or more modified nucleosides are 2′ modified nucleotides, optionally wherein the one or more 2′ modified nucleosides are selected from: 2′-fluoro (2′-F), 2′-O-methyl(2′—O-Me), 2′-O-methoxyethyl(2′-MOE), 2′-O-aminopropyl(2′—O-AP), 2′-O-dimethylaminoethyl(2′—O-DMAOE), 2′-O-dimethylaminopropyl(2′—O-DMAP), 2′-O-dimethylaminoethyloxyethyl(2′—O-DMAEOE), 2′-O—N-methylacetamido (2′-O-NMA)).

9. The complex of embodiment 8, wherein each 2′ modified nucleotide is 2′-O-methyl(2′—O-Me) or 2′-fluoro (2′-F).

10. The complex of any one of embodiments 1-9, wherein the oligonucleotide comprises one or more phosphorothioate internucleoside linkages.

11. The complex of embodiment 10, wherein the one or more phosphorothioate internucleoside linkage are present on the antisense strand of the oligonucleotide.

12. The complex of embodiment 11, wherein the two internucleoside linkages at the 3′ end of the antisense strands are phosphorothioate internucleoside linkages.

13. The complex of embodiment 11 or embodiment 12, wherein the two internucleoside linkages at the 5′ end of the antisense strands are phosphorothioate internucleoside linkages.

14. The complex of any one of embodiments 1-13, wherein one or more cytidines of the oligonucleotide is a 2′-modified 5-methyl-cytidine, optionally wherein the 2′-modified 5-methyl-cytidine is a 2′-O-Me modified 5-methyl-cytidine or a 2′-F modified 5-methyl-cytidine.

15. The complex of any one of embodiments 1-14, wherein the antisense strand is selected from the modified versions of SEQ ID NOs: 236-266 (e.g., MAS1-MAS4) listed in Table 8.

16. The complex of any one of embodiments 5-15, wherein the sense strand is selected from the modified versions of SEQ ID NOs: 205-235 (e.g., MS1-MS6) listed in Table 8.

17. The complex of embodiments 1-16, wherein the oligonucleotide is an siRNA molecule selected from the siRNAs listed in Table 8.

18. The complex of any one of embodiments 2-17, wherein the anti-TfR1 antibody comprises a heavy chain complementarity determining region 1 (CDR-H1), a heavy chain complementarity determining region 2 (CDR-H2), a heavy chain complementarity determining region 3 (CDR-H3), a light chain complementarity determining region 1 (CDR-L1), a light chain complementarity determining region 2 (CDR-L2), a light chain complementarity determining region 3 (CDR-L3) of any of the anti-TfR1 antibodies listed in Table 2.

19. The complex of any one of embodiments 2-17, wherein the anti-TfR1 antibody comprises a heavy chain variable region (VH) and a light chain variable region (VL) of any of the anti-TfR1 antibodies listed in Table 3.

20. The complex of any one of embodiments 2-17, wherein the anti-TfR1 antibody is a Fab, optionally wherein the Fab comprises a heavy chain and a light chain of any of the anti-TfR1 Fabs listed in Table 5.

21. The complex of any one of embodiments 2-20, wherein the anti-TfR1 antibody comprises:
(i) a CDR-H1 comprising the amino acid sequence of SEQ ID NO: 27, a CDR-H2 comprising the amino acid sequence of SEQ ID NO: 28, a CDR-H3 comprising the amino acid sequence of SEQ ID NO: 29, a CDR-L1 comprising the amino acid sequence of SEQ ID NO: 30, a CDR-L2 comprising the amino acid sequence of SEQ ID NO: 31, and a CDR-L3 comprising the amino acid sequence of SEQ ID NO: 32;
(ii) a CDR-H1 comprising the amino acid sequence of SEQ ID NO: 33, a CDR-H2 comprising the amino acid sequence of SEQ ID NO: 34, a CDR-H3 comprising the amino acid sequence of SEQ ID NO: 35, a CDR-L1 comprising the amino acid sequence of SEQ ID NO: 36, a CDR-L2 comprising the amino acid sequence of SEQ ID NO: 37, and a CDR-L3 comprising the amino acid sequence of SEQ ID NO: 32; or
(ii) a CDR-H1 comprising the amino acid sequence of SEQ ID NO: 38, a CDR-H2 comprising the amino acid sequence of SEQ ID NO: 39, a CDR-H3 comprising the amino acid sequence of SEQ ID NO: 40, a CDR-L1 comprising the amino acid sequence of SEQ ID NO: 41, a CDR-L2 comprising the amino acid sequence of SEQ ID NO: 31, and a CDR-L3 comprising the amino acid sequence of SEQ ID NO: 42.

22. The complex of embodiment 21, wherein the anti-TfR1 antibody comprises a VH comprising the amino acid sequence of SEQ ID NO: 76, and a VL comprising the amino acid sequence of SEQ ID NO: 75.

23. The complex of embodiment 22, wherein the anti-TfR1 antibody is a Fab and comprises a heavy chain comprising the amino acid sequence of SEQ ID NO: 101 and a light chain comprising the amino acid sequence of SEQ ID NO: 90.

24. The complex of any one of embodiments 1-23, wherein the muscle targeting agent and the antisense oligonucleotide are covalently linked via a linker, optionally wherein the linker comprises a valine-citrulline sequence.

25. A method of reducing DUX4 expression in a muscle cell, the method comprising contacting the muscle cell with an effective amount of the complex of any one of embodiments 1-25 for promoting internalization of the oligonucleotide to the muscle cell.

26. The method of embodiment 25, wherein reducing DUX4 expression comprises reducing DUX4 protein and/or mRNA levels.

27. A method of treating Facioscapulohumeral muscular dystrophy (FSHD), the method comprising administering to a subject in need thereof an effective amount of the complex of any one of embodiments 1-24.

28. The method of embodiment 27, wherein the subject has aberrant production of DUX4 protein.

29. An oligonucleotide comprising an siRNA oligonucleotide selected from the siRNA oligonucleotides listed in Table 8.

30. A method of producing a complex comprising an anti-transferrin receptor 1 (TfR1) antibody covalently linked to an oligonucleotide, the method comprising:
(i) obtaining a compound comprising a structure of formula (B), wherein the oligonucleotide comprises a sense strand of a siRNA oligonucleotide, optionally wherein the siRNA oligonucleotide targets a DUX4 RNA;
(ii) annealing an antisense strand of the siRNA oligonucleotide to the sense strand;
(iii) reacting the compound comprising a structure of formula (B) with a compound comprising a structure of formula (C) to obtain a compound comprising a structure of formula (D); and
(iv) covalently linking an anti-TfR1 antibody to the compound comprising the structure of formula (D) with a muscle target agent to obtain a compound comprising a structure of formula (E).

31. The method of embodiment 30, wherein the annealing of step (ii) is performed at 30 °C. 32. The method of embodiment 30 or embodiment 31, further comprising isolating the compound comprising a structure of formula (D) after step (iii) and before step (iv).

33. The method of embodiment 30 or embodiment 31, wherein the compound comprising a structure of formula (D) is not isolated prior to step (iv).

34. A method of producing a complex comprising an anti-transferrin receptor 1 (TfR1) antibody covalently linked to an oligonucleotide, the method comprising:
(i) obtaining a compound comprising a structure of formula (B), wherein the oligonucleotide comprises a sense strand of a siRNA oligonucleotide, optionally wherein the siRNA oligonucleotide targets a DUX4 RNA;
(ii) annealing an antisense strand of the siRNA oligonucleotide to the sense strand;
(iii) covalently linking an anti-TfR1 antibody to a compound comprising a structure of formula (C) to obtain a compound comprising a structure of formula (F); and
(iv) reacting the compound comprising the structure of formula (F) with the compound comprising a structure of formula (B) to obtain a compound comprising a structure of formula (E).

35. The method of any one of embodiments 30-34, wherein the antisense strand is 18-25 nucleotides in length and comprises a region of complementarity to a target sequence as set forth in SEQ ID NOs: 189, 191, 200, 186, 190, 174-185, 187, 188, 192-199, and 201-235, and wherein the region of complementarity is at least 16 consecutive nucleosides in length.

36. The method of any one of embodiments 30-35, wherein the anti-TfR1 antibody comprises a heavy chain complementarity determining region 1 (CDR-H1), a heavy chain complementarity determining region 2 (CDR-H2), a heavy chain complementarity determining region 3 (CDR-H3), a light chain complementarity determining region 1 (CDR-L1), a light chain complementarity determining region 2 (CDR-L2), a light chain complementarity determining region 3 (CDR-L3) of any of the anti-TfR1 antibodies listed in Table 2.

37. The method of any one of embodiments 30-36, wherein the anti-TfR1 antibody is covalently linked to the 5′ end of the sense strand of the siRNA oligonucleotide targeting a DUX4 RNA.

### Equivalents and Terminology

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure. Thus, it should be understood that although the present disclosure has been specifically disclosed by preferred embodiments, optional features, modification and variation of the concepts herein disclosed may be resorted to by those skilled in the art, and that such modifications and variations are considered to be within the scope of this disclosure.

In addition, where features or aspects of the disclosure are described in terms of Markush groups or other grouping of alternatives, those skilled in the art will recognize that the disclosure is also thereby described in terms of any individual member or subgroup of members of the Markush group or other group.

It should be appreciated that, in some embodiments, sequences presented in the sequence listing may be referred to in describing the structure of an oligonucleotide or other nucleic acid. In such embodiments, the actual oligonucleotide or other nucleic acid may have one or more alternative nucleotides or nucleosides (e.g., an RNA counterpart of a DNA nucleoside or a DNA counterpart of an RNA nucleoside) and/or (e.g., and) one or more modified nucleotides/nucleosides and/or (e.g., and) one or more modified internucleoside linkages and/or (e.g., and) one or more other modification compared with the specified sequence while retaining essentially same or similar complementary properties as the specified sequence.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein. Variations of those embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Those skilled in the art will recognize, or be able to ascertain using no more than routine experimentation, many equivalents to the specific embodiments of the invention described herein. Such equivalents are intended to be encompassed by the following claims.

What is claimed is:

1. A complex comprising a muscle-targeting agent covalently linked to an oligonucleotide targeting a double homeobox 4 (DUX4) RNA, wherein the oligonucleotide comprises an antisense strand of 18-25 nucleotides in length and comprises a region of complementarity to a target sequence as set forth in SEQ ID NOs: 200, 191, 189, 186, 190, 174-185, 187, 188, 192-199, and 201-235, and wherein the region of complementarity is at least 16 consecutive nucleosides in length.

2. The complex of claim 1, wherein the muscle-targeting agent is an anti-transferrin receptor 1 (TfR1) antibody.

3. The complex of claim 1 or claim 2, wherein the oligonucleotide is an RNAi oligonucleotide.

4. The complex of any one of claims 1-3, wherein the antisense strand comprises the nucleotide sequence of any one of SEQ ID NOs: 262, 253, 251, 262, 248, 252, 236-247, 249, 250, 254-261, 263-266.

5. The complex of any one of claims 1-4, wherein the oligonucleotide further comprises a sense strand which comprises at least 18 consecutive nucleosides complementary to the antisense strand, optionally wherein the sense strand comprises 21 consecutive nucleosides complementary to the antisense strand.

6. The complex of claim 5, wherein the sense strand comprises the nucleotide sequence of any one of SEQ ID NOs: 231, 222, 220, 217, 221, 205-216, 218, 219, 223, 230, 232-235.

7. The complex of claim 5 or claim 6, wherein the muscle-targeting agent is covalently linked to the 5′ end or the 3′ end of the sense strand.

8. The complex of any one of claims 1-7, wherein the antisense strand further comprises a 5′-(E)-vinylphosphonate.

9. The complex of any one of claims 1-8, wherein the oligonucleotide comprises one or more modified nucleosides.

10. The complex of claim 9, wherein the one or more modified nucleosides are 2′ modified nucleotides, optionally wherein the one or more 2′ modified nucleosides are selected from: 2′-fluoro (2′-F), 2′-O-methyl(2′—O-Me), 2′-O-methoxyethyl(2′-MOE), 2′-O-aminopropyl(2′—O-AP), 2′-O-dimethylaminoethyl(2′—O-DMAOE), 2′-O-dimethylaminopropyl(2′—O-DMAP), 2′-O-dimethylaminoethyloxyethyl(2′—O-DMAEOE), 2′—O—N-methylacetamido (2′-O-NMA)).

11. The complex of claim 10, wherein each 2′ modified nucleotide is 2′-O-methyl(2′—O-Me) or 2′-fluoro (2′-F).

12. The complex of any one of claims 1-11, wherein the oligonucleotide comprises one or more phosphorothioate internucleoside linkages.

13. The complex of claim 12, wherein the one or more phosphorothioate internucleoside linkage are present on the antisense strand of the oligonucleotide, optionally wherein the two internucleoside linkages at the 3′ end of the antisense strands are phosphorothioate internucleoside linkages and/or wherein the two internucleoside linkages at the 5′ end of the antisense strands are phosphorothioate internucleoside linkages.

14. The complex of claim 12 or claim 13, wherein the one or more phosphorothioate internucleoside linkage are present on the sense strand of the oligonucleotide, optionally wherein the two internucleoside linkages at the 3′ end of the sense strands are phosphorothioate internucleoside linkages, and/or wherein the two internucleoside linkages at the 5′ end of the sense strands are phosphorothioate internucleoside linkages.

15. The complex of any one of claims 6-14, wherein the antisense strand is selected from the modified versions of SEQ ID NOs: 236-266 (e.g., MAS1-MAS4) listed in Table 8 and/or wherein the sense strand is selected from the modified versions of SEQ ID NOs: 205-235 (e.g., MS1-MS6) listed in Table 8.

16. The complex of any one of claims 6-14, wherein the antisense strand is selected from the modified versions of SEQ ID NOs: 262, 253, 251, 248, and 252 (e.g., VP-MAS5, VP-MAS6, and VP-MAS7) listed in Table 9 and/or wherein the sense strand is selected from the modified versions of SEQ ID NOs: 231, 222, 220, 217, and 221 (e.g., MS7-MS9) listed in Table 9.

17. The complex of claim 1-15, wherein the oligonucleotide is an siRNA molecule selected from the siRNAs listed in Table 8.

18. The complex of claim 1-14 or 16, wherein the oligonucleotide is an siRNA molecule selected from the siRNAs listed in Table 9.

19. The complex of any one of claims 2-18, wherein the anti-TfR1 antibody comprises a heavy chain complementarity determining region 1 (CDR-H1), a heavy chain complementarity determining region 2 (CDR-H2), a heavy chain complementarity determining region 3 (CDR-H3), a light chain complementarity determining region 1 (CDR-L1), a light chain complementarity determining region 2 (CDR-L2), a light chain complementarity determining region 3 (CDR-L3) of any of the anti-TfR1 antibodies listed in Table 2.

20. The complex of any one of claims 2-18, wherein the anti-TfR1 antibody comprises a heavy chain variable region (VH) and a light chain variable region (VL) of any of the anti-TfR1 antibodies listed in Table 3.

21. The complex of any one of claims 2-18, wherein the anti-TfR1 antibody is a Fab, optionally wherein the Fab comprises a heavy chain and a light chain of any of the anti-TfR1 Fabs listed in Table 5.

22. The complex of any one of claims 2-21, wherein the anti-TfR1 antibody comprises:
(i) a CDR-H1 comprising the amino acid sequence of SEQ ID NO: 27, a CDR-H2 comprising the amino acid sequence of SEQ ID NO: 28, a CDR-H3 comprising the amino acid sequence of SEQ ID NO: 29, a CDR-L1 comprising the amino acid sequence of SEQ ID NO: 30, a CDR-L2 comprising the amino acid sequence of SEQ ID NO: 31, and a CDR-L3 comprising the amino acid sequence of SEQ ID NO: 32;
(ii) a CDR-H1 comprising the amino acid sequence of SEQ ID NO: 33, a CDR-H2 comprising the amino acid sequence of SEQ ID NO: 34, a CDR-H3 comprising the amino acid sequence of SEQ ID NO: 35, a CDR-L1 comprising the amino acid sequence of SEQ ID NO: 36, a CDR-L2 comprising the amino acid sequence of SEQ ID NO: 37, and a CDR-L3 comprising the amino acid sequence of SEQ ID NO: 32; or
(ii) a CDR-H1 comprising the amino acid sequence of SEQ ID NO: 38, a CDR-H2 comprising the amino acid sequence of SEQ ID NO: 39, a CDR-H3 comprising the amino acid sequence of SEQ ID NO: 40, a CDR-L1 comprising the amino acid sequence of SEQ ID NO: 41, a CDR-L2 comprising the amino acid sequence of SEQ ID NO: 31, and a CDR-L3 comprising the amino acid sequence of SEQ ID NO: 42.

23. The complex of claim 22, wherein the anti-TfR1 antibody comprises a VH comprising the amino acid sequence of SEQ ID NO: 76, and a VL comprising the amino acid sequence of SEQ ID NO: 75.

24. The complex of claim 23, wherein the anti-TfR1 antibody is a Fab and comprises a heavy chain comprising the amino acid sequence of SEQ ID NO: 101 and a light chain comprising the amino acid sequence of SEQ ID NO: 90.

25. The complex of any one of claims 1-24, wherein the muscle targeting agent and the antisense oligonucleotide are covalently linked via a linker, optionally wherein the linker comprises a valine-citrulline sequence.

26. A method of reducing DUX4 expression in a muscle cell, the method comprising contacting the muscle cell with an effective amount of the complex of any one of claims 1-25 for promoting internalization of the oligonucleotide to the muscle cell.

27. The method of claim 26, wherein reducing DUX4 expression comprises reducing DUX4 protein and/or mRNA levels.

28. A method of treating Facioscapulohumeral muscular dystrophy (FSHD), the method comprising administering to a subject in need thereof an effective amount of the complex of any one of claims 1-25, wherein the subject has aberrant production of DUX4 protein.

29. An oligonucleotide comprising an siRNA oligonucleotide selected from the siRNA oligonucleotides listed in Table 8.

30. An oligonucleotide comprising an siRNA oligonucleotide selected from the siRNA oligonucleotides listed in Table 9.

31. A method of producing a complex comprising an anti-transferrin receptor 1 (TfR1) antibody covalently linked to an oligonucleotide, the method comprising:
(i) obtaining a compound comprising a structure of formula (B), wherein the oligonucleotide comprises a sense strand of a siRNA oligonucleotide, optionally wherein the siRNA oligonucleotide targets a DUX4 RNA;
(ii) annealing an antisense strand of the siRNA oligonucleotide to the sense strand;
(iii) reacting the compound comprising a structure of formula (B) with a compound comprising a structure of formula (C) to obtain a compound comprising a structure of formula (D); and
(iv) covalently linking an anti-TfR1 antibody to the compound comprising the structure of formula (D) with a muscle target agent to obtain a compound comprising a structure of formula (E),
optionally wherein the annealing of step (ii) is performed at 30 °C,
further optionally wherein the method further comprises isolating the compound comprising a structure of formula (D) after step (iii) and before step (iv);
further optionally wherein the anti-TfR1 antibody is covalently linked to the 5′ end of the sense strand of the siRNA oligonucleotide targeting a DUX4 RNA.

32. A method of producing a complex comprising an anti-transferrin receptor 1 (TfR1) antibody covalently linked to an oligonucleotide, the method comprising:
(i) obtaining a compound comprising a structure of formula (B), wherein the oligonucleotide comprises a sense strand of a siRNA oligonucleotide, optionally wherein the siRNA oligonucleotide targets a DUX4 RNA;
(ii) annealing an antisense strand of the siRNA oligonucleotide to the sense strand;
(iii) covalently linking an anti-TfR1 antibody to a compound comprising a structure of formula (C) to obtain a compound comprising a structure of formula (F); and
(iv) reacting the compound comprising the structure of formula (F) with the compound comprising a structure of formula (B) to obtain a compound comprising a structure of formula (E),
optionally wherein the annealing of step (ii) is performed at 30 °C,
further optionally wherein the anti-TfR1 antibody is covalently linked to the 5′ end of the sense strand of the siRNA oligonucleotide targeting a DUX4 RNA.


SEQUENCE LISTING (266 sequences)

SEQ ID NO: 1 - protein, 8 aa, synthetic construct
  REGION 1..8: note = Synthetic
GFNIKDDY

SEQ ID NO: 2 - protein, 8 aa, synthetic construct
  REGION 1..8: note = Synthetic
IDPENGDT

SEQ ID NO: 3 - protein, 10 aa, synthetic construct
  REGION 1..10: note = Synthetic
TLWLRRGLDY

SEQ ID NO: 4 - protein, 11 aa, synthetic construct
  REGION 1..11: note = Synthetic
KSLLHSNGYTY

SEQ ID NO: 5 - skipped

SEQ ID NO: 6 - protein, 9 aa, synthetic construct
  REGION 1..9: note = Synthetic
MQHLEYPFT

SEQ ID NO: 7 - protein, 5 aa, synthetic construct
  REGION 1..5: note = Synthetic
DDYMY

SEQ ID NO: 8 - protein, 17 aa, synthetic construct
  REGION 1..17: note = Synthetic
WIDPENGDTEYASKFQD

SEQ ID NO: 9 - protein, 8 aa, synthetic construct
  REGION 1..8: note = Synthetic
WLRRGLDY

SEQ ID NO: 10 - protein, 16 aa, synthetic construct
  REGION 1..16: note = Synthetic
RSSKSLLHSNGYTYLF

SEQ ID NO: 11 - protein, 7 aa, synthetic construct
  REGION 1..7: note = Synthetic
RMSNLAS

SEQ ID NO: 12 - protein, 7 aa, synthetic construct
  REGION 1..7: note = Synthetic
GFNIKDD

SEQ ID NO: 13 - skipped

SEQ ID NO: 14 - protein, 6 aa, synthetic construct
  REGION 1..6: note = Synthetic
LRRGLD

SEQ ID NO: 15 - protein, 12 aa, synthetic construct
  REGION 1..12: note = Synthetic
SKSLLHSNGYTY

SEQ ID NO: 16 - protein, 6 aa, synthetic construct
  REGION 1..6: note = Synthetic
HLEYPF

SEQ ID NO: 17 - protein, 117 aa, synthetic construct
  REGION 1..117: note = Synthetic
EVQLQQSGAELVRPGASVKLSCTASGFNIKDDYMYWVKQRPEQGLEWIGWIDPENGDTEYASKFQDKATVTADTSSNTAYLQLSSLTSEDTAVYYCTLWLRRGLDYWGQGTSVTVSS

SEQ ID NO: 18 - protein, 112 aa, synthetic construct
  REGION 1..112: note = Synthetic
DIVMTQAAPSVPVTPGESVSISCRSSKSLLHSNGYTYLFWFLQRPGQSPQLLIYRMSNLASGVPDRFSGSGSGTAFTLRISRVEAEDVGVYYCMQHLEYPFTFGGGTKLEIK

SEQ ID NO: 19 - protein, 8 aa, synthetic construct
  REGION 1..8: note = Synthetic
IDPETGDT

SEQ ID NO: 20 - protein, 17 aa, synthetic construct
  REGION 1..17: note = Synthetic
WIDPETGDTEYASKFQD

SEQ ID NO: 21 - skipped

SEQ ID NO: 22 - protein, 117 aa, synthetic construct
  REGION 1..117: note = Synthetic
EVQLQQSGAELVRPGASVKLSCTASGFNIKDDYMYWVKQRPEQGLEWIGWIDPETGDTEYASKFQDKATVTADTSSNTAYLQLSSLTSEDTAVYYCTLWLRRGLDYWGQGTSVTVSS

SEQ ID NO: 23 - protein, 8 aa, synthetic construct
  REGION 1..8: note = Synthetic
IDPESGDT

SEQ ID NO: 24 - protein, 17 aa, synthetic construct
  REGION 1..17: note = Synthetic
WIDPESGDTEYASKFQD

SEQ ID NO: 25 - skipped

SEQ ID NO: 26 - protein, 117 aa, synthetic construct
  REGION 1..117: note = Synthetic
EVQLQQSGAELVRPGASVKLSCTASGFNIKDDYMYWVKQRPEQGLEWIGWIDPESGDTEYASKFQDKATVTADTSSNTAYLQLSSLTSEDTAVYYCTLWLRRGLDYWGQGTSVTVSS

SEQ ID NO: 27 - protein, 9 aa, synthetic construct
  REGION 1..9: note = Synthetic
GYSITSGYY

SEQ ID NO: 28 - protein, 7 aa, synthetic construct
  REGION 1..7: note = Synthetic
ITFDGAN

SEQ ID NO: 29 - protein, 12 aa, synthetic construct
  REGION 1..12: note = Synthetic
TRSSYDYDVLDY

SEQ ID NO: 30 - protein, 6 aa, synthetic construct
  REGION 1..6: note = Synthetic
QDISNF

SEQ ID NO: 31 - skipped

SEQ ID NO: 32 - protein, 9 aa, synthetic construct
  REGION 1..9: note = Synthetic
QQGHTLPYT

SEQ ID NO: 33 - protein, 6 aa, synthetic construct
  REGION 1..6: note = Synthetic
SGYYWN

SEQ ID NO: 34 - protein, 16 aa, synthetic construct
  REGION 1..16: note = Synthetic
YITFDGANNYNPSLKN

SEQ ID NO: 35 - protein, 10 aa, synthetic construct
  REGION 1..10: note = Synthetic
SSYDYDVLDY

SEQ ID NO: 36 - protein, 11 aa, synthetic construct
  REGION 1..11: note = Synthetic
RASQDISNFLN

SEQ ID NO: 37 - protein, 7 aa, synthetic construct
  REGION 1..7: note = Synthetic
YTSRLHS

SEQ ID NO: 38 - protein, 8 aa, synthetic construct
  REGION 1..8: note = Synthetic
GYSITSGY

SEQ ID NO: 39 - skipped

SEQ ID NO: 40 - protein, 8 aa, synthetic construct
  REGION 1..8: note = Synthetic
SYDYDVLD

SEQ ID NO: 41 - protein, 7 aa, synthetic construct
  REGION 1..7: note = Synthetic
SQDISNF

SEQ ID NO: 42 - protein, 6 aa, synthetic construct
  REGION 1..6: note = Synthetic
GHTLPY

SEQ ID NO: 43 - protein, 119 aa, synthetic construct
  REGION 1..119: note = Synthetic
DVQLQESGPGLVKPSQSLSLTCSVTGYSITSGYYWNWIRQFPGNKLEWMGYITFDGANNYNPSLKNRISITRDTSKNQFFLKLTSVTTEDTATYYCTRSSYDYDVLDYWGQGTTLTVSS

SEQ ID NO: 44 - protein, 107 aa, synthetic construct
  REGION 1..107: note = Synthetic
DIQMTQTTSSLSASLGDRVTISCRASQDISNFLNWYQQRPDGTVKLLIYYTSRLHSGVPSRFSGSGSGTDFSLTVSNLEQEDIATYFCQQGHTLPYTFGGGTKLEIK

SEQ ID NO: 45 - protein, 8 aa, synthetic construct
  REGION 1..8: note = Synthetic
GYSFTDYC

SEQ ID NO: 46 - protein, 8 aa, synthetic construct
  REGION 1..8: note = Synthetic
IYPGSGNT

SEQ ID NO: 47 - protein, 13 aa, synthetic construct
  REGION 1..13: note = Synthetic
AREDYYPYHGMDY

SEQ ID NO: 48 - protein, 10 aa, synthetic construct
  REGION 1..10: note = Synthetic
ESVDGYDNSF

SEQ ID NO: 49 - skipped

SEQ ID NO: 50 - protein, 9 aa, synthetic construct
  REGION 1..9: note = Synthetic
QQSSEDPWT

SEQ ID NO: 51 - protein, 5 aa, synthetic construct
  REGION 1..5: note = Synthetic
DYCIN

SEQ ID NO: 52 - protein, 17 aa, synthetic construct
  REGION 1..17: note = Synthetic
WIYPGSGNTRYSERFKG

SEQ ID NO: 53 - protein, 11 aa, synthetic construct
  REGION 1..11: note = Synthetic
EDYYPYHGMDY

SEQ ID NO: 54 - protein, 15 aa, synthetic construct
  REGION 1..15: note = Synthetic
RASESVDGYDNSFMH

SEQ ID NO: 55 - protein, 7 aa, synthetic construct
  REGION 1..7: note = Synthetic
RASNLES

SEQ ID NO: 56 - protein, 7 aa, synthetic construct
  REGION 1..7: note = Synthetic
GYSFTDY

SEQ ID NO: 57 - skipped

SEQ ID NO: 58 - protein, 9 aa, synthetic construct
  REGION 1..9: note = Synthetic
DYYPYHGMD

SEQ ID NO: 59 - protein, 11 aa, synthetic construct
  REGION 1..11: note = Synthetic
SESVDGYDNSF

SEQ ID NO: 60 - protein, 6 aa, synthetic construct
  REGION 1..6: note = Synthetic
SSEDPW

SEQ ID NO: 61 - protein, 120 aa, synthetic construct
  REGION 1..120: note = Synthetic
QIQLQQSGPELVRPGASVKISCKASGYSFTDYCINWVNQRPGQGLEWIGWIYPGSGNTRYSERFKGKATLTVDTSSNTAYMQLSSLTSEDSAVYFCAREDYYPYHGMDYWGQGTSVTVSS

SEQ ID NO: 62 - protein, 111 aa, synthetic construct
  REGION 1..111: note = Synthetic
DIVLTQSPTSLAVSLGQRATISCRASESVDGYDNSFMHWYQQKPGQPPKLLIFRASNLESGIPARFSGSGSRTDFTLTINPVEAADVATYYCQQSSEDPWTFGGGTKLEIK

SEQ ID NO: 63 - protein, 8 aa, synthetic construct
  REGION 1..8: note = Synthetic
GYSFTDYY

SEQ ID NO: 64 - protein, 5 aa, synthetic construct
  REGION 1..5: note = Synthetic
DYYIN

SEQ ID NO: 65 - protein, 120 aa, synthetic construct
  REGION 1..120: note = Synthetic
QIQLQQSGPELVRPGASVKISCKASGYSFTDYYINWVNQRPGQGLEWIGWIYPGSGNTRYSERFKGKATLTVDTSSNTAYMQLSSLTSEDSAVYFCAREDYYPYHGMDYWGQGTSVTVSS

SEQ ID NO: 66 - protein, 8 aa, synthetic construct
  REGION 1..8: note = Synthetic
GYSFTDYD

SEQ ID NO: 67 - protein, 5 aa, synthetic construct
  REGION 1..5: note = Synthetic
DYDIN

SEQ ID NO: 68 - protein, 120 aa, synthetic construct
  REGION 1..120: note = Synthetic
QIQLQQSGPELVRPGASVKISCKASGYSFTDYDINWVNQRPGQGLEWIGWIYPGSGNTRYSERFKGKATLTVDTSSNTAYMQLSSLTSEDSAVYFCAREDYYPYHGMDYWGQGTSVTVSS

SEQ ID NO: 69 - protein, 117 aa, synthetic construct
  REGION 1..117: note = Synthetic
EVQLVQSGSELKKPGASVKVSCTASGFNIKDDYMYWVRQPPGKGLEWIGWIDPETGDTEYASKFQDRVTVTADTSTNTAYMELSSLRSEDTAVYYCTLWLRRGLDYWGQGTLVTVSS

SEQ ID NO: 70 - protein, 112 aa, synthetic construct
  REGION 1..112: note = Synthetic
DIVMTQSPLSLPVTPGEPASISCRSSKSLLHSNGYTYLFWFQQRPGQSPRLLIYRMSNLASGVPDRFSGSGSGTDFTLKISRVEAEDVGVYYCMQHLEYPFTFGGGTKVEIK

SEQ ID NO: 71 - protein, 117 aa, synthetic construct
  REGION 1..117: note = Synthetic
EVQLVQSGSELKKPGASVKVSCTASGFNIKDDYMYWVRQPPGKGLEWIGWIDPESGDTEYASKFQDRVTVTADTSTNTAYMELSSLRSEDTAVYYCTLWLRRGLDYWGQGTLVTVSS

SEQ ID NO: 72 - protein, 117 aa, synthetic construct
  REGION 1..117: note = Synthetic
EVQLVQSGSELKKPGASVKVSCTASGFNIKDDYMYWVRQPPGKGLEWIGWIDPENGDTEYASKFQDRVTVTADTSTNTAYMELSSLRSEDTAVYYCTLWLRRGLDYWGQGTLVTVSS

SEQ ID NO: 73 - protein, 119 aa, synthetic construct
  REGION 1..119: note = Synthetic
QVQLQESGPGLVKPSQTLSLTCSVTGYSITSGYYWNWIRQPPGKGLEWMGYITFDGANNYNPSLKNRVSISRDTSKNQFSLKLSSVTAEDTATYYCTRSSYDYDVLDYWGQGTTVTVSS

SEQ ID NO: 74 - protein, 107 aa, synthetic construct
  REGION 1..107: note = Synthetic
DIQMTQSPSSLSASVGDRVTITCRASQDISNFLNWYQQKPGQPVKLLIYYTSRLHSGVPSRFSGSGSGTDFTLTISSLQPEDFATYFCQQGHTLPYTFGQGTKLEIK

SEQ ID NO: 75 - protein, 107 aa, synthetic construct
  REGION 1..107: note = Synthetic
DIQMTQSPSSLSASVGDRVTITCRASQDISNFLNWYQQKPGQPVKLLIYYTSRLHSGVPSRFSGSGSGTDFTLTISSLQPEDFATYYCQQGHTLPYTFGQGTKLEIK

SEQ ID NO: 76 - protein, 119 aa, synthetic construct
  REGION 1..119: note = Synthetic
QVQLQESGPGLVKPSQTLSLTCTVTGYSITSGYYWNWIRQPPGKGLEWIGYITFDGANNYNPSLKNRVSISRDTSKNQFSLKLSSVTAEDTATYYCTRSSYDYDVLDYWGQGTTVTVSS

SEQ ID NO: 77 - protein, 120 aa, synthetic construct
  REGION 1..120: note = Synthetic
QVQLVQSGAEVKKPGASVKVSCKASGYSFTDYYINWVRQAPGQGLEWMGWIYPGSGNTRYSERFKGRVTITRDTSASTAYMELSSLRSEDTAVYYCAREDYYPYHGMDYWGQGTLVTVSS

SEQ ID NO: 78 - protein, 111 aa, synthetic construct
  REGION 1..111: note = Synthetic
DIVLTQSPDSLAVSLGERATINCRASESVDGYDNSFMHWYQQKPGQPPKLLIFRASNLESGVPDRFSGSGSRTDFTLTISSLQAEDVAVYYCQQSSEDPWTFGQGTKLEIK

SEQ ID NO: 79 - protein, 120 aa, synthetic construct
  REGION 1..120: note = Synthetic
QVQLVQSGAEVKKPGASVKVSCKASGYSFTDYDINWVRQAPGQGLEWMGWIYPGSGNTRYSERFKGRVTITRDTSASTAYMELSSLRSEDTAVYYCAREDYYPYHGMDYWGQGTLVTVSS

SEQ ID NO: 80 - protein, 111 aa, synthetic construct
  REGION 1..111: note = Synthetic
DIVMTQSPDSLAVSLGERATINCRASESVDGYDNSFMHWYQQKPGQPPKLLIFRASNLESGVPDRFSGSGSGTDFTLTISSLQAEDVAVYYCQQSSEDPWTFGQGTKLEIK

SEQ ID NO: 81 - protein, 330 aa, Homo sapiens [sequence omitted]

SEQ ID NO: 82 - protein, 330 aa, synthetic construct
  REGION 1..330: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 107 aa, synthetic construct [sequence omitted]

SEQ ID NO: 84 - protein, 447 aa, synthetic construct
  REGION 1..447: note = Synthetic
EVQLVQSGSELKKPGASVKVSCTASGFNIKDDYMYWVRQPPGKGLEWIGWIDPETGDTEYASKFQDRVTVTADTSTNTAYMELSSLRSEDTAVYYCTLWLRRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 219 aa, synthetic construct
  REGION 1..219: note = Synthetic
DIVMTQSPLSLPVTPGEPASISCRSSKSLLHSNGYTYLFWFQQRPGQSPRLLIYRMSNLASGVPDRFSGSGSGTDFTLKISRVEAEDVGVYYCMQHLEYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 447 aa, synthetic construct
  REGION 1..447: note = Synthetic
EVQLVQSGSELKKPGASVKVSCTASGFNIKDDYMYWVRQPPGKGLEWIGWIDPESGDTEYASKFQDRVTVTADTSTNTAYMELSSLRSEDTAVYYCTLWLRRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 447 aa, synthetic construct
  REGION 1..447: note = Synthetic
EVQLVQSGSELKKPGASVKVSCTASGFNIKDDYMYWVRQPPGKGLEWIGWIDPENGDTEYASKFQDRVTVTADTSTNTAYMELSSLRSEDTAVYYCTLWLRRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 449 aa, synthetic construct
  REGION 1..449: note = Synthetic
QVQLQESGPGLVKPSQTLSLTCSVTGYSITSGYYWNWIRQPPGKGLEWMGYITFDGANNYNPSLKNRVSISRDTSKNQFSLKLSSVTAEDTATYYCTRSSYDYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 214 aa, synthetic construct
  REGION 1..214: note = Synthetic
DIQMTQSPSSLSASVGDRVTITCRASQDISNFLNWYQQKPGQPVKLLIYYTSRLHSGVPSRFSGSGSGTDFTLTISSLQPEDFATYFCQQGHTLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 214 aa, synthetic construct
  REGION 1..214: note = Synthetic
DIQMTQSPSSLSASVGDRVTITCRASQDISNFLNWYQQKPGQPVKLLIYYTSRLHSGVPSRFSGSGSGTDFTLTISSLQPEDFATYYCQQGHTLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 449 aa, synthetic construct
  REGION 1..449: note = Synthetic
QVQLQESGPGLVKPSQTLSLTCTVTGYSITSGYYWNWIRQPPGKGLEWIGYITFDGANNYNPSLKNRVSISRDTSKNQFSLKLSSVTAEDTATYYCTRSSYDYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 450 aa, synthetic construct
  REGION 1..450: note = Synthetic
QVQLVQSGAEVKKPGASVKVSCKASGYSFTDYYINWVRQAPGQGLEWMGWIYPGSGNTRYSERFKGRVTITRDTSASTAYMELSSLRSEDTAVYYCAREDYYPYHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 218 aa, synthetic construct
  REGION 1..218: note = Synthetic
DIVLTQSPDSLAVSLGERATINCRASESVDGYDNSFMHWYQQKPGQPPKLLIFRASNLESGVPDRFSGSGSRTDFTLTISSLQAEDVAVYYCQQSSEDPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 450 aa, synthetic construct
  REGION 1..450: note = Synthetic
QVQLVQSGAEVKKPGASVKVSCKASGYSFTDYDINWVRQAPGQGLEWMGWIYPGSGNTRYSERFKGRVTITRDTSASTAYMELSSLRSEDTAVYYCAREDYYPYHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 218 aa, synthetic construct
  REGION 1..218: note = Synthetic
DIVMTQSPDSLAVSLGERATINCRASESVDGYDNSFMHWYQQKPGQPPKLLIFRASNLESGVPDRFSGSGSGTDFTLTISSLQAEDVAVYYCQQSSEDPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 108 aa, synthetic construct
  REGION 1..108: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 97 - protein, 225 aa, synthetic construct
  REGION 1..225: note = Synthetic
EVQLVQSGSELKKPGASVKVSCTASGFNIKDDYMYWVRQPPGKGLEWIGWIDPETGDTEYASKFQDRVTVTADTSTNTAYMELSSLRSEDTAVYYCTLWLRRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 98 - protein, 225 aa, synthetic construct
  REGION 1..225: note = Synthetic
EVQLVQSGSELKKPGASVKVSCTASGFNIKDDYMYWVRQPPGKGLEWIGWIDPESGDTEYASKFQDRVTVTADTSTNTAYMELSSLRSEDTAVYYCTLWLRRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 99 - protein, 225 aa, synthetic construct
  REGION 1..225: note = Synthetic
EVQLVQSGSELKKPGASVKVSCTASGFNIKDDYMYWVRQPPGKGLEWIGWIDPENGDTEYASKFQDRVTVTADTSTNTAYMELSSLRSEDTAVYYCTLWLRRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 100 - protein, 227 aa, synthetic construct
  REGION 1..227: note = Synthetic
QVQLQESGPGLVKPSQTLSLTCSVTGYSITSGYYWNWIRQPPGKGLEWMGYITFDGANNYNPSLKNRVSISRDTSKNQFSLKLSSVTAEDTATYYCTRSSYDYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 101 - protein, 227 aa, synthetic construct
  REGION 1..227: note = Synthetic
QVQLQESGPGLVKPSQTLSLTCTVTGYSITSGYYWNWIRQPPGKGLEWIGYITFDGANNYNPSLKNRVSISRDTSKNQFSLKLSSVTAEDTATYYCTRSSYDYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 102 - protein, 228 aa, synthetic construct
  REGION 1..228: note = Synthetic
QVQLVQSGAEVKKPGASVKVSCKASGYSFTDYYINWVRQAPGQGLEWMGWIYPGSGNTRYSERFKGRVTITRDTSASTAYMELSSLRSEDTAVYYCAREDYYPYHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 103 - protein, 228 aa, synthetic construct
  REGION 1..228: note = Synthetic
QVQLVQSGAEVKKPGASVKVSCKASGYSFTDYDINWVRQAPGQGLEWMGWIYPGSGNTRYSERFKGRVTITRDTSASTAYMELSSLRSEDTAVYYCAREDYYPYHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 104 - protein, 19 aa, synthetic construct
  REGION 1..19: note = Synthetic
MGWSCIILFLVATATGVHS

SEQ ID NO: 105 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 106 - protein, 760 aa, Macaca mulatta
MMDQARSAFSNLFGGEPLSYTRFSLARQVDGDNSHVEMKLGVDEEENTDNNTKPNGTKPKRCGGNICYGTIAVIIFFLIGFMIGYLGYCKGVEPKTECERLAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 107 - protein, 760 aa, Macaca fascicularis
MMDQARSAFSNLFGGEPLSYTRFSLARQVDGDNSHVEMKLGVDEEENTDNNTKANGTKPKRCGGNICYGTIAVIIFFLIGFMIGYLGYCKGVEPKTECERLAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 108 - protein, 763 aa, Mus musculus
MMDQARSAFSNLFGGEPLSYTRFSLARQVDGDNSHVEMKLAADEEENADNNMKASVRKPKRFNGRLCFAAIALVIFFLIGFMSGYLGYCKRVEQKEECVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 109 - protein, 197 aa, synthetic construct
  REGION 1..197: note = Synthetic
FVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKE

SEQ ID NO: 110 - protein, 5 aa, synthetic construct
  REGION 1..5: note = Synthetic
SYWMH

SEQ ID NO: 111 - protein, 17 aa, synthetic construct
  REGION 1..17: note = Synthetic
EINPTNGRTNYIEKFKS

SEQ ID NO: 112 - protein, 7 aa, synthetic construct
  REGION 1..7: note = Synthetic
GTRAYHY

SEQ ID NO: 113 - protein, 11 aa, synthetic construct
  REGION 1..11: note = Synthetic
RASDNLYSNLA

SEQ ID NO: 114 - protein, 7 aa, synthetic construct
  REGION 1..7: note = Synthetic
DATNLAD

SEQ ID NO: 115 - protein, 9 aa, synthetic construct
  REGION 1..9: note = Synthetic
QHFWGTPLT

SEQ ID NO: 116 - protein, 7 aa, synthetic construct
  REGION 1..7: note = Synthetic
GYTFTSY

SEQ ID NO: 117 - protein, 6 aa, synthetic construct
  REGION 1..6: note = Synthetic
NPTNGR

SEQ ID NO: 118 - protein, 6 aa, synthetic construct
  REGION 1..6: note = Synthetic
TSYWMH

SEQ ID NO: 119 - protein, 13 aa, synthetic construct
  REGION 1..13: note = Synthetic
WIGEINPTNGRTN

SEQ ID NO: 120 - protein, 6 aa, synthetic construct
  REGION 1..6: note = Synthetic
ARGTRA

SEQ ID NO: 121 - protein, 7 aa, synthetic construct
  REGION 1..7: note = Synthetic
YSNLAWY

SEQ ID NO: 122 - protein, 10 aa, synthetic construct
  REGION 1..10: note = Synthetic
LLVYDATNLA

SEQ ID NO: 123 - protein, 8 aa, synthetic construct
  REGION 1..8: note = Synthetic
QHFWGTPL

SEQ ID NO: 124 - protein, 116 aa, synthetic construct
  REGION 1..116: note = Synthetic
QVQLQQPGAELVKPGASVKLSCKASGYTFTSYWMHWVKQRPGQGLEWIGEINPTNGRTNYIEKFKSKATLTVDKSSSTAYMQLSSLTSEDSAVYYCARGTRAYHYWGQGTSVTVSS

SEQ ID NO: 125 - protein, 107 aa, synthetic construct
  REGION 1..107: note = Synthetic
DIQMTQSPASLSVSVGETVTITCRASDNLYSNLAWYQQKQGKSPQLLVYDATNLADGVPSRFSGSGSGTQYSLKINSLQSEDFGTYYCQHFWGTPLTFGAGTKLELK

SEQ ID NO: 126 - protein, 9 aa, synthetic construct
  REGION 1..9: note = Synthetic
QHFAGTPLT

SEQ ID NO: 127 - protein, 8 aa, synthetic construct
  REGION 1..8: note = Synthetic
QHFAGTPL

SEQ ID NO: 128 - protein, 116 aa, synthetic construct
  REGION 1..116: note = Synthetic
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSS

SEQ ID NO: 129 - protein, 107 aa, synthetic construct
  REGION 1..107: note = Synthetic
DIQMTQSPSSLSASVGDRVTITCRASDNLYSNLAWYQQKPGKSPKLLVYDATNLADGVPSRFSGSGSGTDYTLTISSLQPEDFATYYCQHFWGTPLTFGQGTKVEIK

SEQ ID NO: 130 - protein, 9 aa, synthetic construct
  REGION 1..9: note = Synthetic
CPKTRRVPC

SEQ ID NO: 131 - protein, 7 aa, synthetic construct
  REGION 1..7: note = Synthetic
DDTRHWG

SEQ ID NO: 132 - protein, 446 aa, synthetic construct
  REGION 1..446: note = Synthetic
QVQLQQPGAELVKPGASVKLSCKASGYTFTSYWMHWVKQRPGQGLEWIGEINPTNGRTNYIEKFKSKATLTVDKSSSTAYMQLSSLTSEDSAVYYCARGTRAYHYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 214 aa, synthetic construct
  REGION 1..214: note = Synthetic
DIQMTQSPASLSVSVGETVTITCRASDNLYSNLAWYQQKQGKSPQLLVYDATNLADGVPSRFSGSGSGTQYSLKINSLQSEDFGTYYCQHFWGTPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 134 - protein, 446 aa, synthetic construct
  REGION 1..446: note = Synthetic
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 214 aa, synthetic construct
  REGION 1..214: note = Synthetic
DIQMTQSPSSLSASVGDRVTITCRASDNLYSNLAWYQQKPGKSPKLLVYDATNLADGVPSRFSGSGSGTDYTLTISSLQPEDFATYYCQHFWGTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 226 aa, synthetic construct
  REGION 1..226: note = Synthetic
QVQLQQPGAELVKPGASVKLSCKASGYTFTSYWMHWVKQRPGQGLEWIGEINPTNGRTNYIEKFKSKATLTVDKSSSTAYMQLSSLTSEDSAVYYCARGTRAYHYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCP

SEQ ID NO: 137 - protein, 226 aa, synthetic construct
  REGION 1..226: note = Synthetic
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCP

SEQ ID NO: 138 - protein, 8 aa, synthetic construct
  REGION 1..8: note = Synthetic
GYSFTSYW

SEQ ID NO: 139 - protein, 8 aa, synthetic construct
  REGION 1..8: note = Synthetic
IYPGDSDT

SEQ ID NO: 140 - protein, 15 aa, synthetic construct
  REGION 1..15: note = Synthetic
ARFPYDSSGYYSFDY

SEQ ID NO: 141 - protein, 6 aa, synthetic construct
  REGION 1..6: note = Synthetic
QSISSY

SEQ ID NO: 142 - skipped

SEQ ID NO: 143 - protein, 9 aa, synthetic construct
  REGION 1..9: note = Synthetic
QQSYSTPLT

SEQ ID NO: 144 - protein, 5 aa, synthetic construct
  REGION 1..5: note = Synthetic
SYWIG

SEQ ID NO: 145 - protein, 18 aa, synthetic construct
  REGION 1..18: note = Synthetic
IIYPGDSDTRYSPSFQGQ

SEQ ID NO: 146 - protein, 13 aa, synthetic construct
  REGION 1..13: note = Synthetic
FPYDSSGYYSFDY

SEQ ID NO: 147 - protein, 11 aa, synthetic construct
  REGION 1..11: note = Synthetic
RASQSISSYLN

SEQ ID NO: 148 - protein, 7 aa, synthetic construct
  REGION 1..7: note = Synthetic
AASSLQS

SEQ ID NO: 149 - protein, 7 aa, synthetic construct
  REGION 1..7: note = Synthetic
GYSFTSY

SEQ ID NO: 150 - skipped

SEQ ID NO: 151 - protein, 11 aa, synthetic construct
  REGION 1..11: note = Synthetic
PYDSSGYYSFD

SEQ ID NO: 152 - protein, 7 aa, synthetic construct
  REGION 1..7: note = Synthetic
SQSISSY

SEQ ID NO: 153 - protein, 6 aa, synthetic construct
  REGION 1..6: note = Synthetic
SYSTPL

SEQ ID NO: 154 - protein, 122 aa, synthetic construct
  REGION 1..122: note = Synthetic
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARFPYDSSGYYSFDYWGQGTLVTVSS

SEQ ID NO: 155 - protein, 107 aa, synthetic construct [sequence omitted]

SEQ ID NO: 156 - protein, 452 aa, synthetic construct
  REGION 1..452: note = Synthetic
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARFPYDSSGYYSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 214 aa, synthetic construct
  REGION 1..214: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 158 - protein, 232 aa, synthetic construct
  REGION 1..232: note = Synthetic
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARFPYDSSGYYSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCP

SEQ ID NO: 159 - protein, 230 aa, synthetic construct
  REGION 1..230: note = Synthetic
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARFPYDSSGYYSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 160 - DNA, 1574 bp, Homo sapiens
  source 1..1574: mol_type = genomic DNA
atggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagaggggatctcccaacctgccccggcgcgcggggatttcgcctacgccgccccggctcctccggacggggcgctctcccaccctcaggctcctcgctggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttaggacgcggggtctaggcccggtgagagactccacaccgcggagaactgccattctttcctgggcatcccggggatcccagagccggcccaggtaccagcagacctgcgcgcagtgcgcaccccggctgacgtgcaagggagctcgctggcctctctgtgcccttgttcttccgtgaaattctggctgaatgtctccccccaccttccgacgctgtctaggcaaacctggattagagttacatctcctggatgattagttcagagatatattaaaatgccccctccctgtggatcctatag

SEQ ID NO: 161 - DNA, 1710 bp, Homo sapiens
  source 1..1710: mol_type = genomic DNA
atggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagaggggatctcccaacctgccccggcgcgcggggatttcgcctacgccgccccggctcctccggacggggcgctctcccaccctcaggctcctcggtggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttaggacgcggggttgggacggggtcgggtggttcggggcagggcggtggcctctctttcgcggggaacacctggctggctacggaggggcgtgtctccgccccgccccctccaccgggctgaccggcctgggattcctgccttctaggtctaggcccggtgagagactccactccgcggagaactgcctttctttcctgggcatcccggggatcccagagccggcccaggtaccagcagacctgcgcgcagtgcgcaccccggctgacgtgcaagggagctcgctggcctctctgtgcccttgttcttccgtgaaattctggctgaatgtctccccccaccttccgacgctgtctaggcaaacctggattagagttacatctcctggatgattagttcagagatatattaaaatgccccctccctgtggatcctatag

SEQ ID NO: 162 - DNA, 2024 bp, Mus musculus
  source 1..2024: mol_type = genomic DNA
atggcagaagctggcagccctgttggtggcagtggtgtggcacgggaatcccggcggcgcaggaagacggtttggcaggcctggcaagagcaggccctgctatcaactttcaagaagaagagatacctgagcttcaaggagaggaaggagctggccaagcgaatgggggtctcagattgccgcatccgcgtgtggtttcagaaccgcaggaatcgcagtggagaggaggggcatgcctcaaagaggtccatcagaggctccaggcggctagcctcgccacagctccaggaagagcttggatccaggccacagggtagaggcatgcgctcatctggcagaaggcctcgcactcgactcacctcgctacagctcaggatcctagggcaagcctttgagaggaacccacgaccaggctttgctaccagggaggagctggcgcgtgacacagggttgcccgaggacacgatccacatatggtttcaaaaccgaagagctcggcggcgccacaggaggggcaggcccacagctcaagatcaagacttgctggcgtcacaagggtcggatggggcccctgcaggtccggaaggcagagagcgtgaaggtgcccaggagaacttgttgccacaggaagaagcaggaagtacgggcatggatacctcgagccctagcgacttgccctccttctgcggagagtcccagcctttccaagtggcacagccccgtggagcaggccaacaagaggcccccactcgagcaggcaacgcaggctctctggaacccctccttgatcagctgctggatgaagtccaagtagaagagcctgctccagcccctctgaatttggatggagaccctggtggcagggtgcatgaaggttcccaggagagcttttggccacaggaagaagcaggaagtacaggcatggatacttctagccccagcgactcaaactccttctgcagagagtcccagccttcccaagtggcacagccctgtggagcgggccaagaagatgcccgcactcaagcagacagcacaggccctctggaactcctcctccttgatcaactgctggacgaagtccaaaaggaagagcatgtgccagtcccactggattggggtagaaatcctggcagcagggagcatgaaggttcccaggacagcttactgcccctggaggaagcagtaaattcgggcatggatacctcgatccctagcatctggccaaccttctgcagagaatcccagcctccccaagtggcacagccctctggaccaggccaagcacaggcccccactcaaggtgggaacacggaccccctggagctcttcctctatcaactgttggatgaagtccaagtagaagagcatgctccagcccctctgaattgggatgtagatcctggtggcagggtgcatgaaggttcgtgggagagcttttggccacaggaagaagcaggaagtacaggcctggatacttcaagccccagcgactcaaactccttcttcagagagtccaagccttcccaagtggcacagcgccgtggagcgggccaagaagatgcccgcactcaagcagacagcacaggccctctggaactcctcctctttgatcaactgctggacgaagtccaaaaggaagagcatgtgccagccccactggattggggtagaaatcctggcagcatggagcatgaaggttcccaggacagcttactgcccctggaggaagcagcaaattcgggcagggatacctcgatccctagcatctggccagccttctgcagaaaatcccagcctccccaagtggcacagccctctggaccaggccaagcacaggcccccattcaaggtgggaacacggaccccctggagctcttccttgatcaactgctgaccgaagtccaacttgaggagcaggggcctgcccctgtgaatgtggaggaaacatgggagcaaatggacacaacacctatctgcctctcacttcagaagaatatcagactcttctagatatgctctga

SEQ ID NO: 163 - RNA, 21 nt, synthetic construct
  misc_feature 1..21: note = Synthetic
  source 1..21: mol_type = other RNA
tcctatgactgtagattttat

SEQ ID NO: 164 - RNA, 23 nt, synthetic construct
  misc_feature 1..23: note = Synthetic
  source 1..23: mol_type = other RNA
ataaaatctacagtcataggaat

SEQ ID NO: 165 - RNA, 21 nt, synthetic construct
  misc_feature 1..21: note = Synthetic
  source 1..21: mol_type = other RNA
tgtaataaccatatctacctt

SEQ ID NO: 166 - RNA, 23 nt, synthetic construct
  misc_feature 1..23: note = Synthetic
  source 1..23: mol_type = other RNA
aaggtagatatggttattacaaa

SEQ ID NO: 167 - protein, 7 aa, synthetic construct
  REGION 1..7: note = Synthetic
ASSLNIA

SEQ ID NO: 168 - protein, 12 aa, synthetic construct
  REGION 1..12: note = Synthetic
SKTFNTHPQSTP

SEQ ID NO: 169 - protein, 12 aa, synthetic construct
  REGION 1..12: note = Synthetic
TARGEHKEEELI

SEQ ID NO: 170 - protein, 9 aa, synthetic construct
  REGION 1..9: note = Synthetic
CQAQGQLVC

SEQ ID NO: 171 - protein, 9 aa, synthetic construct
  REGION 1..9: note = Synthetic
CSERSMNFC

SEQ ID NO: 172 - protein, 20 aa, synthetic construct
  REGION 1..20: note = Synthetic
WLSEAGPVVTVRALRGTGSW

SEQ ID NO: 173 - protein, 9 aa, synthetic construct
  REGION 1..9: note = Synthetic
CMQHSMRVC

SEQ ID NO: 174 - DNA, 20 bp, synthetic construct
  misc_feature 1..20: note = Synthetic
  source 1..20: mol_type = other DNA
gatttggtttcagaatgaga

SEQ ID NO: 175 - DNA, 20 bp, synthetic construct
  misc_feature 1..20: note = Synthetic
  source 1..20: mol_type = other DNA
tttgagaaggatcgctttcc

SEQ ID NO: 176 - DNA, 20 bp, synthetic construct
  misc_feature 1..20: note = Synthetic
  source 1..20: mol_type = other DNA
ctcagcgaggaagaataccg

SEQ ID NO: 177 - DNA, 20 bp, synthetic construct
  misc_feature 1..20: note = Synthetic
  source 1..20: mol_type = other DNA
tgcccttgttcttccgtgaa

SEQ ID NO: 178 - DNA, 20 bp, synthetic construct
  misc_feature 1..20: note = Synthetic
  source 1..20: mol_type = other DNA
tcctggatgattagttcaga

SEQ ID NO: 179 - DNA, 20 bp, synthetic construct
  misc_feature 1..20: note = Synthetic
  source 1..20: mol_type = other DNA
gtctaggcaaacctggatta

SEQ ID NO: 180 - DNA, 20 bp, synthetic construct
  misc_feature 1..20: note = Synthetic
  source 1..20: mol_type = other DNA
acgctgtctaggcaaacctg

SEQ ID NO: 181 - DNA, 20 bp, synthetic construct
  misc_feature 1..20: note = Synthetic
  source 1..20: mol_type = other DNA
atctcctggatgattagttc

SEQ ID NO: 182 - DNA, 20 bp, synthetic construct
  misc_feature 1..20: note = Synthetic
  source 1..20: mol_type = other DNA
ctgtgcccttgttcttccgt

SEQ ID NO: 183 - DNA, 20 bp, synthetic construct
  misc_feature 1..20: note = Synthetic
  source 1..20: mol_type = other DNA
ctggatgattagttcagaga

SEQ ID NO: 184 - DNA, 20 bp, synthetic construct
  misc_feature 1..20: note = Synthetic
  source 1..20: mol_type = other DNA
cccttgttcttccgtgaaat

SEQ ID NO: 185 - DNA, 20 bp, synthetic construct
  misc_feature 1..20: note = Synthetic
  source 1..20: mol_type = other DNA
ggcaaacctggattagagtt

SEQ ID NO: 186 - DNA, 20 bp, synthetic construct
  misc_feature 1..20: note = Synthetic
  source 1..20: mol_type = other DNA
taggcaaacctggattagag

SEQ ID NO: 187 - DNA, 20 bp, synthetic construct
  misc_feature 1..20: note = Synthetic
  source 1..20: mol_type = other DNA
aggcaaacctggattagagt

SEQ ID NO: 188 - DNA, 20 bp, synthetic construct
  misc_feature 1..20: note = Synthetic
  source 1..20: mol_type = other DNA
acctggattagagttacatc

SEQ ID NO: 189 - DNA, 20 bp, synthetic construct
  misc_feature 1..20: note = Synthetic
  source 1..20: mol_type = other DNA
gatgattagttcagagatat

SEQ ID NO: 190 - DNA, 20 bp, synthetic construct
  misc_feature 1..20: note = Synthetic
  source 1..20: mol_type = other DNA
ttagagttacatctcctgga

SEQ ID NO: 191 - DNA, 20 bp, synthetic construct
  misc_feature 1..20: note = Synthetic
  source 1..20: mol_type = other DNA
caaacctggattagagttac

SEQ ID NO: 192 - DNA, 20 bp, synthetic construct
  misc_feature 1..20: note = Synthetic
  source 1..20: mol_type = other DNA
ccttgttcttccgtgaaatt

SEQ ID NO: 193 - DNA, 20 bp, synthetic construct
  misc_feature 1..20: note = Synthetic
  source 1..20: mol_type = other DNA
tacatctcctggatgattag

SEQ ID NO: 194 - DNA, 20 bp, synthetic construct
  misc_feature 1..20: note = Synthetic
  source 1..20: mol_type = other DNA
taccagcagacctgcgcgca

SEQ ID NO: 195 - DNA, 20 bp, synthetic construct
  misc_feature 1..20: note = Synthetic
  source 1..20: mol_type = other DNA
agagttacatctcctggatg

SEQ ID NO: 196 - DNA, 20 bp, synthetic construct
  misc_feature 1..20: note = Synthetic
  source 1..20: mol_type = other DNA
cctggattagagttacatct

SEQ ID NO: 197 - DNA, 20 bp, synthetic construct
  misc_feature 1..20: note = Synthetic
  source 1..20: mol_type = other DNA
catctcctggatgattagtt

SEQ ID NO: 198 - DNA, 20 bp, synthetic construct
  misc_feature 1..20: note = Synthetic
  source 1..20: mol_type = other DNA
ggattagagttacatctcct

SEQ ID NO: 199 - DNA, 20 bp, synthetic construct
  misc_feature 1..20: note = Synthetic
  source 1..20: mol_type = other DNA
tgtgcccttgttcttccgtg

SEQ ID NO: 200 - DNA, 20 bp, synthetic construct
  misc_feature 1..20: note = Synthetic
  source 1..20: mol_type = other DNA
ttcttccgtgaaattctggc

SEQ ID NO: 201 - DNA, 20 bp, synthetic construct
  misc_feature 1..20: note = Synthetic
  source 1..20: mol_type = other DNA
gctgtctaggcaaacctgga

SEQ ID NO: 202 - DNA, 20 bp, synthetic construct
  misc_feature 1..20: note = Synthetic
  source 1..20: mol_type = other DNA
acatctcctggatgattagt

SEQ ID NO: 203 - DNA, 20 bp, synthetic construct
  misc_feature 1..20: note = Synthetic
  source 1..20: mol_type = other DNA
gattagagttacatctcctg

SEQ ID NO: 204 - DNA, 20 bp, synthetic construct
  misc_feature 1..20: note = Synthetic
  source 1..20: mol_type = other DNA
attctggctgaatgtctccc

SEQ ID NO: 205 - RNA, 21 nt, synthetic construct
  misc_feature 1..21: note = Synthetic
  source 1..21: mol_type = other RNA
gatttggtttcagaatgagaa

SEQ ID NO: 206 - RNA, 21 nt, synthetic construct
  misc_feature 1..21: note = Synthetic
  source 1..21: mol_type = other RNA
tttgagaaggatcgctttcca

SEQ ID NO: 207 - RNA, 21 nt, synthetic construct
  misc_feature 1..21: note = Synthetic
  source 1..21: mol_type = other RNA
ctcagcgaggaagaataccga

SEQ ID NO: 208 - RNA, 21 nt, synthetic construct
  misc_feature 1..21: note = Synthetic
  source 1..21: mol_type = other RNA
tgcccttgttcttccgtgaaa

SEQ ID NO: 209 - RNA, 21 nt, synthetic construct
  misc_feature 1..21: note = Synthetic
  source 1..21: mol_type = other RNA
tcctggatgattagttcagaa

SEQ ID NO: 210 - RNA, 21 nt, synthetic construct
  misc_feature 1..21: note = Synthetic
  source 1..21: mol_type = other RNA
gtctaggcaaacctggattaa

SEQ ID NO: 211 - RNA, 21 nt, synthetic construct
  misc_feature 1..21: note = Synthetic
  source 1..21: mol_type = other RNA
acgctgtctaggcaaacctga

SEQ ID NO: 212 - RNA, 21 nt, synthetic construct
  misc_feature 1..21: note = Synthetic
  source 1..21: mol_type = other RNA
atctcctggatgattagttca

SEQ ID NO: 213 - RNA, 21 nt, synthetic construct
  misc_feature 1..21: note = Synthetic
  source 1..21: mol_type = other RNA
ctgtgcccttgttcttccgta

SEQ ID NO: 214 - RNA, 21 nt, synthetic construct
  misc_feature 1..21: note = Synthetic
  source 1..21: mol_type = other RNA
ctggatgattagttcagagaa

SEQ ID NO: 215 - RNA, 21 nt, synthetic construct
  misc_feature 1..21: note = Synthetic
  source 1..21: mol_type = other RNA
cccttgttcttccgtgaaata

SEQ ID NO: 216 - RNA, 21 nt, synthetic construct
  misc_feature 1..21: note = Synthetic
  source 1..21: mol_type = other RNA
ggcaaacctggattagagtta

SEQ ID NO: 217 - RNA, 21 nt, synthetic construct
  misc_feature 1..21: note = Synthetic
  source 1..21: mol_type = other RNA
taggcaaacctggattagaga

SEQ ID NO: 218 - RNA, 21 nt, synthetic construct
  misc_feature 1..21: note = Synthetic
  source 1..21: mol_type = other RNA
aggcaaacctggattagagta

SEQ ID NO: 219 - RNA, 21 nt, synthetic construct
  misc_feature 1..21: note = Synthetic
  source 1..21: mol_type = other RNA
acctggattagagttacatca

SEQ ID NO: 220 - RNA, 21 nt, synthetic construct
  misc_feature 1..21: note = Synthetic
  source 1..21: mol_type = other RNA
gatgattagttcagagatata

SEQ ID NO: 221 - RNA, 21 nt, synthetic construct
  misc_feature 1..21: note = Synthetic
  source 1..21: mol_type = other RNA
ttagagttacatctcctggaa

SEQ ID NO: 222 - RNA, 21 nt, synthetic construct
  misc_feature 1..21: note = Synthetic
  source 1..21: mol_type = other RNA
caaacctggattagagttaca

SEQ ID NO: 223 - RNA, 21 nt, synthetic construct
  misc_feature 1..21: note = Synthetic
  source 1..21: mol_type = other RNA
ccttgttcttccgtgaaatta

SEQ ID NO: 224 - RNA, 21 nt, synthetic construct
  misc_feature 1..21: note = Synthetic
  source 1..21: mol_type = other RNA
tacatctcctggatgattaga

SEQ ID NO: 225 - RNA, 21 nt, synthetic construct
  misc_feature 1..21: note = Synthetic
  source 1..21: mol_type = other RNA
taccagcagacctgcgcgcaa

SEQ ID NO: 226 - RNA, 21 nt, synthetic construct
  misc_feature 1..21: note = Synthetic
  source 1..21: mol_type = other RNA
agagttacatctcctggatga

SEQ ID NO: 227 - RNA, 21 nt, synthetic construct
  misc_feature 1..21: note = Synthetic
  source 1..21: mol_type = other RNA
cctggattagagttacatcta

SEQ ID NO: 228 - RNA, 21 nt, synthetic construct
  misc_feature 1..21: note = Synthetic
  source 1..21: mol_type = other RNA
catctcctggatgattagtta

SEQ ID NO: 229 - RNA, 21 nt, synthetic construct
  misc_feature 1..21: note = Synthetic
  source 1..21: mol_type = other RNA
ggattagagttacatctccta

SEQ ID NO: 230 - RNA, 21 nt, synthetic construct
  misc_feature 1..21: note = Synthetic
  source 1..21: mol_type = other RNA
tgtgcccttgttcttccgtga

SEQ ID NO: 231 - RNA, 21 nt, synthetic construct
  misc_feature 1..21: note = Synthetic
  source 1..21: mol_type = other RNA
ttcttccgtgaaattctggca

SEQ ID NO: 232 - RNA, 21 nt, synthetic construct
  misc_feature 1..21: note = Synthetic
  source 1..21: mol_type = other RNA
gctgtctaggcaaacctggaa

SEQ ID NO: 233 - RNA, 21 nt, synthetic construct
  misc_feature 1..21: note = Synthetic
  source 1..21: mol_type = other RNA
acatctcctggatgattagta

SEQ ID NO: 234 - RNA, 21 nt, synthetic construct
  misc_feature 1..21: note = Synthetic
  source 1..21: mol_type = other RNA
gattagagttacatctcctga

SEQ ID NO: 235 - RNA, 21 nt, synthetic construct
  misc_feature 1..21: note = Synthetic
  source 1..21: mol_type = other RNA
attctggctgaatgtctccca

SEQ ID NO: 236 - RNA, 23 nt, synthetic construct
  misc_feature 1..23: note = Synthetic
  source 1..23: mol_type = other RNA
ttctcattctgaaaccaaatctg

SEQ ID NO: 237 - RNA, 23 nt, synthetic construct
  misc_feature 1..23: note = Synthetic
  source 1..23: mol_type = other RNA
tggaaagcgatccttctcaaagg

SEQ ID NO: 238 - RNA, 23 nt, synthetic construct
  misc_feature 1..23: note = Synthetic
  source 1..23: mol_type = other RNA
tcggtattcttcctcgctgaggg

SEQ ID NO: 239 - RNA, 23 nt, synthetic construct
  misc_feature 1..23: note = Synthetic
  source 1..23: mol_type = other RNA
tttcacggaagaacaagggcaca

SEQ ID NO: 240 - RNA, 23 nt, synthetic construct
  misc_feature 1..23: note = Synthetic
  source 1..23: mol_type = other RNA
ttctgaactaatcatccaggaga

SEQ ID NO: 241 - RNA, 23 nt, synthetic construct
  misc_feature 1..23: note = Synthetic
  source 1..23: mol_type = other RNA
ttaatccaggtttgcctagacag

SEQ ID NO: 242 - RNA, 23 nt, synthetic construct
  misc_feature 1..23: note = Synthetic
  source 1..23: mol_type = other RNA
tcaggtttgcctagacagcgtcg

SEQ ID NO: 243 - RNA, 23 nt, synthetic construct
  misc_feature 1..23: note = Synthetic
  source 1..23: mol_type = other RNA
tgaactaatcatccaggagatgt

SEQ ID NO: 244 - RNA, 23 nt, synthetic construct
  misc_feature 1..23: note = Synthetic
  source 1..23: mol_type = other RNA
tacggaagaacaagggcacagag

SEQ ID NO: 245 - RNA, 23 nt, synthetic construct
  misc_feature 1..23: note = Synthetic
  source 1..23: mol_type = other RNA
ttctctgaactaatcatccagga

SEQ ID NO: 246 - RNA, 23 nt, synthetic construct
  misc_feature 1..23: note = Synthetic
  source 1..23: mol_type = other RNA
tatttcacggaagaacaagggca

SEQ ID NO: 247 - RNA, 23 nt, synthetic construct
  misc_feature 1..23: note = Synthetic
  source 1..23: mol_type = other RNA
taactctaatccaggtttgccta

SEQ ID NO: 248 - RNA, 23 nt, synthetic construct
  misc_feature 1..23: note = Synthetic
  source 1..23: mol_type = other RNA
tctctaatccaggtttgcctaga

SEQ ID NO: 249 - RNA, 23 nt, synthetic construct
  misc_feature 1..23: note = Synthetic
  source 1..23: mol_type = other RNA
tactctaatccaggtttgcctag

SEQ ID NO: 250 - RNA, 23 nt, synthetic construct
  misc_feature 1..23: note = Synthetic
  source 1..23: mol_type = other RNA
tgatgtaactctaatccaggttt

SEQ ID NO: 251 - RNA, 23 nt, synthetic construct
  misc_feature 1..23: note = Synthetic
  source 1..23: mol_type = other RNA
tatatctctgaactaatcatcca

SEQ ID NO: 252 - RNA, 23 nt, synthetic construct
  misc_feature 1..23: note = Synthetic
  source 1..23: mol_type = other RNA
ttccaggagatgtaactctaatc

SEQ ID NO: 253 - RNA, 23 nt, synthetic construct
  misc_feature 1..23: note = Synthetic
  source 1..23: mol_type = other RNA
tgtaactctaatccaggtttgcc

SEQ ID NO: 254 - RNA, 23 nt, synthetic construct
  misc_feature 1..23: note = Synthetic
  source 1..23: mol_type = other RNA
taatttcacggaagaacaagggc

SEQ ID NO: 255 - RNA, 23 nt, synthetic construct
  misc_feature 1..23: note = Synthetic
  source 1..23: mol_type = other RNA
tctaatcatccaggagatgtaac

SEQ ID NO: 256 - RNA, 23 nt, synthetic construct
  misc_feature 1..23: note = Synthetic
  source 1..23: mol_type = other RNA
ttgcgcgcaggtctgctggtacc

SEQ ID NO: 257 - RNA, 23 nt, synthetic construct
  misc_feature 1..23: note = Synthetic
  source 1..23: mol_type = other RNA
tcatccaggagatgtaactctaa

SEQ ID NO: 258 - RNA, 23 nt, synthetic construct
  misc_feature 1..23: note = Synthetic
  source 1..23: mol_type = other RNA
tagatgtaactctaatccaggtt

SEQ ID NO: 259 - RNA, 23 nt, synthetic construct
  misc_feature 1..23: note = Synthetic
  source 1..23: mol_type = other RNA
taactaatcatccaggagatgta

SEQ ID NO: 260 - RNA, 23 nt, synthetic construct
  misc_feature 1..23: note = Synthetic
  source 1..23: mol_type = other RNA
taggagatgtaactctaatccag

SEQ ID NO: 261 - RNA, 23 nt, synthetic construct
  misc_feature 1..23: note = Synthetic
  source 1..23: mol_type = other RNA
tcacggaagaacaagggcacaga

SEQ ID NO: 262 - RNA, 23 nt, synthetic construct
  misc_feature 1..23: note = Synthetic
  source 1..23: mol_type = other RNA
tgccagaatttcacggaagaaca

SEQ ID NO: 263 - RNA, 23 nt, synthetic construct
  misc_feature 1..23: note = Synthetic
  source 1..23: mol_type = other RNA
ttccaggtttgcctagacagcgt

SEQ ID NO: 264 - RNA, 23 nt, synthetic construct
  misc_feature 1..23: note = Synthetic
  source 1..23: mol_type = other RNA
tactaatcatccaggagatgtaa

SEQ ID NO: 265 - RNA, 23 nt, synthetic construct
  misc_feature 1..23: note = Synthetic
  source 1..23: mol_type = other RNA
tcaggagatgtaactctaatcca

SEQ ID NO: 266 - RNA, 23 nt, synthetic construct
  misc_feature 1..23: note = Synthetic
  source 1..23: mol_type = other RNA
tgggagacattcagccagaattt